(19) United States
(12) Patent Application Publication
(10) Pub. No.: US 2007/0274992 A1
(43) Pub. Date: Nov. 29, 2007

(54) Secreted Protein Family

(76) Inventors: David Michalovich, London (GB); Iain McKendrick, London (GB); Richard Joseph Fagan, London (GB); Christine Power, Thoiry (FR); Melanie Yorke, Confignon (CH)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/554,816

(22) PCT Filed: Apr. 30, 2004
(86) PCT No.: PCT/GB04/01890

§ 371 Date: Jun. 26, 2007

(30) Foreign Application Priority Data
Apr. 30, 2003 (GB) 0309916.5

(51) Int. Cl. G06F 19/00; C07H 21/04

(52) U.S. Cl. 424/139.1; 424/185.1; 424/93.2; 435/243; 435/29; 435/320.1; 435/6; 435/7.1; 436/86; 436/94; 514/12; 514/2; 514/44; 530/300; 530/324; 530/326; 530/350; 530/387.9; 536/23.1; 702/19; 800/13; 800/3

(57) ABSTRACT

This invention relates to a new family of secreted proteins, termed the SECFAM3 family, its family members including the novel human proteins INSP123, INSP124, and INSP125, herein identified as secreted proteins containing a von Willebrand Factor type C (vWFC) domain, and to the use to these proteins and nucleic acid sequences from the encoding genes in the diagnosis, prevention, and treatment of disease.

This invention relates to a new family of proteins, termed the SECFAM3 family, its family members including the novel proteins INSP123, INSP124 and INSP125, herein identified as secreted proteins containing a von Willebrand Factor type C (vWFC) domain, ranging from 50 to 60 amino acids in length and containing ten conserved cysteine residues and to the use of these proteins and nucleic acid sequences from the encoding genes in the diagnosis, prevention and treatment of disease.

AU publications, patents and patent applications cited herein are incorporated in full by reference.

### BACKGROUND

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

### INTRODUCTION

Secreted Proteins

The ability of cells to make and secrete extracellular proteins is central to many biological processes. Enzymes, growth factors, extracellular matrix proteins and signalling molecules are all secreted by cells. This is through fusion of a secretory vesicle with the plasma membrane. In most cases, but not all, proteins are directed to the endoplasmic reticulum and into secretory vesicles by a signal peptide. Signal peptides are cis-acting sequences that affect the transport of polypeptide chains from the cytoplasm to a membrane bound compartment such as a secretory vesicle.

Polypeptides that are targeted to the secretory vesicles are either secreted into the extracellular matrix or are retained in the plasma membrane. The polypeptides that are retained in the plasma membrane will have one or more transmembrane domains. Examples of secreted proteins that play a central role in the functioning of a cell are cytokines, hormones, extracellular matrix proteins (adhesion molecules), proteases, and growth and differentiation factors.

Von Willebrand Type C Domain Containing Proteins

The von Willebrand Factor type C (vWFC) domain is characterised by a conserved spatial pattern of 10 cysteines within a region of about 56 amino acids in length. These domains are a common feature in large, extracellular, multi-domain proteins, including Chordin, Thombospondin, Type IIA procollagen and Ventroptin. They are also found in smaller proteins associated with the regulation of development, such as SOG (Short Gastrulation). vWFC domains were first characterised in the von Willebrand Factor protein. This protein was seen to be important in blood clotting at the site of vessel damage by participating in platelet-vessel endothelial cell interactions through the formation of a non-covalent complex with coagulation factor VIII at the site of the wound. In this case, vWFC domains were thought to be involved in protein oligomerization events. The fact that this domain is also found in other complex forming proteins points towards a role in protein-protein interaction during the formation of complexes.

The role of vWFC domains in the developmental process has also been highlighted. Sandell L J et al., (2002) J Musculoskel. Interact 2(6):521-523 noted that the proteins chordin and type IIA collagen both bind antagonistically to Bone Morphogenic Proteins (BMPs) via their vWFC domains. This antagonistic binding may play a regulatory role in the development of cartilage and bone during skeletal development. It has also been suggested that BMPs may have a role to play in conditions of excessive cartilage and bone growth, such as osteoarthritis. Thus, it may be possible that therapeutic proteins containing vWFC domains may help to control the progression of such conditions.

Increasing knowledge of these domains is therefore of extreme importance in increasing the understanding of the underlying pathways that lead to the disease states and associated disease states mentioned above, and in developing more effective gene and/or drug therapies to treat these disorders.

Detailed herein is the identification of an entirely novel family of secreted protein ligands. The definition of a secreted protein ligand is a protein that is secreted from a particular cell and elicits a phenotypic response in the same/or another cell by modulating (including ligand-antagonism, as demonstrated by the Dan family) the activity of a cognate receptor and downstream signal transduction pathway. An example of an already known secreted protein ligand family is the glycoprotein hormone family.

Follicle-stimulating hormone (FSH) is a member of the glycoprotein hormone family. In males, FSH is secreted by the cells of the anterior lobe of the pituitary gland, enters the bloodstream, and then binds cognate receptors on the Sertoli cells of the testes to regulate the process of spermatogenesis. In females FSH binds receptors on the thecal, stromal and granulosa cells of the ovary to regulate ovulation. FSH deficiencies can lead to infertility problems in both men and women. Restoring the levels of FSH by administering FSH in the form of a protein therapeutic can be used to combat FSH-triggered infertility. FSH is available as GONAL-f™ (Serono).

By analogy to this example, it can be seen that the identification of a novel secreted ligand protein family paves the way to the delineation of novel ligand-receptor pathways, and critically, to elucidation of the phenotypic consequences of ligand binding. If human disorders are identified which are a consequence of dysfunction of any member of the novel secreted ligand protein family, then that member can be administered as a protein therapeutic to combat the disorder.

### THE INVENTION

The invention is based on the discovery that the INSP123, INSP124 and INSP125 polypeptides are secreted proteins, more specifically, vWFC-domain containing secreted proteins. Together, INSP123, INSP124 and INSP125 form part of a family of proteins herein identified as the SECFAM3 family of proteins. INSP123, INSP124 and INSP125 are predicted to be splice variants with variant functions, such as different affinities for their binding partners.

Annotation of the SECFAM3 Family of Proteins

The proteins of the present invention have no associated publicly available annotation, contain a strong secretory protein signature in the form of a signal peptide, and can be clustered with similar proteins, supported by orthologues from other animal species. Further examination has permitted the construction of a hitherto uncharacterised family of proteins comprising 2 human genes and which, including vertebrate and chordate orthologues, presently comprises 22 sequences in total. This cluster of related sequences will herein be referred to as the “SECFAM3 family.”

These 22 sequences all display a strong signal peptide region of a variable composition with the remainder of the sequences displaying a high degree of similarity.

In one embodiment of the first aspect of the invention, there is provided a method of identifying a member of the SECFAM3 family comprising:

searching a database of translated nucleic acid sequences or polypeptide sequences to identify a polypeptide sequence that matches the following sequence profile:

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | M | −2 | −2 | −3 | −4 | −2 | −1 | −3 | −4 | −3 | 0 | 2 | −2 | 8 | 0 | −3 | −2 | −2 | −2 | −2 | 0 |
| 2 | A | 3 | −1 | −3 | −3 | −1 | −1 | −2 | −2 | −3 | 0 | 0 | 1 | 2 | 1 | −2 | −1 | −1 | −3 | −1 | 1 |
| 3 | L | 1 | −2 | −2 | −3 | −2 | −2 | −2 | −2 | 2 | 0 | 2 | −2 | 0 | −2 | 0 | 1 | −1 | −3 | −2 | 2 |
| 4 | H | −1 | −1 | 1 | −1 | −3 | −1 | −1 | 3 | 6 | −3 | −1 | −1 | −2 | −2 | −2 | 2 | −1 | −3 | −1 | −3 |
| 5 | I | 0 | −2 | −3 | −3 | −2 | 0 | 0 | −3 | −3 | 3 | 2 | −2 | 0 | −1 | −3 | −2 | −1 | −3 | −2 | 2 |
| 6 | H | 0 | −2 | −1 | −2 | −2 | −1 | −1 | −3 | 7 | 0 | 0 | −2 | −1 | −2 | 0 | 0 | −1 | −3 | 0 | 2 |
| 7 | E | 0 | −2 | −2 | −1 | −2 | −1 | 2 | −3 | −1 | 0 | 1 | −1 | 2 | 2 | −3 | 0 | −1 | −2 | 2 | 0 |
| 8 | A | 2 | −2 | −1 | −2 | 3 | −2 | −2 | 2 | −2 | −2 | −2 | −2 | −2 | −3 | −2 | 3 | 1 | −3 | −3 | −2 |
| 9 | C | 0 | −3 | −2 | −3 | 7 | −2 | −3 | −3 | −3 | 0 | −1 | −2 | 3 | 3 | −3 | 0 | −1 | −2 | −1 | 0 |
| 10 | I | −1 | −2 | −2 | −2 | −2 | −2 | 0 | 0 | −2 | 3 | 2 | −2 | 0 | 0 | −3 | 0 | 0 | −2 | 3 | 0 |
| 11 | L | −2 | 0 | −3 | −4 | −2 | −2 | −3 | −4 | −3 | 0 | 4 | −2 | 3 | 3 | −4 | −2 | −2 | −2 | 0 | 0 |
| 12 | L | 0 | 0 | −3 | −4 | −2 | −2 | −3 | −3 | −3 | 0 | 3 | −2 | 0 | −1 | 1 | −2 | −1 | −3 | −2 | 1 |
| 13 | L | −1 | −2 | −2 | −2 | −2 | −2 | 0 | −3 | −3 | 0 | 3 | −2 | 0 | 1 | −3 | 1 | 1 | −3 | −1 | 1 |
| 14 | V | 0 | 0 | −3 | −3 | 4 | 0 | −2 | −3 | −3 | 0 | 0 | −2 | 2 | −2 | −3 | −2 | −1 | −3 | −2 | 4 |
| 15 | I | −1 | 0 | −2 | −3 | 4 | −2 | −2 | −3 | 2 | 2 | 1 | −2 | 0 | −1 | −3 | 0 | 0 | 4 | −1 | 0 |
| 16 | P | 0 | 0 | −2 | −2 | −2 | −2 | −2 | −3 | 4 | −1 | 0 | −2 | −1 | 2 | 3 | 0 | −1 | −2 | 0 | 0 |
| 17 | G | 0 | −3 | −2 | −3 | −2 | −2 | −3 | 2 | −3 | 1 | 0 | −3 | 3 | 3 | −3 | −1 | −2 | −2 | 0 | 1 |
| 18 | L | 1 | −3 | −3 | −4 | −1 | −2 | −3 | −3 | −3 | 0 | 4 | −2 | 0 | −1 | −3 | −2 | −1 | −3 | −2 | 2 |
| 19 | V | 2 | −1 | −1 | −2 | −2 | 3 | −1 | −2 | −2 | 0 | −1 | −1 | −1 | −3 | −2 | 1 | 1 | −3 | −2 | 2 |
| 20 | T | −1 | −2 | −2 | −3 | 4 | −2 | −2 | −3 | −3 | 0 | 3 | −2 | 0 | −2 | −3 | 1 | 3 | −3 | −2 | 0 |
| 21 | S | 0 | −2 | −1 | −2 | 4 | −2 | −2 | 1 | −3 | −1 | 1 | −2 | 2 | −2 | 0 | 2 | −1 | −3 | −3 | −1 |
| 22 | A | 3 | −2 | −2 | −2 | −1 | −2 | −2 | −1 | −2 | −1 | 0 | −2 | −1 | 1 | 2 | 2 | −1 | −3 | −2 | 0 |
| 23 | A | 2 | −2 | −3 | −2 | 5 | 0 | 0 | −2 | −3 | −1 | 0 | −2 | −1 | −3 | 2 | −1 | −1 | −3 | −2 | 1 |
| 24 | I | 1 | 1 | −2 | −2 | −2 | −1 | −2 | −2 | −2 | 2 | 0 | −1 | −1 | −1 | −2 | 2 | −1 | −3 | 1 | 1 |
| 25 | S | −1 | −1 | 2 | −1 | −2 | 1 | −1 | −2 | 4 | 2 | −1 | −1 | −1 | −2 | −2 | 3 | −1 | −4 | −1 | −1 |
| 26 | H | 0 | −2 | 3 | −1 | −3 | −1 | −1 | −2 | 5 | −2 | −2 | −1 | −2 | 0 | 3 | 0 | −1 | −3 | −1 | 0 |
| 27 | E | −1 | −1 | 0 | 1 | −3 | 0 | 5 | −2 | −1 | −3 | −3 | 0 | −2 | −3 | −1 | 1 | 2 | −3 | −2 | −2 |
| 28 | D | 1 | −2 | 0 | 4 | −2 | −1 | 0 | −1 | −2 | −2 | 0 | −1 | −1 | −2 | −1 | 2 | 0 | −4 | −3 | −2 |
| 29 | Y | −2 | −1 | −2 | −2 | −3 | 2 | −1 | −3 | 0 | 0 | −1 | −1 | −1 | 0 | 3 | −1 | −2 | 0 | 5 | 0 |
| 30 | P | 0 | −1 | −1 | −1 | −2 | −1 | −1 | 0 | 5 | −3 | −3 | −1 | −2 | −3 | 4 | 0 | 0 | −3 | −1 | −2 |
| 31 | A | 3 | −2 | −2 | −2 | −1 | −1 | −2 | −1 | −3 | 0 | 2 | −1 | 0 | −2 | 0 | 0 | 0 | −3 | −2 | 0 |
| 32 | D | −2 | −1 | 0 | 5 | −3 | 0 | 2 | −2 | −1 | −2 | −3 | 2 | −2 | −3 | −1 | 0 | −1 | −4 | −3 | 0 |
| 33 | E | 0 | −1 | −2 | 0 | −2 | 0 | 3 | −3 | −1 | 0 | 0 | −1 | 0 | 3 | −2 | −1 | −1 | −2 | 0 | 0 |
| 34 | G | 1 | −2 | 0 | −1 | −2 | −1 | −2 | 4 | −2 | −2 | −2 | −2 | 0 | −2 | −2 | 1 | 1 | −2 | −3 | 0 |
| 35 | D | 0 | 2 | 0 | 2 | −2 | 0 | 0 | −2 | −1 | −2 | 0 | 0 | −1 | −3 | 2 | −1 | −1 | −3 | −2 | −2 |
| 36 | Q | 0 | 3 | 0 | −1 | −2 | 3 | 0 | −2 | −1 | −2 | −2 | 0 | −1 | −3 | −1 | 1 | 3 | −3 | −2 | −2 |
| 37 | I | 0 | −2 | −3 | −2 | −1 | −2 | −2 | −2 | −3 | 1 | 0 | −2 | 0 | −2 | 4 | −1 | −1 | −3 | −2 | 2 |
| 38 | S | −1 | −1 | 0 | 1 | −2 | 0 | 2 | −2 | −1 | 2 | −1 | 1 | −1 | −2 | −2 | 2 | −1 | −3 | −2 | 0 |
| 39 | S | 3 | −1 | −1 | −1 | −1 | −1 | −1 | −1 | −2 | −1 | 0 | −1 | −1 | −2 | 2 | 3 | 0 | −3 | −2 | −1 |
| 40 | N | 0 | −2 | 2 | −1 | −3 | −1 | 1 | 3 | −1 | 0 | 0 | −1 | −1 | −3 | 1 | −1 | −1 | −3 | −2 | 0 |
| 41 | D | 1 | −2 | −1 | 3 | −2 | 0 | 2 | −2 | −2 | −1 | 0 | −1 | −1 | 0 | −2 | 0 | −1 | −3 | −2 | −1 |
| 42 | N | −1 | −1 | 2 | 1 | −3 | 0 | 3 | 0 | −1 | −2 | 0 | 0 | −1 | −2 | −2 | 1 | −1 | −3 | −2 | −2 |
| 43 | L | 1 | −3 | −3 | −3 | 4 | −2 | −3 | −3 | −1 | 0 | 1 | −3 | 0 | 3 | −3 | −2 | −2 | 0 | 5 | 0 |
| 44 | I | 1 | −1 | −1 | 0 | −2 | 0 | 2 | −2 | −2 | 1 | −1 | 1 | −1 | −2 | −2 | 2 | −1 | −4 | −2 | 0 |
| 45 | F | −2 | −3 | −2 | 1 | −2 | −2 | −2 | −3 | −1 | 2 | 0 | −3 | 0 | 4 | −3 | −2 | −2 | −1 | 4 | 1 |
| 46 | D | 1 | −2 | −1 | 2 | −2 | −1 | 0 | 0 | −2 | −3 | −3 | −1 | −2 | −3 | 4 | 1 | −1 | −4 | −3 | −2 |
| 47 | D | 0 | −2 | 0 | 5 | −3 | −1 | 0 | 1 | −2 | −3 | −4 | −1 | −3 | −3 | 2 | 1 | −1 | −4 | −3 | −3 |
| 48 | Y | −2 | 3 | −1 | −2 | −3 | 2 | −1 | −3 | 0 | −2 | −2 | 0 | −1 | 0 | −3 | −2 | −2 | 0 | 6 | −2 |
| 49 | R | −2 | 4 | 0 | −2 | −4 | 2 | 0 | −2 | 5 | −4 | −3 | 0 | −2 | −3 | 2 | −1 | −2 | −3 | −1 | −3 |
| 50 | G | −1 | −2 | 0 | 2 | −3 | −1 | 1 | 4 | −2 | −2 | −3 | −1 | −2 | −3 | −2 | 1 | −1 | −3 | −3 | 0 |
| 51 | K | −1 | 2 | 1 | −1 | −3 | 0 | 0 | 1 | −1 | −3 | −3 | 4 | −2 | −3 | −2 | 1 | −1 | −3 | −3 | −3 |
| 52 | G | −1 | −2 | −1 | 2 | 4 | −2 | −1 | 3 | −2 | −2 | −3 | −2 | −2 | 0 | −2 | 0 | 2 | −2 | −2 | −2 |
| 53 | C | −1 | −4 | −4 | −4 | 10 | −4 | −4 | −4 | −3 | −1 | −1 | −4 | −1 | 2 | −4 | −2 | −2 | −2 | −1 | −1 |
| 54 | V | −1 | −2 | −1 | 0 | −2 | −1 | 1 | −2 | −2 | 0 | 0 | −1 | 3 | −2 | −2 | 1 | 0 | −3 | −2 | 3 |
| 55 | D | 0 | −2 | 0 | 6 | −3 | 0 | 1 | −1 | −1 | −3 | −4 | −1 | −3 | −3 | −1 | 0 | −1 | −4 | −3 | −3 |
| 56 | D | −2 | −1 | 3 | 4 | −4 | 0 | 2 | 2 | −1 | −4 | −4 | −1 | −3 | −4 | −2 | 0 | −1 | −4 | −3 | −3 |
| 57 | S | 0 | −1 | 2 | 2 | −3 | 1 | 0 | 2 | −1 | −3 | −3 | −1 | −2 | −3 | −2 | 3 | 0 | −3 | −3 | −3 |
| 58 | G | 0 | 1 | 0 | −1 | −3 | −1 | −2 | 5 | −2 | −4 | −4 | −1 | −3 | −3 | −2 | 1 | −2 | −3 | −3 | −3 |
| 59 | F | −1 | −4 | −4 | −4 | −2 | −3 | −3 | −4 | −2 | 3 | 0 | −3 | 0 | 5 | −4 | −2 | −1 | −1 | 0 | 3 |
| 60 | V | −1 | −3 | −3 | −2 | −2 | −1 | 1 | −3 | −1 | 0 | 0 | −2 | 0 | 3 | −3 | −2 | −1 | −1 | 4 | 3 |
| 61 | Y | −2 | −2 | −2 | −3 | −2 | −2 | −2 | −3 | 0 | −1 | −1 | −2 | −1 | 4 | −3 | 0 | −2 | 0 | 7 | −2 |
| 62 | K | 1 | −1 | −2 | −2 | −2 | −1 | −1 | 0 | −3 | 0 | −1 | 1 | −1 | −2 | 2 | −1 | −1 | −4 | −2 | 3 |
| 63 | L | −1 | −3 | −2 | −3 | −2 | −3 | −3 | 4 | −3 | 4 | 1 | −3 | 0 | −1 | −3 | −1 | −2 | −3 | −2 | 0 |
| 64 | G | −1 | −1 | 0 | 0 | −4 | 0 | 3 | 5 | −1 | −4 | −4 | −1 | −3 | −4 | −2 | 0 | −2 | −3 | −3 | −3 |
| 65 | E | −2 | −1 | −1 | 0 | −3 | 3 | 3 | −3 | −1 | −2 | −1 | 0 | 3 | −1 | −2 | −1 | −2 | 7 | −1 | −2 |
| 66 | R | −2 | 2 | −2 | −3 | −2 | 0 | −1 | −3 | 0 | 1 | −1 | 1 | −1 | 3 | −3 | −2 | −2 | −1 | 4 | 0 |
| 67 | F | −2 | −3 | −3 | −3 | −2 | −3 | −3 | −3 | −1 | −1 | −1 | −3 | −1 | 6 | −4 | −2 | 1 | 6 | 3 | −1 |
| 68 | F | 0 | 1 | −2 | −3 | −2 | −1 | −2 | −2 | −1 | −1 | 1 | −1 | 0 | 2 | −3 | 0 | 0 | −1 | 2 | −1 |
| 69 | P | −2 | −2 | −1 | 2 | −4 | 0 | 2 | −2 | −2 | −4 | −4 | −1 | −3 | −4 | 6 | −1 | −1 | −4 | −3 | −3 |
| 70 | G | 0 | −2 | 0 | −1 | −2 | −1 | −1 | 5 | −2 | −4 | −4 | −1 | −2 | −3 | −2 | 3 | −1 | −3 | −3 | −3 |
| 71 | H | −2 | −2 | 0 | 5 | −3 | −1 | 0 | −2 | 4 | −3 | −4 | −1 | −3 | −3 | 3 | 0 | −1 | −4 | −2 | −3 |
| 72 | S | −1 | −1 | 0 | −1 | −2 | −1 | −1 | −2 | 5 | −3 | −3 | −1 | −2 | −3 | 4 | 3 | 1 | −3 | −1 | −2 |
| 73 | N | 2 | −2 | 2 | −2 | 8 | −2 | −2 | −2 | −2 | −2 | −2 | −2 | −2 | −3 | −3 | 0 | 0 | −3 | −2 | −1 |
| 74 | C | −1 | −2 | −1 | −1 | 8 | −1 | 3 | −3 | −2 | −2 | −2 | −1 | −2 | −3 | −2 | 0 | 2 | −3 | −2 | −1 |
| 75 | P | −1 | 2 | −2 | −2 | −3 | 3 | 0 | −3 | −2 | −2 | 0 | 0 | −1 | −3 | 5 | −1 | −1 | −3 | −2 | −2 |
| 76 | C | 0 | −4 | −4 | −4 | 10 | −4 | −5 | −4 | −4 | −1 | −1 | −4 | −1 | −2 | −4 | −1 | −1 | −2 | −2 | −1 |
| 77 | V | −1 | −2 | −2 | −1 | −2 | −1 | 2 | −3 | −2 | 0 | 2 | −1 | 0 | −1 | −2 | −1 | 0 | −3 | −2 | 3 |
| 78 | C | 0 | −4 | −4 | −4 | 10 | −4 | −5 | −4 | −4 | −1 | −1 | −4 | −1 | −2 | −4 | −1 | −1 | −2 | −2 | −1 |
| 79 | A | 2 | −1 | −1 | −1 | −1 | 1 | −1 | −2 | −2 | −2 | −2 | −1 | −1 | −3 | −1 | 0 | 4 | −3 | −2 | −1 |
| 80 | L | 1 | −1 | −1 | 0 | −2 | 0 | 2 | −2 | −2 | −1 | 0 | −1 | −1 | −2 | −2 | 1 | 1 | −3 | −2 | −1 |
| 81 | D | −2 | −2 | 0 | 5 | −4 | 0 | 4 | −2 | −1 | −3 | −4 | −1 | −3 | −3 | −2 | 0 | 1 | −4 | −3 | −3 |
| 82 | G | 0 | −3 | 0 | −2 | −4 | −3 | −3 | 7 | −3 | −5 | −5 | −3 | −4 | −4 | −3 | 0 | −3 | −3 | −4 | −4 |
| 83 | P | −1 | −2 | −2 | −1 | −3 | −1 | −1 | −2 | −2 | −3 | −3 | −1 | −2 | −4 | 7 | 1 | −1 | −4 | −3 | −3 |
| 84 | V | 0 | −2 | −2 | −3 | −2 | 1 | −1 | −3 | −3 | 1 | 1 | −2 | 0 | −2 | −2 | 0 | −1 | −3 | −2 | 4 |
| 85 | C | 0 | −3 | −3 | −3 | 10 | −3 | −4 | −3 | −3 | −2 | −2 | −3 | −2 | −3 | −3 | 0 | −1 | −3 | −3 | −2 |
| 86 | D | 1 | −2 | −1 | 3 | 4 | −1 | −1 | −2 | −2 | −1 | −2 | −2 | 0 | 0 | −2 | 1 | 0 | −3 | −2 | 0 |
| 87 | Q | −1 | 3 | −1 | −2 | −3 | 4 | 0 | −3 | −1 | −2 | −2 | 2 | −1 | −3 | −2 | −1 | −1 | −3 | −2 | 0 |
| 88 | P | −1 | −2 | −1 | −2 | −2 | −2 | −2 | −3 | −3 | −2 | −2 | −2 | −2 | −4 | 6 | 0 | 4 | −4 | −3 | −1 |
| 89 | E | −2 | 2 | 0 | 2 | −4 | 0 | 5 | −2 | −1 | −4 | −3 | 1 | −2 | −4 | −2 | −1 | −2 | −4 | −3 | −3 |
| 90 | C | 0 | −4 | −4 | −4 | 10 | −4 | −5 | −4 | −4 | −2 | −2 | −4 | −2 | −3 | −4 | −2 | −2 | −3 | −3 | −2 |
| 91 | P | −1 | −2 | −2 | −2 | −3 | −2 | −2 | −3 | −3 | −1 | −2 | −2 | −2 | −3 | 6 | −1 | 2 | −4 | −3 | 0 |
| 92 | K | 0 | 3 | 0 | 0 | −3 | 0 | 3 | −2 | −1 | −3 | −3 | 3 | −2 | −3 | −2 | 0 | −1 | −3 | −2 | −2 |
| 93 | I | −1 | −3 | −3 | −4 | −1 | −2 | −3 | −4 | −3 | 3 | 3 | −2 | 0 | −1 | −3 | −2 | 1 | −3 | −2 | 1 |
| 94 | H | −1 | −1 | 0 | −1 | 4 | −1 | −1 | −2 | 7 | −3 | −3 | −1 | −2 | −2 | 2 | 1 | −1 | −3 | 0 | −3 |
| 95 | P | 0 | −2 | −2 | −1 | −3 | −1 | 1 | −2 | −2 | −3 | −3 | −1 | −2 | −4 | 7 | −1 | −1 | −4 | −3 | −2 |
| 96 | K | 1 | 3 | −1 | −2 | −2 | 0 | 0 | −2 | 0 | −2 | −2 | 2 | −1 | −1 | −2 | 1 | −1 | −2 | 2 | −2 |
| 97 | C | 0 | −3 | −3 | −3 | 10 | −3 | −4 | −3 | −3 | −2 | −2 | −3 | −2 | −3 | 2 | −1 | −1 | −3 | −3 | −2 |
| 98 | T | −1 | −3 | −2 | −3 | −1 | −2 | −3 | −4 | −3 | 4 | 0 | −3 | 1 | −1 | −3 | −1 | 3 | −3 | −2 | 2 |
| 99 | K | −2 | 1 | 0 | −1 | −4 | 0 | 2 | −2 | 7 | −3 | −3 | 2 | −2 | −2 | −2 | −1 | −2 | −3 | 0 | −3 |
| 100 | V | −1 | −3 | −3 | −3 | −1 | −3 | −3 | −4 | −3 | 3 | 0 | −3 | 0 | −1 | −3 | −2 | −1 | −3 | −1 | 5 |
| 101 | E | 1 | −1 | 0 | 3 | −2 | 0 | 2 | −1 | −1 | −3 | −3 | 1 | −2 | −3 | −1 | 2 | 0 | −4 | −3 | −2 |
| 102 | H | −2 | 2 | 1 | −1 | −3 | 0 | −1 | −2 | 7 | −3 | −2 | 0 | −2 | −1 | −2 | −1 | 1 | −2 | 3 | −2 |
| 103 | N | −1 | 0 | 3 | −1 | −2 | 0 | 0 | −1 | 4 | −2 | −2 | 1 | −2 | 0 | −2 | 1 | 0 | −1 | 4 | −2 |
| 104 | G | −1 | −1 | 0 | 2 | −3 | 4 | 2 | 2 | −1 | −4 | −3 | 0 | −2 | −3 | −2 | 0 | −1 | −3 | −2 | −3 |
| 105 | C | 0 | −3 | −3 | −3 | 10 | −3 | −5 | −3 | −3 | −1 | −1 | −3 | −1 | −2 | −3 | −1 | −1 | −2 | −2 | −1 |
| 106 | C | 0 | −3 | −3 | −3 | 10 | −3 | −5 | −3 | −3 | −1 | −1 | −3 | −1 | −2 | −3 | −1 | −1 | −2 | −2 | −1 |
| 107 | P | −1 | −3 | −3 | −2 | −3 | −2 | −2 | −3 | −3 | −1 | −2 | −2 | −2 | −4 | 7 | −2 | −1 | −4 | −3 | 0 |
| 108 | E | −1 | 2 | 0 | 2 | −3 | 4 | 2 | −2 | −1 | −2 | −1 | 0 | −1 | −3 | −2 | −1 | −1 | −3 | −2 | 0 |
| 109 | C | 0 | −3 | −3 | −3 | 10 | −3 | −5 | −3 | −3 | −1 | −1 | −3 | −1 | −2 | −3 | −1 | −1 | −2 | −2 | −1 |
| 110 | K | −1 | 0 | 0 | 0 | −3 | 0 | 3 | −2 | −1 | −1 | −2 | 3 | −1 | −3 | −2 | 1 | −1 | −3 | −2 | 1 |
| 111 | E | 2 | 2 | −1 | 0 | −3 | 0 | 4 | −2 | −1 | −3 | −2 | 2 | −2 | −3 | −2 | 0 | −1 | −3 | −2 | −2 |
| 112 | V | −1 | 1 | −1 | −1 | −3 | −1 | 1 | 1 | −2 | 1 | −1 | 1 | −1 | −2 | −2 | −1 | −1 | −3 | −2 | 2 |
| 113 | K | 0 | 0 | 0 | −1 | −3 | 0 | 0 | 4 | −2 | −4 | −3 | 4 | −2 | −3 | −2 | 1 | −1 | −3 | −3 | −3 |
| 114 | N | −2 | 0 | 6 | 0 | −3 | 0 | 0 | 0 | 0 | −3 | −3 | 2 | −2 | −3 | −2 | 0 | 0 | −4 | −2 | −3 |
| 115 | F | −1 | −3 | −3 | −3 | −2 | −2 | −2 | −3 | 0 | 0 | 0 | −2 | 0 | 4 | −3 | −2 | −1 | 0 | 6 | 1 |
| 116 | C | 0 | −3 | −3 | −3 | 10 | −3 | −5 | −3 | −3 | −1 | −1 | −3 | −1 | −2 | −3 | −1 | −1 | −2 | −2 | −1 |
| 117 | E | −1 | −1 | 0 | 3 | −3 | 0 | 4 | −2 | −1 | −2 | 0 | 0 | −2 | −3 | −2 | 0 | 1 | −3 | −3 | −2 |
| 118 | Y | −2 | −2 | −2 | −2 | −3 | −1 | 1 | −3 | 0 | −1 | −1 | −2 | −1 | 5 | −3 | −2 | −2 | 0 | 6 | −2 |
| 119 | H | −2 | 5 | 2 | −2 | −3 | 0 | −1 | −2 | 4 | −2 | −1 | 0 | 3 | −2 | −2 | −1 | −1 | −3 | −1 | −2 |
| 120 | G | −1 | −2 | 2 | −1 | −3 | −2 | −2 | 6 | −2 | −4 | −4 | −2 | −3 | −3 | −2 | 0 | −2 | −3 | −3 | −3 |
| 121 | K | −1 | 4 | −1 | −2 | −3 | 0 | 0 | −3 | −1 | −1 | −2 | 5 | −1 | −3 | −2 | −1 | −1 | −3 | −2 | 0 |
| 122 | N | −1 | −2 | 2 | −2 | −2 | −2 | −2 | −2 | −2 | 2 | 0 | −2 | 0 | −2 | −2 | 0 | 4 | −3 | −2 | 1 |
| 123 | Y | −2 | −3 | −3 | −3 | −2 | −2 | −3 | −3 | 0 | −1 | −1 | −3 | −1 | 4 | −4 | −2 | −2 | 1 | 8 | −1 |
| 124 | K | 0 | 1 | 0 | −1 | −3 | 2 | 2 | −2 | 4 | −3 | −2 | 2 | −1 | −1 | −2 | −1 | −2 | −2 | 3 | −2 |
| 125 | I | −1 | −2 | 2 | −2 | −2 | −2 | −2 | −2 | −2 | 2 | 1 | −2 | 0 | −1 | −2 | 0 | 1 | −3 | −2 | 2 |
| 126 | L | −1 | −2 | 2 | −2 | −2 | −2 | −2 | 3 | −2 | 0 | 3 | −2 | 0 | −1 | −3 | −1 | −1 | −3 | −2 | −1 |
| 127 | E | −1 | 0 | 0 | 1 | −4 | 3 | 6 | −2 | 0 | −3 | −3 | 0 | −2 | −3 | −1 | 0 | −1 | −3 | −2 | −2 |
| 128 | E | −1 | −1 | 2 | 0 | −3 | 0 | 4 | −2 | 0 | −3 | −3 | 0 | −2 | −1 | −2 | 0 | 1 | −2 | 3 | −2 |
| 129 | F | −2 | −3 | −3 | −3 | −2 | −3 | −3 | −3 | −1 | 0 | 0 | −3 | 0 | 7 | −5 | −2 | −2 | 0 | 2 | −1 |
| 130 | K | −1 | 1 | 2 | −1 | −3 | 2 | 0 | −2 | −1 | −1 | 0 | 2 | 1 | −2 | −2 | −1 | −1 | −3 | −2 | 0 |
| 131 | P | −1 | −3 | −3 | −3 | −2 | −2 | −2 | −3 | −3 | 1 | 2 | −2 | 0 | −2 | 3 | −2 | 0 | −3 | −2 | 3 |
| 132 | S | 0 | 0 | 1 | 2 | 2 | −1 | 0 | −1 | −1 | −3 | −3 | −1 | −2 | −3 | 1 | 4 | 1 | −4 | −3 | −2 |
| 133 | P | −1 | −2 | −2 | −1 | −3 | −1 | 1 | −3 | −2 | −2 | −3 | −1 | −2 | −4 | 7 | −1 | −1 | −4 | −3 | 0 |
| 134 | C | 0 | −3 | −3 | −3 | 10 | −3 | −4 | −3 | −3 | −1 | −1 | −3 | −1 | −2 | −3 | −1 | 0 | −2 | −2 | −1 |
| 135 | E | −1 | 1 | −1 | 0 | −3 | 0 | 5 | −3 | −1 | −1 | 1 | 0 | −1 | −2 | −2 | −1 | −1 | −3 | −2 | −1 |
| 136 | W | −1 | 2 | −1 | −2 | −3 | 2 | 0 | −2 | 3 | −2 | 0 | 1 | −1 | −2 | −2 | 1 | −1 | 4 | −1 | −2 |
| 137 | C | 0 | 0 | −3 | −3 | 10 | −3 | −4 | −3 | −3 | 0 | −1 | −3 | −1 | −2 | −3 | −1 | 0 | −3 | −2 | −1 |
| 138 | R | −1 | 4 | −1 | −2 | −2 | 0 | −1 | −3 | −1 | 2 | −1 | 2 | −1 | −1 | −2 | −1 | 1 | −2 | 1 | 0 |
| 139 | C | 0 | −4 | −4 | −4 | 10 | −4 | −5 | −4 | −4 | −1 | −1 | −4 | −1 | −2 | −4 | −1 | −1 | −2 | −2 | −1 |
| 140 | E | −1 | −1 | 0 | 2 | −3 | 0 | 5 | −3 | −1 | −2 | 0 | 0 | −1 | −3 | −2 | 0 | 1 | −3 | −2 | −2 |
| 141 | P | 1 | −2 | 1 | −2 | −2 | −1 | −1 | −2 | −2 | 0 | 0 | −1 | −1 | −2 | 2 | 1 | 1 | −3 | −2 | 0 |
| 142 | S | −1 | −1 | 4 | 2 | −3 | 1 | 0 | 2 | −1 | −3 | −3 | −1 | −2 | −3 | −2 | 2 | −1 | −4 | −3 | −3 |
| 143 | N | −1 | 1 | 2 | −1 | −3 | −1 | −1 | 4 | −1 | −4 | −4 | 1 | −2 | −3 | −2 | 1 | −1 | −3 | −3 | −3 |
| 144 | E | −1 | 0 | −1 | 0 | −3 | 0 | 4 | −3 | −1 | 1 | −1 | 0 | −1 | −2 | −2 | −1 | 1 | −3 | −2 | 1 |
| 145 | V | 3 | −2 | −3 | −3 | −1 | −2 | −2 | −2 | −3 | 0 | 0 | −2 | 0 | −2 | −2 | −1 | −1 | −3 | −2 | 3 |
| 146 | H | −2 | 1 | −2 | −3 | −2 | −1 | −2 | −3 | 3 | 0 | 2 | −1 | 0 | 2 | −3 | −2 | −2 | −1 | 4 | −1 |
| 147 | C | −1 | 0 | −3 | −3 | 10 | −3 | −3 | −3 | −3 | −2 | −2 | −2 | −2 | −3 | 1 | −1 | −1 | −3 | −3 | −2 |
| 148 | V | 0 | −2 | −1 | −2 | −1 | −1 | −2 | −2 | −3 | 0 | 1 | −1 | 0 | −2 | −2 | 2 | 3 | −3 | −2 | 2 |
| 149 | V | −1 | −3 | −3 | −3 | 1 | −2 | −2 | −3 | −3 | 2 | 0 | −2 | 0 | −2 | 2 | −2 | −1 | −4 | −2 | 5 |
| 150 | A | 3 | −2 | −2 | −2 | 2 | −1 | −1 | −1 | −2 | 0 | −1 | −1 | −1 | −2 | −2 | 2 | 0 | −3 | −2 | 2 |
| 151 | D | 1 | −2 | 0 | 5 | −2 | 1 | 0 | 0 | −2 | −3 | −3 | −1 | −2 | −3 | −2 | 1 | −1 | −4 | −3 | −2 |
| 152 | C | −1 | −3 | −3 | −3 | 10 | −3 | −4 | 0 | −3 | −2 | −2 | −3 | −1 | 1 | −4 | −1 | −2 | −2 | −1 | −2 |
| 153 | A | 2 | −2 | −2 | −2 | −2 | −1 | 0 | −2 | −2 | −1 | 0 | −1 | −1 | 1 | 4 | −1 | −1 | −3 | −1 | −1 |
| 154 | V | 2 | −1 | −2 | −1 | −2 | 4 | 1 | −2 | −2 | −1 | −2 | 0 | −1 | −3 | 3 | −1 | −1 | −3 | −2 | 0 |
| 155 | P | −1 | −2 | −1 | 1 | −2 | −2 | −1 | −2 | −2 | 1 | 0 | −2 | −1 | −2 | 4 | 0 | 2 | −4 | −3 | 0 |
| 156 | E | −2 | 1 | −1 | 0 | −4 | 0 | 3 | −3 | 2 | −3 | −3 | 1 | −2 | 2 | 3 | −1 | −2 | −3 | −1 | −3 |
| 157 | C | −1 | −3 | −3 | −3 | 9 | 1 | −3 | −3 | −3 | −2 | −1 | −3 | −1 | −2 | −3 | −2 | −2 | 5 | −2 | −2 |
| 158 | V | −1 | 0 | −2 | −3 | −2 | 0 | −2 | −3 | −3 | 2 | 0 | −2 | 0 | 1 | −2 | −2 | 1 | −2 | −1 | 4 |
| 159 | N | −2 | 1 | 4 | 4 | −3 | 0 | 0 | −1 | 0 | −3 | −3 | −1 | −2 | −1 | −2 | 0 | −1 | 3 | 3 | −3 |
| 160 | P | −1 | −2 | −2 | −1 | −3 | −1 | −1 | −2 | −2 | −1 | −3 | 1 | −2 | −4 | 7 | 0 | −1 | −4 | −3 | −2 |
| 161 | V | −1 | −3 | −3 | −3 | −2 | 0 | 0 | −4 | −3 | 4 | 0 | −2 | 0 | 2 | −3 | −2 | −1 | −2 | 0 | 3 |
| 162 | Y | −2 | 1 | −1 | −3 | −3 | −1 | −1 | −3 | 4 | −2 | 0 | −1 | −1 | 0 | −3 | 0 | −2 | 0 | 6 | −2 |
| 163 | E | −1 | −1 | −1 | 0 | −3 | 3 | 4 | −3 | −1 | −2 | −1 | 0 | −1 | −3 | 3 | 0 | 0 | −3 | −2 | −2 |
| 164 | P | −1 | −1 | −2 | −1 | −3 | 0 | 1 | −3 | −1 | −2 | −1 | 0 | −2 | −2 | 5 | −1 | −1 | −2 | 2 | −2 |
| 165 | E | −1 | −2 | 1 | 2 | −3 | −1 | 1 | 3 | −1 | −3 | −3 | −1 | −3 | −1 | −2 | 0 | −1 | −2 | 2 | −3 |
| 166 | Q | −2 | 0 | −1 | −1 | −3 | 4 | 2 | −3 | 3 | −3 | 0 | 2 | −1 | −2 | −2 | −1 | −2 | 5 | 0 | −2 |
| 167 | C | 0 | −3 | −3 | −4 | 9 | −3 | −4 | −4 | −3 | 0 | 0 | −3 | −1 | −2 | −3 | −2 | −1 | −2 | −2 | 0 |
| 168 | C | 0 | −3 | −3 | −3 | 9 | −3 | −3 | −3 | −3 | 0 | −1 | −3 | −1 | −2 | −3 | 0 | −1 | −3 | −2 | 1 |
| 169 | P | 0 | −2 | −3 | −2 | −3 | −1 | −2 | −2 | −3 | −2 | 0 | −1 | −1 | −3 | 7 | −1 | −1 | −4 | −3 | −1 |
| 170 | V | −1 | −2 | −2 | −2 | −2 | −1 | 1 | −3 | −2 | 3 | 0 | 1 | 0 | −2 | −2 | −2 | −1 | −3 | −2 | 4 |
| 171 | C | −1 | −2 | −3 | −3 | 9 | −2 | −3 | −3 | −3 | −1 | 0 | 1 | −1 | −2 | −3 | −1 | −1 | −3 | −2 | −1 |
| 172 | K | −1 | 1 | 0 | −1 | −3 | 0 | 0 | −2 | −1 | −3 | −2 | 6 | −1 | −3 | −1 | 0 | −1 | −3 | −2 | −2 |
| 173 | N | 2 | −1 | 3 | 2 | −2 | −1 | 0 | 2 | −1 | −3 | −3 | −1 | −2 | −3 | −2 | 1 | −1 | −4 | −3 | −2 |
| 174 | G | 0 | −1 | 0 | −1 | −3 | −2 | −2 | 5 | −2 | −4 | −3 | 0 | −3 | −3 | −2 | 0 | −2 | −2 | −3 | −3 |
| 175 | P | −1 | −2 | 1 | −1 | −3 | −1 | 0 | −2 | −2 | −3 | −3 | −1 | −2 | −4 | 6 | −1 | −1 | −4 | −3 | −2 |
| 176 | N | −1 | −1 | 5 | 0 | −3 | −1 | −1 | −1 | 0 | −3 | −3 | −1 | −2 | −2 | −2 | 1 | 0 | 6 | −1 | −3 |
| 177 | C | −1 | −2 | −2 | −2 | 8 | −2 | −2 | −3 | 3 | −2 | −2 | −2 | −2 | −2 | 1 | −1 | 0 | −3 | −1 | −2 |
| 178 | F | −2 | −2 | −1 | −2 | −3 | −2 | −2 | 1 | 4 | −2 | −2 | 0 | −1 | 4 | 0 | −1 | −2 | 0 | 3 | −2 |
| 179 | A | 2 | −2 | −2 | −3 | −1 | −2 | −2 | 2 | −3 | 0 | 1 | −2 | 0 | 0 | −2 | −1 | −1 | −3 | −1 | 1 |
| 180 | G | 0 | −1 | 0 | 0 | −2 | −1 | 1 | 3 | −2 | −2 | 0 | −1 | −1 | −2 | 1 | 1 | 1 | −3 | −2 | −1 |
| 181 | T | 0 | −2 | −1 | −1 | −1 | −1 | −1 | −2 | −2 | 1 | −1 | −1 | −1 | −2 | 1 | 0 | 5 | −3 | −2 | 0 |
| 182 | T | 1 | −1 | 0 | −1 | −1 | 2 | 0 | −1 | −2 | −1 | 0 | −1 | 0 | −2 | −1 | 1 | 3 | −3 | −2 | −1 |
| 183 | I | 1 | −3 | −2 | −3 | −1 | −2 | −2 | −2 | −3 | 3 | 0 | −2 | 0 | −1 | −2 | 0 | 0 | −3 | −1 | 3 |
| 184 | I | −1 | −3 | −3 | −3 | −2 | −3 | −3 | 1 | −3 | 5 | 0 | −3 | 0 | 2 | −3 | −2 | −1 | −2 | 0 | 1 |
| 185 | P | −1 | −2 | −2 | −1 | −3 | 0 | 1 | −2 | −2 | −2 | 0 | −1 | −1 | −3 | 6 | −1 | −1 | −4 | −3 | −2 |
| 186 | A | 4 | 1 | −2 | −2 | 0 | −1 | −1 | 0 | −2 | −1 | −1 | 0 | −1 | −2 | −1 | 0 | 0 | −3 | −2 | 0 |
| 187 | G | 0 | −2 | 0 | −1 | −3 | −2 | −2 | 6 | −2 | −2 | −3 | −2 | −2 | −3 | −2 | 0 | −2 | −2 | −3 | 0 |
| 188 | I | −1 | 2 | −1 | 0 | −3 | 0 | 3 | −3 | −1 | 2 | −1 | 0 | −1 | −2 | 1 | −1 | −1 | −3 | −2 | 0 |
| 189 | E | −1 | −1 | −1 | 0 | −3 | 0 | 4 | −2 | −1 | 0 | −2 | 0 | −1 | −3 | 3 | −1 | −1 | −3 | −2 | −1 |
| 190 | V | 0 | 0 | −1 | 1 | −2 | −1 | −1 | −2 | −2 | 0 | 0 | −1 | 0 | −2 | −2 | −1 | 2 | −3 | −2 | 3 |
| 191 | K | −1 | 0 | −1 | −1 | −3 | 0 | 1 | −2 | −1 | −2 | −2 | 4 | −1 | −2 | −2 | −1 | −1 | 7 | −1 | 0 |
| 192 | V | 0 | −1 | −1 | −2 | −2 | 1 | 0 | −3 | −2 | 0 | 0 | 1 | 0 | −2 | −2 | −1 | 2 | −3 | −1 | 3 |
| 193 | D | −2 | −2 | 0 | 6 | −3 | 0 | 1 | −1 | −1 | −3 | −4 | −1 | −3 | −3 | −1 | 0 | 1 | −4 | −3 | −3 |
| 194 | E | 1 | −1 | −1 | 1 | −2 | 0 | 4 | −2 | −1 | −1 | −2 | 0 | −1 | −2 | −1 | 0 | −1 | −3 | −2 | 1 |
| 195 | C | 0 | −3 | −3 | −3 | 10 | −3 | −4 | −3 | −3 | −1 | −1 | −3 | −1 | −2 | −3 | −1 | −1 | −2 | −2 | −1 |
| 196 | N | −1 | −1 | 3 | −1 | −2 | −1 | −1 | −1 | −1 | −1 | −1 | −1 | −1 | 2 | −2 | 0 | 4 | −2 | 0 | −1 |
| 197 | I | −1 | −1 | −2 | −3 | 5 | −2 | −2 | −3 | −3 | 4 | 0 | 1 | 0 | −1 | −3 | −1 | −1 | −3 | −1 | 1 |
| 198 | C | 0 | −3 | −2 | −3 | 9 | −3 | −3 | −3 | −3 | −1 | −1 | −3 | −1 | −2 | −3 | −1 | 1 | −2 | −2 | −1 |
| 199 | H | −2 | 2 | 0 | −2 | −3 | 0 | 0 | −2 | 7 | −3 | −2 | 0 | −2 | 0 | −2 | −1 | −2 | −1 | 3 | −3 |
| 200 | C | 0 | −3 | −3 | −3 | 10 | −3 | −4 | −3 | −3 | −1 | −1 | −3 | −1 | −2 | −3 | −1 | −1 | −2 | −2 | −1 |
| 201 | H | −1 | 0 | 0 | −1 | −2 | 0 | 0 | −2 | 7 | −2 | −2 | −1 | −2 | −1 | −2 | 0 | 3 | −2 | 0 | −2 |
| 202 | N | −2 | −1 | 4 | 3 | −3 | 0 | 0 | −1 | 0 | −2 | −2 | −1 | −2 | 0 | −2 | 0 | −1 | −1 | 4 | −2 |
| 203 | G | 0 | −1 | 0 | 0 | −3 | 0 | 3 | 5 | −1 | −4 | −4 | −1 | −3 | −3 | −2 | 0 | −2 | −2 | −3 | −3 |
| 204 | D | −1 | 0 | 0 | 4 | −3 | 3 | 3 | −2 | 0 | −3 | −3 | 0 | −2 | −3 | −1 | 0 | −1 | −3 | −2 | −2 |
| 205 | W | −1 | −2 | 0 | 3 | −3 | −1 | 0 | 3 | −2 | −3 | −3 | −2 | −2 | −1 | −2 | −1 | −2 | 8 | 0 | −3 |
| 206 | W | −2 | −3 | −2 | −3 | −2 | −2 | −3 | 3 | −2 | −3 | −3 | −3 | −1 | 0 | −3 | −2 | −2 | 11 | 0 | −3 |
| 207 | K | −1 | 3 | 0 | −2 | −3 | 0 | 0 | −2 | 0 | −2 | −2 | 3 | −1 | 0 | −2 | −1 | −1 | −1 | 4 | −2 |
| 208 | P | −1 | −1 | −1 | −1 | −2 | 3 | 0 | −3 | −1 | −1 | 1 | 0 | 0 | −2 | 4 | −1 | −1 | −3 | −2 | −1 |
| 209 | A | 5 | −1 | −2 | −2 | 0 | −1 | −1 | 0 | −2 | −1 | −1 | −1 | −1 | −2 | −1 | 0 | 0 | −3 | −2 | 0 |
| 210 | Q | −1 | 0 | −1 | −1 | −2 | 3 | 0 | −2 | −1 | −1 | 0 | 0 | 4 | −1 | −1 | 0 | 2 | −2 | −1 | 0 |
| 211 | C | 0 | −3 | −3 | −3 | 10 | −3 | −4 | −3 | −3 | −1 | −1 | −3 | −1 | −2 | −3 | −1 | −1 | −2 | −2 | −1 |
| 212 | S | 0 | −1 | 0 | 0 | −1 | 0 | 0 | −1 | −1 | −2 | −2 | 0 | −1 | −2 | −1 | 4 | 3 | −3 | −2 | −1 |
| 213 | K | −1 | 5 | 0 | −2 | −3 | 0 | 0 | −2 | 0 | −3 | −2 | 4 | −1 | −3 | −2 | −1 | −1 | −3 | −2 | −3 |
| 214 | R | −1 | 3 | 0 | −2 | −2 | 0 | −1 | −3 | 6 | −1 | 1 | 0 | 0 | −1 | −2 | −1 | −1 | −2 | 0 | −1 |
| 215 | E | −1 | 0 | 0 | 1 | −4 | 1 | 5 | −2 | 0 | −3 | −3 | 3 | −2 | −3 | −1 | 0 | −1 | −3 | −2 | −2 |
| 216 | C | 0 | −2 | 3 | −1 | 9 | −2 | −2 | −2 | −1 | −1 | −1 | −2 | −1 | −2 | −3 | 0 | −1 | −3 | −2 | −1 |
| 217 | Q | −1 | 2 | −1 | −1 | 6 | 3 | 0 | −2 | −1 | −2 | −2 | 1 | −1 | −3 | −2 | 0 | −1 | −2 | −2 | −2 |
| 218 | G | −1 | 0 | 3 | 0 | −3 | 3 | 0 | 3 | 0 | −3 | −3 | 0 | −1 | −3 | −2 | 0 | −1 | −3 | −2 | −3 |
| 219 | K | −1 | 0 | 0 | −2 | −2 | 0 | 0 | −2 | 5 | 0 | 0 | 3 | 3 | −1 | −2 | −1 | −1 | −2 | 0 | 0 |
| 220 | Q | −1 | 0 | 0 | 0 | −3 | 4 | 4 | −2 | 0 | −3 | −2 | 0 | −1 | −3 | −1 | 0 | −1 | −2 | −1 | −2 |
| 221 | T | 0 | −1 | 0 | −1 | −2 | −1 | −1 | 4 | −2 | −2 | −2 | −1 | −2 | −2 | −1 | 0 | 4 | −2 | −2 | −1 |
| 222 | V | 0 | −2 | −2 | −3 | −1 | −1 | −2 | −3 | −2 | 2 | 1 | −1 | 4 | 0 | −2 | −1 | 0 | −2 | −1 | 3 |

wherein, when this profile is input as query sequence into the search program BLAST, using the default parameters specified by the NCBI (the National Center for Biotechnology Information; http://www.ncbi.nlm.nih.gov/) [Blosum 62 matrix; gap open penalty=11 and gap extension penalty=1], members of the SECFAM3 family are those which have an E value of 10⁻² or less.

A “member of the SECFAM3 family” is thus to be interpreted herein as a polypeptide sequence that satisfies the profile described above with a maximum threshold E value of 10⁻² when used as a query sequence in BLAST using the parameters described above. Preferably, the polypeptide sequence has a minimum threshold E value of 10⁻⁵ or less, 10⁻¹⁰ or less, 10⁻⁵⁰ or less, most preferably, 10⁻⁷⁰ or less. For example, when the family member INSP124 (SEQ ID NO:12) is compared to the profile of the first aspect of the invention, the E value generated is e⁻¹⁴³. An E value represents the expected number of better or equally good matches found in a database at random, or alternatively may be described as the probability that a match has occurred at random. Accordingly, all hits are ranked according to their E-values, which, in turn, depend on a) the number of candidates available for each sequence position (20 in the case of amino acids), the length of the sequence or matching region, and the size of the database searched. Shorter sequences such as the members of the SECFAM3 family therefore tend to have larger E-values than a comparable match between two longer sequences.

The above profile takes into account the existence of a signal sequence and a vWFC domain. The profile allows for a higher degree of variability in the amino acid sequence of the signal peptide region (amino acids 1 to 23) compared to the vWFC domain. “Variability” in this context, relates to the degree of similarity and identity between the amino acid sequences. This reflects the situation found with the 22 members of the SECFAM3 family that are identified herein. The high degree of similarity shared in the vWFC-like domains between the fifteen members also suggests that the vWFC-like domain is likely to be involved in an important function of the molecule. If this domain was of less importance, the degree of conservation amongst its members would not be so high.

The database of translated nucleic acid sequences that is searched, may include, but is not limited to, translated nucleic acid sequences derived from cDNAs, ESTs, mRNAs, whole or partial genome databases.

In the second aspect of the invention, there is provided an isolated polypeptide which:

i) comprises or consists of a polypeptide sequence that has an E value of 10⁻² or less when the profile below is input as query sequence into the search program BLAST, using the default parameters specified by the NCBI (the National Center for Biotechnology Information, http://www.ncbi.nlm.nih.gov/) [Blosum 62 matrix; gap open penalty=11 and gap extension penalty=1]

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | M | −2 | −2 | −3 | −4 | −2 | −1 | −3 | −4 | −3 | 0 | 2 | −2 | 8 | 0 | −3 | −2 | −2 | −2 | −2 | 0 |
| 2 | A | 3 | −1 | −3 | −3 | −1 | −1 | −2 | −2 | −3 | 0 | 0 | 1 | 2 | 1 | −2 | −1 | −1 | −3 | −1 | 1 |
| 3 | L | 1 | −2 | −2 | −3 | −2 | −2 | −2 | −2 | 2 | 0 | 2 | −2 | 0 | −2 | 0 | 1 | −1 | −3 | −2 | 2 |
| 4 | H | 1 | −1 | 1 | −1 | −3 | −1 | −1 | 3 | 6 | −3 | −1 | −1 | −2 | −2 | −2 | 2 | −1 | −3 | −1 | −3 |
| 5 | I | 0 | −2 | −3 | −3 | −2 | 0 | 0 | −3 | −3 | 3 | 2 | −2 | 0 | −1 | −3 | −2 | −1 | −3 | −2 | 2 |
| 6 | H | 0 | −2 | −1 | −2 | −2 | −1 | −1 | −3 | 7 | 0 | 0 | −2 | −1 | −2 | 0 | 0 | −1 | −3 | 0 | 2 |
| 7 | E | 0 | −2 | −2 | −1 | −2 | −1 | 2 | −3 | −1 | 0 | 1 | −1 | 2 | 2 | −3 | 0 | −1 | −2 | 2 | 0 |
| 8 | A | 2 | −2 | −1 | −2 | 3 | −2 | −2 | 2 | −2 | −2 | −2 | −2 | −2 | −3 | −2 | 3 | 1 | −3 | −3 | −2 |
| 9 | C | 0 | −3 | −2 | −3 | 7 | −2 | −3 | −3 | −3 | 0 | −1 | −2 | 3 | 3 | −3 | 0 | −1 | −2 | −1 | 0 |
| 10 | I | −1 | −2 | −2 | −2 | −2 | −2 | 0 | 0 | −2 | 3 | 2 | −2 | 0 | 0 | −3 | 0 | 0 | −2 | 3 | 0 |
| 11 | L | −2 | 0 | −3 | −4 | −2 | −2 | −3 | −4 | −3 | 0 | 4 | −2 | 3 | 3 | −4 | −2 | −2 | −2 | 0 | 0 |
| 12 | L | 0 | 0 | −3 | −4 | −2 | −2 | −3 | −3 | −3 | 0 | 3 | −2 | 0 | −1 | 1 | −2 | −1 | −3 | −2 | 1 |
| 13 | L | −1 | −2 | −2 | −2 | −2 | −2 | 0 | −3 | −3 | 0 | 3 | −2 | 0 | 1 | −3 | 1 | 1 | −3 | −1 | 1 |
| 14 | V | 0 | 0 | −3 | −3 | 4 | 0 | −2 | −3 | −3 | 0 | 0 | −2 | 2 | −2 | −3 | −2 | −1 | −3 | −2 | 4 |
| 15 | I | −1 | 0 | −2 | −3 | 4 | −2 | −2 | −3 | 2 | 2 | 1 | −2 | 0 | −1 | −3 | 0 | 0 | 4 | −1 | 0 |
| 16 | P | 0 | 0 | −2 | −2 | −2 | −2 | −2 | −3 | 4 | −1 | 0 | −2 | −1 | 2 | 3 | 0 | −1 | −2 | 0 | 0 |
| 17 | G | 0 | −3 | −2 | −3 | −2 | −2 | −3 | 2 | −3 | 1 | 0 | −3 | 3 | 3 | −3 | −1 | −2 | −2 | 0 | 1 |
| 18 | L | 1 | −3 | −3 | −4 | −1 | −2 | −3 | −3 | −3 | 0 | 4 | −2 | 0 | −1 | −3 | −2 | −1 | −3 | −2 | 2 |
| 19 | V | 2 | −1 | −1 | −2 | −2 | 3 | −1 | −2 | −2 | 0 | −1 | −1 | −1 | −3 | −2 | 1 | 1 | −3 | −2 | 2 |
| 20 | T | −1 | −2 | −2 | −3 | 4 | −2 | −2 | −3 | −3 | 0 | 3 | −2 | 0 | −2 | −3 | 1 | 3 | −3 | −2 | 0 |
| 21 | S | 0 | −2 | −1 | −2 | 4 | −2 | −2 | 1 | −3 | −1 | 1 | −2 | 2 | −2 | 0 | 2 | −1 | −3 | −3 | −1 |
| 22 | A | 3 | −2 | −2 | −2 | −1 | −2 | −2 | −1 | −2 | −1 | 0 | −2 | −1 | 1 | 2 | 2 | −1 | −3 | −2 | 0 |
| 23 | A | 2 | −2 | −3 | −2 | 5 | 0 | 0 | −2 | −3 | −1 | 0 | −2 | −1 | −3 | 2 | −1 | −1 | −3 | −2 | 1 |
| 24 | I | 1 | 1 | −2 | −2 | −2 | −1 | −2 | −2 | −2 | 2 | 0 | −1 | −1 | −1 | −2 | 2 | −1 | −3 | 1 | 1 |
| 25 | S | −1 | −1 | 2 | −1 | −2 | 1 | −1 | −2 | 4 | 2 | −1 | −1 | −1 | −2 | −2 | 3 | −1 | −4 | −1 | −1 |
| 26 | H | 0 | −2 | 3 | −1 | −3 | −1 | −1 | −2 | 5 | −2 | −2 | −1 | −2 | 0 | 3 | 0 | −1 | −3 | −1 | 0 |
| 27 | E | −1 | −1 | 0 | 1 | −3 | 0 | 5 | −2 | −1 | −3 | −3 | 0 | −2 | −3 | −1 | 1 | 2 | −3 | −2 | −2 |
| 28 | D | 1 | −2 | 0 | 4 | −2 | −1 | 0 | −1 | −2 | −2 | 0 | −1 | −1 | −2 | −1 | 2 | 0 | −4 | −3 | −2 |
| 29 | Y | −2 | −1 | −2 | −2 | −3 | 2 | −1 | −3 | 0 | 0 | −1 | −1 | −1 | 0 | 3 | −1 | −2 | 0 | 5 | 0 |
| 30 | P | 0 | −1 | −1 | −1 | −2 | −1 | −1 | 0 | 5 | −3 | −3 | −1 | −2 | −3 | 4 | 0 | 0 | −3 | −1 | −2 |
| 31 | A | 3 | −2 | −2 | −2 | −1 | −1 | −2 | −1 | −3 | 0 | 2 | −1 | 0 | −2 | 0 | 0 | 0 | −3 | −2 | 0 |
| 32 | D | 2 | −1 | 0 | 5 | −3 | 0 | 2 | −2 | −1 | −2 | −3 | 2 | −2 | −3 | −1 | 0 | −1 | −4 | −3 | 0 |
| 33 | E | 0 | −1 | −2 | 0 | −2 | 0 | 3 | −3 | −1 | 0 | 0 | −1 | 0 | 3 | −2 | −1 | −1 | −2 | 0 | 0 |
| 34 | G | 1 | −2 | 0 | −1 | −2 | −1 | −2 | 4 | −2 | −2 | −2 | −2 | 0 | −2 | −2 | 1 | 1 | −2 | −3 | 0 |
| 35 | D | 0 | 2 | 0 | 2 | −2 | 0 | 0 | −2 | −1 | −2 | 0 | 0 | −1 | −3 | 2 | −1 | −1 | −3 | −2 | −2 |
| 36 | Q | 0 | 3 | 0 | −1 | −2 | 3 | 0 | −2 | −1 | −2 | −2 | 0 | −1 | −3 | −1 | 1 | 3 | −3 | −2 | −2 |
| 37 | I | 0 | −2 | −3 | −2 | −1 | −2 | −2 | −2 | −3 | 1 | 0 | −2 | 0 | −2 | 4 | −1 | −1 | −3 | −2 | 2 |
| 38 | S | 1 | −1 | 0 | 1 | −2 | 0 | 2 | −2 | −1 | 2 | −1 | 1 | −1 | −2 | −2 | 2 | −1 | −3 | −2 | 0 |
| 39 | S | 3 | −1 | −1 | −1 | −1 | −1 | −1 | −1 | −2 | −1 | 0 | −1 | −1 | −2 | 2 | 3 | 0 | −3 | −2 | −1 |
| 40 | N | 0 | −2 | 2 | −1 | −3 | −1 | 1 | 3 | −1 | 0 | 0 | −1 | −1 | −3 | 1 | −1 | −1 | −3 | −2 | 0 |
| 41 | D | 1 | −2 | −1 | 3 | −2 | 0 | 2 | −2 | −2 | −1 | 0 | −1 | −1 | 0 | −2 | 0 | −1 | −3 | −2 | 1 |
| 42 | N | −1 | −1 | 2 | 1 | −3 | 0 | 3 | 0 | −1 | −2 | 0 | 0 | −1 | −2 | −2 | 1 | −1 | −3 | −2 | −2 |
| 43 | L | 1 | −3 | −3 | −3 | 4 | −2 | −3 | −3 | −1 | 0 | 1 | −3 | 0 | 3 | −3 | −2 | −2 | 0 | 5 | 0 |
| 44 | I | 1 | −1 | −1 | 0 | −2 | 0 | 2 | −2 | −2 | 1 | −1 | 1 | −1 | −2 | −2 | 2 | −1 | −4 | −2 | 0 |
| 45 | F | −2 | −3 | −2 | 1 | −2 | −2 | −2 | −3 | −1 | 2 | 0 | −3 | 0 | 4 | −3 | −2 | −2 | −1 | 4 | 1 |
| 46 | D | 1 | −2 | −1 | 2 | −2 | −1 | 0 | 0 | −2 | −3 | −3 | −1 | −2 | −3 | 4 | 1 | −1 | −4 | −3 | −2 |
| 47 | D | 0 | −2 | 0 | 5 | −3 | −1 | 0 | 1 | −2 | −3 | −4 | −1 | −3 | −3 | 2 | 1 | −1 | −4 | −3 | −3 |
| 48 | Y | −2 | 3 | −1 | −2 | −3 | 2 | −1 | −3 | 0 | −2 | −2 | 0 | −1 | 0 | −3 | −2 | −2 | 0 | 6 | −2 |
| 49 | R | −2 | 4 | 0 | −2 | −4 | 2 | 0 | −2 | 5 | −4 | −3 | 0 | −2 | −3 | 2 | −1 | −2 | −3 | −1 | −3 |
| 50 | G | −1 | −2 | 0 | 2 | −3 | −1 | 1 | 4 | −2 | −2 | −3 | −1 | −2 | −3 | −2 | 1 | −1 | −3 | −3 | 0 |
| 51 | K | −1 | 2 | 1 | −1 | −3 | 0 | 0 | 1 | −1 | −3 | −3 | 4 | −2 | −3 | −2 | 1 | −1 | −3 | −3 | −3 |
| 52 | G | −1 | −2 | −1 | 2 | 4 | −2 | −1 | 3 | −2 | −2 | −3 | −2 | −2 | 0 | −2 | 0 | 2 | −2 | −2 | −2 |
| 53 | C | −1 | −4 | −4 | −4 | 10 | −4 | −4 | −4 | −3 | −1 | −1 | −4 | −1 | 2 | −4 | −2 | −2 | −2 | −1 | −1 |
| 54 | V | −1 | −2 | −1 | 0 | −2 | −1 | 1 | −2 | −2 | 0 | 0 | −1 | 3 | −2 | −2 | 1 | 0 | −3 | −2 | 3 |
| 55 | D | 0 | −2 | 0 | 6 | −3 | 0 | 1 | −1 | −1 | −3 | −4 | −1 | −3 | −3 | −1 | 0 | −1 | −4 | −3 | −3 |
| 56 | D | −2 | −1 | 3 | 4 | −4 | 0 | 2 | 2 | −1 | −4 | −4 | −1 | −3 | −4 | −2 | 0 | −1 | −4 | −3 | −3 |
| 57 | S | 0 | −1 | 2 | 2 | −3 | 1 | 0 | 2 | −1 | −3 | −3 | −1 | −2 | −3 | −2 | 3 | 0 | −3 | −3 | −3 |
| 58 | G | 0 | 1 | 0 | −1 | −3 | −1 | −2 | 5 | −2 | −4 | −4 | −1 | −3 | −3 | −2 | 1 | −2 | −3 | −3 | −3 |
| 59 | F | −1 | −4 | −4 | −4 | −2 | −3 | −3 | −4 | −2 | 3 | 0 | −3 | 0 | 5 | −4 | −2 | −1 | −1 | 0 | 3 |
| 60 | V | −1 | −3 | −3 | −2 | −2 | −1 | 1 | −3 | −1 | 0 | 0 | −2 | 0 | 3 | −3 | −2 | −1 | −1 | 4 | 3 |
| 61 | Y | −2 | −2 | −2 | −3 | −2 | −2 | −2 | −3 | 0 | −1 | −1 | −2 | −1 | 4 | −3 | 0 | −2 | 0 | 7 | −2 |
| 62 | K | 1 | −1 | −2 | −2 | −2 | −1 | −1 | 0 | −3 | 0 | −1 | 1 | −1 | −2 | 2 | −1 | −1 | −4 | −2 | 3 |
| 63 | L | −1 | −3 | −2 | −3 | −2 | −3 | −3 | 4 | −3 | 4 | 1 | −3 | 0 | −1 | −3 | −1 | −2 | −3 | −2 | 0 |
| 64 | G | −1 | −1 | 0 | 0 | −4 | 0 | 3 | 5 | −1 | −4 | −4 | −1 | −3 | −4 | −2 | 0 | −2 | −3 | −3 | −3 |
| 65 | E | −2 | −1 | −1 | 0 | −3 | 3 | 3 | −3 | −1 | −2 | −1 | 0 | 3 | −1 | −2 | −1 | −2 | 7 | −1 | −2 |
| 66 | R | −2 | 2 | −2 | −3 | −2 | 0 | −1 | −3 | 0 | 1 | −1 | 1 | −1 | 3 | −3 | −2 | −2 | −1 | 4 | 0 |
| 67 | F | −2 | −3 | −3 | −3 | −2 | −3 | −3 | −3 | −1 | −1 | −1 | −3 | −1 | 6 | −4 | −2 | 1 | 6 | 3 | −1 |
| 68 | F | 0 | 1 | −2 | −3 | −2 | −1 | −2 | −2 | −1 | −1 | 1 | −1 | 0 | 2 | −3 | 0 | 0 | −1 | 2 | −1 |
| 69 | P | −2 | −2 | −1 | 2 | −4 | 0 | 2 | −2 | −2 | −4 | −4 | −1 | −3 | −4 | 6 | −1 | −1 | −4 | −3 | −3 |
| 70 | G | 0 | −2 | 0 | −1 | −2 | −1 | −1 | 5 | −2 | −4 | −4 | −1 | −2 | −3 | −2 | 3 | −1 | −3 | −3 | −3 |
| 71 | H | −2 | −2 | 0 | 5 | −3 | −1 | 0 | −2 | 4 | −3 | −4 | −1 | −3 | −3 | 3 | 0 | −1 | −4 | −2 | −3 |
| 72 | S | −1 | −1 | 0 | −1 | −2 | −1 | −1 | −2 | 5 | −3 | −3 | −1 | −2 | −3 | 4 | 3 | 1 | −3 | −1 | −2 |
| 73 | N | 2 | −2 | 2 | −2 | 8 | −2 | −2 | −2 | −2 | −2 | −2 | −2 | −2 | −3 | −3 | 0 | 0 | −3 | −2 | −1 |
| 74 | C | −1 | −2 | −1 | −1 | 8 | −1 | 3 | −3 | −2 | −2 | −2 | −1 | −2 | −3 | −2 | 0 | 2 | −3 | −2 | −1 |
| 75 | P | −1 | 2 | −2 | −2 | −3 | 3 | 0 | −3 | −2 | −2 | 0 | 0 | −1 | −3 | 5 | −1 | −1 | −3 | −2 | −2 |
| 76 | C | 0 | −4 | −4 | −4 | 10 | −4 | −5 | −4 | −4 | −1 | −1 | −4 | −1 | −2 | −4 | −1 | −1 | −2 | −2 | −1 |
| 77 | V | −1 | −2 | −2 | −1 | −2 | −1 | 2 | −3 | −2 | 0 | 2 | −1 | 0 | −1 | −2 | −1 | 0 | −3 | −2 | 3 |
| 78 | C | 0 | −4 | −4 | −4 | 10 | −4 | −5 | −4 | −4 | −1 | −1 | −4 | −1 | −2 | −4 | −1 | −1 | −2 | −2 | −1 |
| 79 | A | 2 | −1 | −1 | −1 | −1 | 1 | −1 | −2 | −2 | −2 | −2 | −1 | −1 | −3 | −1 | 0 | 4 | −3 | −2 | −1 |
| 80 | L | 1 | −1 | −1 | 0 | −2 | 0 | 2 | −2 | −2 | −1 | 0 | −1 | −1 | −2 | −2 | 1 | 1 | −3 | −2 | −1 |
| 81 | D | −2 | −2 | 0 | 5 | −4 | 0 | 4 | −2 | −1 | −3 | −4 | −1 | −3 | −3 | −2 | 0 | 1 | −4 | −3 | −3 |
| 82 | G | 0 | −3 | 0 | −2 | −4 | −3 | −3 | 7 | −3 | −5 | −5 | −3 | −4 | −4 | −3 | 0 | −3 | −3 | −4 | −4 |
| 83 | P | −1 | −2 | −2 | −1 | −3 | −1 | −1 | −2 | −2 | −3 | −3 | −1 | −2 | −4 | 7 | 1 | −1 | −4 | −3 | −3 |
| 84 | V | 0 | −2 | −2 | −3 | −2 | 1 | −1 | −3 | −3 | 1 | 1 | −2 | 0 | −2 | −2 | 0 | −1 | −3 | −2 | 4 |
| 85 | C | 0 | −3 | −3 | −3 | 10 | −3 | −4 | −3 | −3 | −2 | −2 | −3 | −2 | −3 | −3 | 0 | −1 | −3 | −3 | −2 |
| 86 | D | 1 | −2 | −1 | 3 | 4 | −1 | −1 | −2 | −2 | −1 | −2 | −2 | 0 | 0 | −2 | 1 | 0 | −3 | −2 | 0 |
| 87 | Q | −1 | 3 | −1 | −2 | −3 | 4 | 0 | −3 | −1 | −2 | −2 | 2 | −1 | −3 | −2 | −1 | −1 | −3 | −2 | 0 |
| 88 | P | −1 | −2 | −1 | −2 | −2 | −2 | −2 | −3 | −3 | −2 | −2 | −2 | −2 | −4 | 6 | 0 | 4 | −4 | −3 | −1 |
| 89 | E | −2 | 2 | 0 | 2 | −4 | 0 | 5 | −2 | −1 | −4 | −3 | 1 | −2 | −4 | −2 | −1 | −2 | −4 | −3 | −3 |
| 90 | C | 0 | −4 | −4 | −4 | 10 | −4 | −5 | −4 | −4 | −2 | −2 | −4 | −2 | −3 | −4 | −2 | −2 | −3 | −3 | −2 |
| 91 | P | −1 | −2 | −2 | −2 | −3 | −2 | −2 | −3 | −3 | −1 | −2 | −2 | −2 | −3 | 6 | −1 | 2 | −4 | −3 | 0 |
| 92 | K | 0 | 3 | 0 | 0 | −3 | 0 | 3 | −2 | −1 | −3 | −3 | 3 | −2 | −3 | −2 | 0 | −1 | −3 | −2 | −2 |
| 93 | I | −1 | −3 | −3 | −4 | −1 | −2 | −3 | −4 | −3 | 3 | 3 | −2 | 0 | −1 | −3 | −2 | 1 | −3 | −2 | 1 |
| 94 | H | −1 | −1 | 0 | −1 | 4 | −1 | −1 | −2 | 7 | −3 | −3 | −1 | −2 | −2 | 2 | 1 | −1 | −3 | 0 | −3 |
| 95 | P | 0 | −2 | −2 | −1 | −3 | −1 | 1 | −2 | −2 | −3 | −3 | −1 | −2 | −4 | 7 | −1 | −1 | −4 | −3 | −2 |
| 96 | K | 1 | 3 | −1 | −2 | −2 | 0 | 0 | −2 | 0 | −2 | −2 | 2 | −1 | −1 | −2 | 1 | −1 | −2 | 2 | −2 |
| 97 | C | 0 | −3 | −3 | −3 | 10 | −3 | −4 | −3 | −3 | −2 | −2 | −3 | −2 | −3 | 2 | −1 | −1 | −3 | −3 | −2 |
| 98 | T | −1 | −3 | −2 | −3 | −1 | −2 | −3 | −4 | −3 | 4 | 0 | −3 | 1 | −1 | −3 | −1 | 3 | −3 | −2 | 2 |
| 99 | K | −2 | 1 | 0 | −1 | −4 | 0 | 2 | −2 | 7 | −3 | −3 | 2 | −2 | −2 | −2 | −1 | −2 | −3 | 0 | −3 |
| 100 | V | −1 | −3 | −3 | −3 | −1 | −3 | −3 | −4 | −3 | 3 | 0 | −3 | 0 | −1 | −3 | −2 | −1 | −3 | −1 | 5 |
| 101 | E | 1 | −1 | 0 | 3 | −2 | 0 | 2 | −1 | −1 | −3 | −3 | 1 | −2 | −3 | −1 | 2 | 0 | −4 | −3 | −2 |
| 102 | H | −2 | 2 | 1 | −1 | −3 | 0 | −1 | −2 | 7 | −3 | −2 | 0 | −2 | −1 | −2 | −1 | 1 | −2 | 3 | −2 |
| 103 | N | −1 | 0 | 3 | −1 | −2 | 0 | 0 | −1 | 4 | −2 | −2 | 1 | −2 | 0 | −2 | 1 | 0 | −1 | 4 | −2 |
| 104 | G | −1 | −1 | 0 | 2 | −3 | 4 | 2 | 2 | −1 | −4 | −3 | 0 | −2 | −3 | −2 | 0 | −1 | −3 | −2 | −3 |
| 105 | C | 0 | −3 | −3 | −3 | 10 | −3 | −5 | −3 | −3 | −1 | −1 | −3 | −1 | −2 | −3 | −1 | −1 | −2 | −2 | −1 |
| 106 | C | 0 | −3 | −3 | −3 | 10 | −3 | −5 | −3 | −3 | −1 | −1 | −3 | −1 | −2 | −3 | −1 | −1 | −2 | −2 | −1 |
| 107 | P | −1 | −3 | −3 | −2 | −3 | −2 | −2 | −3 | −3 | −1 | −2 | −2 | −2 | −4 | 7 | −2 | −1 | −4 | −3 | 0 |
| 108 | E | −1 | 2 | 0 | 2 | −3 | 4 | 2 | −2 | −1 | −2 | −1 | 0 | −1 | −3 | −2 | −1 | −1 | −3 | −2 | 0 |
| 109 | C | 0 | −3 | −3 | −3 | 10 | −3 | −5 | −3 | −3 | −1 | −1 | −3 | −1 | −2 | −3 | −1 | −1 | −2 | −2 | −1 |
| 110 | K | −1 | 0 | 0 | 0 | −3 | 0 | 3 | −2 | −1 | −1 | −2 | 3 | −1 | −3 | −2 | 1 | −1 | −3 | −2 | 1 |
| 111 | E | 2 | 2 | −1 | 0 | −3 | 0 | 4 | −2 | −1 | −3 | −2 | 2 | −2 | −3 | −2 | 0 | −1 | −3 | −2 | −2 |
| 112 | V | −1 | 1 | −1 | −1 | −3 | −1 | 1 | 1 | −2 | 1 | −1 | 1 | −1 | −2 | −2 | −1 | −1 | −3 | −2 | 2 |
| 113 | K | 0 | 0 | 0 | −1 | −3 | 0 | 0 | 4 | −2 | −4 | −3 | 4 | −2 | −3 | −2 | 1 | −1 | −3 | −3 | −3 |
| 114 | N | −2 | 0 | 6 | 0 | −3 | 0 | 0 | 0 | 0 | −3 | −3 | 2 | −2 | −3 | −2 | 0 | 0 | −4 | −2 | −3 |
| 115 | F | −1 | −3 | −3 | −3 | −2 | −2 | −2 | −3 | 0 | 0 | 0 | −2 | 0 | 4 | −3 | −2 | −1 | 0 | 6 | 1 |
| 116 | C | 0 | −3 | −3 | −3 | 10 | −3 | −5 | −3 | −3 | −1 | −1 | −3 | −1 | −2 | −3 | −1 | −1 | −2 | −2 | −1 |
| 117 | E | −1 | −1 | 0 | 3 | −3 | 0 | 4 | −2 | −1 | −2 | 0 | 0 | −2 | −3 | −2 | 0 | 1 | −3 | −3 | −2 |
| 118 | Y | −2 | −2 | −2 | −2 | −3 | −1 | 1 | −3 | 0 | −1 | −1 | −2 | −1 | 5 | −3 | −2 | −2 | 0 | 6 | −2 |
| 119 | H | −2 | 5 | 2 | −2 | −3 | 0 | −1 | −2 | 4 | −2 | −1 | 0 | 3 | −2 | −2 | −1 | −1 | −3 | −1 | −2 |
| 120 | G | −1 | −2 | 2 | −1 | −3 | −2 | −2 | 6 | −2 | −4 | −4 | −2 | −3 | −3 | −2 | 0 | −2 | −3 | −3 | −3 |
| 121 | K | −1 | 4 | −1 | −2 | −3 | 0 | 0 | −3 | −1 | −1 | −2 | 5 | −1 | −3 | −2 | −1 | −1 | −3 | −2 | 0 |
| 122 | N | −1 | −2 | 2 | −2 | −2 | −2 | −2 | −2 | −2 | 2 | 0 | −2 | 0 | −2 | −2 | 0 | 4 | −3 | −2 | 1 |
| 123 | Y | −2 | −3 | −3 | −3 | −2 | −2 | −3 | −3 | 0 | −1 | −1 | −3 | −1 | 4 | −4 | −2 | −2 | 1 | 8 | −1 |
| 124 | K | 0 | 1 | 0 | −1 | −3 | 2 | 2 | −2 | 4 | −3 | −2 | 2 | −1 | −1 | −2 | −1 | −2 | −2 | 3 | −2 |
| 125 | I | −1 | −2 | 2 | −2 | −2 | −2 | −2 | −2 | −2 | 2 | 1 | −2 | 0 | −1 | −2 | 0 | 1 | −3 | −2 | 2 |
| 126 | L | −1 | −2 | 2 | −2 | −2 | −2 | −2 | 3 | −2 | 0 | 3 | −2 | 0 | −1 | −3 | −1 | −1 | −3 | −2 | −1 |
| 127 | E | −1 | 0 | 0 | 1 | −4 | 3 | 6 | −2 | 0 | −3 | −3 | 0 | −2 | −3 | −1 | 0 | −1 | −3 | −2 | −2 |
| 128 | E | −1 | −1 | 2 | 0 | −3 | 0 | 4 | −2 | 0 | −3 | −3 | 0 | −2 | −1 | −2 | 0 | 1 | −2 | 3 | −2 |
| 129 | F | −2 | −3 | −3 | −3 | −2 | −3 | −3 | −3 | −1 | 0 | 0 | −3 | 0 | 7 | −5 | −2 | −2 | 0 | 2 | −1 |
| 130 | K | −1 | 1 | 2 | −1 | −3 | 2 | 0 | −2 | −1 | −1 | 0 | 2 | 1 | −2 | −2 | −1 | −1 | −3 | −2 | 0 |
| 131 | P | −1 | −3 | −3 | −3 | −2 | −2 | −2 | −3 | −3 | 1 | 2 | −2 | 0 | −2 | 3 | −2 | 0 | −3 | −2 | 3 |
| 132 | S | 0 | 0 | 1 | 2 | 2 | −1 | 0 | −1 | −1 | −3 | −3 | −1 | −2 | −3 | 1 | 4 | 1 | −4 | −3 | −2 |
| 133 | P | −1 | −2 | −2 | −1 | −3 | −1 | 1 | −3 | −2 | −2 | −3 | −1 | −2 | −4 | 7 | −1 | −1 | −4 | −3 | 0 |
| 134 | C | 0 | −3 | −3 | −3 | 10 | −3 | −4 | −3 | −3 | −1 | −1 | −3 | −1 | −2 | −3 | −1 | 0 | −2 | −2 | −1 |
| 135 | E | −1 | 1 | −1 | 0 | −3 | 0 | 5 | −3 | −1 | −1 | 1 | 0 | −1 | −2 | −2 | −1 | −1 | −3 | −2 | −1 |
| 136 | W | −1 | 2 | −1 | −2 | −3 | 2 | 0 | −2 | 3 | −2 | 0 | 1 | −1 | −2 | −2 | 1 | −1 | 4 | −1 | −2 |
| 137 | C | 0 | 0 | −3 | −3 | 10 | −3 | −4 | −3 | −3 | 0 | −1 | −3 | −1 | −2 | −3 | −1 | 0 | −3 | −2 | −1 |
| 138 | R | −1 | 4 | −1 | −2 | −2 | 0 | −1 | −3 | −1 | 2 | −1 | 2 | −1 | −1 | −2 | −1 | 1 | −2 | 1 | 0 |
| 139 | C | 0 | −4 | −4 | −4 | 10 | −4 | −5 | −4 | −4 | −1 | −1 | −4 | −1 | −2 | −4 | −1 | −1 | −2 | −2 | −1 |
| 140 | E | −1 | −1 | 0 | 2 | −3 | 0 | 5 | −3 | −1 | −2 | 0 | 0 | −1 | −3 | −2 | 0 | 1 | −3 | −2 | −2 |
| 141 | P | 1 | −2 | 1 | −2 | −2 | −1 | −1 | −2 | −2 | 0 | 0 | −1 | −1 | −2 | 2 | 1 | 1 | −3 | −2 | 0 |
| 142 | S | −1 | −1 | 4 | 2 | −3 | 1 | 0 | 2 | −1 | −3 | −3 | −1 | −2 | −3 | −2 | 2 | −1 | −4 | −3 | −3 |
| 143 | N | −1 | 1 | 2 | −1 | −3 | −1 | −1 | 4 | −1 | −4 | −4 | −1 | −2 | −3 | −2 | −1 | −1 | −3 | −3 | −3 |
| 144 | E | −1 | 0 | −1 | 0 | −3 | 0 | 4 | −3 | −1 | 1 | −1 | 0 | −1 | −2 | −2 | −1 | 1 | −3 | −2 | 1 |
| 145 | V | 3 | −2 | −3 | −3 | −1 | −2 | −2 | −2 | −3 | 0 | 0 | −2 | 0 | −2 | −2 | −1 | −1 | −3 | −2 | 3 |
| 146 | H | −2 | 1 | −2 | −3 | −2 | −1 | −2 | −3 | 3 | 0 | 2 | −1 | 0 | 2 | −3 | −2 | −2 | −1 | 4 | −1 |
| 147 | C | −1 | 0 | −3 | −3 | 10 | −3 | −3 | −3 | −3 | −2 | −2 | −2 | −2 | −3 | 1 | −1 | −1 | −3 | −3 | −2 |
| 148 | V | 0 | −2 | −1 | −2 | −1 | −1 | −2 | −2 | −3 | 0 | 1 | −1 | 0 | −2 | −2 | 2 | 3 | −3 | −2 | 2 |
| 149 | V | −1 | −3 | −3 | −3 | 1 | −2 | −2 | −3 | −3 | 2 | 0 | −2 | 0 | −2 | 2 | −2 | −1 | −4 | −2 | 5 |
| 150 | A | 3 | −2 | −2 | −2 | 2 | −1 | −1 | −1 | −2 | 0 | −1 | −1 | −1 | −2 | −2 | 2 | 0 | −3 | −2 | 2 |
| 151 | D | 1 | −2 | 0 | 5 | −2 | 1 | 0 | 0 | −2 | −3 | −3 | −1 | −2 | −3 | −2 | 1 | −1 | −4 | −3 | −2 |
| 152 | C | −1 | −3 | −3 | −3 | 10 | −3 | −4 | 0 | −3 | −2 | −2 | −3 | −1 | 1 | −4 | −1 | −2 | −2 | −1 | −2 |
| 153 | A | 2 | −2 | −2 | −2 | −2 | −1 | 0 | −2 | −2 | −1 | 0 | −1 | −1 | 1 | 4 | −1 | −1 | −3 | −1 | −1 |
| 154 | V | 2 | −1 | −2 | −1 | −2 | 4 | 1 | −2 | −2 | −1 | −2 | 0 | −1 | −3 | 3 | −1 | −1 | −3 | −2 | 0 |
| 155 | P | −1 | −2 | −1 | 1 | −2 | −2 | −1 | −2 | −2 | 1 | 0 | −2 | −1 | −2 | 4 | 0 | 2 | −4 | −3 | 0 |
| 156 | E | −2 | 1 | −1 | 0 | −4 | 0 | 3 | −3 | 2 | −3 | −3 | 1 | −2 | 2 | 3 | −1 | −2 | −3 | −1 | −3 |
| 157 | C | −1 | −3 | −3 | −3 | 9 | 1 | −3 | −3 | −3 | −2 | −1 | −3 | −1 | −2 | −3 | −2 | −2 | 5 | −2 | −2 |
| 158 | V | −1 | 0 | −2 | −3 | −2 | 0 | −2 | −3 | −3 | 2 | 0 | −2 | 0 | 1 | −2 | −2 | 1 | −2 | −1 | 4 |
| 159 | N | −2 | 1 | 4 | 4 | −3 | 0 | 0 | −1 | 0 | −3 | −3 | −1 | −2 | −1 | −2 | 0 | −1 | 3 | 3 | −3 |
| 160 | P | −1 | −2 | −2 | −1 | −3 | −1 | −1 | −2 | −2 | −1 | −3 | 1 | −2 | −4 | 7 | 0 | −1 | −4 | −3 | −2 |
| 161 | V | −1 | −3 | −3 | −3 | −2 | 0 | 0 | −4 | −3 | 4 | 0 | −2 | 0 | 2 | −3 | −2 | −1 | −2 | 0 | 3 |
| 162 | Y | −2 | 1 | −1 | −3 | −3 | −1 | −1 | −3 | 4 | −2 | 0 | −1 | −1 | 0 | −3 | 0 | −2 | 0 | 6 | −2 |
| 163 | E | −1 | −1 | −1 | 0 | −3 | 3 | 4 | −3 | −1 | −2 | −1 | 0 | −1 | −3 | 3 | 0 | 0 | −3 | −2 | −2 |
| 164 | P | −1 | −1 | −2 | −1 | −3 | 0 | 1 | −3 | −1 | −2 | −1 | 0 | −2 | −2 | 5 | −1 | −1 | −2 | 2 | −2 |
| 165 | E | −1 | −2 | 1 | 2 | −3 | −1 | 1 | 3 | −1 | −3 | −3 | −1 | −3 | −1 | −2 | 0 | −1 | −2 | 2 | −3 |
| 166 | Q | −2 | 0 | −1 | −1 | −3 | 4 | 2 | −3 | 3 | −3 | 0 | 2 | −1 | −2 | −2 | −1 | −2 | 5 | 0 | −2 |
| 167 | C | 0 | −3 | −3 | −4 | 9 | −3 | −4 | −4 | −3 | 0 | 0 | −3 | −1 | −2 | −3 | −2 | −1 | −2 | −2 | 0 |
| 168 | C | 0 | −3 | −3 | −3 | 9 | −3 | −3 | −3 | −3 | 0 | −1 | −3 | −1 | −2 | −3 | 0 | −1 | −3 | −2 | 1 |
| 169 | P | 0 | −2 | −3 | −2 | −3 | −1 | −2 | −2 | −3 | −2 | 0 | −1 | −1 | −3 | 7 | −1 | −1 | −4 | −3 | −1 |
| 170 | V | −1 | −2 | −2 | −2 | −2 | −1 | 1 | −3 | −2 | 3 | 0 | 1 | 0 | −2 | −2 | −2 | −1 | −3 | −2 | 4 |
| 171 | C | −1 | −2 | −3 | −3 | 9 | −2 | −3 | −3 | −3 | −1 | 0 | 1 | −1 | −2 | −3 | −1 | −1 | −3 | −2 | −1 |
| 172 | K | −1 | 1 | 0 | −1 | −3 | 0 | 0 | −2 | −1 | −3 | −2 | 6 | −1 | −3 | −1 | 0 | −1 | −3 | −2 | −2 |
| 173 | N | 2 | −1 | 3 | 2 | −2 | −1 | 0 | 2 | −1 | −3 | −3 | −1 | −2 | −3 | −2 | 1 | −1 | −4 | −3 | −2 |
| 174 | G | 0 | −1 | 0 | −1 | −3 | −2 | −2 | 5 | −2 | −4 | −3 | 0 | −3 | −3 | −2 | 0 | −2 | −2 | −3 | −3 |
| 175 | P | −1 | −2 | 1 | −1 | −3 | −1 | 0 | −2 | −2 | −3 | −3 | −1 | −2 | −4 | 6 | −1 | −1 | −4 | −3 | −2 |
| 176 | N | −1 | −1 | 5 | 0 | −3 | −1 | −1 | −1 | 0 | −3 | −3 | −1 | −2 | −2 | −2 | 1 | 0 | 6 | −1 | −3 |
| 177 | C | −1 | −2 | −2 | −2 | 8 | −2 | −2 | −3 | 3 | −2 | −2 | −2 | −2 | −2 | 1 | −1 | 0 | −3 | −1 | −2 |
| 178 | F | −2 | −2 | −1 | −2 | −3 | −2 | −2 | 1 | 4 | −2 | −2 | 0 | −1 | 4 | 0 | −1 | −2 | 0 | 3 | −2 |
| 179 | A | 2 | −2 | −2 | −3 | −1 | −2 | −2 | 2 | −3 | 0 | 1 | −2 | 0 | 0 | −2 | −1 | −1 | −3 | −1 | 1 |
| 180 | G | 0 | −1 | 0 | 0 | −2 | −1 | 1 | 3 | −2 | −2 | 0 | −1 | −1 | −2 | 1 | 1 | 1 | −3 | −2 | −1 |
| 181 | T | 0 | −2 | −1 | −1 | −1 | −1 | −1 | −2 | −2 | 1 | −1 | −1 | −1 | −2 | 1 | 0 | 5 | −3 | −2 | 0 |
| 182 | T | 1 | −1 | 0 | −1 | −1 | 2 | 0 | −1 | −2 | −1 | 0 | −1 | 0 | −2 | −1 | 1 | 3 | −3 | −2 | −1 |
| 183 | I | 1 | −3 | −2 | −3 | −1 | −2 | −2 | −2 | −3 | 3 | 0 | −2 | 0 | −1 | −2 | 0 | 0 | −3 | −1 | 3 |
| 184 | I | −1 | −3 | −3 | −3 | −2 | −3 | −3 | 1 | −3 | 5 | 0 | −3 | 0 | 2 | −3 | −2 | −1 | −2 | 0 | 1 |
| 185 | P | −1 | −2 | −2 | −1 | −3 | 0 | 1 | −2 | −2 | −2 | 0 | −1 | −1 | −3 | 6 | −1 | −1 | −4 | −3 | −2 |
| 186 | A | 4 | 1 | −2 | −2 | 0 | −1 | −1 | 0 | −2 | −1 | −1 | 0 | −1 | −2 | −1 | 0 | 0 | −3 | −2 | 0 |
| 187 | G | 0 | −2 | 0 | −1 | −3 | −2 | −2 | 6 | −2 | −2 | −3 | −2 | −2 | −3 | −2 | 0 | −2 | −2 | −3 | 0 |
| 188 | I | −1 | 2 | −1 | 0 | −3 | 0 | 3 | −3 | −1 | 2 | −1 | 0 | −1 | −2 | 1 | −1 | −1 | −3 | −2 | 0 |
| 189 | E | −1 | −1 | −1 | 0 | −3 | 0 | 4 | −2 | −1 | 0 | −2 | 0 | −1 | −3 | 3 | −1 | −1 | −3 | −2 | −1 |
| 190 | V | 0 | 0 | −1 | 1 | −2 | −1 | −1 | −2 | −2 | 0 | 0 | −1 | 0 | −2 | −2 | −1 | 2 | −3 | −2 | 3 |
| 191 | K | −1 | 0 | −1 | −1 | −3 | 0 | 1 | −2 | −1 | −2 | −2 | 4 | −1 | −2 | −2 | −1 | −1 | 7 | −1 | 0 |
| 192 | V | 0 | −1 | −1 | −2 | −2 | 1 | 0 | −3 | −2 | 0 | 0 | 1 | 0 | −2 | −2 | −1 | 2 | −3 | −1 | 3 |
| 193 | D | −2 | −2 | 0 | 6 | −3 | 0 | 1 | −1 | −1 | −3 | −4 | −1 | −3 | −3 | −1 | 0 | 1 | −4 | −3 | −3 |
| 194 | E | 1 | −1 | −1 | 1 | −2 | 0 | 4 | −2 | −1 | −1 | −2 | 0 | −1 | −2 | −1 | 0 | −1 | −3 | −2 | 1 |
| 195 | C | 0 | −3 | −3 | −3 | 10 | −3 | −4 | −3 | −3 | −1 | −1 | −3 | −1 | −2 | −3 | −1 | −1 | −2 | −2 | −1 |
| 196 | N | −1 | −1 | 3 | −1 | −2 | −1 | −1 | −1 | −1 | −1 | −1 | −1 | −1 | 2 | −2 | 0 | 4 | −2 | 0 | −1 |
| 197 | I | −1 | −1 | −2 | −3 | 5 | −2 | −2 | −3 | −3 | 4 | 0 | 1 | 0 | −1 | −3 | −1 | −1 | −3 | −1 | 1 |
| 198 | C | 0 | −3 | −2 | −3 | 9 | −3 | −3 | −3 | −3 | −1 | −1 | −3 | −1 | −2 | −3 | −1 | 1 | −2 | −2 | −1 |
| 199 | H | −2 | 2 | 0 | −2 | −3 | 0 | 0 | −2 | 7 | −3 | −2 | 0 | −2 | 0 | −2 | −1 | −2 | −1 | 3 | −3 |
| 200 | C | 0 | −3 | −3 | −3 | 10 | −3 | −4 | −3 | −3 | −1 | −1 | −3 | −1 | −2 | −3 | −1 | −1 | −2 | −2 | −1 |
| 201 | H | −1 | 0 | 0 | −1 | −2 | 0 | 0 | −2 | 7 | −2 | −2 | −1 | −2 | −1 | −2 | 0 | 3 | −2 | 0 | −2 |
| 202 | N | −2 | −1 | 4 | 3 | −3 | 0 | 0 | −1 | 0 | −2 | −2 | −1 | −2 | 0 | −2 | 0 | −1 | −1 | 4 | −2 |
| 203 | G | 0 | −1 | 0 | 0 | −3 | 0 | 3 | 5 | −1 | −4 | −4 | −1 | −3 | −3 | −2 | 0 | −2 | −2 | −3 | −3 |
| 204 | D | −1 | 0 | 0 | 4 | −3 | 3 | 3 | −2 | 0 | −3 | −3 | 0 | −2 | −3 | −1 | 0 | −1 | −3 | −2 | −2 |
| 205 | W | −1 | −2 | 0 | 3 | −3 | −1 | 0 | 3 | −2 | −3 | −3 | −2 | −2 | −1 | −2 | −1 | −2 | 8 | 0 | −3 |
| 206 | W | −2 | −3 | −2 | −3 | −2 | −2 | −3 | 3 | −2 | −3 | −3 | −3 | −1 | 0 | −3 | −2 | −2 | 11 | 0 | −3 |
| 207 | K | −1 | 3 | 0 | −2 | −3 | 0 | 0 | −2 | 0 | −2 | −2 | 3 | −1 | 0 | −2 | −1 | −1 | −1 | 4 | −2 |
| 208 | P | −1 | −1 | −1 | −1 | −2 | 3 | 0 | −3 | −1 | −1 | 1 | 0 | 0 | −2 | 4 | −1 | −1 | −3 | −2 | −1 |
| 209 | A | 5 | −1 | −2 | −2 | 0 | −1 | −1 | 0 | −2 | −1 | −1 | −1 | −1 | −2 | −1 | 0 | 0 | −3 | −2 | 0 |
| 210 | Q | −1 | 0 | −1 | −1 | −2 | 3 | 0 | −2 | −1 | −1 | 0 | 0 | 4 | −1 | −1 | 0 | 2 | −2 | −1 | 0 |
| 211 | C | 0 | −3 | −3 | −3 | 10 | −3 | −4 | −3 | −3 | −1 | −1 | −3 | −1 | −2 | −3 | −1 | −1 | −2 | −2 | −1 |
| 212 | S | 0 | −1 | 0 | 0 | −1 | 0 | 0 | −1 | −1 | −2 | −2 | 0 | −1 | −2 | −1 | 4 | 3 | −3 | −2 | −1 |
| 213 | K | −1 | 5 | 0 | −2 | −3 | 0 | 0 | −2 | 0 | −3 | −2 | 4 | −1 | −3 | −2 | −1 | −1 | −3 | −2 | −3 |
| 214 | R | −1 | 3 | 0 | −2 | −2 | 0 | −1 | −3 | 6 | −1 | 1 | 0 | 0 | −1 | −2 | −1 | −1 | −2 | 0 | −1 |
| 215 | E | −1 | 0 | 0 | 1 | −4 | 1 | 5 | −2 | 0 | −3 | −3 | 3 | −2 | −3 | −1 | 0 | −1 | −3 | −2 | −2 |
| 216 | C | 0 | −2 | 3 | −1 | 9 | −2 | −2 | −2 | −1 | −1 | −1 | −2 | −1 | −2 | −3 | 0 | −1 | −3 | −2 | −1 |
| 217 | Q | −1 | 2 | −1 | −1 | 6 | 3 | 0 | −2 | −1 | −2 | −2 | 1 | −1 | −3 | −2 | 0 | −1 | −2 | −2 | −2 |
| 218 | G | −1 | 0 | 3 | 0 | −3 | 3 | 0 | 3 | 0 | −3 | −3 | 0 | −1 | −3 | −2 | 0 | −1 | −3 | −2 | −3 |
| 219 | K | −1 | 0 | 0 | −2 | −2 | 0 | 0 | −2 | 5 | 0 | 0 | 3 | 3 | −1 | −2 | −1 | −1 | −2 | 0 | 0 |
| 220 | Q | −1 | 0 | 0 | 0 | −3 | 4 | 4 | −2 | 0 | −3 | −2 | 0 | −1 | −3 | −1 | 0 | −1 | −2 | −1 | −2 |
| 221 | T | 0 | −1 | 0 | −1 | −2 | −1 | −1 | 4 | −2 | −2 | −2 | −1 | −2 | −2 | −1 | 0 | 4 | −2 | −2 | −1 |
| 222 | V | 0 | −2 | −2 | −3 | −1 | −1 | −2 | −3 | −2 | 2 | 1 | −1 | 4 | 0 | −2 | −1 | 0 | −2 | −1 | 3 |

(ii) is a fragment thereof which is a member of the vWFC domain containing protein family, or has an antigenic determinant in common with the polypeptides of (i); or
(iii) is a functional equivalent of (i) or (ii).

Preferably, a polypeptide according to the invention is a member of the vWFC-domain containing secreted protein family. Preferably, in the above test, the polypeptide gives a maximum threshold E value of 10⁻². More preferably, the polypeptide sequence has a minimum threshold E value of 10⁻⁵ or less, 10⁻¹¹ or less, 10⁻⁵⁰ or less, most preferably, 10⁻⁷⁰ or less. Lowering the threshold value acts as a more stringent filter to separate polypeptides comprising a signal peptide and vWFC domain from the general background polypeptide sequences.

In a third embodiment of the second aspect of the invention, there is provided an isolated polypeptide which

(i) comprises a polypeptide satisfying the consensus amino acid sequence:

[GTDFC] (0, 1)-[CF] (0, 1)-[VMSED] (0, 1)-[DENG]
(0, 1)-[SQNDG] (0, 1)-[SGR] (0, 1)-[FIV] (0, 1)-
[VYFE] (0, 1)-[YFS] (0, 1)-[KVAGP] (0, 1)-[LIG]
(0, 1)-[GE] (0, 1)-[EWQM) (0, 1)-[RKYFQVI] (0, 1)-
[FYWT] (0, 1)-[FALYRTS] (0, 1)-[PED] (0, 1)-[GS]
(0, 1)-[HPDS] (0, 1)-[STHP] (0, 1)-[CNAT] (0, 1)-
[CTE] (0, 1)-[PQRL] (0, 1)-C(0, 1)-[VELT] (0, 1)-
C(0, 1)-[TAQ] (0, 1)-[ELATSD] (0, 1)[EDT]-G-[PS]-
[VLAQS]-[CS]-[DAMSTFCV]-[QRKV]-R(0, 1)-[PT]-
[ERDK]-C-[PTV]-[KERSA]-[LIVT]-[HPSC]-[PAE]-
[KRASY]-[CP]-[TIVM]-[HKRE]-[VI]-[DESAKP]-[HTNRYG]-
[NSTYHK] (0, 1)-[PA] (0, 1)-[TG] (0, 1)-[QGDES]-C-
C-[PV]-[EQRDLV]-C;

(ii) is a fragment thereof which is a member of the vWFC domain containing protein family, or bas an antigenic determinant in common with the polypeptides of (i); or
(iii) is a functional equivalent of (i) or (ii).

Preferably, a polypeptide according to the invention is a member of the vWFC-domain containing secreted protein family. The sequence recited in this embodiment of the invention covers the high identity region from INSP124 (SEQ ID NO:12) amino acid position 54-171 (amino acids 155-279 of the alignment, see FIG. 1).

In a fourth embodiment, the polypeptide comprises or consists of a polypeptide satisfying the consensus amino acid sequence

[GTDFC] (0, 1)-[CF] (0, 1)-[VMSED] (0, 1)-[DEA]
(0, 1)-[SQNDG] (0, 1)-[SGR] (0, 1)-[FIV] (0, 1)-
[VYFE] (0, 1)-[YFS] (0, 1)-[KVAGP] (0, 1)-[LIG]
(0, 1)-[GE] (0, 1)-[EWQM] (0, 1)-[RKYFQVI] (0, 1)-
[FYWT] (0, 1)-[FALYRTS] (0, 1)-[PED] (0, 1)-[GS]
(0, 1)-[HPDS] (0, 1)-[STHP] (0, 1)-[CNAT] (0, 1)-
[CTE] (0, 1)-[PQRL] (0, 1)-C(0, 1)-[VELT] (0, 1)-
C (0, 1)-[TAQ] (0, 1)-[ELATSD] (0, 1)-[EDT]-G-
[PS]-[VLAQS]-[CS]-DAMSTFCV]-[QRKV]-R (0, 1)-[PT]-
[ERDK]-C-[PTV]-[KERSA]-[LIVT]-[HPSC]-[PAE]-
[KRASY]-[CP]-[TIVM]-[HKRE]-[VI]-[DESAKP]-[HTNRYG]-
[NSTYHK] (0, 1)-[PA] (0, 1)-[TG] (0, 1)-[QGDES]-C-
C-[PV]-[EQRDLV]-C-[KERSV]-[EKRA]-[VIRKEG]-[KGS]-
[NK]-[FYV]-C-[EDLT]-[YFE]-[HRNM]-[GN]-[KRV]-
[NTVLI]-[YF]-[KQHREAY]-[ILVTSN]-[LGN]-[EQ]-[ENYT]-
F-P(0, 1)-S(0, 1)-[KRVMLQN]-[PVLIT]-[SNTCRDP]-
[PVE] (0, 1)-[CT] (0, 1)-[ELR] (0, 1)-[WRHKQSL]
(0, 1)-[CTIR] (0, 1)-[RTYIK] (0, 1)-C-[EDTL]-
[PAVLSNT] (0, 1)-[SNQDG] (0, 1-[GNRKS] (0, 1)-
[EIVTR]-[VAL]-[RHLYF] (0, 1)-[CRP]-[TSVL] (0, 1)-
[VICP]-[ASVC]-Q(0, 1)-A (0, 1)-C(0, 1)-[DAPQGS]-
[CQGF]-[APTFLE] (0, 1)-[QVAPE]-[TPSLID]-[EPRHKF]-
[CWQ]-[VQTFI]-[NDQRY]-[PLKS]-[VILEF]-[YLSHR]-
[EQTSP]-[PKLYE]-[DEGIYN]-[QSWKHE]-[CAL]-[CV]-[FL]-
[VIKES]-[CK].

In a fifth embodiment of the second aspect of the invention, there is provided an isolated polypeptide which consists of a polypeptide satisfying the consensus amino acid sequence

[GTDFC] (0, 1)-[CF] (0, 1)-[VMSED] (0, 1)-[DEA]
(0, 1)-[DENG] (0, 1)-[SQNDG] (0, 1)-[SGR] (0, 1)-
[FIV] (0, 1)-[VYFE] (0, 1)-[YFS] (0, 1)-[KVAGP]
(0, 1)-[LIG] (0, 1)-[GE] (0, 1)-[EWQN] (0, 1)-
[RKYFQVI] (0, 1)-[FYWT] (0, 1)-[FALYRTS] (0, 1)-
[PED] (0, 1)-[GS] (0, 1)-[HPDS] (0, 1)-[STHP]
(0, 1)-[CNAT] (0, 1)-[CTE] (0, 1)-[PQRL] (0, 1)-C
(0, 1)-[VELT] (0, 1)-C(0, 1)-[TAQ] (0, 1)-[ELATSD]
(0, 1)-[EDT]-G-[PS]-[VLAQS]-[CS]-[DAMSTFCV]-
[QRKV]-R (0, 1)-[PT]-[ERDK]-C-[PTV]-[KERSA]-
[LIVT]-[HPSC]-[PAE]-[KRASY]-[CP]-[TIVM]-[HKRE]-
[VI]-[DESAKP]-[HTNRYG]-[NSTYHK] (0, 1)-[PA]
(0, 1)-[TG] (0, 1)-[QGDES]-C-C-[PV]-[EQRDLV]-C;

In a sixth embodiment of the second aspect of the invention, there is provided an isolated polypeptide of the third embodiment of the second aspect of the invention, wherein the isolated polypeptide comprises one or more, preferably, all of 10 cysteine residues at amino acid positions 2, 23, 25, 27, 34, 40, 47, 57, 58 and 61 of the consensus amino acid sequence of the third to fifth embodiments of the second aspect of the invention. In a further embodiment, the isolated polypeptide comprises one or more, preferably, all of 10 cysteine residues at amino acid positions 68, 88, 91, 93, 101, 109, 114, 124, 125 and 128 of the consensus amino acid sequence of the fourth embodiment of the second aspect of the invention. In yet a further embodiment, the isolated polypeptide comprises one or more, preferably, all of the cysteine residues at amino acid positions 2, 23, 25, 27, 34, 40, 47, 57, 58, 61, 68, 88, 91, 93, 101, 109, 114, 124, 125 and 128 of the consensus amino acid sequence of the fourth embodiment of the second aspect of the invention.

The amino acid sequences of the third to fifth embodiments of the second aspect of the invention are written in PROSITE (protein sites and patterns) notation, with the amino acids being represented by their one-letter codes (Bairoch, A., Bucher, P., and Hofmann, K., (1997). The PROSITE Database: Its status in 1997. Nucl. Acids Res. 25, 217-221). Briefly, a peptide comprising the following formula:
A(1)−x(i1,j1)−A2−x(i2,j2)− . . . A{p−1}−x(i{p−1},j{p−1})−Ap
is to be interpreted in the following manner.

A(k) is a component, either specifying one amino acid, e.g. C, or a set of possible amino acids, e.g. [ILVF]. A component A(k) is an identity component if it specifies exactly one amino acid (for instance C or L) or an ambiguous component if it specifies more than one (for instance [ILVF] or [FWY]). i(k), j(k) are integers so that i(k)<=j(k) for all k. The part x(ik,jk) specifies a wildcard region of the pattern matching between ik and jk arbitrary amino acids. A wildcard region x(ik,jk) is “flexible” if jk is bigger than ik (for example x(2,3). The flexibility of such a region is jk-ik.br> For example the flexibility of x(2,3) is 1. The wildcard region is fixed if j(k) is equal to i(k), e.g., x(2,2) which can be written as x(2). The product of flexibility for a pattern is the product of the flexibilities of the flexible wildcard regions in the pattern, if any, otherwise it is defined to be one.

For example, C-x(2)-H is a pattern with two components (C and H) and one fixed wildcard region. It matches any sequence containing a C followed by any two arbitrary amino acids followed by an H. Amino acid sequences ChgHyw and liChgHlyw would be included in the formula. C-x(2,3)-H is a pattern with two components (C and H) and one flexible wildcard region. It matches any sequence containing a C followed by any two or three arbitrary amino acids followed by an H such as aaChgHywk and liChgaHlyw. C-x(2,3)-[ILV] is a pattern with two components (C and [ILV]) and one flexible wildcard region. It matches any sequence containing a C followed by any two or three arbitrary amino acids followed by an I, L or V.

Although the Applicant does not wish to be bound by this theory, it is postulated that the polypeptides of the above-described embodiments of the invention all possess signal peptide sequences. Accordingly, mature forms of the described polypeptides which lack the signal peptides form a further aspect of the present invention.

In one embodiment of the third aspect of the invention, there is provided a polypeptide which:
(i) comprises the amino acid sequence as recited in SEQ ID NO:2, SEQ ID NO:4, SEQ ID NO:39, SEQ ID NO:41, SEQ ID NO:43 and/or SEQ ID NO:45;
(ii) is a fragment thereof which is a member of the vWFC domain containing protein family, or has an antigenic determinant in common with the polypeptides of (i); or
(ii) is a functional equivalent of (i) or (ii).

According to a second embodiment of this third aspect of the invention, there is provided a polypeptide which consists of the amino acid sequence as recited in SEQ ID NO:2 SEQ ID NO:4, SEQ ID NO:39, SEQ ID NO:41, SEQ ID NO:43 and/or SEQ ID NO:45.

The polypeptide having the sequence recited in SEQ ID NO:2 is referred to hereafter as the “INSP123 polypeptide”.

A small amount of EST data, mostly from rodent ESTs, suggests that the INSP123 sequence should be found in brain cDNA templates or nerve tissue.

Although the Applicant does not wish to be bound by this theory, it is postulated that the first 23 amino acids of the INSP123 polypeptide form a signal peptide. The INSP123 full length polypeptide sequence with and without the signal sequence are recited in SEQ ID NO: 2 and SEQ ID NO:4, respectively. The polypeptide having the sequence recited in SEQ ID NO:4 is referred to hereafter as “the INSP123 mature polypeptide”.

Alternatively, although the Applicant does not wish to be bound by this theory, it is postulated that the first 22 amino acids of the INSP123 cloned polypeptide form a signal peptide. The INSP123 cloned full length polypeptide sequence with and without the signal sequence are recited in SEQ ID NO: 39 and SEQ ID NO:41, respectively. The polypeptide having the sequence recited in SEQ ID NO:39 is referred to hereafter as “the INSP123 cloned polypeptide”. The polypeptide having the sequence recited in SEQ ID NO:41 is referred to hereafter as “the INSP123 cloned mature polypeptide 1”.

Alternatively and preferably, although the Applicant does not wish to be bound by this theory, it is postulated that the first 21 amino acids of the INSP123 cloned polypeptide form a signal peptide. The INSP123 cloned full length polypeptide sequence with and without the signal sequence are recited in SEQ ID NO: 39 and SEQ ID NO:43, respectively. The polypeptide having the sequence recited in SEQ ID NO:39 is referred to hereafter as “the INSP123 cloned polypeptide”. The polypeptide having the sequence recited in SEQ ID NO:43 is referred to hereafter as “the INSP123 cloned mature polypeptide 2”.

Alternatively and preferably, although the Applicant does not wish to be bound by this theory, it is postulated that the first 31 amino acids of the INSP123 cloned polypeptide form a signal peptide. The INSP123 cloned full length polypeptide sequence with and without the signal sequence are recited in SEQ ID NO: 39 and SEQ ID NO:45, respectively. The polypeptide having the sequence recited in SEQ ID NO:39 is referred to hereafter as “the INSP123 cloned polypeptide”. The polypeptide having the sequence recited in SEQ ID NO:45 is referred to hereafter as “the INSP123 cloned mature polypeptide 3”.

Preferably, the antigenic determinant, fragment or functional equivalent of the second embodiment of the third aspect of the invention comprises one or more of the ten cysteine residues at amino acid positions 53, 74, 76, 78, 85, 90, 97, 105, 106 and 107 of SEQ ID NO:2. More preferably, one or more of these cysteine residues participate in disulphide bond formation under physiological conditions. In this aspect of the invention, by “physiological conditions” is meant the natural environment in which the native or wildtype form of the polypeptide would be found. Disulphide bond formation is often integral to the correct conformation of a protein and thus, its function. Disulphide bond formation is often integral to the correct conformation of a protein and thus, its function. It is therefore important that such cysteine residues be conserved.

In a third embodiment of the third aspect of the invention, there is provided a polypeptide which:
(i) comprises the amino acid sequence as recited in SEQ ID NO:6, SEQ ID NO:8, SEQ ID NO:10, SEQ ID NO:12, SEQ ID NO:14, SEQ ID NO:16, SEQ ID NO:47, SEQ ID NO:49, SEQ ID NO:51 and/or SEQ ID NO:53;
(ii) is a fragment thereof which is a member of the vWFC domain containing protein or having an antigenic determinant in common with the polypeptides of (i); or
(iii) is a functional equivalent of (i) or (ii).

Preferably, the polypeptide according to this third aspect of the invention consists of the amino acid sequence as recited in SEQ ID NO:12, SEQ ID NO:16, SEQ ID NO:47, SEQ ID NO:49, SEQ ID NO:51 and/or SEQ ID NO:53,

Preferably, the antigenic determinant, fragment or functional equivalent of the fourth embodiment of the third aspect of the invention comprises one or more of the ten cysteine residues at amino acid positions 53, 74, 76, 78, 85, 90, 97, 105, 106 and 109 of SEQ ID NO:12. More preferably, one or more of these cysteine residues participate in disulphide bond formation under physiological conditions. Even more preferably, said antigenic determinant, fragment or function equivalent further comprises one or more of the ten cysteine residues at amino acid positions 116, 134, 137, 139, 147, 152, 157, 167, 168 and 171.

The polypeptide having the sequence recited in SEQ ID NO:6 is referred to hereafter as “the INSP124 exon 1 polypeptide”. The polypeptide having the sequence recited in SEQ ID NO:8 is referred to hereafter as “the INSP124 exon 2 polypeptide”. The polypeptide having the sequence recited in SEQ ID NO:10 is referred to hereafter as “the INSP124 exon 3 polypeptide”.

The polypeptide having the sequence recited in SEQ ID NO:12 is referred to hereafter as “the INSP124 polypeptide”.

INSP124 is predicted to be similar to Ventroptin, also known as Neuralin, a BMP-4 (bone morphogenetic protein 4) antagonist expressed in a double gradient pattern in the retina. BMPs are multifunctional secreted proteins which signal through specific receptors. They have a key role in chondrogenesis deduced by their ability to induce ectopic chondrogenesis in adult animals (Chimal-Monroy J et al., Dev Biol. 2003 May 15; 257(2):292-301).

Ventroptin is a member of the chordin family and known to antagonize BMP2 as well as BMP4 (Takahashi H et al., Development. 2003 November; 130(21):5203-15). Misexpression of Ventroptin altered expression patterns of several topographic genes in the retina and projection of the retinal axons to the tectum along both axes. Thus, topographic retinotectal projection appears to be specified by the double-gradient molecule Ventroptin along the two axes (Sakuta H et al., Science. 2001 Jul. 6; 293(5527):111-5). During organogenesis, ventroptin presents a broad expression pattern in many tissues such as dorsal root ganglia, gut, condensing cartilages of the skeleton and developing hair follicles. (Coffinier C et al., Mech Dev. 2001 January; 100(1): 119-22).

A novel chordin-like protein, CHL2, which is structurally most homologous to ventroptin has recently been identified. When injected into Xenopus embryos, CHL2 RNA induced a secondary axis. It is postulated that CHL2 may play negative roles in the (re)generation and maturation of articular chondrocytes in the hyaline cartilage of both developing and degenerated joints. (Nakayama N. et al., (2004) January; 131(1):229-40).

A small amount of EST data, mostly from rodent ESTs, suggests that the INSP124 sequence should be found in nerve tissue.

Although the Applicant does not wish to be bound by this theory, it is postulated that the first 23 amino acids of INSP124 exon 1 polypeptide form a signal peptide. The INSP124 exon 1 and full length polypeptide sequences without the signal sequence are recited in SEQ ID NO: 14 and SEQ ID NO:16, respectively. The polypeptide having the sequence recited in SEQ ID NO:14 is referred to hereafter as “the INSP124 exon 1 mature polypeptide”. The polypeptide having the sequence recited in SEQ ID NO:16 is referred to hereafter as “the INSP124 mature polypeptide”.

Alternatively, although the Applicant does not wish to be bound by this theory, it is postulated that the first 22 amino acids of the INSP124 cloned polypeptide form a signal peptide. The INSP124 cloned full length polypeptide sequence with and without the signal sequence are recited in SEQ ID NO:47 and SEQ ID NO:49, respectively. The polypeptide having the sequence recited in SEQ ID NO:47 is referred to hereafter as “the INSP124 cloned polypeptide”. The polypeptide having the sequence recited in SEQ ID NO:49 is referred to hereafter as “the INSP124 cloned mature polypeptide 1”.

Alternatively and preferably, although the Applicant does not wish to be bound by this theory, it is postulated that the first 21 amino acids of the INSP124 cloned polypeptide form a signal peptide. The INSP124 cloned full length polypeptide sequence with and without the signal sequence are recited in SEQ ID NO:47 and SEQ ID NO:51, respectively. The polypeptide having the sequence recited in SEQ ID NO:47 is referred to hereafter as “the INSP124 cloned polypeptide”. The polypeptide having the sequence recited in SEQ ID NO:51 is referred to hereafter as “the INSP124 cloned mature polypeptide 2”.

Alternatively and preferably, although the Applicant does not wish to be bound by this theory, it is postulated that the first 31 amino acids of the INSP124 cloned polypeptide form a signal peptide. The INSP124 cloned full length polypeptide sequence with and without the signal sequence are recited in SEQ ID NO:47 and SEQ ID NO:53, respectively. The polypeptide having the sequence recited in SEQ ID NO:47 is referred to hereafter as “the INSP124 cloned polypeptide”. The polypeptide having the sequence recited in SEQ ID NO:53 is referred to hereafter as “the INSP124 cloned mature polypeptide 3”.

The term “INSP124 exon polypeptides” as used herein includes polypeptides comprising the INSP124 exon 1 polypeptide, the INSP124 exon 2 polypeptide, the INSP124 exon 1 mature polypeptide, the INSP124 exon 3 polypeptide, the INSP124 polypeptide, the INSP124 mature polypeptide 1, the INSP124 mature polypeptide 2, or the INSP124 mature polypeptide 3, as well as polypeptides consisting of the INSP124 exon 1 polypeptide, the INSP124 exon 1 mature polypeptide, the INSP124 exon 2 polypeptide, the INSP124 exon 3 polypeptide, the INSP124 polypeptide or the INSP124 mature polypeptide.

In a fifth embodiment of the third aspect of the invention, there is provided a polypeptide which:
(i) comprises the amino acid sequence as recited in SEQ ID NO:18, SEQ ID NO:20, SEQ ID NO:22, SEQ ID NO:24, SEQ ID NO:26, SEQ ID NO:28, SEQ ID NO:30, SEQ ID NO:55, SEQ ID NO:57, SEQ ID NO:59 and/or SEQ ID NO:61;
(ii) is a fragment thereof which is a member of the vWFC domain containing protein or having an antigenic determinant in common with the polypeptides of (i); or
(iii) is a functional equivalent of (i) or (ii).

According to a sixth embodiment of the third aspect of the invention, there is provided a polypeptide which consists of the amino acid sequence as recited in SEQ ID NO:18, SEQ ID NO:20, SEQ ID NO:22, SEQ ID NO:24, SEQ ID NO:26, SEQ ID NO:28, SEQ ID NO:30, SEQ ID NO:47, SEQ ID NO:49, SEQ ID NO:51 and/or SEQ ID NO:53;

Preferably, the antigenic determinant, fragment or functional equivalent of the sixth embodiment of the third aspect of the invention comprises one or more of the ten cysteine residues at amino acid positions 69, 82, 90, 92, 100, 105, 110, 120, 121 and 124 of SEQ ID NO:26. More preferably, one or more of these cysteine residues participate in disulphide bond formation under physiological conditions. Disulphide bond formation is often integral to the correct conformation of a protein and thus, its function. It is therefore important that such cysteine residues be conserved.

The polypeptide having the sequence recited in SEQ ID NO:18 is referred to hereafter as “the INSP125 exon 1 polypeptide”. The polypeptide having the sequence recited in SEQ ID NO:20 is referred to hereafter as “the INSP125 exon 2 polypeptide”. The polypeptide having the sequence recited in SEQ ID NO:22 is referred to hereafter as “the INSP125 exon 3 polypeptide”. The polypeptide having the sequence recited in SEQ ID NO:24 is referred to hereafter as “the INSP125 exon 4 polypeptide”.

The polypeptide having the sequence recited in SEQ ID NO:26 is referred to hereafter as “the INSP125 polypeptide”.

A small amount of EST data, mostly from rodent ESTs, suggests that the INSP125 sequence should be found in nerve tissue.

Although the Applicant does not wish to be bound by this theory, it is postulated that the first 23 amino acids of INSP125 exon 1 polypeptide form the signal peptide. The INSP125 exon 1 and full length polypeptide sequences without the signal sequence are recited in SEQ ID NO:28 and SEQ ID NO:30, respectively. The polypeptide having the sequence recited in SEQ ID NO:28 is referred to hereafter as “the INSP124 exon 1 mature polypeptide”. The polypeptide having the sequence recited in SEQ ID NO:30 is referred to hereafter as “the INSP125 mature polypeptide”.

Alternatively, although the Applicant does not wish to be bound by this theory, it is postulated that the first 22 amino acids of INSP125 cloned polypeptide form the signal peptide. The INSP125 cloned full length polypeptide sequences with and without the signal sequence are recited in SEQ ID NO:55 and SEQ ID NO:57, respectively. The polypeptide having the sequence recited in SEQ ID NO:55 is referred to hereafter as “the INSP125 cloned polypeptide”. The polypeptide having the sequence recited in SEQ ID NO:57 is referred to hereafter as “the INSP125 cloned mature polypeptide 1”.

Alternatively and preferably, although the Applicant does not wish to be bound by this theory, it is postulated that the first 21 amino acids of INSP125 cloned polypeptide form the signal peptide. The INSP125 cloned full length polypeptide sequences with and without the signal sequence are recited in SEQ ID NO:55 and SEQ ID NO:59, respectively. The polypeptide having the sequence recited in SEQ ID NO:55 is referred to hereafter as “the INSP125 cloned polypeptide”. The polypeptide having the sequence recited in SEQ ID NO:59 is referred to hereafter as “the INSP125 cloned mature polypeptide 2”.

Alternatively and preferably, although the Applicant does not wish to be bound by this theory, it is postulated that the first 31 amino acids of INSP125 cloned polypeptide form the signal peptide. The INSP125 cloned full length polypeptide sequences with and without the signal sequence are recited in SEQ ID NO:55 and SEQ ID NO:61, respectively. The polypeptide having the sequence recited in SEQ ID NO:55 is referred to hereafter as “the INSP125 cloned polypeptide”. The polypeptide having the sequence recited in SEQ ID NO:61 is referred to hereafter as “the INSP125 cloned mature polypeptide 3”.

The term “INSP125 exon polypeptides” as used herein includes polypeptides comprising the INSP125 exon 1 polypeptide, INSP125 exon 1 mature polypeptide, the INSP125 exon 2 polypeptide, the, the INSP125 exon 3 polypeptide, the INSP125 exon 4 polypeptide, the INSP125 polypeptide, INSP125 mature polypeptide 1, INSP125 mature polypeptide 2, or the INSP125 mature polypeptide 3, as well as polypeptides consisting of the INSP125 exon 1 polypeptide, the INSP125 exon 1 mature polypeptide, the INSP125 exon 2 polypeptide, the INSP125 exon 3 polypeptide, the INSP125 exon 4 polypeptide, the INSP125 polypeptide or the INSP125 mature polypeptide.

As already explained in the first aspect of the invention, the identification of novel proteins comprising vWFC domains is useful since such domains have been found to play an important role in a broad-cross section of diseases including those diseases associated with developmental processes such as those relating to cartilage and bone skeletal development.

In a fourth aspect, the invention provides a purified nucleic acid molecule which encodes a polypeptide of the second or third aspect of the invention.

Preferably, the purified nucleic acid molecule comprises the nucleic acid sequence as recited in SEQ ID NO:1 (encoding the INSP123 polypeptide), SEQ ID NO:3 (encoding the INSP123 mature polypeptide), SEQ ID NO:5 (encoding the INSP124 exon 1 polypeptide), SEQ ID NO:7 (encoding the INSP124 exon 2 polypeptide), SEQ ID NO:9 (encoding the INSP124 exon 3 polypeptide), SEQ ID NO:11 (encoding the INSP124 polypeptide), SEQ ID NO:13 (encoding the INSP124 mature polypeptide), SEQ ID NO:15 (encoding the INSP124 exon 1 mature polypeptide), SEQ ID NO:17 (encoding the INSP125 exon 1 polypeptide), SEQ ID NO:19 (encoding the INSP125 exon 2 polypeptide), SEQ ID NO:21 (encoding the INSP125 exon 3 polypeptide), SEQ BD NO:23 (encoding the INSP125 exon 4 polypeptide), SEQ ID NO:25 (encoding the INSP125 polypeptide), SEQ ID NO:27 (encoding the INSP125 exon 1 mature polypeptide), SEQ ID NO:29 (encoding the INSP125 mature polypeptide), SEQ ID NO:38 (encoding the INSP123 cloned polypeptide), SEQ ID NO:40 (encoding the INSP123 cloned mature polypeptide 1), SEQ ID NO:42 (encoding the INSP123 cloned mature polypeptide 2), SEQ ID NO:44 (encoding the INSP123 cloned mature polypeptide 3), SEQ ID NO:46 (encoding the INSP124 cloned polypeptide), SEQ ID NO:48 (encoding the INSP124 cloned mature polypeptide 1), SEQ ID NO:50 (encoding the INSP124 cloned mature polypeptide 2), SEQ ID NO:52 (encoding the INSP124 cloned mature polypeptide 3), SEQ ID NO:54 (encoding the INSP125 cloned polypeptide), SEQ ID NO:56 (encoding the INSP125 cloned mature polypeptide 1), SEQ ID NO:58 (encoding the INSP125 cloned mature polypeptide 2), and/or SEQ ID NO:60 (encoding the INSP125 cloned mature polypeptide), or is a redundant equivalent or fragment of any one of these sequences.

The invention further provides that the purified nucleic acid molecule consists of the nucleic acid sequence as recited in SEQ ID NO:1 (encoding the INSP123 polypeptide), SEQ ID NO:3 (encoding the INSP123 mature polypeptide), SEQ ID NO:5 (encoding the INSP124 exon 1 polypeptide), SEQ ID NO:7 (encoding the INSP124 exon 2 polypeptide), SEQ ID NO:9 (encoding the INSP124 exon 3 polypeptide), SEQ ID NO:11 (encoding the INSP124 polypeptide), SEQ ID NO:13 (encoding the INSP124 mature polypeptide), SEQ ID NO:15 (encoding the INSP124 exon 1 mature polypeptide), SEQ ID NO:17 (encoding the INSP125 exon 1 polypeptide), SEQ ID NO:19 (encoding the INSP125 exon 2 polypeptide), SEQ ID NO:21 (encoding the INSP125 exon 3 polypeptide), SEQ ID NO:23 (encoding the INSP125 exon 4 polypeptide), SEQ ID NO:25 (encoding the INSP125 polypeptide), SEQ ID NO:27 (encoding the INSP125 exon 1 mature polypeptide), SEQ ID NO:29 (encoding the INSP125 mature polypeptide), SEQ ID NO:38 (encoding the INSP123 cloned polypeptide), SEQ ID NO:40 (encoding the INSP123 cloned mature polypeptide 1), SEQ ID NO:42 (encoding the INSP123 cloned mature polypeptide 2), SEQ ID NO:44 (encoding the INSP123 cloned mature polypeptide 3), SEQ ID NO:46 (encoding the INSP124 cloned polypeptide), SEQ ID NO:48 (encoding the INSP124 cloned mature polypeptide 1), SEQ ID NO:50 (encoding the INSP124 cloned mature polypeptide 2), SEQ ID NO:52 (encoding the INSP124 cloned mature polypeptide 3), SEQ ID NO:54 (encoding the INSP125 cloned polypeptide), SEQ ID NO:56 (encoding the INSP125 cloned mature polypeptide 1), SEQ ID NO:58 (encoding the INSP125 cloned mature polypeptide 2), and/or SEQ ID NO:60 (encoding the INSP125 cloned mature polypeptide 3), or is a redundant equivalent or fragment of any one of these sequences.

According to one embodiment of this aspect of the invention, the purified nucleic acid molecule excludes the signal peptide located at the start of the INSP123 polypeptide (amino acids 1 to 23 of SEQ ID NO:2), the INSP124 exon 1 polypeptide (amino acids 1 to 23 of SEQ ID NO:6), the INSP124 polypeptide (amino acids 1 to 23 of SEQ ID NO:12), the INSP125 exon 1 polypeptide (amino acids 1 to 23 of SEQ ID NO:18) or the INSP125 polypeptide (amino acids 1 to 23 of SEQ ID NO:26). According to this embodiment, the purified nucleic acid molecule preferably comprises nucleotides 70 to 417 of SEQ ID NO:1 (shown in SEQ ID NO:3, encoding the INSP123 mature polypeptide), nucleotides 70 to 390 of SEQ ID NO:5 (shown in SEQ ID NO:13, encoding the INSP124 exon 1 mature polypeptide), nucleotides 70 to 669 of SEQ ID NO:11 (shown in SEQ ID NO:15, encoding the INSP124 mature polypeptide), nucleotides 70 to 100 of SEQ ID NO:17, (shown in SEQ ID NO:27, encoding the INSP125 exon 1 mature polypeptide) or nucleotides 70 to 528 of SEQ ID NO:25 (shown in SEQ ID NO:29, encoding the INSP125 mature polypeptide).

Alternatively, according to a second embodiment of this aspect of the invention, the purified nucleic acid molecule excludes the signal peptide located at the start of the INSP123 polypeptide (amino acids 1 to 22 of SEQ ID NO:39), the INSP124 polypeptide (amino acids 1 to 22 of SEQ ID NO:43), or the INSP125 polypeptide (amino acids 1 to 22 of SEQ ID NO:47). According to this embodiment, the purified nucleic acid molecule preferably comprises nucleotides 67 to 414 of SEQ ID NO:38 (shown in SEQ ID NO:40, encoding the INSP123 cloned mature polypeptide 1), nucleotides 67 to 666 of SEQ ID NO:46 (shown in SEQ ID NO:48, encoding the INSP124 cloned mature polypeptide 1), or nucleotides 67 to 525 of SEQ ID NO:54 (shown in SEQ ID NO:56, encoding the INSP125 cloned mature polypeptide 1).

Alternatively and preferably, according to a third embodiment of this aspect of the invention, the purified nucleic acid molecule excludes the signal peptide located at the start of the INSP123 polypeptide (amino acids 1 to 21 of SEQ ID NO:39), the INSP124 polypeptide (amino acids 1 to 21 of SEQ ID NO:47), or the INSP125 polypeptide (amino acids 1 to 21 of SEQ ID NO:55). According to this embodiment, the purified nucleic acid molecule preferably comprises nucleotides 64 to 414 of SEQ ID NO:38 (shown in SEQ ID NO:42, encoding the INSP123 cloned mature polypeptide 2), nucleotides 44 to 666 of SEQ ID NO:46 (shown in SEQ ID NO:50, encoding the INSP124 cloned mature polypeptide 2), or nucleotides 64 to 525 of SEQ ID NO:54 (shown in SEQ ID NO:58, encoding the INSP125 cloned mature polypeptide 2).

Alternatively and preferably, according to a fourth embodiment of this aspect of the invention, the purified nucleic acid molecule excludes the signal peptide located at the start of the INSP123 polypeptide (amino acids 1 to 31 of SEQ ID NO:39), the INSP124 polypeptide (amino acids 1 to 31 of SEQ ID NO:47), or the INSP125 polypeptide (amino acids 1 to 31 of SEQ ID NO:55). According to this embodiment, the purified nucleic acid molecule preferably comprises nucleotides 94 to 414 of SEQ ID NO:38 (shown in SEQ ID NO:44, encoding the INSP123 cloned mature polypeptide 3), nucleotides 94 to 666 of SEQ ID NO:46 (shown in SEQ ID NO:52, encoding the INSP124 cloned mature polypeptide 3), or nucleotides 94 to 525 of SEQ ID NO:54 (shown in SEQ ID NO:60, encoding the INSP125 cloned mature polypeptide 3).

The invention further provides a purified nucleic acid molecule consisting of nucleotides 70 to 417 of SEQ ID NO:1 (shown in SEQ ID NO:3, encoding the INSP123 mature polypeptide), nucleotides 70 to 390 of SEQ ID NO:5 (shown in SEQ ID NO:13, encoding the INSP124 exon 1 mature polypeptide), nucleotides 70 to 669 of SEQ ID NO:11 (shown in SEQ ID NO:15, encoding the INSP124 mature polypeptide), nucleotides 70 to 100 of SEQ ID NO:17, (shown in SEQ ID NO:27, encoding the INSP125 exon 1 mature polypeptide), nucleotides 70 to 528 of SEQ ID NO:25 (shown in SEQ ID NO:29, encoding the INSP125 mature polypeptide), nucleotides 67 to 414 of SEQ ID NO:38 (shown in SEQ ID NO:40, encoding the INSP123 cloned mature polypeptide 1), nucleotides 64 to 414 of SEQ ID NO:38 (shown in SEQ ID NO:42, encoding the INSP123 cloned mature polypeptide 2), nucleotides 94 to 414 of SEQ ID NO:38 (shown in SEQ ID NO:44, encoding the INSP123 cloned mature polypeptide 3), nucleotides 67 to 666 of SEQ ID NO:46 (shown in SEQ ID NO:48, encoding the INSP124 cloned mature polypeptide 1), nucleotides 44 to 666 of SEQ ID NO:46 (shown in SEQ ID NO:50, encoding the INSP124 cloned mature polypeptide 2), nucleotides 94 to 666 of SEQ ID NO:46 (shown in SEQ ID NO:52, encoding the INSP124 cloned mature polypeptide 3), nucleotides 67 to 525 of SEQ ID NO:54 (shown in SEQ ID NO:56, encoding the INSP125 cloned mature polypeptide 1), nucleotides 64 to 525 of SEQ ID NO:54 (shown in SEQ ID NO:58, encoding the INSP125 cloned mature polypeptide 2), or nucleotides 94 to 525 of SEQ ID NO:54 (shown in SEQ ID NO:60, encoding the INSP125 cloned mature polypeptide 3).

In a fifth aspect, the invention provides a purified nucleic acid molecule which hybridizes under high stringency conditions with a nucleic acid molecule of the fourth aspect of the invention.

In a sixth aspect, the invention provides a vector, such as an expression vector, that contains a nucleic acid molecule of the fourth or fifth aspect of the invention. Preferred vectors include pCR4-TOPO-INSP123 (FIG. 9), pDONR (FIG. 10), pEAK12d (FIG. 11), pDEST12.2 (FIG. 12), pENTR-INSP123-6HIS (FIG. 13), pEAK12d-INSP123-6HIS (FIG. 14), pDEST12.2-INSP123-6HIS (FIG. 15), pCR4-BluntII-TOPO-INSP124 (FIG. 19), pDONR 221 (FIG. 20), pEAK12d (FIG. 21), pDEST12.2 (FIG. 22), pENTR_INSP124-6HIS (FIG. 23), pEAK12d_INSP124-6HIS (FIG. 24), pDEST12.2_INSP124-6HIS (FIG. 25), pCR4-TOPO-INSP125 (FIG. 29), pDONR 221 (FIG. 30), pEAK12d (FIG. 31), pDEST12.2 (FIG. 32), pENTR_INSP125-6HIS (FIG. 33), pEAK12d-INSP125-6HIS (FIG. 34) and pDEST12.2_INSP125-6HIS (FIG. 35) expression vectors.

In a seventh aspect, the invention provides a host cell transformed with a vector of the sixth aspect of the invention.

In an eighth aspect, the invention provides a ligand which binds specifically to a member of the vWFC domain containing protein family of the second or third aspect of the invention.

In a ninth aspect, the invention provides a compound that is effective to alter the expression of a natural gene which encodes a polypeptide of the second or third aspect of the invention or to regulate the activity of a polypeptide of the second or third aspect of the invention.

A compound of the ninth aspect of the invention may either increase (agonise) or decrease (antagonise) the level of expression of the gene or the activity of the polypeptide.

Importantly, the identification of the function of the INSP123, INSP124 and INSP125 polypeptides allows for the design of screening methods capable of identifying compounds that are effective in the treatment and/or diagnosis of disease. Ligands and compounds according to the eighth and ninth aspects of the invention may be identified using such methods. These methods are included as aspects of the present invention.

In a tenth aspect, the invention provides a polypeptide of the second or third aspect of the invention, or a nucleic acid molecule of the fourth or fifth aspect of the invention, or a vector of the sixth aspect of the invention, or a host cell of the seventh aspect of the invention, or a ligand of the eighth aspect of the invention, or a compound of the ninth aspect of the invention, for use in therapy or diagnosis of diseases in which members of the vWFC domain containing protein family are implicated. Such diseases may include cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders such as those relating to cartilage and bone skeletal development, including osteoarthritis; metabolic disorders including diabetes mellitus, osteoporosis, and obesity, AIDS and renal disease; infections including viral infection, bacterial infection, fungal infection and parasitic infection and other pathological conditions. Preferably, the diseases are those in which vWFC domain containing proteins are implicated. These molecules may also be used in the manufacture of a medicament for the treatment of such diseases. These molecules may also be used in contraception or for the treatment of reproductive disorders including infertility.

In an eleventh aspect, the invention provides a method of diagnosing a disease in a patient, comprising assessing the level of expression of a natural gene encoding a polypeptide of the second or third aspect of the invention or the activity of a polypeptide of the second or third aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

A preferred method for detecting polypeptides of the second or third aspect of the invention comprises the steps of: (a) contacting a ligand, such as an antibody, of the eighth aspect of the invention with a biological sample under conditions suitable for the formation of a ligand-polypeptide complex; and (b) detecting said complex.

A number of different such methods according to the eleventh aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a twelfth aspect, the invention provides for the use of a polypeptide of the second or third aspect of the invention as a vWFC domain containing protein. Suitable uses of the polypeptides of the invention as vWFC domain containing proteins include use as a regulator of cellular growth, metabolism or differentiation, use as part of a receptor/ligand pair and use as a diagnostic marker for a physiological or pathological condition.

In an thirteenth aspect, the invention provides a pharmaceutical composition comprising a polypeptide of the second or third aspect of the invention, or a nucleic acid molecule of the fourth or fifth aspect of the invention, or a vector of the sixth aspect of the invention, or a host cell of the seventh aspect of the invention, or a ligand of the eighth aspect of the invention, or a compound of the ninth aspect of the invention, in conjunction with a pharmaceutically-acceptable carrier.

In a fourteenth aspect, the present invention provides a polypeptide of the second or third aspect of the invention, or a nucleic acid molecule of the fourth or fifth aspect of the invention, or a vector of the sixth aspect of the invention, or a host cell of the seventh aspect of the invention, or a ligand of the eighth aspect of the invention, or a compound of the ninth aspect of the invention, for use in the manufacture of a medicament for the diagnosis or treatment of a disease.

In a fifteenth aspect, the invention provides a method of treating a disease in a patient comprising administering to the patient a polypeptide of the second or third aspect of the invention, or a nucleic acid molecule of the fourth or fifth aspect of the invention, or a vector of the sixth aspect of the invention, or a host cell of the seventh aspect of the invention, or a ligand of the eighth aspect of the invention, or a compound of the ninth aspect of the invention.

For diseases in which the expression of a natural gene encoding a polypeptide of the second or third aspect of the invention, or in which the activity of a polypeptide of the second or third aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

In a sixteenth aspect, the invention provides transgenic or knockout non-human animals that have been transformed to express higher, lower or absent levels of a polypeptide of the second or third aspect of the invention. Such transgenic animals are very useful models for the study of disease and may also be used in screening regimes for the identification of compounds that are effective in the treatment or diagnosis of such a disease.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification Principles and Practice, Second Edition (Springer Verlag, N.Y.); and Handbook of Experimental Immunology, Volumes I-IV (D. M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro-protein that can be activated by cleavage of the pre-, pro- or prepro-portion to produce an active mature polypeptide. In such polypeptides, the pre-, pro- or prepro-sequence may be a leader or secretory sequence or may be a sequence that is employed for purification of the mature polypeptide sequence.

The polypeptide of the second or third aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The functionally-equivalent polypeptides of the third aspect of the invention may be polypeptides that are homologous to the INSP123, INSP124 and INSP125 polypeptides. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

Homologous polypeptides therefore include natural biological variants (for example, allelic variants or geographical variations within the species from which the polypeptides are derived) and mutants (such as mutants containing amino acid substitutions, insertions or deletions) of the INSP123, INSP124 and INSP125 polypeptides. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. Such mutants also include polypeptides in which one or more of the amino acid residues includes a substituent group.

Typically, greater than 30% identity between two polypeptides is considered to be an indication of functional equivalence. Preferably, functionally equivalent polypeptides of the second or third aspect of the invention have a degree of sequence identity with the INSP123, INSP124 or INSP125 polypeptides, or with active fragments thereof, of greater than 80%. More preferred polypeptides have degrees of identity of greater than 85%, 90%, 95%, 98% or 99%, respectively.

The functionally-equivalent polypeptides of the second or third aspect of the invention may also be polypeptides which have been identified using one or more techniques of structural alignment. For example, the Inpharmatica Genome Threader technology that forms one aspect of the search tools used to generate the Biopendium™ search database may be used (see PCT application WO 01/69507) to identify polypeptides of presently-unknown function which, while having low sequence identity as compared to the INSP123, INSP124 and INSP125 polypeptides, are predicted to be members of the vWFC domain containing protein family, by virtue of sharing significant structural homology with the INSP123, INSP124 and INSP125 polypeptide sequences. By “significant structural homology” is meant that the Inpharmatica Genome Threader predicts two proteins to share structural homology with a certainty of 10% and above.

The polypeptides of the second or third aspect of the invention also include fragments of the INSP123, INSP124 and INSP125 polypeptides and fragments of the functional equivalents of the INSP123, INSP124 and INSP125 polypeptides, provided that those fragments are members of the vWFC containing protein family or have an antigenic determinant in common with the INSP123, INSP124 and INSP125 polypeptides.

As used herein, the term “fragment” refers to a polypeptide having an amino acid sequence that is the same as part, but not all, of the amino acid sequence of the INSP123, INSP124, and INSP125 polypeptides or one of their functional equivalents. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant.

Fragments of the full length INSP123, INSP124 and INSP125 polypeptides may consist of combinations of 2, 3 or 4 of neighbouring exon sequences in the INSP123, INSP124, and INSP125 polypeptide sequences, respectively.

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “protein” means a type of polypeptide including, but not limited to those that function as enzymes. Preferably, the protein or polypeptide of the present invention functions as a ligand. A ligand, in this context means a molecule that binds to another molecule, such as a receptor. A ligand may be a co-factor for an enzyme. The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)₂ and Fv, which are capable of binding to the antigenic determinant in question. Such antibodies thus bind to the polypeptides of the second or third aspect of the invention.

If polyclonal antibodies are desired, a selected mammal, such as a mouse, rabbit, goat or horse, may be immunised with a polypeptide of the second or third aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet haemocyanin. The coupled polypeptide is then used to immunise the animal. Serum from the immunised animal is collected and treated according to known procedures, for example by immunoaffinity chromatography.

Monoclonal antibodies to the polypeptides of the second or third aspect of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985).

Panels of monoclonal antibodies produced against the polypeptides of the second or third aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239, 1534 (1988); Kabat et al., J. Immunol., 147, 1709 (1991); Queen et al., Proc. Natl. Acad. Sci. USA, 86, 10029 (1989); Gorman et al., Proc. Natl. Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays, radioimmunoassays (RIA) or enzyme-inked immunosorbent assays (ELISA). In these applications, the antibodies can be labelled with an analytically-detectable reagent such as a radioisotope, a fluorescent molecule or an enzyme.

Preferred nucleic acid molecules of the fourth and fifth aspects of the invention are those which encode a polypeptide sequence as recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59 and SEQ ID NO:61 and functionally equivalent polypeptides. These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones, and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8:53-63).

A nucleic acid molecule which encodes a polypeptide of this invention may be identical to the coding sequence of one or more of the nucleic acid molecules disclosed herein.

These molecules also may have a different sequence which, as a result of the degeneracy of the genetic code, encodes a polypeptide SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30 SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59 and/or SEQ ID NO:61. Such nucleic acid molecules may include, but are not limited to, the coding sequence for the mature polypeptide by itself; the coding sequence for the mature polypeptide and additional coding sequences, such as those encoding a leader or secretory sequence, such as a pro-, pre- or prepro-polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the fourth and fifth aspects of the invention may also encode the fragments or the functional equivalents of the polypeptides and fragments of the second or third aspect of the invention. Such a nucleic acid molecule may be a naturally-occurring variant such as a naturally-occurring allelic variant, or the molecule may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the second or third aspect of the invention may be ligated to a heterologous sequence so that the combined nucleic acid molecule encodes a fusion protein. Such combined nucleic acid molecules are included within the fourth or fifth aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridize to the encoding nucleic acid molecules (hybridization). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J. S., Trends in Pharm. Sci., 10, 435 (1989), Okano, J. Neurochem. 56, 560 (1991); O'Connor, J. Neurochem 56, 560 (1991); Lee et al., Nucleic Acids Res 6, 3073 (1979); Cooney et al., Science 241, 456 (1988); Dervan et al., Science 251, 1360 (1991).

The term “hybridization” as used here refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. [supra]).

The inhibition of hybridization of a completely complementary molecule to a target molecule may be examined using a hybridization assay, as known in the art (see, for example, Sambrook et al. [supra]). A substantially homologous molecule will then compete for and inhibit the binding of a completely homologous molecule to the target molecule under various conditions of stringency, as taught in Wahl, G. M. and S. L. Berger (1987; Methods Enzymol. 152:399-407) and Kimmel, A. R. (1987; Methods Enzymol. 152:507-511).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C. Low stringency conditions involve the hybridisation reaction being carried out at 35° C. (see Sambrook et at [supra]). Preferably, the conditions used for hybridization are those of high stringency.

Preferred embodiments of this aspect of the invention are nucleic acid molecules that are at least 70% identical over their entire length to a nucleic acid molecule encoding the INSP123, INSP124 or INSP125 polypeptides and nucleic acid molecules that are substantially complementary to such nucleic acid molecules. Preferably, a nucleic acid molecule according to this aspect of the invention comprises a region that is at least 80% identical over its entire length to such coding sequences, or is a nucleic acid molecule that is complementary thereto. In this regard, nucleic acid molecules at least 90%, preferably at least 95%, more preferably at least 98%, 99% or more identical over their entire length to the same are particularly preferred. Preferred embodiments in this respect are nucleic acid molecules that encode polypeptides which retain substantially the same biological function or activity as the INSP123, INSP124 and INSP125 polypeptides.

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed.

As discussed additionally below in connection with assays that may be utilised according to the invention, a nucleic acid molecule as described above may be used as a hybridization probe for RNA, cDNA or genomic DNA, in order to isolate full-length cDNAs and genomic clones encoding the INSP123, INSP124 and INSP125 polypeptides and to isolate cDNA and genomic clones of homologous or orthologous genes that have a high sequence similarity to the gene encoding this polypeptide.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase I, Sequenase (US Biochemical Corp, Cleveland, Ohio), Taq polymerase Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; MJ Research, Watertown, Mass.) and the ABI Catalyst and 373 and 377 DNA Sequencers (Perkin Elmer).

One method for isolating a nucleic acid molecule encoding a polypeptide with an equivalent function to that of the INSP123, INSP124 and INSP125 polypeptides is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989, 1992). Probes comprising at least 15, preferably at least 30, and more preferably at least 50, contiguous bases that correspond to, or are complementary to, nucleic acid sequences from the appropriate encoding gene (SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58 and SEQ ID NO:60), are particularly useful probes. Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., PNAS USA 85, 8998-9002, 1988). Recent modifications of this technique, exemplified by the Marathon™ technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T. et al. (1988) Nucleic Acids Res. 16:8186). Another method which may be used is capture PCR which involves PCR amplification of DNA fragments adjacent a known sequence in human and yeast artificial chromosome DNA (Lagerstrom, M. et al. (1991) PCR Methods Applic., 1, 111-119). Another method which may be used to retrieve unknown sequences is that of Parker, J. D. et al (1991); Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridize with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

The vectors of the present invention comprise nucleic acid molecules of the invention and may be cloning or expression vectors. The host cells of the invention, which may be transformed, transfected or transduced with the vectors of the invention may be prokaryotic or eukaryotic.

The polypeptides of the invention may be prepared in recombinant form by expression of their encoding nucleic acid molecules in vectors contained within a host cell. Such expression methods are well known to those of skill in the art and many are described in detail by Sambrook et al. (supra) and Fernandez & Hoeffler (1998, eds. “Gene expression systems. Using nature for the art of expression”. Academic Press, San Diego, London, Boston, New York, Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid. The vectors pCR4-TOPO-INSP123 (FIG. 9), pDONR (FIG. 10), pEAK12d (FIG. 11), pDEST12.2 (FIG. 12), pENTR-INSP123-6HIS (FIG. 13), pEAK12d-INSP123-6HIS (FIG. 14), pDEST12.2-INSP123-6HIS (FIG. 15), pCR4-BluntII-TOPO-INSP124 (FIG. 19), pDONR 221 (FIG. 20), pEAK12d (FIG. 21), pDEST12.2 (FIG. 22), pENTR_INSP124-6HIS (FIG. 23), pEAK12d-INSP124-6HIS (FIG. 24), pDEST12.2_INSP124-6HIS (FIG. 25), pCR4-TOPO-INSP125 (FIG. 29), pDONR 221 (FIG. 30), pEAK12d (FIG. 31), pDEST12.2 (FIG. 32), pENTR_INSP125-6HIS (FIG. 33), pEAK12d_INSP125-6HIS (FIG. 34) and pDEST12.2_INSP125-6HIS (FIG. 35) are preferred examples of suitable vectors for use in accordance with the invention.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al., Basic Methods in Molecular Biology (1986) and Sambrook et al., (supra). Particularly suitable methods include calcium phosphate transfection, DEAE-dextran mediated transfection, transfection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out transcription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSportl™ plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; and U.S. Pat. No. 5,608,143. Additional examples of genetic expression in plant cell culture has been described by Zenk, Phytochemistry 30, 3861-3863 (1991).

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli, Streptomyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk⁻ or aprt^{±} cells, respectively.

Also, antimetabolite, antibiotic or herbicide resistance can be used as the basis for selection; for example, dihydrofolate reductase (DHFR) that confers resistance to methotrexate (Wigler, M. et al. (1980) Proc. Natl. Acad. Sci. 77:3567-70); npt, which confers resistance to the aminoglycosides neomycin and G418 (Colbere-Garapin, F. et al. (1981) J. Mol. Biol. 150:1-14) and als or pat, which confer resistance to chlorsulfuron and phosphinotricin acetyltransferase, respectively. Additional selectable genes have been described, examples of which will be clear to those of skill in the art.

Although the presence or absence of marker gene expression suggests that the gene of interest is also present, its presence and expression may need to be confined. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single promoter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridizations and protein bioassays, for example, fluorescence activated cell sorting (FACS) or immunoassay techniques (such as the enzyme-linked immunosorbent assay [ELISA] and radioimmunoassay [RIA]), that include membrane, solution, or chip based technologies for the detection and/or quantification of nucleic acid or protein (see Hampton, R. et al. (1990) Serological Methods, a Laboratory Manual, APS Press, St Paul, Minn.) and Maddox, D. E. et al. (1983) J. Exp. Med, 158, 1211-1216).

A wide variety of labels and conjugation techniques are known by those skilled in the art and may be used in various nucleic acid and amino acid assays. Means for producing labelled hybridization or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison Wis.); and U.S. Biochemical Corp., Cleveland, Ohio)).

Suitable reporter molecules or labels, which may be used for ease of detection, include radionuclides, enzymes and fluorescent, chemiluminescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography is particularly useful for purification. Well known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immobilised metals, protein A domains that allow purification on immobilised immunoglobulin, and the domain utilised in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterokinase cleavage site. The histidine residues facilitate purification by IMAC (immobilised metal ion affinity chromatography as described in Porath, J. et al. (1992), Prot. Exp. Purif. 3: 263-281) while the thioredoxin or enterokinase cleavage site provides a means for purifying the polypeptide from the fusion protein. A discussion of vectors which contain fusion proteins is provided in Kroll, D. J. et al. (1993; DNA Cell Biol. 12:441 A53).

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

The polypeptide of the invention can be used to screen libraries of compounds in any of a variety of drug screening techniques. Such compounds may activate (agonise) or inhibit (antagonise) the level of expression of the gene or the activity of the polypeptide of the invention and form a further aspect of the present invention. Preferred compounds are effective to alter the expression of a natural gene which encodes a polypeptide of the second or third aspect of the invention or to regulate the activity of a polypeptide of the second or third aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

A preferred method for identifying an agonist or antagonist compound of a polypeptide of the present invention comprises:

(a) contacting a cell expressing on the surface thereof the polypeptide according to the second or third aspect of the invention, the polypeptide being associated with a second component capable of providing a detectable signal in response to the binding of a compound to the polypeptide, with a compound to be screened under conditions to permit binding to the polypeptide; and

(b) determining whether the compound binds to and activates or inhibits the polypeptide by measuring the level of a signal generated from the interaction of the compound with the polypeptide.

A further preferred method for identifying an agonist or antagonist of a polypeptide of the invention comprises:

(a) contacting a cell expressing on the surface thereof the polypeptide, the polypeptide being associated with a second component capable of providing a detectable signal in response to the binding of a compound to the polypeptide, with a compound to be screened under conditions to permit binding to the polypeptide; and

(b) determining whether the compound binds to and activates or inhibits the polypeptide by comparing the level of a signal generated from the interaction of the compound with the polypeptide with the level of a signal in the absence of the compound.

In further preferred embodiments, the general methods that are described above may further comprise conducting the identification of agonist or antagonist in the presence of labelled or unlabelled ligand for the polypeptide.

In another embodiment of the method for identifying an agonist or antagonist of a polypeptide of the present invention comprises:

determining the inhibition of binding of a ligand such as a receptor to cells which have a polypeptide of the invention on the surface thereof, or to cell membranes containing such a polypeptide, in the presence of a candidate compound under conditions to permit binding to the polypeptide, and determining the amount of ligand bound to the polypeptide. A compound capable of causing reduction of binding of a ligand is considered to be an agonist or antagonist. Preferably the ligand is labelled.

More particularly, a method of screening for a polypeptide antagonist or agonist compound comprises the steps of:

(a) incubating a labelled ligand with a whole cell expressing a polypeptide according to the invention on the cell surface, or a cell membrane containing a polypeptide of the invention,

(b) measuring the amount of labelled ligand bound to the whole cell or the cell membrane;

(c) adding a candidate compound to a mixture of labelled ligand and the whole cell or the cell membrane of step (a) and allowing the mixture to attain equilibrium;

(d) measuring the amount of labelled ligand bound to the whole cell or the cell membrane after step (c); and

(e) comparing the difference in the labelled ligand bound in step (b) and (d), such that the compound which causes the reduction in binding in step (d) is considered to be an agonist or antagonist.

The INSP123, INSP124 and INSP125 polypeptides of the present invention may modulate cellular growth and differentiation. Thus, the biological activity of the INSP123, INSP124 and INSP125 polypeptides can be examined in systems that allow the study of cellular growth and differentiation such as organ culture assays or in colony assay systems in agarose culture. Stimulation or inhibition of cellular proliferation may be measured by a variety of assays.

For example, for observing cell growth inhibition, one can use a solid or liquid medium. In a solid medium, cells undergoing growth inhibition can easily be selected from the subject cell group by comparing the sizes of colonies formed. In a liquid medium, growth inhibition can be screened by measuring culture medium turbidity or incorporation of labelled thymidine in DNA. Typically, the incorporation of a nucleoside analog into newly synthesised DNA may be employed to measure proliferation (i.e., active cell growth) in a population of cells. For example, bromodeoxyuridine (BrdU) can be employed as a DNA labelling reagent and anti-BrdU mouse monoclonal antibodies can be employed as a detection reagent. This antibody binds only to cells containing DNA which has incorporated bromodeoxyuridine. A number of detection methods may be used in conjunction with this assay including immunofluorescence, immunohistochemical, ELISA, and colorimetric methods. Kits that include bromodeoxyuridine (BrdU) and anti-BrdU mouse monoclonal antibody are commercially available from Boehringer Mannheim (Indianapolis, Ind.).

The effect of the INSP123, INSP124 and INSP125 polypeptides upon cellular differentiation can be measured by contacting stem cells or embryonic cells with various amounts of the INSP123, INSP124 and INSP125 polypeptides and observing the effect upon differentiation of the stem cells or embryonic cells. Tissue-specific antibodies and microscopy may be used to identify the resulting cells.

The INSP123, INSP124 and INSP125 polypeptides may also be found to modulate immune and/or nervous system cell proliferation and differentiation in a dose-dependent manner in the above-described assays. Thus, the “functional equivalents” of the INSP123, INSP124, and INSP125 polypeptides include polypeptides that exhibit any of the same growth and differentiation regulating activities in the above-described assays in a dose-dependent manner. Although the degree of dose-dependent activity need not be identical to that of the INSP123, INSP124 and INSP125 polypeptides, preferably the “functional equivalents” will exhibit substantially similar dose-dependence in a given activity assay compared to the INSP123, INSP124 and INSP125 polypeptides.

In certain of the embodiments described above, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

The polypeptide of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [X] is “substantially free of” impurities [herein, Y] when at least 85% by weight of the total X+Y in the composition is X. Preferably, X comprises at least about 90% by weight of the total of X+Y in the composition, more preferably at least about 95%, 98% or even 99% by weight.

The pharmaceutical compositions should preferably comprise a therapeutically effective amount of the polypeptide, nucleic acid molecule, ligand, or compound of the invention. The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intra-arterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression-blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter. 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent.

Vaccines according to the invention may either be prophylactic (i.e. to prevent infection) or therapeutic (i.e. to treat disease after infection). Such vaccines comprise immunising antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

This invention also relates to the use of nucleic acid molecules according to the present invention as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al., Nature, 324, 163-166 (1986); Bej, et al, Crit. Rev. Biochem. Molec. Biol., 26, 301-334 (1991); Birkenmeyer et al., J. Virol. Meth., 35, 117-126 (1991); Van Brunt, J., Bio/Technology, 8, 291-294 (1990)) prior to analysis.

In one embodiment, this aspect of the invention provides a method of diagnosing a disease in a patient, comprising assessing the level of expression of a natural gene encoding a polypeptide according to the invention and comparing said level of expression to a control level, wherein a level that is different to said control level is indicative of disease. The method may comprise the steps of:
a) contacting a sample of tissue from the patient with a nucleic acid probe under stringent conditions that allow the formation of a hybrid complex between a nucleic acid molecule of the invention and the probe;
b) contacting a control sample with said probe under the same conditions used in step a);
c) and detecting the presence of hybrid complexes in said samples;
   wherein detection of levels of the hybrid complex in the patient sample that differ from levels of the hybrid complex in the control sample is indicative of disease.

A further aspect of the invention comprises a diagnostic method comprising the steps of:
a) obtaining a tissue sample from a patient being tested for disease;
b) isolating a nucleic acid molecule according to the invention from said tissue sample; and
c) diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridizing amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and S1 protection or the chemical cleavage method (see Cotton et al., Proc. Natl. Acad. Sci. USA (1985) 85: 4397-4401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. Fluorescence in situ hybridization (FISH) is presently the most commonly applied method and numerous reviews of FISH have appeared (see, for example, Trachuck et al., Science, 250, 559-562 (1990), and Trask et al., Trends, Genet., 7, 149-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M. Chee et al., Science (1996), Vol 274, pp 610-613).

In one embodiment, the array is prepared and used according to the methods described in PCT application WO95/11995 (Chee et al); Lockhart, D. J. et al. (1996) Nat. Biotech. 14: 1675-1680); and Schena, M. et al. (1996) Proc. Natl. Acad. Sci. 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application WO95/25116 (Baldeschweiler et al). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation. The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:
(a) a nucleic acid molecule of the present invention;
(b) a polypeptide of the present invention; or
(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or susceptibility to disease in members of the vWFC domain containing protein family are implicated. Such diseases may include cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders such as those relating to cartilage and bone skeletal development, including osteoarthritis; metabolic disorders including diabetes mellitus, osteoporosis, and obesity, AIDS and renal disease; infections including viral infection, bacterial infection, fungal infection and parasitic infection and other pathological conditions. Preferably, the diseases are those in which lymphocyte antigens are implicated. Such kits may also be used for the detection of reproductive disorders including infertility.

Various aspects and embodiments of the present invention will now be described in more detail by way of example, with particular reference to the INSP123, INSP124 and INSP125 polypeptides.

It will be appreciated that modification of detail may be made without departing from the scope of the invention.

### BRIEF DESCRIPTION OF THE FIGURES

FIG. 1: Alignment of the SECFAM3 family. Von Willebrand Factor type C (vWFC) domain 1 spans the region 155-214aa of the alignment and vWFC domain 2 spans the region 221-281aa. INSP123, 124 and 125 have been shaded in grey in the “Id” column. Sequence number 14 and 15, labelled Chordate, in the alignment represent translated EST sequences from Ciona intestinalis species.

FIG. 2: INSP123, 124 and 125 were all predicted to be secreted proteins based on the prediction of a signal peptide common to all three isoforms (FIG. 2).

FIG. 3: Splicing patterns predicted for the coding exons of this gene (not to scale). INSP123 and INSP125 were based on mouse and macaque cDNA sequences, while INSP124 was a predicted possible splicing pattern that incorporated both von Willebrand Factor type C domains. The effect that this splicing had at the sequence level may be seen in FIG. 1.

FIG. 4: Alignment of INSP124 predicted domain 1 and domain 2 (highlighted) against characterized von Willebrand Factor type C domains from a variety of proteins. Darker shading indicates greater sequence conservation.

FIG. 5: Position-specific probability matrix profile of the family based on INSP124.

FIG. 6: Family consensus sequence in PROSITE format based on INSP124 amino acids 53 to 171 (SEQ ID NO:12) Key: −=a spacer between each alignment position; G=100% conserved G residue; [VI]=either a V or an I at that alignment position; P(0,1)=a P residue found once or not at all at this alignment position.

FIG. 7: Nucleotide sequence of INSP123 prediction with translation.

FIG. 8: Nucleotide sequence with translation of INSP123 PCR product cloned using primers INSP123-CP1 and INSP123-CP2.

FIG. 9: Map of pCR4-TOPO-INSP123.

FIG. 10: Map of pDONR 221.

FIG. 11: Map of expression vector pEAK12d.

FIG. 12: Map of Expression vector pDEST12.2.

FIG. 13: Map of pENTR-INSP123-6HIS.

FIG. 14: Map of pEAK12d-INSP1236HIS.

FIG. 15: Map of pDEST12.2-INSP1236HIS.

FIG. 16: Nucleotide sequence of INSP124 prediction with translation of the coding sequence.

FIG. 17: INSP124 coding exon organization in genomic DNA and position of PCR primers.

FIG. 18: Nucleotide sequence of cloned INSP124 product with translation of the ORF.

FIG. 19: Map of pCR-BluntII-TOPO-INSP124.

FIG. 20: Map of pDONR 221.

FIG. 21: Map of Expression vector pEAK12d.

FIG. 22: Map of Expression vector pDEST12.2.

FIG. 23: Map of pENTR_INSP124-6HIS.

FIG. 24: Map of pEAK12d_INSP124-6HIS.

FIG. 25: Map of pDEST12.2_INSP124-6HIS.

FIG. 26: Nucleotide sequence of INSP125 prediction with translation of the coding sequence

FIG. 27: INSP125 coding exon organization in genomic DNA and position of PCR primers.

FIG. 28: Nucleotide sequence of cloned INSP125 product with translation of the ORF.

FIG. 29: Map of pCR4-TOPO-INSP125.

FIG. 30: Map of pDONR 221.

FIG. 31: Map of Expression vector pEAK12d.

FIG. 32: Map of Expression vector pDEST12.2.

FIG. 33: Map of pENTR_INSP125-6HIS.

FIG. 34: Map of pEAK12d_INSP125-6HIS.

FIG. 35: Map of pDEST12.2_INSP125-6HIS.

FIG. 36: N-terminal sequencing results for INSP125-6HIS

### EXAMPLES

### Example 1

### Selecting and Aligning the SECFAM3 Family Members

INSP123, INSP124 and INSP125 have no publicly available annotation, contain a strong secretory protein signature in the form of a signal peptide, and can be clustered with similar proteins such as orthologues from other animal species.

Further examination permitted the construction of an uncharacterised family of proteins consisting of 22 sequences: 2 human genes (and their isoforms) and their vertebrate and chordate orthologues. A list of the 22 family members is given in Table 1.

TABLE 1
All of the sequences of the SECFAM3 family with peptide length and,
where possible, tissue distribution information included.

| Identifier in alignment | Sequence accession number |
|---|---|
| 1_[MACAQUE] | BAB60802.1 [adult brain] (138aa) |
| 2_INSP123 | ENSG00000174453 (Ensemble gene prediction) (138aa) |
| 3_[RAT] | Inpharmatica prediction (SEQ ID 30) (131aa) |
| 4_[MOUSE] | Inpharmatica prediction (SEQ ID 28) (131aa) |
| 5_[MOUSE] | XP_194760.2 (324aa) |
| 6_[HUMAN] | AAY96732 (Derwent sequence: “nell homologue”) No equivalent |
| | in NCBI. (325aa) |
| 7_[RAT] | Inpharmatica prediction (SEQ ID 31) (324aa) |
| 8_[CHICKEN] | BU281449.1 (EST: translated in frame +1) [adult brain - not |
| | cerebellum or cerebrum] (183aa) |
| 9_[CHICKEN] | BU361615.1 (EST: translated in frame +2) [adult cerebrum] |
| | (209aa) |
| 10_[ZEBRAFISH] | BM156647.1 (EST: translated in frame +3) [adult male whole |
| | body] (190aa) |
| 11_[MOUSE] | W41229.1 (EST: translation frame +3) [19.5 days post conception |
| | whole foetus] (86aa) |
| 12_[SALMON] | CA039900.1 (EST: translation frame +2) [spleen] (153aa) |
| 13_[FROG] | AL635358.1 (EST: translation frame +2) [nurula embryonic stage] |
| | (100aa) |
| 14_[Ciona intestinalis] | BW255450.1 (EST: translation frame +1) [cleaving embryo whole |
| | body] (182aa) |
| 15_[Ciona intestinalis] | AV674424.1 (EST: translation frame +2) [tail bud stage, whole |
| | body] (139aa) |
| 16_[CHICKEN] | BG711876.1 (EST: translation frame +1) [normalized liver] |
| | (134aa) |
| 17_[FUGU] | Inpharmatica prediction (SEQ ID 34) (131aa) |
| 18_[FUGU] | Inpharmatica prediction (SEQ ID 33) (247aa) |
| 19_[FUGU] | Inpharmatica prediction (SEQ ID 32) (223aa) |
| 20_[MOUSE] | Inpharmatica prediction (SEQ ID 29) (222aa) |
| 21_INSP124 | Inpharmatica prediction (SEQ Ids 5-16) (222aa) |
| 22_INSP125 | Inpharmatica prediction (SEQ Ids 17-27) (175aa) |

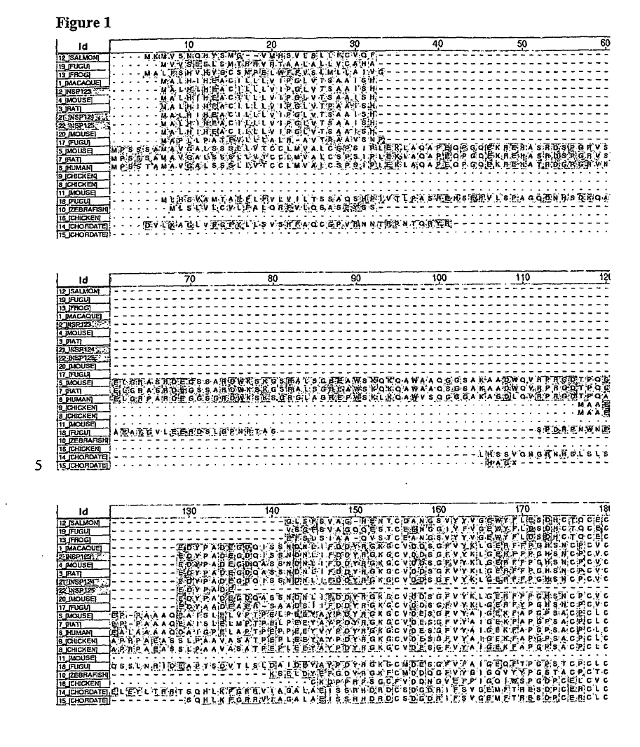

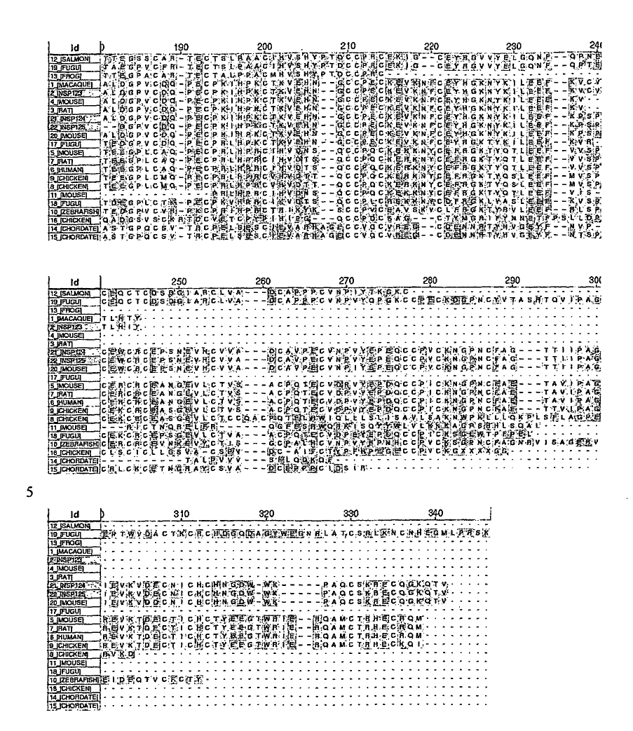

These sequences were aligned using the ClustalW tool (Thompson, J. D., Higgins, D. G., Gibson T. J. Nucleic Acids Res 1994 Nov. 11; 22(22):4673-80) (FIG. 1). From this alignment, the similarities and differences in the sequences can be clearly seen. Each of the proteins share a strong secretory protein signature in the form of a signal peptide and at least one vWFC domain.

Of the human sequences, INSP123 (SEQ ID NO:2), INSP124 (SEQ ID NO:6) and INSP125 (SEQ ID NO:26) are novel predictions that are not represented in the public or patent databases (e.g. NCBI, DDBJ and Derwent). No human cDNA encoding any of these three proteins has yet been identified. However, close homology to macaque and mouse cDNA sequences offers strong supporting evidence that the three INSP sequences disclosed are the human equivalent of the macaque and mouse sequences.

### Example 2

### Supporting Evidence for the Existence of INSP123, INSP124 and INSP125 Polypeptides

Macaque (Macaca fascicularis) cDNA

AB063096.1 (cDNA sequence), BAB60802.1 (protein sequence). Full insert sequence cDNA clone. (Adult male brain (right temporal lobe).)

Length=138aa.

INSP123 (SEQ ID NO: 2): 99% ID, Query 1-138aa, Target 1-138aa, e=7e-84.

Identical length with one amino acid difference.

INSP124: 100% ID, Query 1-130aa, Target 1-130aa, e=3e-79.

INSP125: 63% ID overall. Split into two regions of 100% ID (Query 1-33aa, Target 1-33aa, and Query 34-83aa, Target 81-130aa)

Mouse (Mus musculus)

AK083856.1 (Mus musculus 12 days embryo spinal ganglion cDNA, RIKEN full-length enriched library, clone:D130026K08 product:hypothetical von Willebrand factor, type C repeat containing protein, full insert sequence.) [NOTE: An extra G nucleotide (G 873) introduced a frame-shift in this sequence which was not supported by the genomic DNA for that region. When corrected, the sequence similarity to the translated Macaque cDNA above, and the restoration of the signal peptide, also adds support for the validity of the correction.]

The statistics for the translation of the corrected sequence are shown below:

Length=131aa.

INSP123: 99% ID, Query 1-131aa, Target 1-131aa, e=4e-79.

Identical length with one amino acid difference.

INSP124: 99% ID, Query 1-130aa, Target 1-130aa, e=1e-78.

INSP125: 63% ID overall. Split into two regions of 100% ID (Query 1-33aa, Target 1-33, and Query 34-83aa, Target 81-130aa).

AK080585.1 (Mus musculus 10 days neonate cortex cDNA, RIKEN full-length enriched library, product:hypothetical von Willebrand factor, type C repeat containing protein, full insert sequence.)

The statistics for the translated product are shown below:

Length=175aa.

INSP123: 63% ID overall. Splits into two regions of 100% ID (Query 1-33, Target 1-33, and Query 81-130aa, Target 34-83aa) with a spliced out region in between.

INSP124: 77% ID overall. Splits into two regions of 100% and 98% ID respectively (Query 1-33aa, Target 1-33aa, and Query 81-222aa, Target 34-175aa).

INSP125: 98% ID, Query 1-175aa, Target 1-175aa, e=e-122. (Identical length with two amino acids conservatively substituted.)

### Example 3

### Identifying the Signal Peptide Sequence

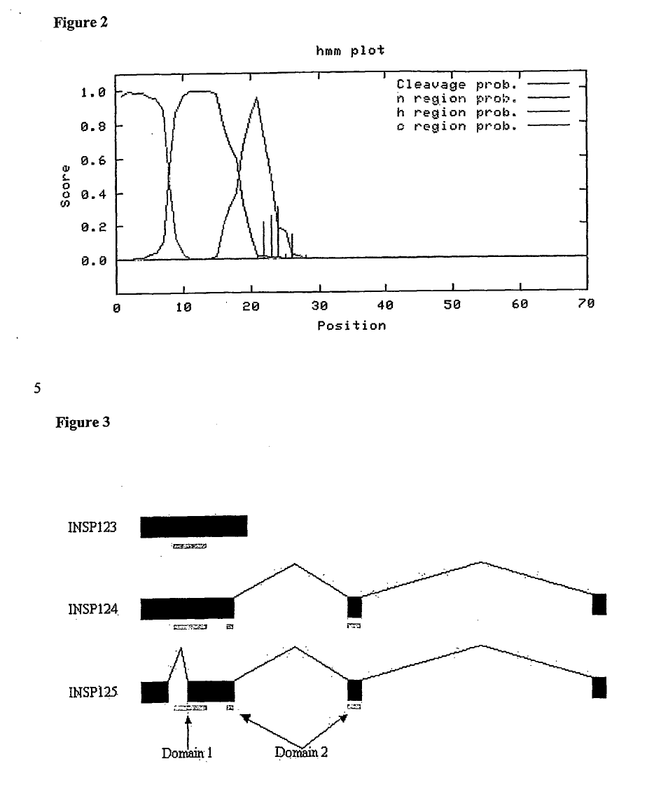

The SignalP program (http://www.cbs.dtu.dk/services/SignalP/) was used to identify the potential signal peptide regions and cleavage sites for the INSP123-125 polypeptides. Since these three polypeptides share the same initial sequence, the SignalP results were identical for the three isoforms, that is, the SignalP results for INSP123 (SEQ ID NO:2), INSP124 (SEQ ID NO:12) and INSP125 (SEQ ID NO:26) all indicate that the cleavage site is most likely to be between positions 23 and 24 (FIG. 2).

### Example 4

### Evidence for the Presence of a vWFC Domain within the SECFAM3 Family

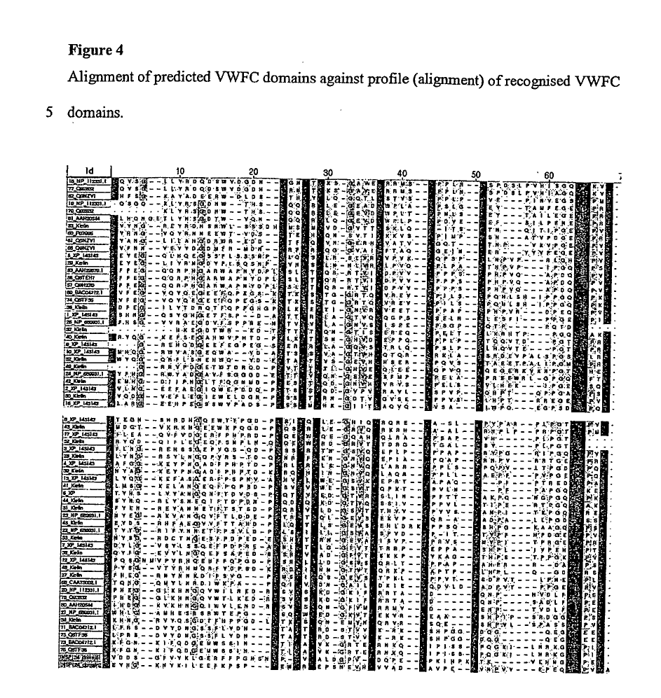

Each sequence in the family was compared to protein domain profiles. This process highlighted a (vWFC) domain at position 221 to 281aa of the SECFAM3 alignment (FIG. 1). A weak hit to the same domain type over alignment range 155 to 214aa indicated that there were two vWFC domains in the longer proteins of this family, and one in the shorter members (such as INSP123). The two predicted vWFC domains of INSP124 were extracted and aligned against a profile of over 50 characterized vWFC domains from a variety of proteins (FIG. 4). The 10-cysteine pattern was conserved in these regions along with some non-cysteine residues, confirming beyond reasonable doubt that both domains were indeed vWFC domain-like at the sequence level. Given the fact that the cysteine pattern is conserved it would seem probable that the structure of these domains would take on a similar shape to known vWFC domains. The two vWFC domains will henceforth be known as “domain 1” and “domain 2,” based on their order of appearance in the alignment.

### Example 5

### Splicing Patterns of INSP123, INSP124 and INSP125

INSP123 contains only domain 1 (53-109aa of SEQ ID NO:2), whereas INSP124 contains both domains (53-109aa and 116-171aa of SEQ ID NO:12). Isoform INSP125 (SEQ ID NO:26) is characterised in that there is a region spliced between positions 136 and 182 in the alignment (FIGS. 1 and 3). This effectively deletes the first four cysteines of domain 1, most likely rendering domain 1 non-functional as a vWFC domain. Domain 2, however, is not corrupted by this splicing event and consequently represents the single vWFC domain seen in this protein (69-124aa of SEQ ID NO:26).

Splice variants of the polypeptides of the invention are predicted to have different biological functions, such as possessing different affinities for binding partners.

### Example 6

### The SECFAM3 Family Profile

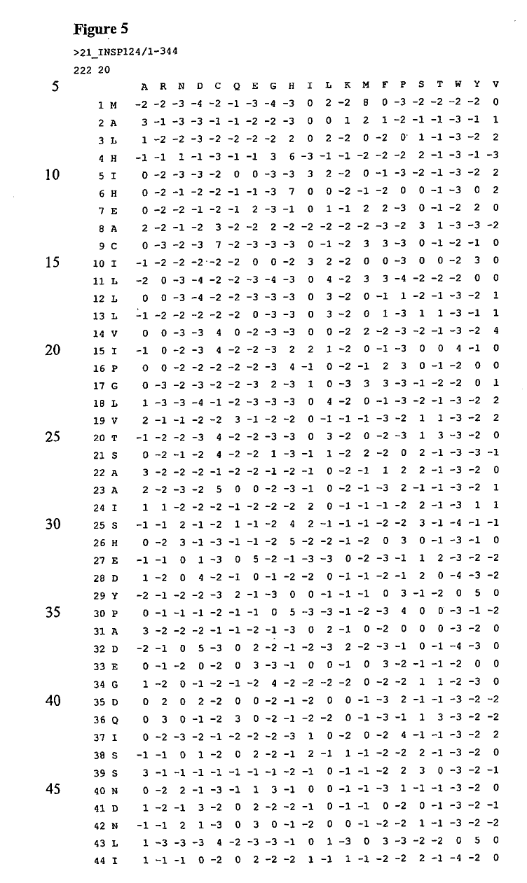

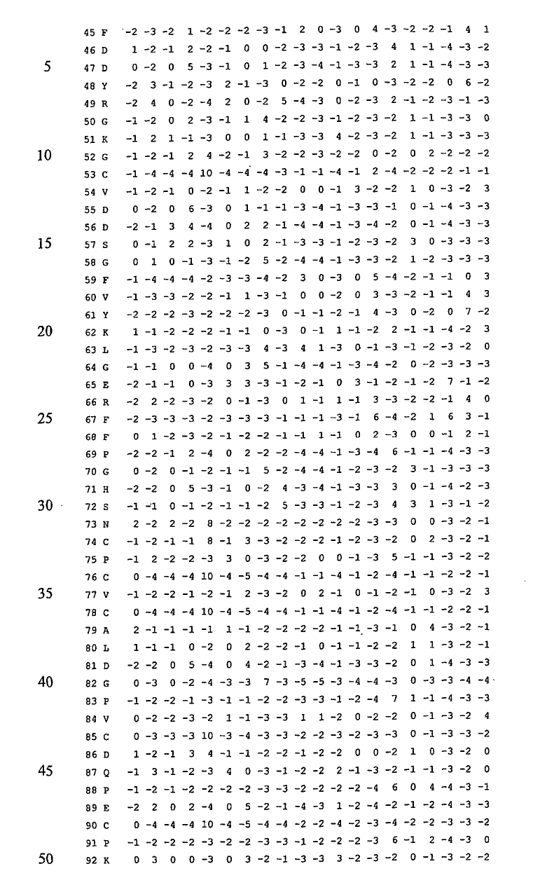

FIG. 5 shows the position-specific score matrix, or profile, for the SECFAM3 family. This represents the unique signature of the family. The profile was generated by first creating a multiple alignment of the sequences. A template sequence was chosen, in this case INSP124, to construct a profile around. The frequency of each of the possible 20 amino acid types was assessed for each column of the family multiple sequence alignment that was occupied by a residue of the template sequence. The score of each amino acid residue type at each position in the family alignment was calculated based on the frequency scores and the likelihood of seeing a substitution of the dominant residue with this residue type, based on the BLOSUM62 position-independent background matrix (Henikoff & Henikoff, 1992. Proc. Natl. Acad. Sci. USA, 89:10915-9). This matrix is based on a large dataset of family alignment blocks (BLOcks SUbstitution Matrix) where amino acid substitution frequencies were assessed based on alignments clustered at 62% identity or greater. In this case, these factors were pooled to give a logarithm-based score for each amino acid type at each position in the SECFAM3 alignment. The highest positive scores represent those amino acids that are most likely to be found at that position. This profile can be used to find an alignment score of a query sequence. At each position, the corresponding value for that amino acid is extracted and the sum of all such scores for each amino acid of the query sequence constitutes the alignment score for that sequence. If this is above a certain threshold value, the query sequence may be significantly related to the family. The profile, then, forms a sensitive statistical standard for the family. BLASTP of INSP124 against itself yields a minimum E-value of e-143.

### Example 7

### Generating a Consensus Sequence in PROSITE Format for the SECFAM3 Family

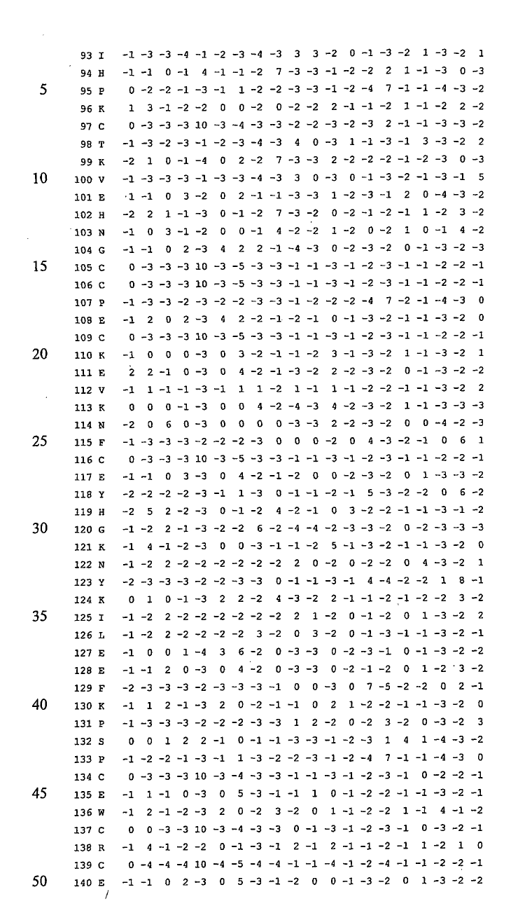

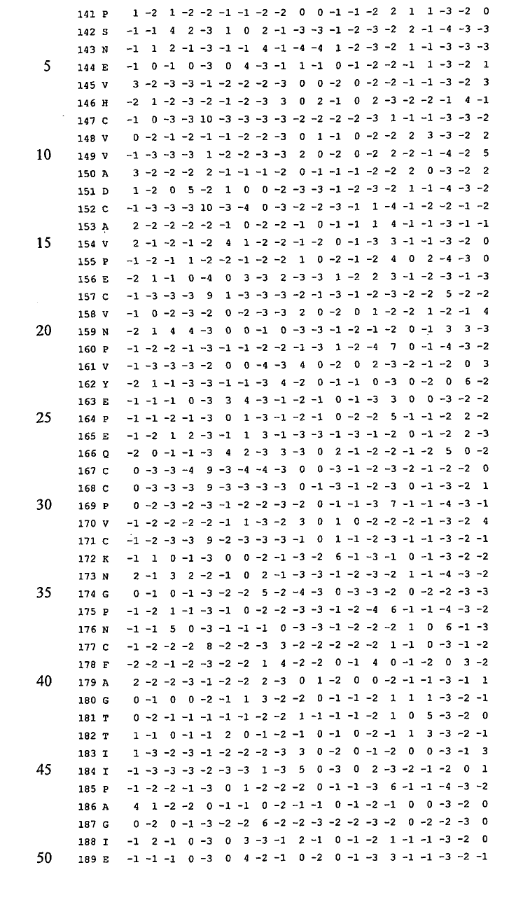

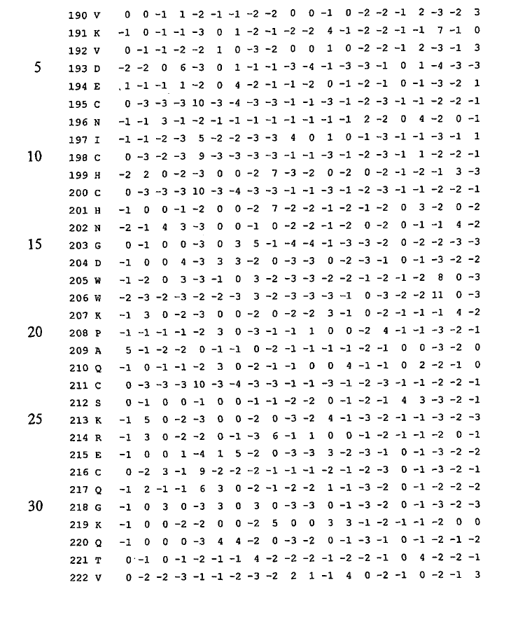

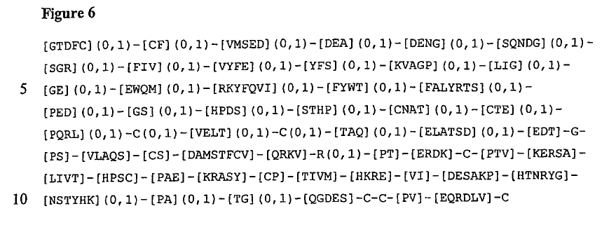

FIG. 6 shows a consensus sequence that represents the first domain of the proteins in the SECFAM3 family. The domain is predicted to be a vWFC domain. The second domain is also annotated as a vWFC domain.

### Example 8

### Cloning of INSP123

Preparation of Human cDNA Templates

First strand cDNA was prepared from a variety of normal human tissue total RNA samples (purchased from Clontech, Stratagene, Ambion, Biochain Institute and prepared in-house) using Superscript II RNase H⁻ Reverse Transcriptase (Invitrogen) according to the manufacturer's protocol. Oligo (dT)₁₅ primer (1 μl at 500 μg/ml) (Promega), 2 μg human total RNA, 1 μl 10 mM dNTP mix (10 mM each of dATP, dGTP, dCTP and dTTP at neutral pH) and sterile distilled water to a final volume of 12 μl were combined in a 1.5 ml Eppendorf tube, heated to 65° C. for 5 min and then chilled on ice. The contents of the tube were collected by brief centrifugation and 4 μl of 5× First-Strand Buffer, 2 μl 0.1 M DTT, and 1 μl RnaseOUT Recombinant Ribonuclease Inhibitor (40 units/μl, Invitrogen) were added. The contents of the tube were mixed gently and incubated at 42° C. for 2 min; then 1 μl (200 units) of SuperScript II enzyme was added and mixed gently by pipeting. The mixture was incubated at 42° C. for 50 min and then inactivated by heating at 70° C. for 15 min. To remove RNA complementary to the cDNA, 1 μl (2 units) of E. coli RNase H (Invitrogen) was added and the reaction mixture incubated at 37° C. for 20 min. The final 21 μl of reaction mix was diluted by adding 179 μl sterile water to give a total volume of 200 μl. The human cDNA sample used as a template for the amplification of INSP123 was derived from brain.

cDNA Libraries

Human cDNA libraries (in bacteriophage lambda (λ) vectors) were purchased from Clontech, Invitrogen, or made in-house in λ GT10 vectors. Bacteriophage X DNA was prepared from small scale cultures of infected E. coli host strain using the Wizard Lambda Preps DNA purification system according to the manufacturer's instructions (Promega, Corporation, Madison Wis.). Human cDNA library samples used as templates for the amplification of INSP123 were derived from fetal brain, adult brain, and a mixed brain-lung-testis library.

Gene Specific Cloning Primers for PCR

A pair of PCR primers having a length of between 18 and 25 bases were designed for amplifying the complete coding sequence of the virtual cDNA using Primer Designer Software (Scientific & Educational Software, PO Box 72045, Durham, N.C. 27722-2045, USA). PCR primers were optimized to have a Tm close to 55±10° C. and a GC content of 40-60%. Primers were selected which had high selectivity for the target sequence (INSP123) with little or no non-specific priming.

PCR Amplification of INSP123 from a Variety of Human cDNA Templates and Phase Library cDNA

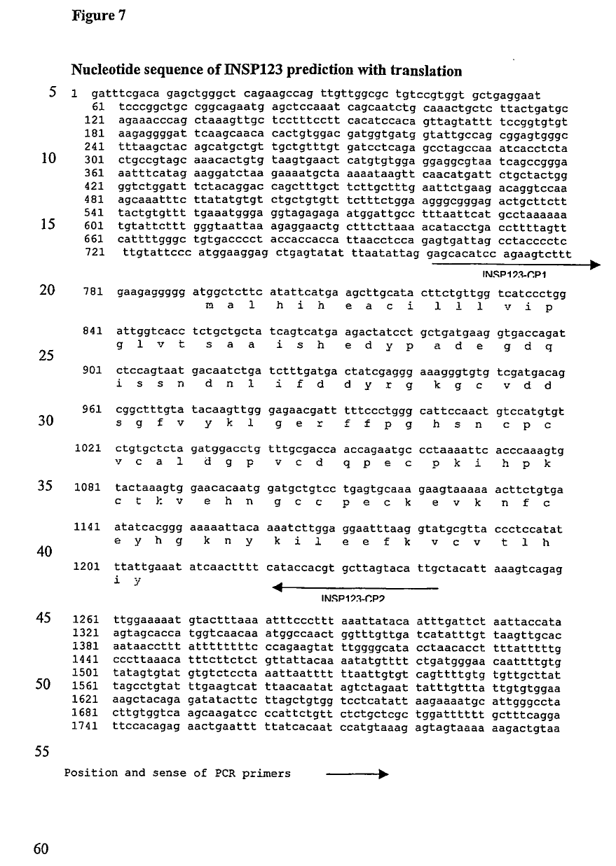

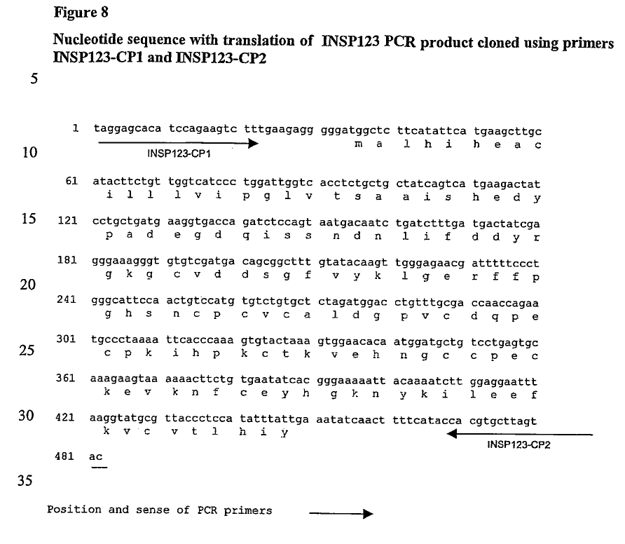

Gene-specific cloning primers (INSP123-CP1 and INSP123-CP2, FIG. 7, FIG. 8 and Table 1) were designed to amplify a cDNA fragment of 482 bp covering the entire 414 bp coding sequence of the INSP123 prediction. Interrogation of public EST sequence databases with the INSP123 prediction suggested that the sequence might be expressed in brain cDNA templates. The gene-specific cloning primers INSP123-CP1 and INSP123CP2 were therefore used with a human cDNA sample from brain and the phage library cDNA samples listed in Section 1.2 as the PCR templates. The PCR was performed in a final volume of 50 μl containing 1× AmpliTaq™ buffer, 200 μM dNTPs, 50 pmoles of each cloning primer, 2.5 units of AmpliTaq™ (Perkin Elmer) and 100 ng of cDNA template using an MJ Research DNA Engine, programmed as follows: 94° C., 2 min; 40 cycles of 94° C., 1 min, 53° C., 1 min, and 72° C., 1 min; followed by 1 cycle at 72° C. for 7 min and a holding cycle at 4° C.

The reaction mixture (50 μl) of each amplification was analysed on a 0.8% agarose gel in 1×TAE buffer (Invitrogen) and a single PCR product was seen migrating at approximately the predicted molecular mass in the sample corresponding to the brain-lung-testis cDNA library template. This PCR product was purified using the Wizard PCR Preps DNA Purification System (Promega). The PCR product was eluted in 50 μl of water and subcloned directly.

TABLE 1
INSP123 cloning and sequencing primers

| | |
|---|---|
| Primer | Sequence (5′-3′) |

| | |
|---|---|
| INSP123-CP1 | TAG GAG CAC ATC CAG AAG TC |
| INSP123-CP2 | GTA CTA AGC ACG TGG TAT GA |
| INSP123-EX1 | AA GCA GGC TTC GCC ACC ATG GCT CTT CAT |
| | ATT CAT GA |
| INSP123-EX2 | GTG ATG GTG ATG GTG ATA AAT ATG GAG GGT |
| | AAC GC |
| GCP Forward | G GGG ACA AGT TTG TAC AAA AAA GCA GGC |
| | TTC GCC ACC |
| GCP Reverse | GGG GAC CAC TTT GTA CAA GAA AGC TGG GTT |
| | TCA ATG GTG ATG GTG ATG GTG |
| pEAK12F | GCC AGC TTG GCA CTT GAT GT |
| pEAK12R | GAT GGA GGT GGA CGT GTC AG |
| 21M13 | TGT AAA ACG ACG GCC AGT |
| M13REV | CAG GAA ACA GCT ATG ACC |
| T7 | TAA TAC GAC TCA CTA TAG G |
| T3 | ATT AAC CCT CAC TAA AGG |

Underlined sequence = Kozak sequence
Bold = Stop codon
Italic sequence = His tag

Subcloning of PCR Products

The PCR product was subcloned into the topoisomerase I modified cloning vector (pCR4-TOPO) using the TA cloning kit purchased from the Invitrogen Corporation using the conditions specified by the manufacturer. Briefly, 4 μl of gel purified PCR product from the brain-lung-testis cDNA library amplification was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm (room temperature) SOC media was added. Samples were incubated with shaking (220 rpm) for 1 h at 37° C. The transformation mixture was then plated on L-broth (LB) plates containing ampicillin (100 μg/ml) and incubated overnight at 37° C.

Colony PCR

Colonies were inoculated into 50 μl sterile water using a sterile toothpick. A 10 μl aliquot of the inoculum was then subjected to PCR in a total reaction volume of 20 μl containing 1× AmpliTaq™ buffer, 200 μM dNTPs, 20 pmoles T7 primer, 20 pmoles of T3 primer, 1 unit of AmpliTaq™ (Perkin Elmer) using an MJ Research DNA Engine. The cycling conditions were as follows: 94° C., 2 min; 30 cycles of 94° C., 30 sec, 48° C., 30 sec and 72° C. for 1 min. Samples were maintained at 4° C. (holding cycle) before further analysis.

PCR reaction products were analyzed on 1% agarose gels in 1×TAE buffer. Colonies which gave the expected PCR product size (482 bp cDNA+105 bp due to the multiple cloning site or MCS) were grown up overnight at 37° C. in 5 ml L-Broth (LB) containing ampicillin (100 μg/ml), with shaking at 220 rpm.

Plasmid DNA Preparation and Sequencing

Miniprep plasmid DNA was prepared from the 5 ml culture using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an Eppendorf BO photometer or Spectramax 190 Photometer (Molecular Devices). Plasmid DNA (100-200 ng) was subjected to DNA sequencing with the T7 primer and T3 primer using the BigDye Terminator system (Applied Biosystems cat. no. 4390246) according to the manufacturer's instructions. The primer sequences are shown in Table 1. Sequencing reactions were purified using Dye-Ex columns (Qiagen) or Montage SEQ 96 cleanup plates (Millipore cat. no. LSKS09624) then analyzed on an Applied Biosystems 3700 sequencer.

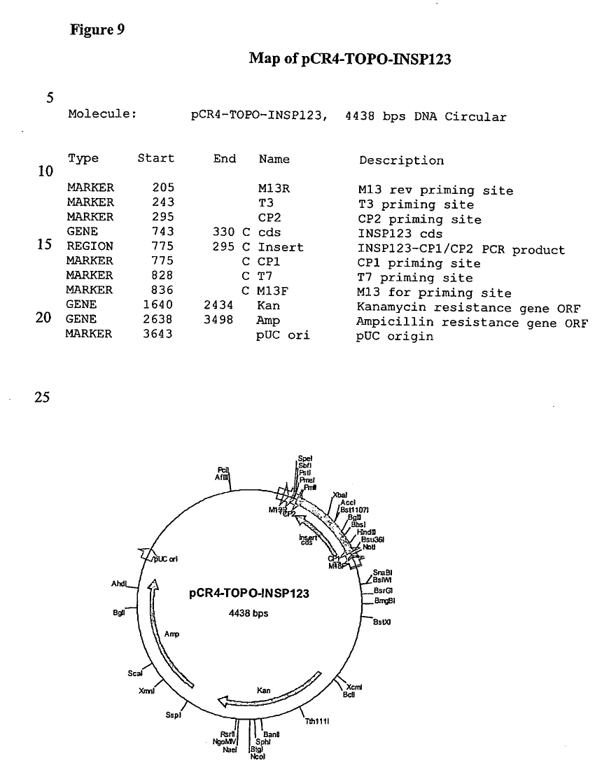

Sequence analysis identified a clone containing a 100% match to the predicted INSP123 sequence. The sequence of the cloned cDNA fragment is shown in FIG. 8. The plasmid map of the cloned PCR product (pCR4-TOPO-INSP123) (plasmid ID.14352) is shown in FIG. 9.

### Example 9

### Construction of Mammalian Cell Expression Vectors for INSP123

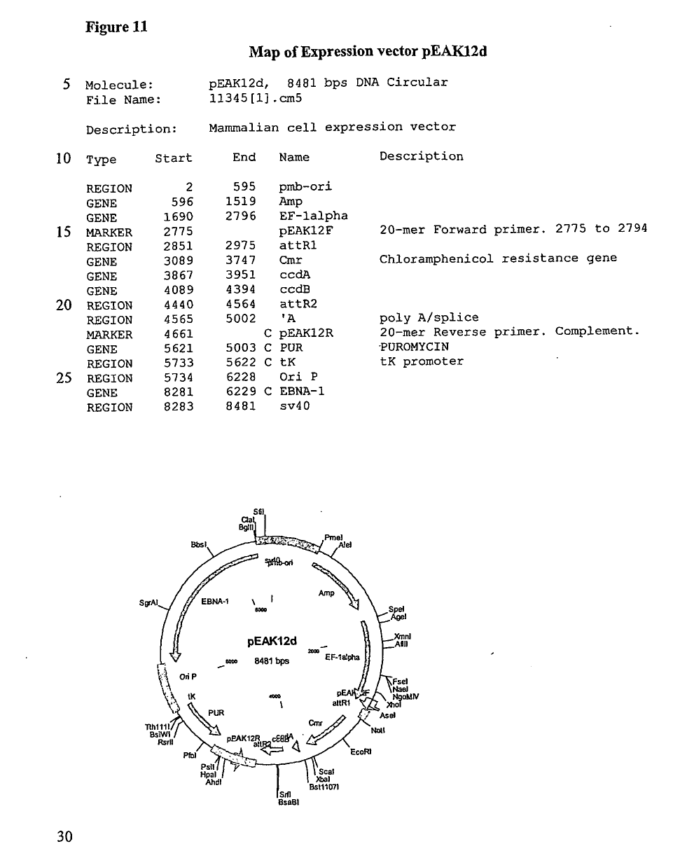

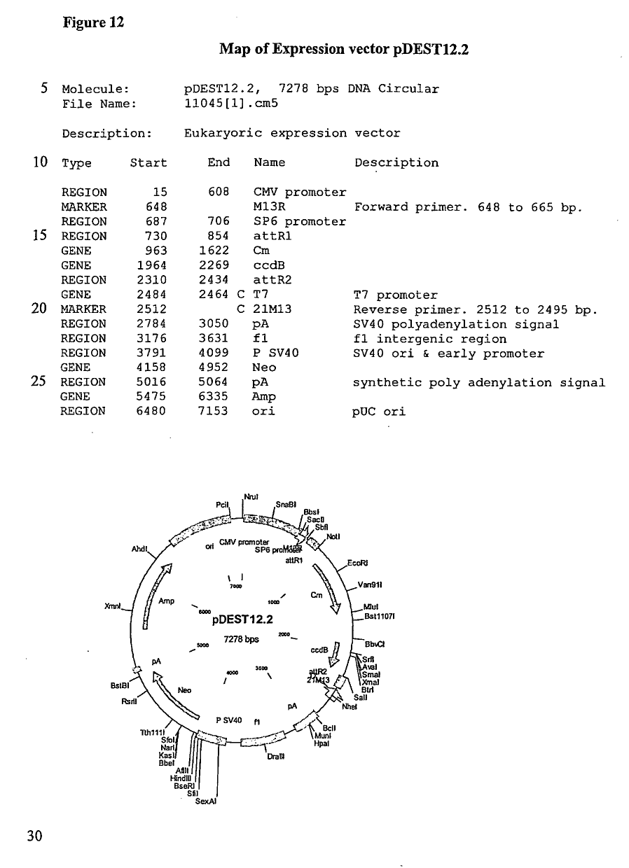

Plasmid 14352 was used as a PCR template to generate pEAK12d (FIG. 11) and pDEST12.2 (FIG. 12) expression clones containing the INSP123 ORF sequence with a 3′ sequence encoding a 6HIS tag using the Gateway™ cloning methodology (Invitrogen).

Generation of Gateway Compatible INSP123 ORF Fused to an in Frame 6HIS Tag Sequence.

The first stage of the Gateway cloning process involves a two step PCR reaction which generates the ORF of INSP123 flanked at the 5′ end by an attB1 recombination site and Kozak sequence, and flanked at the 3′ end by a sequence encoding an in-frame 6 histidine (6HIS) tag, a stop codon and the attB2 recombination site (Gateway compatible cDNA). The first PCR reaction (in a final volume of 50 μl) contains: 1 μl (40 ng) of plasmid 14352, 1.5 μl dNTPs (10 mM), 10 μl of 10× Pfx polymerase buffer, 1 μl MgSO4 (50 mM), 0.5 μl each of gene specific primer (100 μm) (INSP123-EX1 and INSP123-EX2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification products were visualized on 0.8% agarose gel in 1×TAE buffer (Invitrogen) and a product migrating at the predicted molecular mass (447 bp) was purified from the gel using the Wizard PCR Preps DNA Purification System (Promega) and recovered in 50 μl sterile water according to the manufacturer's instructions.

The second PCR reaction (in a final volume of 50 μl) contained 10 μl purified PCR 1 product, 1.5 μl dNTPs (10 mM), 5 μl of 10×Pfx polymerase buffer, 1 μl MgSO4 (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. PCR products were gel purified using the Wizard PCR prep DNA purification system (Promega) according to the manufacturer's instructions.

Subcloning of Gateway Compatible INSP123 ORF into Gateway Entry Vector pDONR 221 and Expression Vectors pEAK12d and pDEST12.2

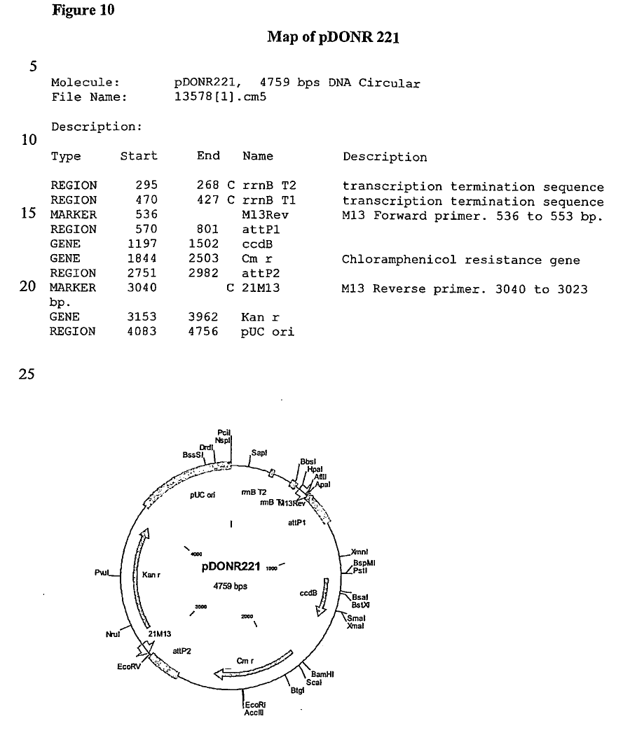

The second stage of the Gateway cloning process involves subcloning of the Gateway modified PCR product into the Gateway entry vector pDONR 221 (Invitrogen, FIG. 10) as follows: 5 μl of purified product from PCR2 were incubated with 1.5 μl pDONR 221 vector (0.1 μg/μl), 2 μl BP buffer and 1.5 μl of BP clonase enzyme mix (Invitrogen) in a final volume of 10 μl at RT for 1 h. The reaction was stopped by addition of proteinase K 1 μl (2 μg/μl) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm), for 1 h at 37° C. Aliquots of the transformation mixture (10 μl and 50 μl) were then plated on L-broth (LB) plates containing kanamycin (40 μg/ml) and incubated overnight at 37° C.

Plasmid mini-prep DNA was prepared from 5 ml cultures from 6 of the resultant colonies using a Qiaprep Turbo 9600 robotic system (Qiagen). Plasmid DNA (150-200 ng) was subjected to DNA sequencing with 21M13 and M13Rev primers using the BigDyeTerminator system (Applied Biosystems cat. no. 4390246) according to the manufacturer's instructions. The primer sequences are shown in Table 1. Sequencing reactions were purified using Dye-Ex columns (Qiagen) or Montage SEQ 96 cleanup plates (Millipore cat. no. LSKS09624) then analyzed on an Applied Biosystems 3700 sequencer.

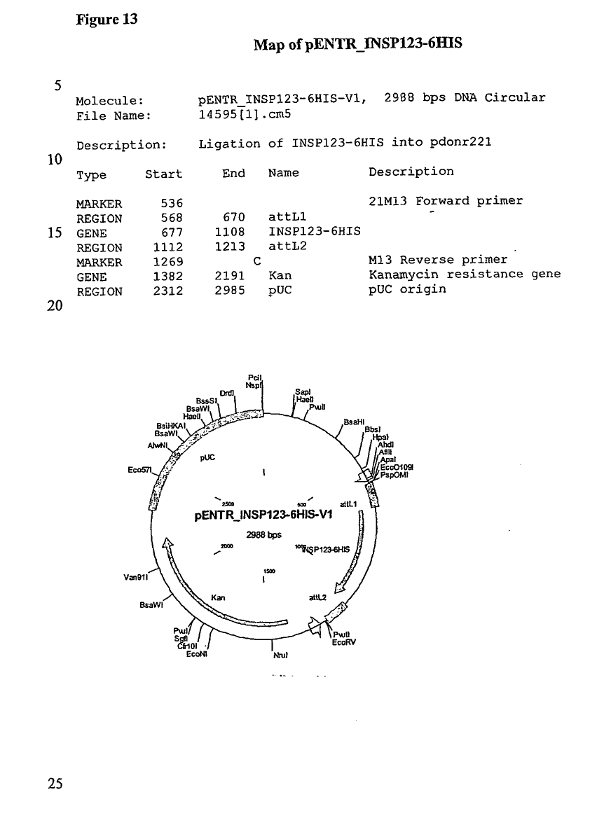

Plasmid eluate (2 μl or approx. 150 ng) from one of the clones which contained the correct sequence (pENTR_INSP123-6HIS, plasmid ID 14595, FIG. 13) was then used in a recombination reaction containing 1.5 μl of either pEAK12d vector or pDEST12.2 vector (FIGS. 11 & 12) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 ul) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep Turbo 9600 robotic system (Qiagen). Plasmid DNA (200-500 ng) in the pEAK12d vector was subjected to DNA sequencing with pEAK12F and pEAK12R primers as described above. Plasmid DNA (200-500 ng) in the pDEST12.2 vector was subjected to DNA sequencing with 21M13 and M13Rev primers as described above. Primer sequences are shown in Table 1.

CsCl gradient purified maxi-prep DNA was prepared from a 500 ml culture of one of each of the sequence verified clones (pEAK12d_INSP123-6HIS, plasmid ID number 14602, FIG. 8, and pDEST12.2_INSP123-6HIS, plasmid ID 14606, FIG. 9) using the method described by Sambrook J. et al., 1989 (in Molecular Cloning, a Laboratory Manual, 2^{nd} edition, Cold Spring Harbor Laboratory Press), Plasmid DNA was resuspended at a concentration of 1 μg/μl in sterile water (or 10 mM Tris-HCl pH 8.5) and stored at −20° C.

### Example 10

### Cloning of INSP124 by Exon Assembly

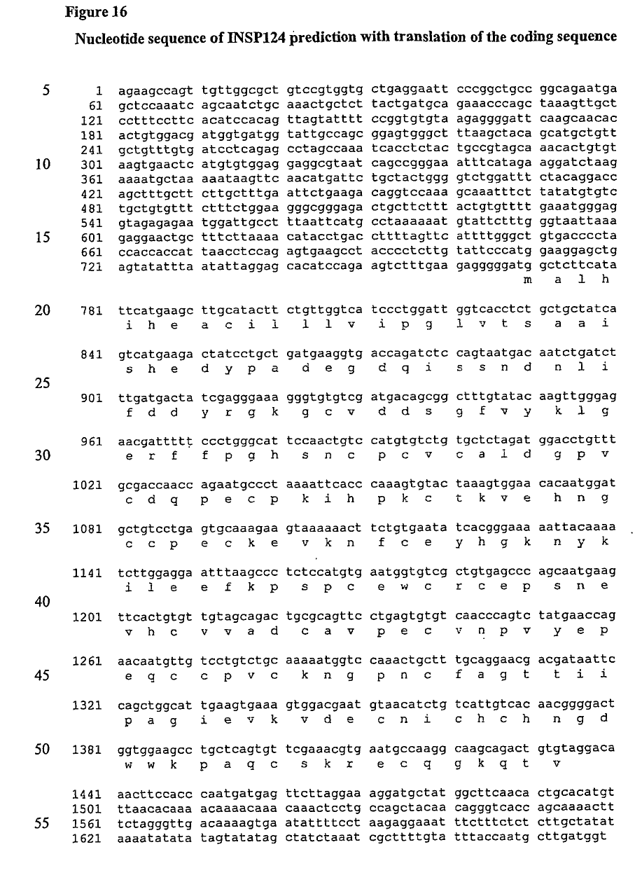

INSP124 is a prediction for a full length SECFAM3 family novel secreted protein of 222 amino acids (666 bp) encoded in three coding exons (FIGS. 16 & 17).

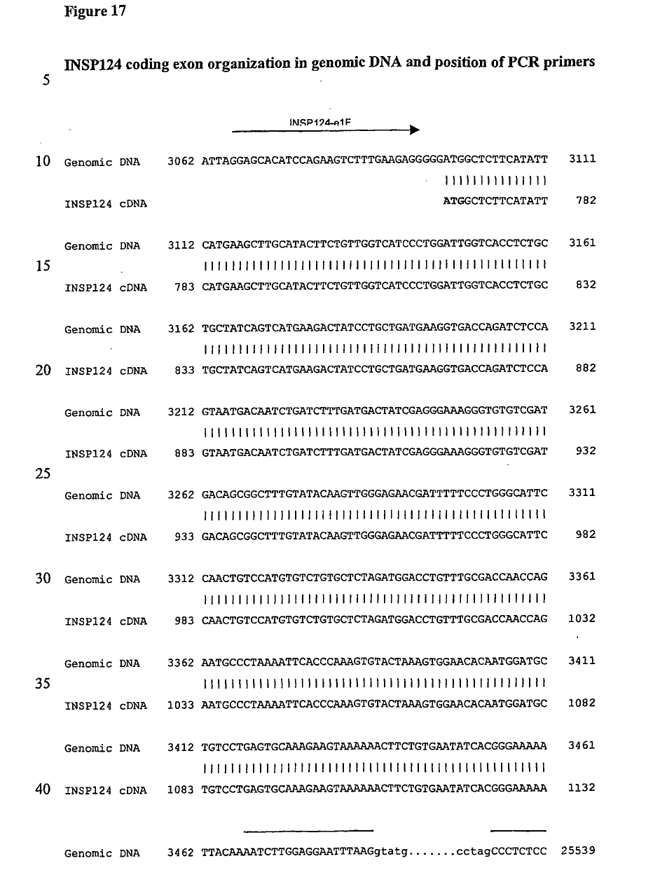

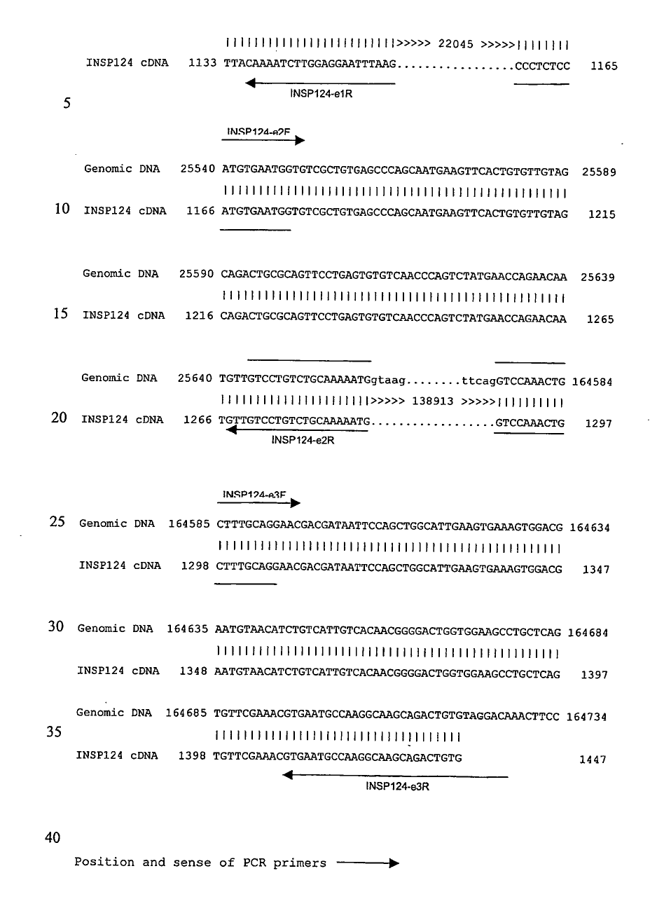

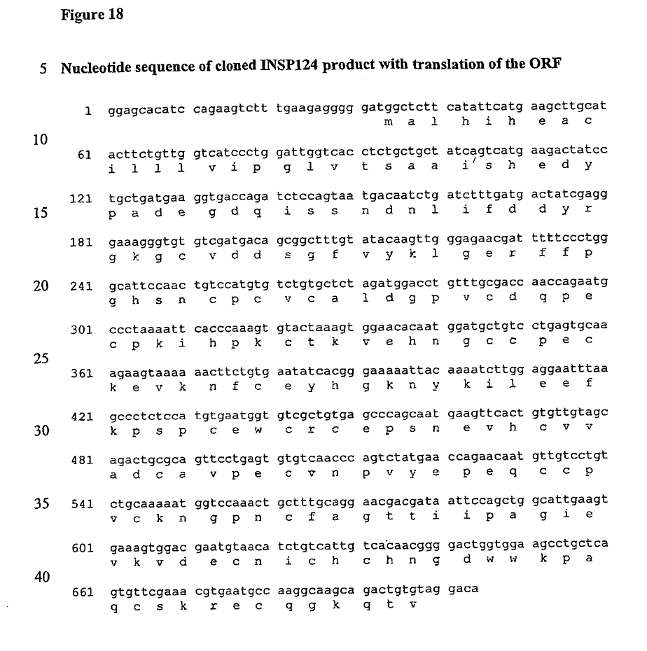

In order to generate INSP124 protein:
Exon 1 was amplified from plasmid ID 14352 (containing INSP123, a splice variant of INSP124) by PCR.
Exons 2 and 3 were amplified from genomic DNA by PCR (FIG. 17).
The gel-purified exons were mixed and a new PCR reaction was performed to amplify the re-assembled DNA.
The full length PCR product corresponding to the INSP124 coding sequence (FIG. 18) was subcloned into pCR-BluntII-TOPO cloning vector (Invitrogen) and then sequentially into pDONR 201 (Gateway entry vector) and expression vectors pEAK12d and pDEST12.2. using the Invitrogen Gateway™ methodology.
   PCR Amplification of Exons Encoding INSP124 from Plasmid or Genomic DNA

PCR primers were designed to amplify exons 1, 2 and 3 of INSP124 (Table 2). The reverse primer for exon 1 (INSP124-e1R) has an overlap of 18 bp with exon 2 of INSP124 at its 5′ end. The forward primer for exon 2 (INSP124-e2F) has an 19 bp overlap with exon 1 of INSP124 at its 5′ end. The reverse primer for exon 2 (INSP124-e2R) has an overlap of 19 bp with exon 3 of INSP124 at its 5′ end. The forward primer for exon 3 (INSP124-e3F) has an 18 bp overlap with exon 2 of INSP124 at its 5′ end.

To generate exon 1 of INSP124, the PCR reaction was performed in a final volume of 50 μl and contained 100 ng of plasmid ID 14352 DNA, 1× AmpliTaq™ buffer, 200 μM dNTPs, 50 pmoles of INSP124-e1F, 50 pmoles of INSP124-e1R, and 2.5 units of AmpliTaq™ (Perkin Elmer) using an MJ Research DNA Engine, programmed as follows: 94° C., 2 min; 30 cycles of 94° C., 30 sec, 63° C., 30 sec, and 72° C., 1 min; followed by 1 cycle at 72° C. for 7 min and a holding cycle at 4° C.

To generate exon 2 of INSP124, the PCR reaction was performed in a final volume of 50 μl and contained 1 μl of genomic DNA (0.1 μg/μl (Novagen Inc.), 1× AmpliTaq™ buffer, 200 μM dNTPs, 50 pmoles of INSP124-e2F, 50 pmoles of INSP124-e2R, and 2.5 units of AmpliTaq™ (Perkin Elmer). To generate exon 3 of INSP124, the PCR reaction was performed in a final volume of 50 μl and contained 1 μl of genomic DNA (0.1 μg/μl (Novagen Inc.), 1× AmpliTaq™ buffer, 200 μM dNTPs, 50 pmoles of INSP124-e3F, 50 pmoles of INSP124-e3R, and 2.5 units of AmpliTaq™ (Perkin Elmer). PCR cycling to generate exon 2 and exon 3 used an MJ Research DNA Engine, programmed as follows: 94° C., 2 min; 30 cycles of 94° C., 30 sec, 65° C., 30 sec, and 72° C., 40 sec; followed by 1 cycle at 72° C. for 5 min and a holding cycle at 4° C.

Reaction products were analysed on a 1.8% agarose gel (1×TAE) and PCR products of the correct size (439 bp, 168 bp, 171 bp for exons 1, 2 and 3, respectively) were gel-purified using the Wizard PCR Preps DNA Purification System (Promega) and eluted in 50 μl of water. Ten μl of each purified PCR product was visualised on a 1.8% agarose gel to estimate the concentration.

TABLE 2
Primers for INSP124 Cloning and sequencing

| | |
|---|---|
| Primer | Sequence (5′-3′) |

| | |
|---|---|
| GCP Forward | G GGG ACA AGT TTG TAC AAA AAA GCA GGC |
| | TTC GCC ACC |
| GCP Reverse | GGG GAC CAC TTT GTA CAA GAA AGC TGG GTT |
| | TCA ATG GTG ATG GTG ATG GTG |
| INSP124-e1F | GGA GCA CAT CCA GAA GTC TTT GAA GAG G |
| INSP124-e1R | [symbol] CTT AAA TTC CTC |
| | CAA GAT TTT G |
| INSP124-e2F | [symbol] CTC TCC ATG TGA |
| | ATG GTG |
| INSP124-e2R | [symbol] CA TTT TTG CAG |
| | ACA GGA CAA C |
| INSP124-e3F | [symbol] GTC CAA ACT GCT |
| | TTG CAG GAA C |
| INSP124-e3R | TGT CCT ACA CAG TCT GCT TGC CTT GGC ATT |
| | CAC |
| INSP124-EX1 | AA GCA GGC TTC GCC ACC ATG GCT CTT CAT |
| | ATT CAT GA |
| INSP124-EX2 | GTG ATG GTG ATG GTG CAC AGT CTG CTT GCC |
| | TTG GC |
| pEAK12-F | GCC AGC TTG GCA CTT GAT GT |
| pEAK12-R | GAT GGA GGT GGA CGT GTC AG |
| pENTR-F | TCG CGT TAA CGC TAG CAT GGA TCT C |
| pENTR-R | GTA ACA TCA GAG ATT TTG AGA CAC |
| T7 | TAA TAC GAC TCA CTA TAG GG |
| SP6 | ATT TAG GTG ACA CTA TAG |

Underlined sequence = Kozak sequence
Bold = Stop codon
Italic sequence = His tag [symbol]
= overlap with adjacent exon

Assembly of Exons 1.2 and 3 to Generate the INSP124 ORF

Exons 1, 2 and 3 were assembled in a 50 μl PCR reaction containing 3 μl of gel purified exon 1, 5 μl of gel purified exon 2, 5 μl of gel purified exon 3, 1.5 μl of 10 mM dNTPs, 1 μl of MgSO₄, 1.5 μl of INSP124-e1F (10 μM), 1.5 μl of INSP124-e3R (10 μM), 5 μl of 10× Platinum Pfx™ buffer, and 0.5 μl of Platinum Pfx™ DNA polymerase (5 U/μl) (Invitrogen). The reaction conditions were: 94° C., 4 min; 10 cycles of 94° C. for 30 s, 48° C. for 30 s and 68° C. for 1 min; 25 cycles of 94° C. for 30 s, 52° C. for 30 s and 68° C. for 1 min; an additional elongation cycle of 68° C. for 10 min; and a holding cycle of 4° C. Reaction products were analysed on a 0.8% agarose gel (1×TAE). PCR products of the correct size (704 bp) were gel-purified using the Wizard PCR Preps DNA Purification System (Promega), eluted in 50 μl of water and subcloned directly.

Subcloning of PCR Products

The PCR product was subcloned into the topoisomerase I modified cloning vector (pCR-BluntII-TOPO) purchased from the Invitrogen Corporation using the conditions specified by the manufacturer. Briefly, 4 μl of gel purified PCR product was incubated for 15 min at room temperature with 1 μl of TOPO vector and 1 μl salt solution. The reaction mixture was then transformed into E. coli strain TOP10 (Invitrogen) as follows: a μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm (room temperature) SOC media was added. Samples were incubated with shaking (220 rpm), for 1 h at 37° C. The transformation mixture was then plated on L-broth (LB) plates containing kanamycin (40 μg/ml) and incubated overnight at 37° C.

Colony PCR

Colonies were inoculated into 50 μl sterile water using a sterile toothpick. A 10 μl aliquot of the inoculum was then subjected to PCR in a total reaction volume of 20 μl containing 1× AmpliTaq™ buffer, 200 μM dNTPs, 20 pmoles T7 primer, 20 pmoles of SP6 primer, 1 unit of AmpliTaq™ (Perkin Elmer) using an MJ Research DNA Engine. The cycling conditions were as follows: 94° C., 2 min; 30 cycles of 94° C., 30 sec. 48° C., 30 sec and 72° C. for 1 min. Samples were maintained at 4° C. (holding cycle) before further analysis.

PCR reaction products were analysed on 1% agarose gels in 1×TAE buffer. Colonies which gave the expected PCR product size (704 bp cDNA+186 bp due to the multiple cloning site or MCS) were grown up overnight at 37° C. in 5 ml L-Broth (LB) containing kanamycin (40 μg/ml), with shaking at 220 rpm.

Plasmid DNA Preparation and Sequencing

Miniprep plasmid DNA was prepared from 5 ml cultures using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an Eppendorf BO photometer or a Spectramax 190 photometer (Molecular Devices). Plasmid DNA (200-500 ng) was subjected to DNA sequencing with the T7 and SP6 primers using the BigDyeTerminator system (Applied Biosystems cat. no. 4390246) according to the manufacturer's instructions. The primer sequences are shown in Table 2.

Sequencing reactions were purified using Dye-Ex columns (Qiagen) or Montage SEQ 96 cleanup plates (Millipore cat. no. LSKS09624) then analyzed on an Applied Biosystems 3700 sequencer.

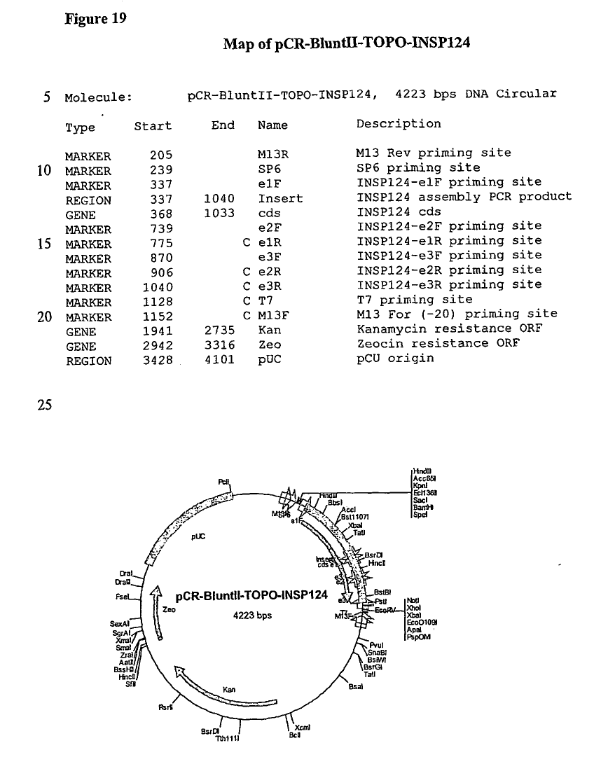

Sequence analysis identified a clone containing 100% match to the predicted INSP124 sequence. The sequence of the cloned cDNA fragment is shown in FIG. 3. The plasmid map of the cloned PCR product (pCR-BluntII-TOPO-INSP124, plasmid ID. 14649) is shown in FIG. 19.

### Example 11

### Construction of Mammalian Cell Expression Vectors for INSP124

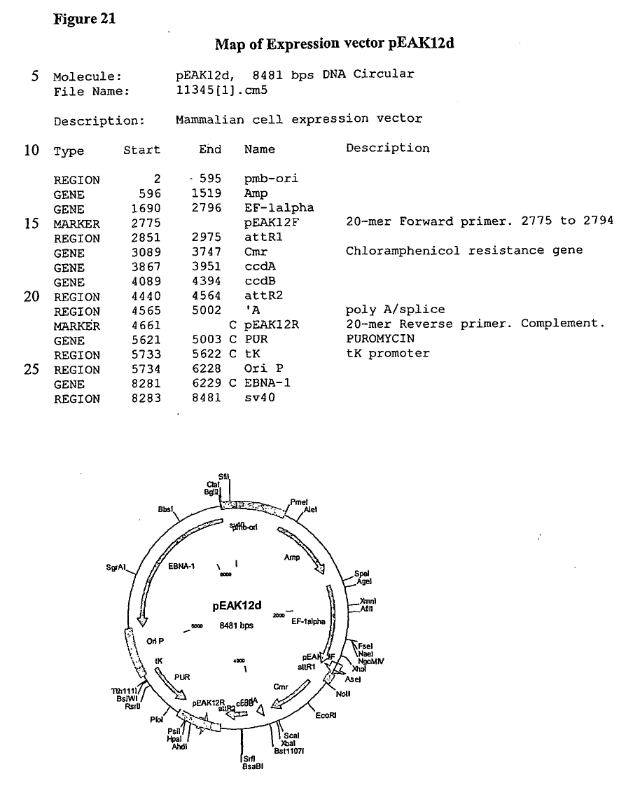

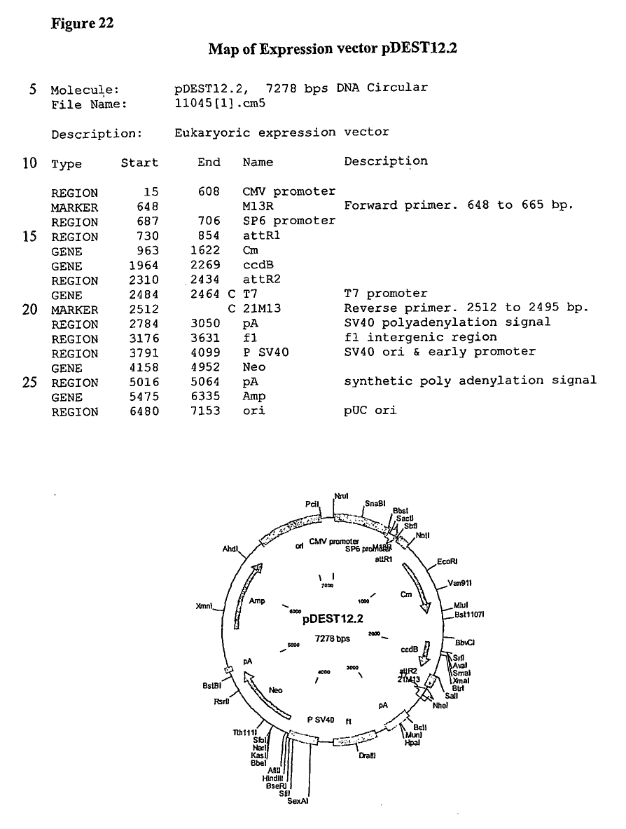

Plasmid 14649 was used as a PCR template to generate pEAK12d (FIG. 21) and pDEST12.2 (FIG. 22) expression clones containing the INSP124 ORF sequence with a 3′ sequence encoding a 6HIS tag using the Gateway™ cloning methodology (Invitrogen).

Generation of Gateway Compatible INSP124 ORF Fused to an in Frame 6HIS Tag Sequence.

The first stage of the Gateway cloning process involves a two step PCR reaction which generates the ORF of INSP124 flanked at the 5′ end by an attB1 recombination site and Kozak sequence, and flanked at the 3′ end by a sequence encoding an in-frame 6 histidine (6HIS) tag, a stop codon and the attB2 recombination site (Gateway compatible cDNA). The first PCR reaction (in a final volume of 50 μl) contains: 1 μl (40 ng) of plasmid 14649, 1.5 μl dNTPs (10 mM), 10 μl of 10×Pfx polymerase buffer, 1 μl MgSO4 (50 mM), 0.5 μl each of gene specific primer (100 μM) (INSP124-EX1 and INSP124-EX2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification products were visualized on 0.8% agarose gel in 1×TAE buffer (Invitrogen) and a product migrating at the predicted molecular mass (699 bp) was purified from the gel using the Wizard PCR Preps DNA Purification System (Promega) and recovered in 50 μl sterile water according to the manufacturer's instructions.

The second PCR reaction (in a final volume of 50 μl) contained 10 μl purified PCR 1 product, 1.5 μl dNTPs (10 mM), 5 μl of 10×Pfx polymerase buffer, 1 μl MgSO4 (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. PCR products were gel purified using the Wizard PCR prep DNA purification system (Promega) according to the manufacturer's instructions.

Subcloning of Gateway Compatible INSP124 ORF into Gateway Entry Vector pDONR221 and Expression Vectors pEAK12d and pDEST12.2

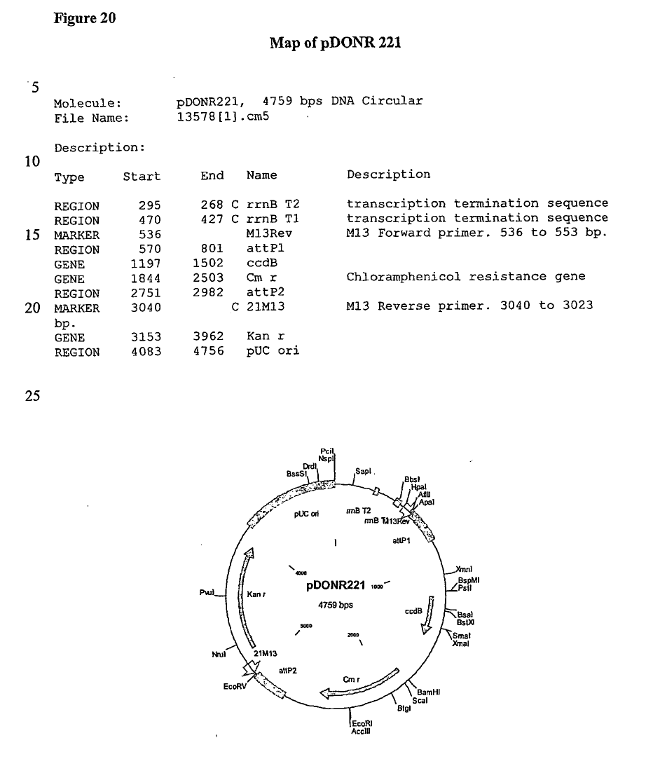

The second stage of the Gateway cloning process involves subcloning of the Gateway modified PCR product into the Gateway entry vector pDONR221 (Invitrogen, FIG. 20) as follows: 5 μl of purified product from PCR2 were incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of proteinase K (1 μl at 2 μg/μl) and incubated at 37° C. for a further 10 min. An aliquot of the reaction (1 μl) was used to transform E. coli DH10B cells by electroporation as follows: a 25 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing kanamycin (40 μg/ml) and incubated overnight at 37° C.

Plasmid mini-prep DNA was prepared from 5 ml cultures from 6 of the resultant colonies using a Qiaprep Turbo 9600 robotic system (Qiagen). Plasmid DNA (150-200 ng) was subjected to DNA sequencing with 21M13 and M13Rev primers using the BigDyeTerminator system (Applied Biosystems cat. no. 4390246) according to the manufacturer's instructions. The primer sequences are shown in Table 1. Sequencing reactions were purified using Dye-Ex columns (Qiagen) or Montage SEQ 96 cleanup plates (Millipore cat. no. LSKS09624) then analyzed on an Applied Biosystems 3700 sequencer.

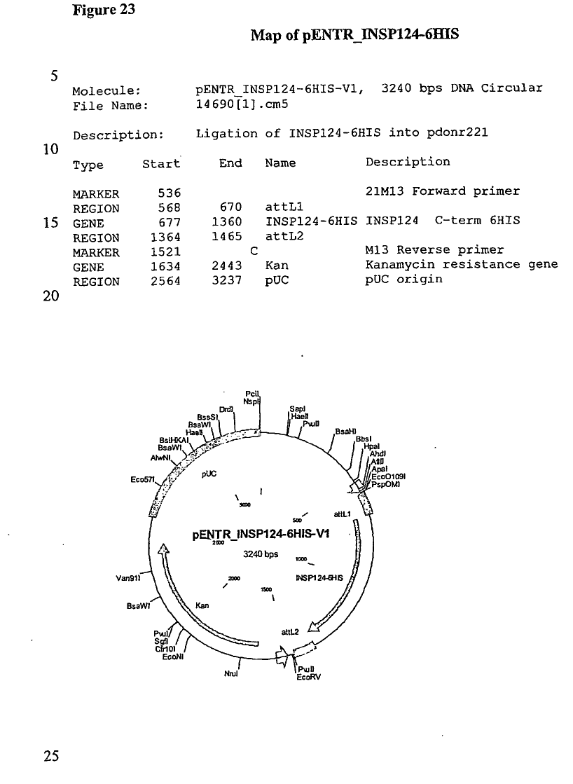

Plasmid eluate (2 μl or approx. 150 ng) from one of the clones which contained the correct sequence (pENTR_INSP124-HIS, plasmid ID 14690, FIG. 23) was then used in a recombination reaction containing 1.5 μl of either pEAK12d vector or pDEST12.2 vector (FIGS. 21 & 22) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 ul) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep Turbo 9600 robotic system (Qiagen). Plasmid DNA (200-500 ng) in the pEAK12d vector was subjected to DNA sequencing with pEAK12F and pEAK12R primers as described above. Plasmid DNA (200-500 ng) in the pDEST12.2 vector was subjected to DNA sequencing with 21M13 and M13Rev primers as described above. Primer sequences are shown in Table 2.

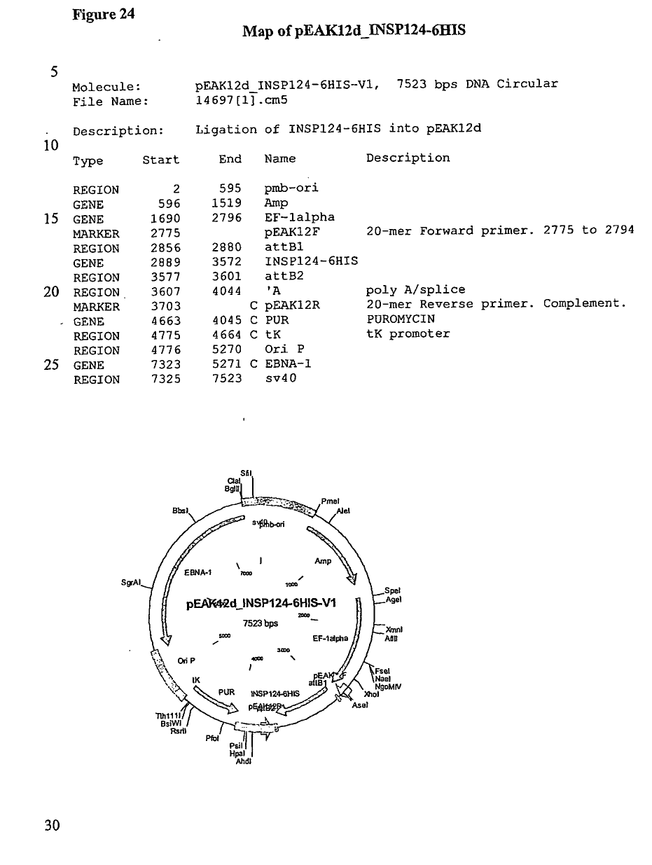

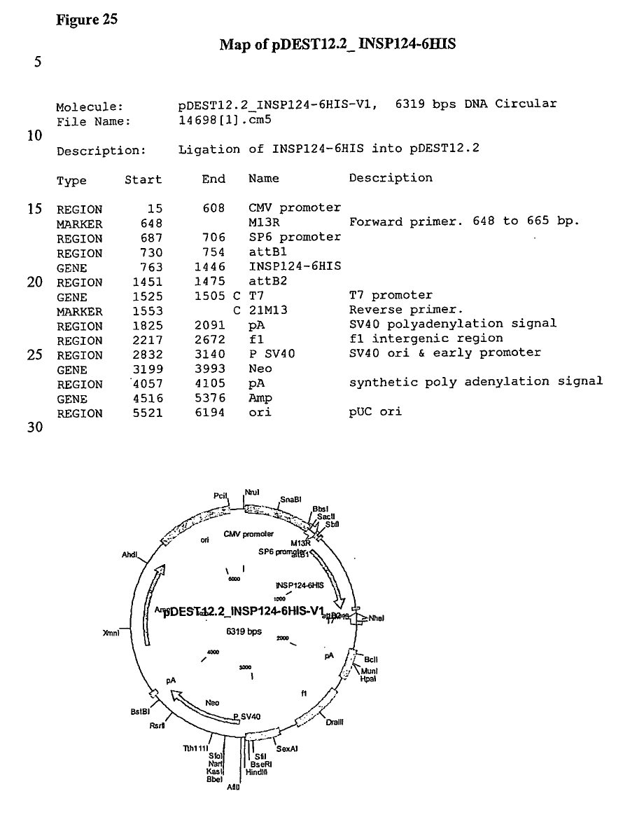

CsCl gradient purified maxi-prep DNA was prepared from a 500 ml culture from one of each of the sequence verified clones (pEAK12d_INSP124-6HIS, plasmid ID number 14697, FIG. 24, and pDEST12.2_INSP124-6HIS, plasmid ID 14698, FIG. 25) using the method described by Sambrook J. et al., 1989 (in Molecular Cloning, a Laboratory Manual, 2^{nd} edition, Cold Spring Harbor Laboratory Press). Plasmid DNA was resuspended at a concentration of 1 μg/μl in sterile water (or 10 mM Tris-HCl pH 8.5) and stored at −20° C.

### Example 12

### Cloning of INSP125 by Exon Assembly

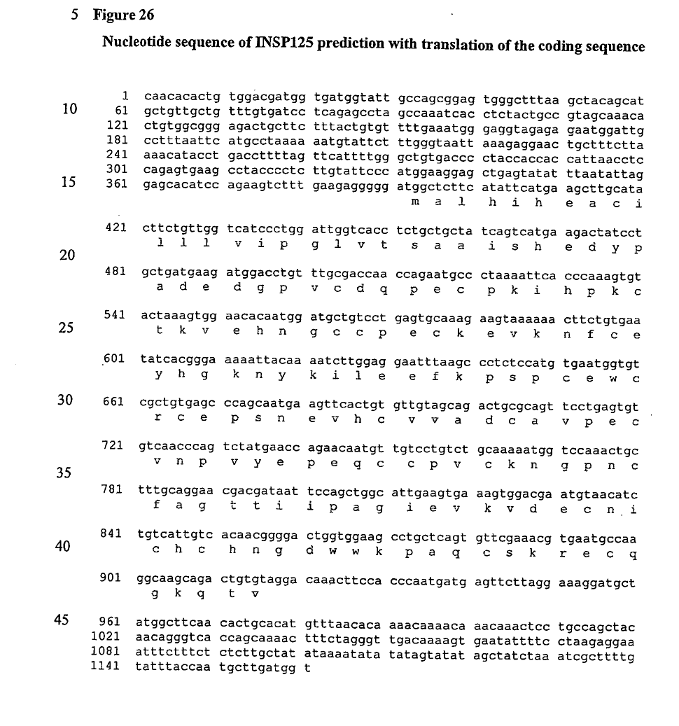

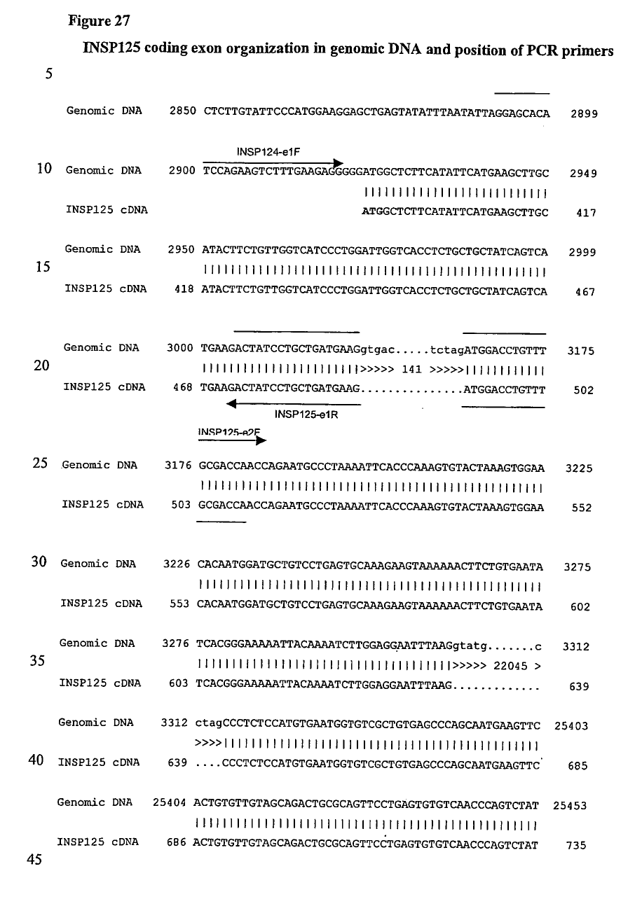

INSP125 is a prediction for a full length SECFAM3 family novel secreted protein of 175 amino acids (525 bp) (FIG. 26). The predicted INSP125 coding sequence was identical to the predicted INSP124 coding sequence except that it contains a 47 amino acid (141 bp) deletion. The INSP124 prediction had previously been cloned (pCR-BluntII-TOPO-INSP124, plasmid ID 14649).

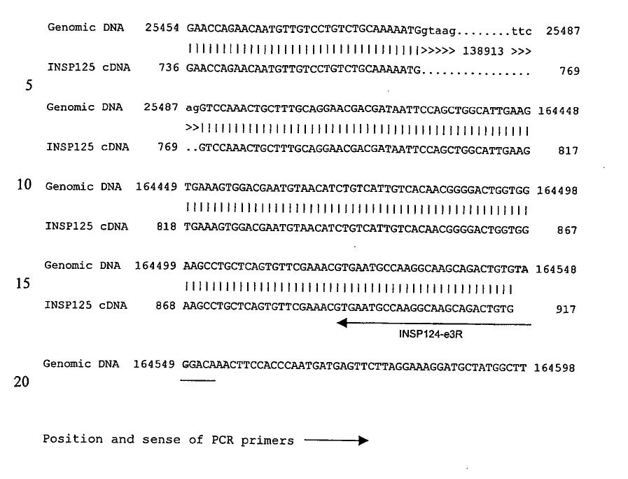

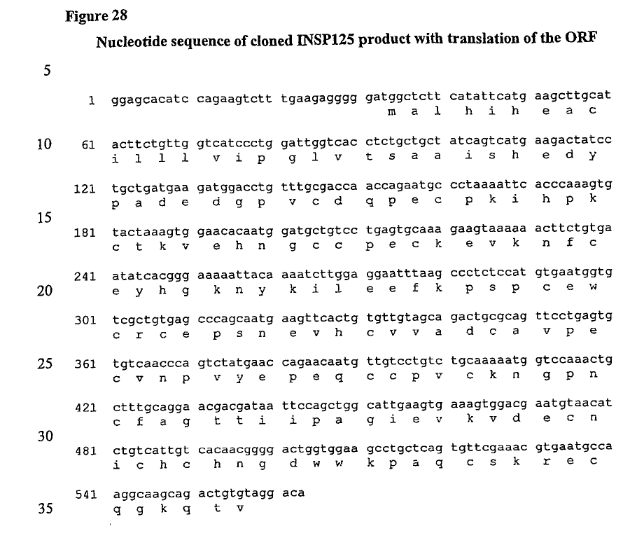

In order to generate INSP125 protein:
INSP125 exon 1 was amplified from plasmid pCR-BluntII-TOPO-INSP124 (plasmid ID 14649) by PCR.
Exons 24 were amplified as a single product from plasmid ID 14649 by PCR.
The gel-purified exons were mixed and a new PCR reaction was performed to amplify the re-assembled DNA.
The full length PCR product corresponding to the INSP125 coding sequence (FIG. 28) was subcloned into pCR4-TOPO cloning vector (Invitrogen) and then sequentially into pDONR 201 (Gateway entry vector) and expression vectors pEAK12d and pDEST12.2 using the Invitrogen Gateway™ methodology.
   PCR Amplification of Exons Encoding INSP125 from Plasmid ID 14649

PCR primers were designed to amplify exon 1 and exons 2-4 of INSP125 (Table 3). The reverse primer for exon 1 (INSP125-e1R) has an overlap of 19 bp with exon 2 of INSP125 at its 5′ end. The forward primer for exon 2 (INSP125-e2F) has an 18 bp overlap with exon 1 of INSP125 at its 5′ end. As the 5′ and 3′ ends of the coding sequence were the same as INSP124, the primers INSP124-e1F and INSP124-e3R were used as the forward and reverse primers to amplify the exon fragments, and ultimately the whole INSP125 coding sequence.

To generate exon 1 of INSP125, the PCR reaction was performed in a final volume of 501 containing 100 ng of plasmid ID 14649 DNA, 1.5 μl of 10 mM dNTPs, 1 μl of MgSO₄, 1.5 μl of INSP124-e1F (10 μM), 1.5 μl of INSP125-e1R (10 μM), 5 μl of 10× Platinum Pfx™ buffer, and 0.5 μl of Platinum Pfx™ DNA polymerase (5 U/μl) (Invitrogen). The reaction conditions were: 94° C., 2 min; 30 cycles of 94° C. for 15 s, 61° C. for 30 s and 68° C. for 1 min; an additional elongation cycle of 68° C. for 7 min; and a holding cycle of 4° C. The expected product size was 150 bp.

To generate exons 24 of INSP125, the PCR reaction was performed exactly as for exon 1 above, except that the amplification primers used were INSP125-e2F and INSP124-e3R. The expected product size was 450 bp.

Reaction products were loaded onto a 1.5% agarose gel (1×TAE) and PCR products of the correct size (150 bp and 450 bp) were gel-purified using the Qiagen MinElute DNA purification system (Qiagen) according to the manufacturer's instructions, and eluted in 10 μl of EB buffer (10 mM Tris.Cl, pH 8.5).

TABLE 3
Primers for INSP125 cloning and sequencing

| | |
|---|---|
| Primer | Sequence (5′-3′) |

| | |
|---|---|
| GCP Forward | G GGG ACA AGT TTG TAC AAA AAA GCA GGC |
| | TTC GCC ACC |
| GCP Reverse | GGG GAC CAC TTT GTA CAA GAA AGC TGG GTT |
| | TCA ATG GTG ATG GTG ATG GTG |
| INSP124-e1F | GGA GCA CAT CCA GAA GTC TTT GAA GAG G |
| INSP125-e1R | [symbol] T TCA TCA |
| | GCA GGA TAG TC |
| INSP125-e2F | [symbol] ATG GAC CTG TTT |
| | GCG ACC AAC C |
| INSP124-e3R | TGT CCT ACA CAG TCT GCT TGC CTT GGC ATT |
| | CAC |
| INSP125-EX1 | AA GCA GGC TTC GCC ACC ATG GCT CTT CAT |
| | ATT CAT GA |
| INSP125-EX2 | GTG ATG GTG ATG GTG CAC AGT CTG CTT GCC |
| | TTG GC |
| pEAK12-F | GCC AGC TTG GCA CTT GAT GT |
| pEAK12-R | GAT GGA GGT GGA CGT GTC AG |
| pENTR-F | TCG CGT TAA CGC TAG CAT GGA TCT C |
| pENTR-R | GTA ACA TCA GAG ATT TTG AGA CAC |
| T7 | TAA TAC GAC TCA CTA TAG GG |
| T3 | CTC CCT TTA GTG AGG GTA ATT |

Underlined sequence = Kozak sequence
Bold = Stop codon
Italic sequence = His tag [symbol]
= overlap with adjacent exon

Assembly of Exons 1, 24 to Generate the INSP125 ORF

Exon 1 and the exon 24 product were assembled in a 50 μl PCR reaction containing 1 μl of gel purified exon 1, 1 μl of gel purified exon 2-4 product, 1 μl of 10 mM dNTPs, 2 μl of MgSO₄, 1 μl of INSP124-e1F (10 μM), 1 μl of INSP124-e3R (10 μM), 5 μl of 10× Platinum Taq HiFi buffer, and 0.5 μl of Platinum Taq HiFi DNA polymerase (5 U/μl) (Invitrogen). The reaction conditions were: 94° C., 2 min; 10 cycles of 94° C. for 30 s, 48° C. for 30 s and 68° C. for 1 min; 25 cycles of 94° C. for 30 s, 52° C. for 30 s and 68° C. for 1 min; an additional elongation cycle of 68° C. for 7 min; and a holding cycle of 4° C. Reaction products were analysed on a 1% agarose gel (1×TAE). PCR products of the correct size (563 bp) were gel-purified using the Wizard PCR Preps DNA Purification System (Promega), eluted in 50 μl of water and subcloned directly.

Subcloning of PCR Products

The PCR product was subcloned into the topoisomerase I modified cloning vector (pCR4-TOPO) purchased from the Invitrogen Corporation using the conditions specified by the manufacturer. Briefly, 4 μl of gel purified PCR product was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm (room temperature) SOC media was added. Samples were incubated with shaking (220 rpm) for 1 h at 37° C. The transformation mixture was then plated on L-broth (LB) plates containing ampicillin (100 μg/ml) and incubated overnight at 37° C.

Colony PCR

Colonies were inoculated into 50 μl sterile water using a sterile toothpick. A 10 μl aliquot of the inoculum was then subjected to PCR in a total reaction volume of 20 μl containing 1× AmpliTaq™ buffer, 200 μM dNTPs, 20 pmoles of T7 primer, 20 pmoles of T3 primer, 1 unit of AmpliTaq™ (Perkin Elmer) using an MJ Research DNA Engine. The cycling conditions were as follows: 94° C., 2 min; 30 cycles of 94° C., 30 sec, 48° C., 30 sec and 72° C. for 1 min. Samples were maintained at 4° C. (holding cycle) before further analysis.

PCR reaction products were analyzed on a 1% agarose gel in 1×TAE buffer. Colonies which gave the expected PCR product size (563 bp cDNA+105 bp due to the multiple cloning site or MCS) were grown up overnight at 37° C. in 5 ml L-Broth (LB) containing ampicillin (100 μg/ml), with shaking at 220 rpm.

Plasmid DNA Preparation and Sequencing

Miniprep plasmid DNA was prepared from the 5 ml culture using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an Eppendorf BO photometer or a Spectramax 190 photometer (Molecular Devices). Plasmid DNA (100-200 ng) was subjected to DNA sequencing with the T7 and T3 primers using the BigDyeTerminator system (Applied Biosystems cat. no. 4390246) according to the manufacturer's instructions. The primer sequences are shown in Table 1. Sequencing reactions were purified using Dye-Ex columns (Qiagen) or Montage SEQ 96 cleanup plates (Millipore cat. no. LSKS09624) then analyzed on an Applied Biosystems 3700 sequencer.

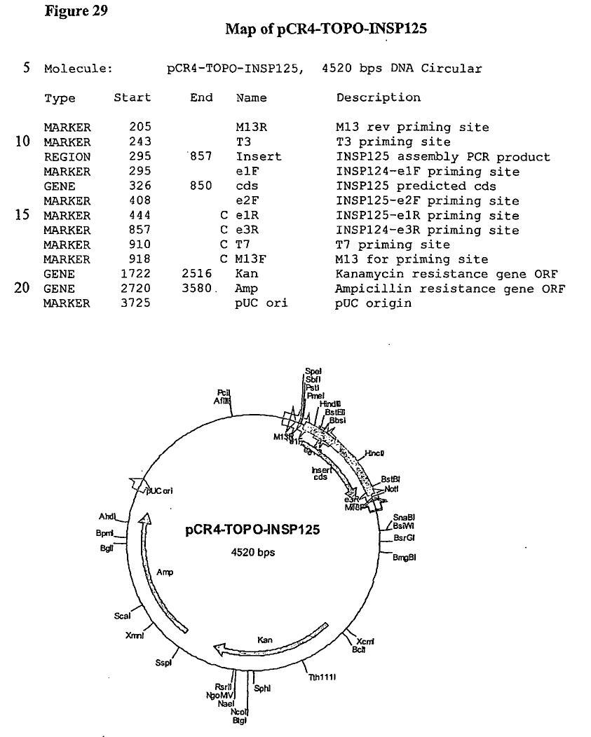

Sequence analysis identified a clone containing 100% match to the predicted INSP125 sequence. The sequence of the cloned cDNA fragment is shown in FIG. 3. The plasmid map of the cloned PCR product (pCR4-TOPO-INSP125, plasmid ID. 14681) is shown in FIG. 29.

### Example 13

### Construction of Mammalian Cell Expression Vectors for INSP125

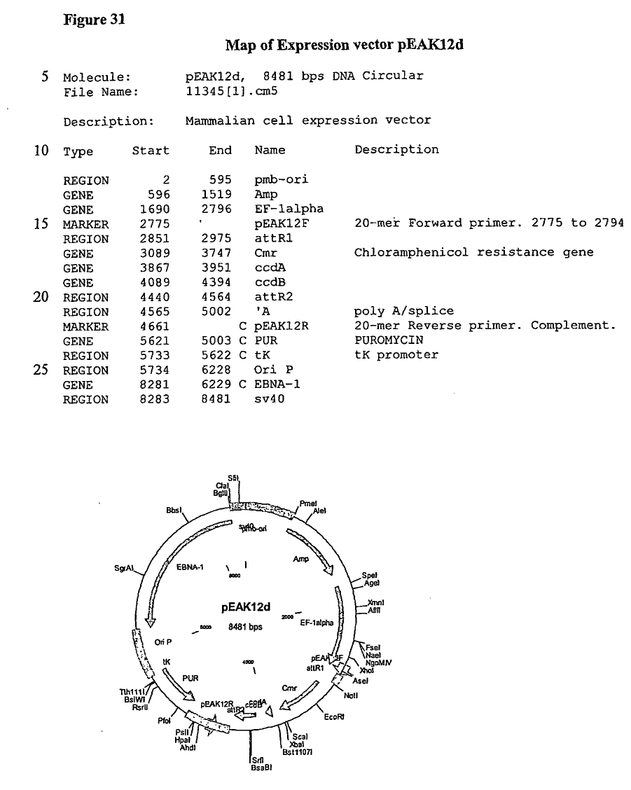

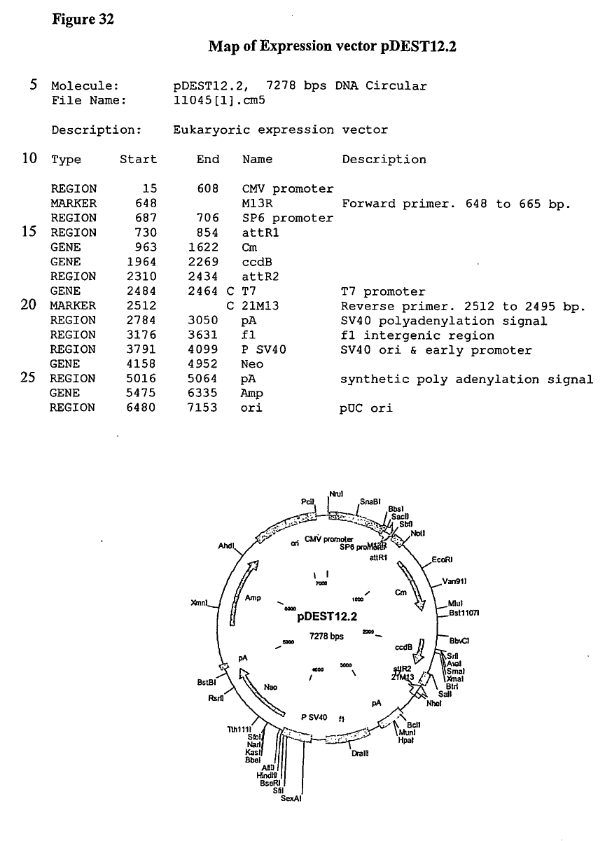

Plasmid 14681 was used as a PCR template to generate pEAK12d (FIG. 31) and pDEST12.2 (FIG. 32) expression clones containing the INSP125 ORF sequence with a 3′ sequence encoding a 6HIS tag using the Gateway™ cloning methodology (Invitrogen).

Generation of Gateway Compatible INSP125 ORF Fused to an in Frame 6HIS Tag Sequence.

The first stage of the Gateway cloning process involves a two step PCR reaction which generates the ORF of INSP125 flanked at the 5′ end by an attB1 recombination site and Kozak sequence, and flanked at the 3′ end by a sequence encoding an in frame 6 histidine (6HIS) tag, a stop codon and the attB2 recombination site (Gateway compatible cDNA). The first PCR reaction (in a final volume of 50 μl) contains: 1 μl (40 ng) of plasmid 14681, 1.5 μl dNTPs (10 mM), 10 μl of 10×Pfx polymerase buffer, 1 μl MgSO4 (50 mM), 0.5 μl each of gene specific primer (100 μM) (INSP125-EX1 and INSP125-EX2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification products were visualized on 0.8% agarose gel in 1×TAE buffer (Invitrogen) and a product migrating at the predicted molecular mass (593 bp) was purified from the gel using the Wizard PCR Preps DNA Purification System (Promega) and recovered in 50 μl sterile water according to the manufacturer's instructions.

The second PCR reaction (in a final volume of 50 μl) contained 10 μl purified PCR 1 product, 1.5 μl dNTPs (10 mM), 5 μl of 10×Pfx polymerase buffer, 1 μl MgSO4 (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. PCR products were gel purified using the Wizard PCR prep DNA purification system (Promega) according to the manufacturer's instructions.

Subcloning of Gateway Compatible INSP125 ORF into Gateway Entry Vector pDONR221 and Expression Vectors pEAK12d and pDEST12.2

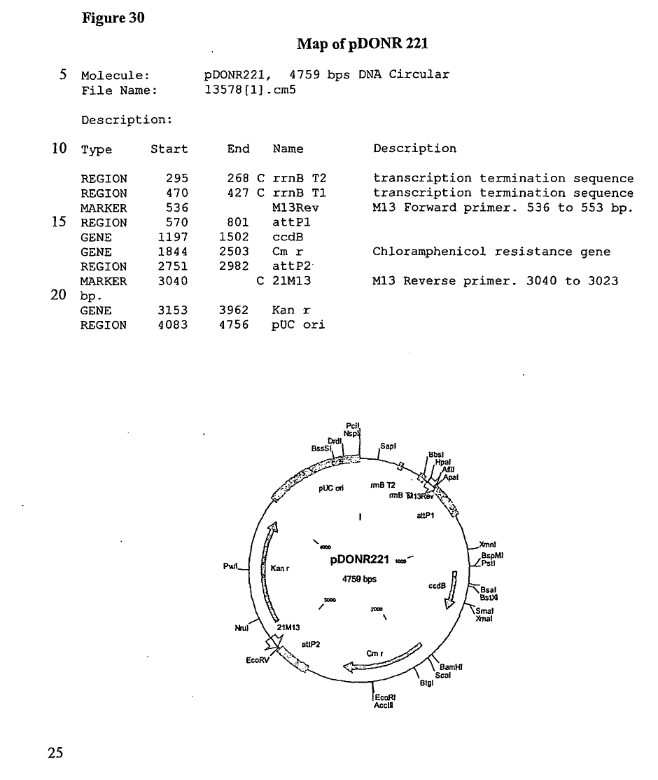

The second stage of the Gateway cloning process involves subcloning of the Gateway modified PCR product into the Gateway entry vector pDONR221 (Invitrogen, FIG. 30) as follows: 5 μl of purified product from PCR2 were incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of proteinase K (1 μl at 2 μg/μl) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 PI) were then plated on L-broth (LB) plates containing kanamycin (40 μg/ml) and incubated overnight at 37° C.

Plasmid mini-prep DNA was prepared from 5 ml cultures from 6 of the resultant colonies using a Qiaprep Turbo 9600 robotic system (Qiagen). Plasmid DNA (150-200 ng) was subjected to DNA sequencing with 21M13 and M13Rev primers using the BigDyeTerminator system (Applied Biosystems cat. no. 4390246) according to the manufacturer's instructions. The primer sequences are shown in Table 3. Sequencing reactions were purified using Dye-Ex columns (Qiagen) or Montage SEQ 96 cleanup plates (Millipore cat. no. LSKS09624) then analyzed on an Applied Biosystems 3700 sequencer.

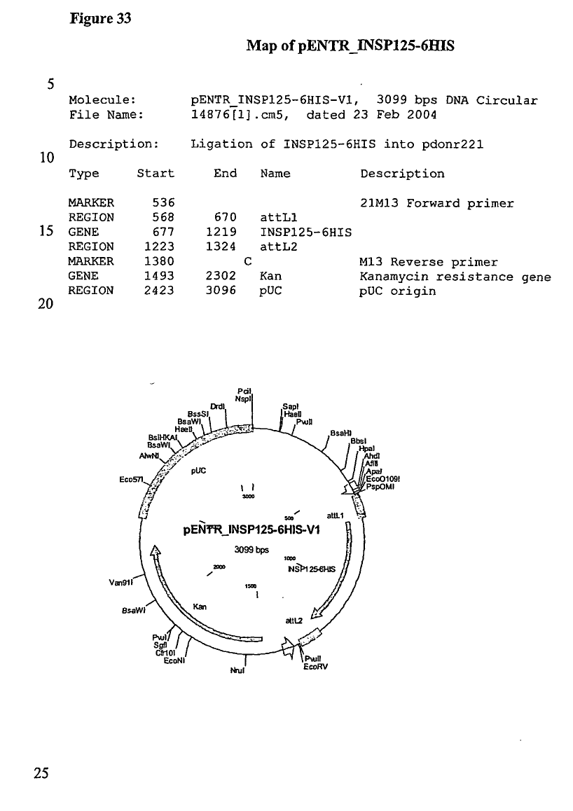

Plasmid eluate (2 μl or approx. 150 ng) from one of the clones which contained the correct sequence (pENTR_INSP125-6HIS, plasmid ID 14876, FIG. 33) was then used in a recombination reaction containing 1.5 μl of either pEAK12d vector or pDEST12.2 vector (FIGS. 31 & 32) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 ul) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep Turbo 9600 robotic system (Qiagen). Plasmid DNA (200-500 ng) in the pEAK12d vector was subjected to DNA sequencing with pEAK12F and pEAK12R primers as described above. Plasmid DNA (200-500 ng) in the pDEST12.2 vector was subjected to DNA sequencing with 21M13 and M13Rev primers as described above. Primer sequences are shown in Table 3.

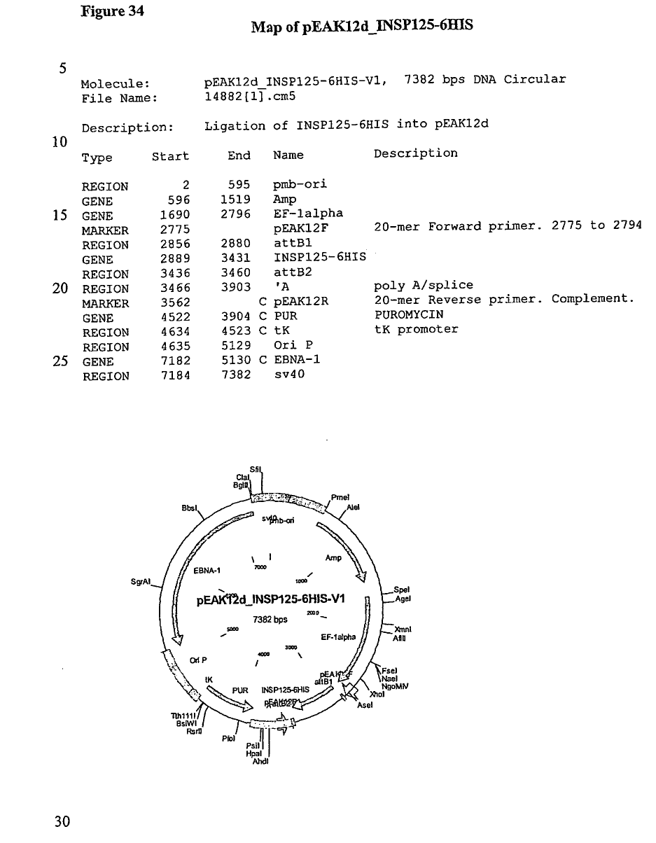

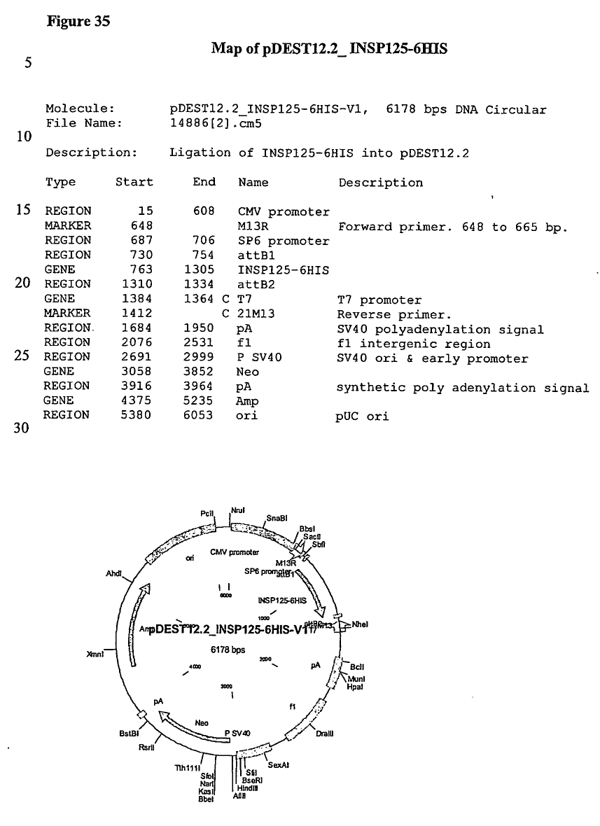

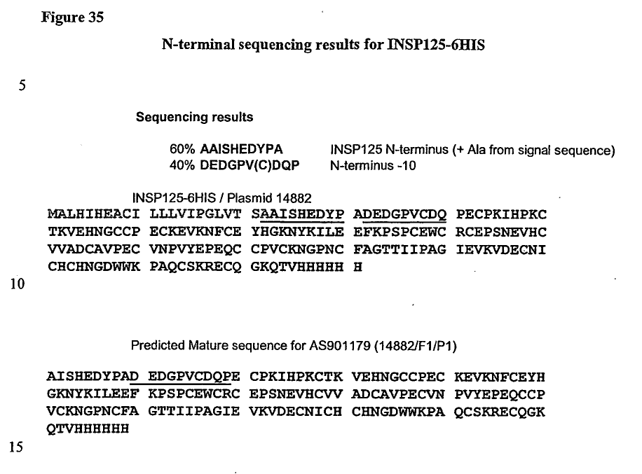

CsCl gradient purified maxi-prep DNA was prepared from a 500 ml culture of one of each of the sequence verified clones (pEAK12d_INSP125-6HIS, plasmid ID number 14882, FIG. 34, and pDEST12.2_INSP125-6HIS, plasmid ID 14886, FIG. 35) using the method described by Sambrook J. et al., 1989 (in Molecular Cloning, a Laboratory Manual, 2^{nd} edition, Cold Spring Harbor Laboratory Press), Plasmid DNA was resuspended at a concentration of 1 μg/μl in sterile water (or 10 mM Tris-HCl pH 8.5) and stored at −20° C.

5′ sequencing was performed on INSP125 to determine the correct mature polypeptide sequence. The sequencing yielded two mature forms for INSP125, one major form starting with AAISE (SEQ ID NO:59, and the other one starting with DEDGPV (SEQ ID NO:61). These results are displayed in FIG. 36.

### Example 14

### Expression and Purification of INSP123, INSP124 and INSP125

Further experiments may now be performed to determine the tissue distribution and expression levels of the INSP123, INSP124 and INSP125 polypeptides in vivo, on the basis of the nucleotide and amino acid sequences disclosed herein.

The presence of the transcripts for INSP123, INSP124 and INSP125 may be investigated by PCR of cDNA from different human tissues. The INSP123, INSP124 and INSP125 transcripts may be present at very low levels in the samples tested. Therefore, extreme care is needed in the design of experiments to establish the presence of a transcript in various human tissues as a small amount of genomic contamination in the RNA preparation will provide a false positive result. Thus, all RNA should be treated with DNAse prior to use for reverse transcription. In addition, for each tissue a control reaction may be set up in which reverse transcription was not undertaken (a −ve RT control).

For example, 1 μg of total RNA from each tissue may be used to generate cDNA using Multiscript reverse transcriptase (ABI) and random hexamer primers. For each tissue, a control reaction is set up in which all the constituents are added except the reverse transcriptase (−ve RT control). PCR reactions are set up for each tissue on the reverse transcribed RNA samples and the minus RT controls. INSP123, INSP124 and INSP125-specific primers may readily be designed on the basis of the sequence information provided herein. The presence of a product of the correct molecular weight in the reverse transcribed sample together with the absence of a product in the minus RT control may be taken as evidence for the presence of a transcript in that tissue. Any suitable cDNA libraries may be used to screen for the INSP123, INSP124 and INSP125 transcripts, not only those generated as described above.

The tissue distribution pattern of the INSP123, INSP124 and INSP125 polypeptides will provide further useful information in relation to the function of those polypeptides.

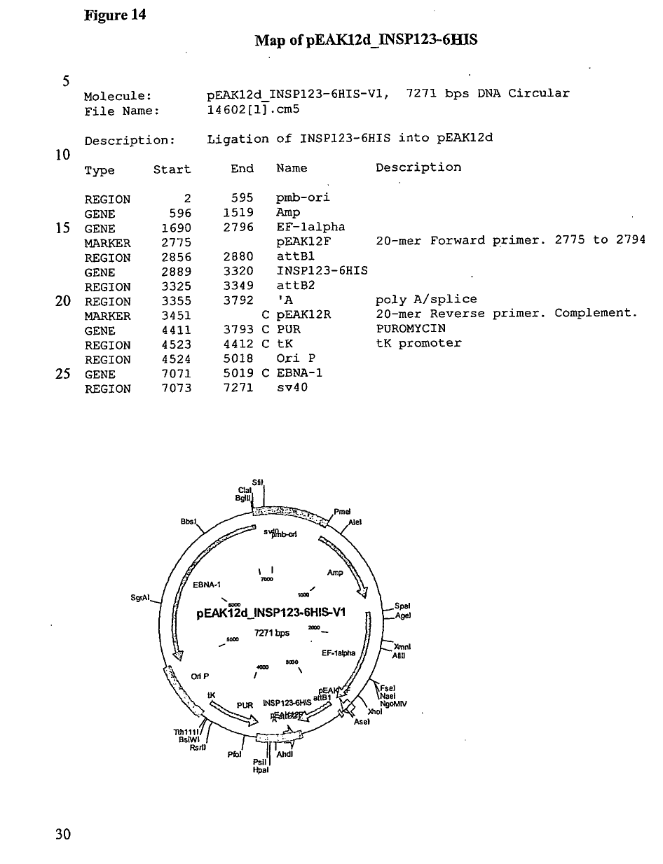

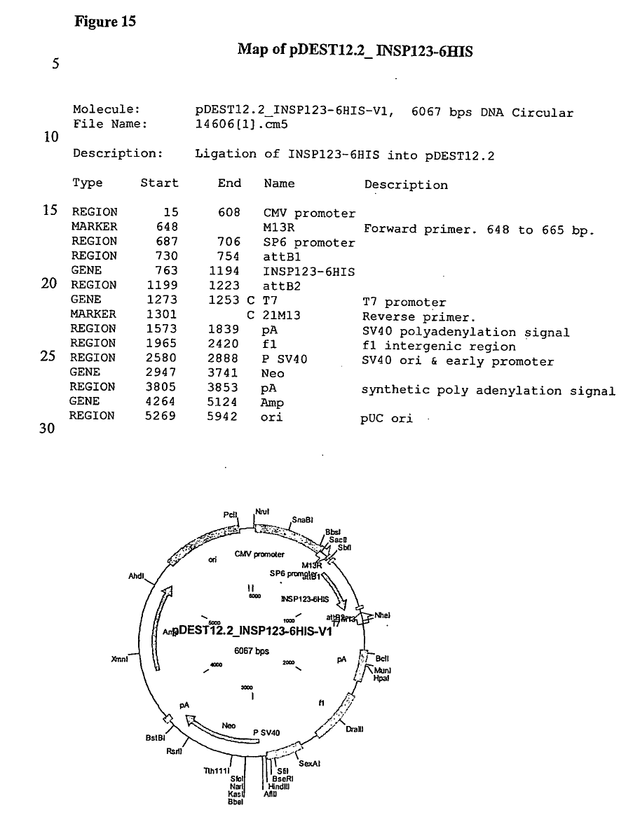

In addition, further experiments may now be performed using the pCR4-TOPO-INSP123 (FIG. 9), pDONR (FIG. 10), pEAK12d (FIG. 11), pDEST12.2 (FIG. 12), pENTR-INSP123-HIS (FIG. 13), pEAK12d-INSP123-6HIS (FIG. 14), pDEST12.2-INSP123-6HIS (FIG. 15), pCR4-BluntII-TOPO-INSP124 (FIG. 19), pDONR 221 (FIG. 20), pEAK12d (FIG. 21), pDEST12.2 (FIG. 22), pENTR_INSP124-6HIS (FIG. 23), pEAK12d-INSP124-6HIS (FIG. 24), pDEST12.2_INSP124-6HIS (FIG. 25), pCR4-TOPO-INSP125 (FIG. 29), pDONR 221 (FIG. 30), pEAK12d (FIG. 31), pDEST12.2 (FIG. 32), pENTR_INSP125-6HIS (FIG. 33), pEAK12d_INSP125-6HIS (FIG. 34) and pDEST12.2_INSP125-6HIS (FIG. 35) expression vectors. Transfection of mammalian cell lines with these vectors may enable the high level expression of the INSP123, INSP124 and INSP125 proteins and thus enable the continued investigation of the functional characteristics of the INSP123, INSP124 and INSP125 polypeptides. The following material and methods are an example of those suitable in such experiments:

Cell Culture

Human Embryonic Kidney 293 cells expressing the Epstein-Barr virus Nuclear Antigen (HEK293-EBNA, Invitrogen) are maintained in suspension in Ex-cell VPRO serum-free medium (seed stock, maintenance medium, JRH). Sixteen to 20 hours prior to transfection (Day-1), cells are seeded in 2×T225 flasks (50 ml per flask in DMEM/F12 (1:1) containing 2% FBS seeding medium (JRH) at a density of 2×10⁵ cells/ml). The next day (transfection day 0) transfection takes place using the JetPEI™ reagent (2 □l/□g of plasmid DNA, PolyPlus-transfection). For each flask, plasmid DNA is co-transfected with GFP (fluorescent reporter gene) DNA. The transfection mix is then added to the 2×T225 flasks and incubated at 37° C. (5% CO₂) for 6 days. Confirmation of positive transfection may be carried out by qualitative fluorescence examination at day 1 and day 6 (Axiovert 10 Zeiss).

On day 6 (harvest day), supernatants from the two flasks are pooled and centrifuged (e.g. 4° C., 400 g) and placed into a pot bearing a unique identifier. One aliquot (500 □l) is kept for QC of the 6His-tagged protein (internal bioprocessing QC).

Scale-up batches may be produced by following the protocol called “PEI transfection of suspension cells”, referenced BP/PEI/HH/02/04, with PolyEthyleneImine from Polysciences as transfection agent.

Purification Process

The culture medium sample containing the recombinant protein with a C-terminal 6His tag is diluted with cold buffer A (50 mM NaH₂PO₄; 600 mM NaCl; 8.7% (w/v) glycerol, pH 7.5). The sample is filtered then through a sterile filter (Millipore) and kept at 4° C. in a sterile square media bottle (Nalgene).

The purification is performed at 4° C. on the VISION workstation (Applied Biosystems) connected to an automatic sample loader (Labomatic). The purification procedure is composed of two sequential steps, metal affinity chromatography on a Poros 20 MC (Applied Biosystems) column charged with Ni ions (4.6×50 nm, 0.83 ml), followed by gel filtration on a Sephadex G-25 medium (Amersham Pharmacia) column (1.0×10 cm).

For the first chromatography step the metal affinity column is regenerated with 30 column volumes of EDTA solution (100 mM EDTA; 1M NaCl; pH 8.0), recharged with Ni ions through washing with 15 column volumes of a 100 mM NiSO₄ solution, washed with 10 column volumes of buffer A, followed by 7 column volumes of buffer B (50 mM NaH₂PO₄; 600 mM NaCl; 8.7% (w/v) glycerol, 400 mM; imidazole, pH 7.5), and finally equilibrated with 15 column volumes of buffer A containing 15 mM imidazole. The sample is transferred, by the Labomatic sample loader, into a 200 ml sample loop and subsequently charged onto the Ni metal affinity column at a flow rate of 10 ml/min. The column is washed with 12 column volumes of buffer A, followed by 28 column volumes of buffer A containing 20 mM imidazole. During the 20 mM imidazole wash loosely attached contaminating proteins are eluted from the column. The recombinant His-tagged protein is finally eluted with 10 column volumes of buffer B at a flow rate of 2 ml/min, and the eluted protein is collected.

For the second chromatography step, the Sephadex G-25 gel-filtration column is regenerated with 2 ml of buffer D (1.137M NaCl; 2.7 mM KCl; 1.5 mM KH₂PO₄; 8 mM Na₂HPO₄; pH 7.2), and subsequently equilibrated with 4 column volumes of buffer C (137 mM NaCl; 2.7 mM KCl; 1.5 mM KH₂PO₄; 8 mM Na₂HPO₄; 20% (w/v) glycerol; pH 7.4). The peak fraction eluted from the Ni-column is automatically loaded onto the Sephadex G-25 column through the integrated sample loader on the VISION and the protein is eluted with buffer C at a flow rate of 2 ml/min. The fraction was filtered through a sterile centrifugation filter (Millipore), frozen and stored at −80° C. An aliquot of the sample is analyzed on SDS-PAGE (4-12% NuPAGE gel; Novex) Western blot with anti-His antibodies. The NuPAGE gel may be stained in a 0.1% Coomassie blue R250 staining solution (30% methanol, 10% acetic acid) at room temperature for 1 h and subsequently destained in 20% methanol, 7.5% acetic acid until the background is clear and the protein bands clearly visible.

Following the electrophoresis the proteins are electrotransferred from the gel to a nitrocellulose membrane. The membrane is blocked with 5% milk powder in buffer E (137 mM NaCl; 2.7 mM KCl; 1.5 mM KH₂PO₄; 8 mM Na₂HPO₄; 0.1% Tween 20, pH 7.4) for 1 h at room temperature, and subsequently incubated with a mixture of 2 rabbit polyclonal anti-His antibodies (G-18 and H-15, 0.2 μg/ml each; Santa Cruz) in 2.5% milk powder in buffer E overnight at 4° C. After a further 1 hour incubation at room temperature, the membrane is washed with buffer E (3×10 min), and then incubated with a secondary HRP-conjugated anti-rabbit antibody (DAKO, HRP 0399) diluted 1/3000 in buffer E containing 2.5% milk powder for 2 hours at room temperature. After washing with buffer E (3×10 minutes), the membrane is developed with the ECL kit (Amersham Pharmacia) for 1 min. The membrane is subsequently exposed to a Hyperfilm (Amersham Pharmacia), the film developed and the western blot image visually analysed.

For samples that showed detectable protein bands by Coomassie staining, the protein concentration may be determined using the BCA protein assay kit (Pierce) with bovine serum albumin as standard.

Furthermore, overexpression or knock-down of the expression of the polypeptides in cell lines may be used to determine the effect on transcriptional activation of the host cell genome. Dimerisation partners, co-activators and co-repressors of the INSP123, INSP124 and INSP125 polypeptide may be identified by immunoprecipitation combined with Western blotting and immunoprecipitation combined with mass spectroscopy.

### Example 15

### Assays for the Detection of Biological Activity Similar to that of Secreted Proteins Containing a von Willebrand Factor Type C

1. Oligodendrocytes-Based Assays

Oligodendrocytes are responsible for myelin formation in the CNS. In multiple sclerosis they are the first cells attacked and their loss leads to major behavioral impairment. In addition to curbing inflammation, enhancing the incomplete remyelination of lesions that occurs in MS has been proposed as a therapeutic strategy for MS. Like neurons, mature oligodendrocytes do not divide but the new oligodendrocytes can arise from progenitors. There are very few of these progenitor cells in adult brain and even in embryos the number of progenitor cells is inadequate for HTS.

Oli-neu is a murine cell line obtained by an immortalization of an oligodendrocyte precursor by the t-neu oncogene. They are well studied and accepted as a representative cell line to study young oligodendrocyte biology.

These cells can be used in two types of assays.

One, to identify factors stimulating oligodendrocytes proliferation, and the other to find factors promoting their differentiation. Both events are key in the perspective of helping renewal and repairing demyelinating diseases.

Another possible cell line is the human cell line, MO3-13. MO3-13 results from the fusion of rabdo-myosarcoma cells with adult human oligodendrocytes. However these cells have a reduced ability to differentiate into oligodendrocytes and their proliferating rate is not sufficient to allow a proliferation assay. Nevertheless, they express certain features of oligodendrocytes and their morphology is well adapted to nuclear translocation studies. Therefore this cell line can be used in assays based on nuclear translocation of three transcription factors, respectively NF-kB, Stat-1 and Stat-2. The Jak/Stats transcription pathway is a complex pathway activated by many factors such as IFN α,β,γ, cytokines (e.g. IL-2, IL-6; IL-5) or hormones (e.g. GH, TPO, EPO). The specificity of the response depends on the combination of activated Stats. For example, it is noticeable that IFN-β activates Stat1, 2 and 3 nuclear translocations meanwhile IFN-γ only activates Stat1. In the same way, many cytokines and growth factors induced NF-kB translocation. In these assays the goal is to get a picture of activated pathways for a given protein.

2. Astrocyte-Based Assays

The biology of astrocytes is very complex, but two general states are recognized. In one state called quiescent, astrocytes regulate the metabolic and excitatory level of neurons by pumping glutamate and providing energetic substratum to neurons and oligodendrocytes. In the activated state, astrocytes produce chemokines and cytokines as well as nitric oxide. The first state could be considered as normal healthy while the second state occurs during inflammation, stroke or neurodegenerative diseases. When this activated state persists it should be regarded as a pathological state.

Many factors and many pathways are known to modulate astrocyte activation. In order to identify new factors modulating astrocyte activation U373 cells, a human cell line of astroglioma origin, can be used. NF-kB, c-Jun as well as Stats are signaling molecules known to play pivotal roles in astrocyte activation.

A series of screens based on the nuclear translocation of NF-kB, c-Jun and Stat1, 2 and 3 can be carried out. Prototypical activators of these pathways are IL-1b, IFN-beta or IFN-gamma. The goal is to identify proteins that could be used as therapeutics in the treatment of CNS diseases.

3. Neuron-Based Assays

Neurons are very complex and diverse cells but they have all in common two things. First they are post-mitotic cells, secondly they are innervating other cells. Their survival is linked to the presence of trophic factors often produced by the innervated target cells. In many neurodegenerative diseases the lost of target innervation leads to cell body atrophy and apoptotic cell death. Therefore identification of trophic factors supplementing target deficiency is very important in treatment of neurodegenerative diseases.

In this perspective a survival assay using NS1 cells, a sub-clone of rat PC12 cells, can be performed. These cells have been used for years and a lot of neurobiology knowledge has been first acquired on these cells before being confirmed on primary neurons including the pathways involved in neuron survival and differentiation (MEK, PI3K, CREB). In contrast the N2A cells, a mouse neuroblastoma, are not responding to classical neurotrophic factors but Jun-kinase inhibitors prevent apoptosis induced by serum deprivation. Therefore assays on these two cell lines will help to find different types of “surviving promoting” proteins.

It is important to note that in the previous assays we will identify factors that promote both proliferation and differentiation. In order to identify factors specifically promoting neuronal differentiation, a NS1 differentiation assay based on neurite outgrowth can be used. Promoting axonal or dendritic sprouting in neurodegenerative diseases could be advantageous for two reasons. It will first help the degenerating neurons to re-grow and reestablish a contact with the target cells. Secondly, it will potentiate the so-called collateral sprouting from healthy fibers, a compensatory phenomenon that delays terminal phases of neurodegenerative such as Parkinson or AD.

4. Endothelial Cell-Based Assays

The blood brain barrier (BBB) between brain and vessels is responsible of differences between cortical spinal fluid and serum compositions. The BBB results from a tight contact between endothelial cells and astrocytes. It maintains an immunotolerant status by preventing leukocytes penetration in brain, and allows the development of two parallels endocrine systems using the same intracellular signaling pathways. However, in many diseases or traumas, the BBB integrity is altered and leukocytes as well as serum proteins enter the brain inducing neuroinflammation. There is no easy in vitro model of BBB, but cultures of primary endothelial cells such as human embryonic umbilical endothelial cells (HUVEC) could mimic some aspect of BBB biology. For example, BBB leakiness could be induced by proteins stimulating intracellular calcium release. In the perspective of identifying proteins that modulate BBB integrity, a calcium mobilization assay with or without thrombin can be performed on HUVEC.

List of SEQFAM3 sequences:

```
SEQ ID 1
(INSP123 nucleotide sequence. Single exon.)
    1 ATGGCTCTTC ATATTCATGA AGCTTGCATA CTTCTGTTGG TCATCCCTGG ATTGGTCACC
  61 TCTGCTGCTA TCAGTCATGA AGACTATCCT GCTGATGAAG GTGACCAGAT CTCCAGTAAT
121 GACAATCTGA TCTTTGATGA CTATCGAGGG AAAGGGTGTG TCGATGACAG CGGCTTTGTA
181 TACAAGTTGG GAGAACGATT TTTCCCTGGG CATTCCAACT GTCCATGTGT CTGTGCTCTA
241 GATGGACCTG TTTGCGACCA ACCAGAATGC CCTAAAATTC ACCCAAAGTG TACTAAAGTG
301 GAACACAATG GATGCTGTCC TGAGTGCAAA GAAGTAAAAA ACTTCTGTGA ATATCACGGG
361 AAAAATTACA AAATCTTGGA GGAATTTAAG GTATGCGTTA CCCTCCATAT TTATTGA
SEQ ID 2
(INSP123 protein sequence. Single exon.)
    1 MALHIHEACI LLLVIPGLVT SAAISHEDYP ADEGDQISSN DNLIFDDYRG KGCVDDSGFV
  61 YKLGERFFPG HSNCPGVCAL DGPVCDQPEC PKIHPKCTKV EHNGCCPECK EVKNFCEYHG
121 KNYKILEEFK VCVTLHIY
SEQ ID 3
(INSP123 mature protein CDS - signal peptide cleaved 23:24aa)
    1 ATCAGTCATG AAGACTATCC TGCTGATGAA GGTGACCAGA TCTCCAGTAA TGACAATCTG
  61 ATCTTTGATG ACTATCGAGG GAAAGGGTGT GTCGATGACA GCGGCTTTGT ATACAAGTTG
121 GGAGAACGAT TTTTCCCTGG GCATTCCAAC TGTCCATGTG TCTGTGCTCT AGATGGACCT
181 GTTTGCGACC AACCAGAATG CCCTAAAATT CACCCAAAGT GTACTAAAGT GGAACACAAT
241 GGATGCTGTC CTGAGTGCAA AGAAGTAAAA AACTTCTGTG AATATCACGG GAAAAATTAC
301 AAAATCTTGG AGGAATTTAA GGTATGCGTT ACCCTCCATA TTTATTGA
SEQ ID 4
(INSP123 mature protein sequence - signal peptide cleaved 23:24aa)
    1 ISHEDYPADE GDQISSNDNL IFDDYRGKGC VDDSGFVYKL GERFFPGHSN GPCVCALDGP
  61 VCDQPECPKI HPKCTKVEHN GCCPECKEVK NFCEYHGKNY KILEEFKVCV TLHIY
SEQ ID 5
(INSP124 nucleotide sequence, first exon)
    1 ATGGCTCTTC ATATTCATGA AGCTTGCATA CTTCTGTTGG TCATCCCTGG ATTGGTCACC
  61 TCTGCTGCTA TCAGTCATGA AGACTATCCT GCTGATGAAG GTGACCAGAT CTCCAGTAAT
121 GACAATCTGA TCTTTGATGA CTATCGAGGG AAAGGGTGTG TCGATGACAG CGGCTTTGTA
181 TACAAGTTGG GAGAACGATT TTTCCCTGGG CATTCCAACT GTCCATGTGT CTGTGCTCTA
241 GATGGACCTG TTTGCGACCA ACCAGAATGC CCTAAAATTC ACCCAAAGTG TACTAAAGTG
301 GAACACAATG GATGCTGTCC TGAGTGCAAA GAAGTAAAAA ACTTCTGTGA ATATCACGGG
361 AAAAATTACA AAATCTTGGA GGAATTTAAG
SEQ ID 6
(INSP124 protein sequence, first exon)
    1 MALHIHEACI LLLVIPGLVT SAAISHEDYP ADEGDQISSN DNLIFDDYRG KGCVDDSGFV
  61 YKLGERFFPG HSNCPCVCAL DGPVCDQPEC PKIHPKCTKV EHNGCCPECK EVKNFCEYHG
121 KNYKILEEFK
SEQ ID 7
(INSP124 nucleotide sequence, second exon)
    1 CCCTCTCCAT GTGAATGGTG TCGCTGTGAG CCCAGCAATG AAGTTCACTG TGTTGTAGCA
  61 GACTGCGCAG TTCCTGAGTG TGTCAACCCA GTCTATGAAC CAGAACAATG TTGTCCTGTC
121 TGCAAAAATG
SEQ ID 8
(INSP124 protein sequence, second exon)
    1 PSPCEWCRCE PSNEVHCVVA DCAVPECVNP VYEPEQCCPV CKNG
SEQ ID 9
(INSP124 nucleotide sequence, third exon)
    1 GTCCAAACTG CTTTGCAGGA ACGACGATAA TTCCAGCTGG CATTGAAGTG AAAGTGGACG
  61 AATGTAACAT CTGTCATTGT CACAACGGGG ACTGGTGGAA GCCTGCTCAG TGTTCGAAAC
121 GTGAATGCCA AGGCAAGCAG ACTGTGTAG
SEQ ID 10
(INSP124 protein sequence, third exon)
    1 PNCFAGTTII PAGIEVKVDE CNICHCHNGD WWKPAQCSKR ECQGKQTV
SEQ ID 11
(INSP124 full coding sequence)
    1 ATGGCTCTTC ATATTCATGA AGCTTGCATA CTTCTGTTGG TCATCCCTGG ATTGGTCACC
  61 TCTGCTGCTA TCAGTCATGA AGACTATCCT GCTGATGAAG GTGACCAGAT CTCCAGTAAT
121 GACAATCTGA TCTTTGATGA CTATCGAGGG AAAGGGTGTG TCGATGACAG CGGCTTTGTA
181 TACAAGTTGG GAGAACGATT TTTCCCTGGG CATTCCAACT GTCCATGTGT CTGTGCTCTA
241 GATGGACCTG TTTGCGACCA ACCAGAATGC CCTAAAATTC ACCCAAAGTG TACTAAAGTG
301 GAACACAATG GATGCTGTCC TGAGTGCAAA GAAGTAAAAA ACTTCTGTGA ATATCACGGG
361 AAAAATTACA AAATCTTGGA GGAATTTAAG CCCTCTCCAT GTGAATGGTG TCGCTGTGAG
421 CCCAGCAATG AAGTTCACTG TGTTGTAGCA GACTGCGCAG TTCCTGAGTG TGTCAACCCA
481 GTCTATGAAC CAGAACAATG TTGTCCTGTC TGCAAAAATG GTCCAAACTG CTTTGCAGGA
541 ACGACGATAA TTCCAGCTGG CATTGAAGTG AAAGTGGACG AATGTAACAT GTGTCATTGT
601 CACAACGGGG ACTGGTGGAA GCCTGCTCAG TGTTCGAAAC GTGAATGCCA AGGCAAGCAG
661 ACTGTGTAG
SEQ ID 12
(INSP124 full protein sequence)
    1 MALHIHEACI LLLVIPGLVT SAAISHEDYP ADEGDQISSN DNLIFDDYRG KGCVDDSGFV
  61 YKLGERFFPG HSNCPCVCAL DGPVCDQPEC PKIHPKCTKV EHNGCCPECK EVKNFCEYHG
121 KNYKILEEFK PSPCEWCRCE PSNEVHCVVA DCAVPECVNP VYEFEQCCPV CKNGPNCFAG
181 TTIIPAGIEV KVDECNICHC HNGDWWKPAQ CSKRECQGKQ TV
SEQ ID 13
(INSP124 mature protein CDS first exon - signal peptide cleaved 23:24aa)
    1 ATCAGTCATG AAGACTATCC TGCTGATGAA GGTGACCAGA TCTCCAGTAA TGACAATCTG
  61 ATCTTTGATG ACTATCGAGG GAAAGGGTGT GTCGATGACA GCGGCTTTGT ATACAAGTTG
121 GGAGAACGAT TTTTCCCTGG GCATTCCAAC TGTCCATGTG TCTGTGCTCT AGATGGACCT
181 GTTTGCGACC AACCAGAATG CCCTAAAATT CACCCAAAGT GTACTAAAGT GGAACACAAT
241 GGATGCTGTC CTGAGTGCAA AGAAGTAAAA AACTTCTGTG AATATCACGG GAAAAATTAC
301 AAAATCTTGG AGGAATTTAA G
SEQ ID 14
(INSP124 mature protein sequence first exon - signal peptide cleaved 23:24aa)
    1 ISHEDYPADE GDQISSNDNL IFDDYRGKGC VDDSGFVYKL GERFFPGHSN CPCVCALDGP
  61 VCDQPECPKI HPKCTKVEHN GCCPECKEVK NFCEYHGKNY KILEEFK
SEQ ID 15
(INSP124 mature protein complete CDS - signal peptide cleaved 23:24aa)
    1 ATCAGTCATG AAGACTATCC TGCTGATGAA GGTGACCAGA TCTCCAGTAA TGACAATCTG
  61 ATCTTTGATG ACTATCGAGG GAAAGGGTGT GTCGATGACA GCGGCTTTGT ATACAAGTTG
121 GGAGAACGAT TTTTCCCTGG GCATTCCAAC TGTCCATGTG TCTGTGCTCT AGATGGACCT
181 GTTTGCGACC AACCAGAATG CCCTAAAATT CACCCAAAGT GTACTAAAGT GGAACACAAT
241 GGATGCTGTC CTGAGTGCAA AGAAGTAAAA AACTTCTGTG AATATCACGG GAAAAATTAC
301 AAAATCTTGG AGGAATTTAA GCCCTCTCCA TGTGAATGGT GTCGCTGTGA GCCCAGCAAT
361 GAAGTTCACT GTGTTGTAGC AGACTGCGCA GTTCCTGAGT GTGTCAACCC AGTCTATGAA
421 CCAGAACAAT GTTGTCCTGT CTGCAAAAAT GGTCCAAACT GCTTTGCAGG AACGACGATA
481 ATTCCAGCTG GCATTGAAGT GAAAGTGGAC GAATGTAACA TCTGTCATTG TCACAACGGG
541 GACTGGTGGA AGCCTGCTCA GTGTTCGAAA CGTGAATGCC AAGGCAAGCA GACTGTGTAG
SEQ ID 16
(INSP124 mature protein complete sequence - signal peptide cleaved 23:24aa)
    1 ISHEDYPADE GDQISSNDNL IFDDYRGKGC VDDSGFVYKL GERFFPGHSN CPCVCALDGP
  61 VCDQPECPKI HPKCTKVEHN GCCPECKEVK NFCEYHGKNY KILEEFKPSP CEWCRCEPSN
121 EVHCVVADCA VPECVNPVYE PEQCCPVCKN GPNCFAGTTI IPAGIEVKVD ECNICHCHNG
181 DWWKPAQCSK RECQGKQTV
SEQ ID 17
(INSP125 nucleotide sequence, first exon)
    1 ATGGCTCTTC ATATTCATGA AGCTTGCATA CTTCTGTTGG TCATCCCTGG ATTGGTCACC
  61 TCTGCTGCTA TCAGTCATGA AGACTATCCT GCTGATGAAG
SEQ ID 18
(INSP125 protein sequence, first exon)
    1 MALHIHEACI LLLVIPGLVT SAAISHEDYP ADED
SEQ ID 19
(INSP125 nucleotide sequence, second exon)
    1 ATGGACCTGT TTGCGACCAA CCAGAATGCC CTAAAATTCA CCCAAAGTGT ACTAAAGTGG
  61 AACACAATGG ATGCTGTCCT GAGTGCAAAG AAGTAAAAAA CTTCTGTGAA TATCACGGGA
121 AAAATTACAA AATCTTGGAG GAATTTAAG
SEQ ID 20
(INSP125 protein sequence, second exon)
    1 GPVCDQPECP KIHPKCTKVE HNGCCPECKE VKNFGEYHGK NYKILEEFK
SEQ ID 21
(INSP125 nucleotide sequence, third exon)
    1 CCCTCTCCAT GTGAATGGTG TCGCTGTGAG CCCAGCAATG AAGTTCACTG TGTTGTAGCA
  61 GACTGCGCAG TTCCTGAGTG TGTCAACCCA GTCTATGAAC CAGAACAATG TTGTCCTGTC
121 TGCAAAAATG
SEQ ID 22
(INSP125 protein sequence, third exon)
    1 PSPCEWCRCE PSNEVHCVVA DCAVPECVNP VYEPEQCCPV CKNG
SEQ ID 23
(INSP125 nucleotide sequence, fourth exon)
    1 GTCCAAACTG CTTTGCAGGA ACGACGATAA TTCCAGCTGG CATTGAAGTG AAAGTGGACG
  61 AATGTAACAT CTGTCATTGT CACAACGGGG ACTGGTGGAA GCCTGCTCAG TGTTCGAAAC
121 GTGAATGCCA AGGCAAGCAG ACTGTGTAG
SEQ ID 24
(INSP125 protein sequence, fourth exon)
    1 PNCFAGTTII PAGIEVKVDE CNICHCHNGD WWKPAQCSKR ECQGKQTV
SEQ ID 25
(INSP125 full coding sequence)
    1 ATGGCTCTTC ATATTCATGA AGCTTGCATA CTTCTGTTGG TCATCCCTGG ATTGGTCACC
  61 TCTGCTGCTA TCAGTCATGA AGACTATCCT GCTGATGAAG ATGGACCTGT TTGCGACCAA
121 CCAGAATGCC CTAAAATTCA CCCAAAGTGT ACTAAAGTGG AACACAATGG ATGCTGTCCT
181 GAGTGCAAAG AAGTAAAAAA CTTCTGTGAA TATCACGGGA AAAATTACAA AATCTTGGAG
241 GAATTTAAGC CCTCTCCATG TGAATGGTGT CGCTGTGAGC CCAGCAATGA AGTTCACTGT
301 GTTGTAGCAG ACTGCGCAGT TCCTGAGTGT GTCAACCCAG TCTATGAACC AGAACAATGT
361 TGTCCTGTCT GCAAAAATGG TCCAAACTGC TTTGCAGGAA CGACGATAAT TCCAGCTGGC
421 ATTGAAGTGA AAGTGGACGA ATGTAACATC TGTCATTGTC ACAACGGGGA CTGGTGGAAG
481 CCTGCTCAGT GTTCGAAACG TGAATGCCAA GGCAAGCAGA CTGTGTAG
SEQ ID 26
(INSP125 full protein sequence)
    1 MALHIHEACI LLLVIPGLVT SAAISHEDYP ADEDGPVCDQ PECPKIHPKC TKVEHNGCCP
  61 ECKEVKNFCE YHGKNYKILE EFKPSPCEWC RCEPSNEVHC VVADCAVPEC VNPVYEPEQC
121 CFVCKNGPNC FAGTTIIPAG IEVKVDECNI CHCHNGDWWK PAQCSKRECQ GKQTV
SEQ ID 27
(INSP125 mature protein CDS first exon - signal peptide cleaved 23:24aa)
    1 ATCAGTCATG AAGACTATCC TGCTGATGAA G
SEQ ID 28
(INSP125 mature protein first exon - signal peptide cleaved 23:24aa)
    1 IPGLVTSAAI SHEDYPADE
SEQ ID 29
(INSP125 mature protein CDS - signal peptide cleaved 23:24aa)
    1 ATCAGTCATG AAGACTATCC TGCTGATGAA GATGGACCTG TTTGCGACCA ACCAGAATGC
  61 CCTAAAATTC ACCCAAAGTG TACTAAAGTG GAACACAATG GATGCTGTCC TGAGTGCAAA
121 GAAGTAAAAA ACTTCTGTGA ATATCACGGG AAAAATTACA AAATCTTGGA GGAATTTAAG
181 CCCTCTCCAT GTGAATGGTG TCGCTGTGAG CCCAGCAATG AAGTTCACTG TGTTGTAGCA
241 GACTGCGCAG TTCCTGAGTG TGTCAACCCA GTCTATGAAC CAGAACAATG TTGTCCTGTC
301 TGCAAAAATG GTCCAAACTG CTTTGCAGGA ACGACGATAA TTCCAGCTGG CATTGAAGTG
361 AAAGTGGACG AATGTAACAT CTGTCATTGT CACAACGGGG ACTGGTGGAA GCCTGCTCAG
421 TGTTCGAAAC GTGAATGCCA AGGCAAGCAG ACTGTGTAG
SEQ ID 30
(INSP125 mature protein sequence - signal peptide cleaved 23:24aa)
    1 ISHEDYPADE DGPVCDQPEC PKIHPKCTKV EHNGCCPECK EVKNFCEYHG KNYKILEEFK
  61 PSPCEWCRCE PSNEVHCVVA DCAVPECVNP VYEPEQCCPV CKNGPNCFAG TTIIPAGIEV
121 KVDECNICHC HNGDWWKPAQ CSKPECQGKQ TV
SEQ ID 31
(Inpharmatica gene prediction of Mouse chr1 orthologue)
    1 MALHIHEACI LLLVIPGLVT SAAISHEDYP ADEGDQASSN DNLIFDDYRG KGCVDDSGFV
  61 YKLGERFFPG HSNCPCVCAL DGPVCDQPEC PKIHPKCTKV EHNGCCPECK EVKNFCEYHG
121 KNYKILEEFK V
SEQ ID 32
(Inpharmatica gene prediction of Mouse chr1 orthologue)
    1 MALHIHEACI LLLVIPGLVT SAAISHEDYP ADEGDQASSN DNLIFDDYRG KGCVDDSGFV
  61 YKLGERFFPG HSNCPCVCAL DGPVCDQPEC PKIHPKCTKV EHNGCCPECK EVKNFCEYHG
121 KNYKILEEFK PSPCEWCRCE PSNEVHCVVA DCAVPECVNP IYEPEQCCPV CKNGPNCFAG
181 TTIIPAGIEV KVDDCNICHC HNGDWWKPAQ CSKRECQGKQ TV
SEQ ID 33
(Inpharmatica gene prediction of Rat chr9 orthologue)
    1 MALHIHEACI LLLVIPGLVT PAAISHEDYP ADEGDQASSN DNLIFDDYRG KGCVDDSGFV
  61 YKLGERFFPG HSNCPCVCAL DGPVCDQPEC PKIHPKCTKV EHNGCCPECK EVKNFCEYHG
121 KNYKILEEFK V
SEQ ID 34
(Inpharmatica gene prediction of Rat chr14 orthologue)
    1 MPSSSAMAVG ALSSSLLVTC CLMVALCSPS IPLEELAQAP EQPGQEKREH ASRDSPGRVS
  61 ELGRASRDEG SSARDWKSKG SRALSGREAW SKQKQAWAAQ SGSAKAADWQ VRPRGDTPQG
121 EPPAAAQEAI SLELMPTPEL PEEYAYPDYR GKGCVDESGF VYAIGEKFAP GPSACPCLCT
181 EEGPLCAQPE CPRLHPRCIH VDTSQCCPQC KERKNYCEFR GKTYQTLEEF VVSPCERCRC
241 EANGEVLCTV SACPQTECVD PVYEPDQCCP ICKNGPNCFA ETAVIPAGRE VKTDECTICH
301 CTYEEGTWRI ERQANCTRHE CRQM
SEQ ID 35
(Inpharmatica gene prediction of Pufferfish gDNA scaffold_631 orthologue)
    1 NVVSESLSMT RRVRTAALAL LVCAHAVSGF SVAGQQESTC EENGGIYFVG EWYFLDSDHC
  61 TQCECTAEGP VCFRTECTSL PAACIHVSHY PTDCCPRCEK IGCEYRGVVY ELGQNFQPTE
121 CEQCTCDSDG IARCLVADCA PPPCVNPVYQ PGKCCPECKD GPNCYVTASR TQVIPAGEPT
181 WVDACTKCRC HDGQDAGYWE GNRLATCSRL KNCNHEGNLP RSK
SEQ ID 36
(Inpharmatica gene prediction of Pufferfish gDNA scaffold_889 orthologue)
    1 MLHSVAMTAE FLFVLVILTS SAQSHPIVTL PASREHSERV LSPAGQDNHS DKQAAEAYGV
  61 LEEROSLGPN RTASSPDRPN WNEQSSLNRI DEAPTSDVTL SLDAIDEYAY PDYRGKGCMD
121 ESGFVFAIGE QFTPGPSTCP CLCTDEGPLC TKPECPKVHP RCIKVDTSQC CPLCREKKNY
181 CDFRGKLYAS LEEFKVSPCE KCRCEPSGEV LCTVAACPQT ECVDPEYEPD QCCPICKSGE
241 WTPFPEL
SEQ ID 37
(Inpharmatica gene prediction of Pufferfish gDNA scaffold_1933 orthologue)
    1 MAPLLPATFV LLLALRAVTP AAVSNPEDYA ADEAERSAAD SIIFDDYRGK GCVDDSGFVY
  61 KLGERFYPGH SNCPCVCTED GPVCDQPECP RLHPKCTKVE HNGCCPECKE VKNFCEYRGK
121 TYKILEEFKV R
SEQ ID 38
(INSP123 cloned nucleotide sequence)
    1 ATGGCTCTTC ATATTCATGA AGCTTGCATA CTTCTGTTGG TCATCCCTGG ATTGGTCACC
  61 TCTGCTGCTA TCAGTCATGA AGACTATCCT GCTGATGAAG GTGACCAGAT CTCCAGTAAT
121 GACAATCTGA TCTTTGATGA CTATCGAGGG AAAGGGTGTG TCGATGACAG CGGCTTTGTA
181 TACAAGTTGG GAGAACGATT TTTCCCTGGG CATTCCAACT GTCCATGTGT CTGTGCTCTA
241 GATGGACCTG TTTGCGACCA ACCAGAATGC CCTAAAATTC ACCCAAAGTG TACTAAAGTG
301 GAACACAATG GATGCTGTCC TGAGTGCAAA GAAGTAAAAA ACTTCTGTGA ATATCACGGG
361 AAAAATTACA AAATCTTGGA GGAATTTAAG GTATGCGTTA CCCTCCATAT TTAT
SEQ ID 39
(INSP123 cloned polypeptide sequence)
    1 MALNIHEACI LLLVIPGLVT SAAISHEDYP ADEGDQISSN DNLIFDDYRG KGCVDDSGFV
  61 YKLGERFFPG HSNCPCVCAL DGPVCDQPEC PKIHPKCTKV EHNGCCPECK EVKNFCEYHG
121 KNYKILEEFK VCVTLHIY
SEQ ID 40
(INSP123 cloned mature nucleotide sequence 1)
    1 GCTATCAGTC ATGAAGACTA TCCTGCTGAT GAAGGTGACC AGATCTCCAG TAATGACAAT
  61 CTGATCTTTG ATGACTATCG AGGGAAAGGG TGTGTGGATG ACAGCGGCTT TGTATACAAG
121 TTGGGAGAAC GATTTTTCCC TGGGCATTCC AACTGTCCAT GTGTCTGTGC TCTAGATGGA
181 CCTGTTTGCG ACCAACCAGA ATGCCCTAAA ATTCACCCAA AGTGTACTAA AGTGGAACAC
241 AATGGATGCT GTCCTGAGTG CAAAGAAGTA AAAAACTTCT GTGAATATCA CGGGAAAAAT
301 TACAAAATCT TGGAGGAATT TAAGGTATGC GTTACCCTCC ATATTTAT
SEQ ID 41
(INSP123 cloned mature polypeptide sequence 1)
    1 AISHEDYPAD EGDQISSNDN LIFDDYRGKG CVDDSGFVYK LGERFFPGHS NCPCVCALDG
  61 PVCDQPECPK IHPKCTKVEH NGCCPECKEV KNFCEYHGKN YKILEEFKVC VTLHIY
SEQ ID 42
(INSP123 cloned mature nucleotide sequence 2)
    1 GCTGCTATCA GTCATGAAGA CTATCCTGCT GATGAAGGTG ACCAGATCTC CAGTAATGAC
  61 AATCTGATCT TTGATGACTA TCGAGGGAAA GGGTGTGTCG ATGACAGCGG CTTTGTATAC
121 AAGTTGGGAG AACGATTTTT CCCTGGGCAT TCCAACTGTC CATGTGTCTG TGCTCTAGAT
181 GGACCTGTTT GCGACCAACC AGAATGCCCT AAAATTCACC CAAAGTGTAC TAAAGTGGAA
241 CACAATGGAT GCTGTCCTGA GTGCAAAGAA GTAAAAAACT TCTGTGAATA TCACGGGAAA
301 AATTACAAAA TCTTGGAGGA ATTTAAGGTA TGCGTTACCC TCCATATTTA T
SEQ ID 43
(INSP123 cloned mature polypeptide sequence 2)
    1 AAISHEDYPA DEGDQISSND NLIFDDYRGK GCVDDSGFVY KLGERFFPGH SNCPCVCALD
  61 GPVCDQPECP KIHPKCTKVE HNGCCPECKE VKNFCEYHGK NYKILEEFKV CVTLHIY
SEQ ID 44
(INSP123 cloned mature nucleotide sequence 3)
    1 GATGAAGGTG ACCAGATCTC CAGTAATGAC AATCTGATCT TTGATGACTA TCGAGGGAAA
  61 GGGTGTGTCG ATGAGAGCGG CTTTGTATAC AAGTTGGGAG AACGATTTTT CCCTGGGCAT
121 TCCAACTGTC CATGTGTCTG TGCTCTAGAT GGACCTGTTT GCGACCAACC AGAATGCCCT
181 AAAATTCACC CAAAGTGTAC TAAAGTGGAA CACAATGGAT GCTGTCCTGA GTGCAAAGAA
241 GTAAAAAACT TCTGTGAATA TCACGGGAAA AATTACAAAA TCTTGGAGGA ATTTAAGGTA
301 TGCGTTACCC TCCATATTTA T
SEQ ID 45
(INSP123 cloned mature polypeptide sequence 3)
    1 DEGDQISSND NLIFDDYRGK GCVDDSGFVY KLGERFFPGH SNCPCVCALD GPVCDQPECP
  61 KIHPKCTKVE HNGCCPECKE VKNFCEYHGK NYKILEEFKV CVTLHIY
SEQ ID 46
(INSP124 cloned nucleotide sequence)
    1 ATGGCTCTTC ATATTCATGA AGCTTGCATA CTTCTGTTGG TCATCCCTGG ATTGGTCACC
  61 TCTGCTGCTA TCAGTCATGA AGACTATCCT GCTGATGAAG GTGACCAGAT CTCCAGTAAT
121 GACAATCTGA TCTTTGATGA CTATCGAGGG AAAGGGTGTG TCGATGACAG CGGCTTTGTA
181 TACAAGTTGG GAGAACGATT TTTCCCTGGG CATTCCAACT GTCCATGTGT CTGTGCTCTA
241 GATGGACCTG TTTGCGACCA ACCAGAATGC CCTAAAATTC ACCCAAAGTG TACTAAAGTG
301 GAACACAATG GATGCTGTCC TGAGTGCAAA GAAGTAAAAA ACTTCTGTGA ATATCACGGG
361 AAAAATTACA AAATCTTGGA GGAATTTAAG CCCTCTCCAT GTGAATGGTG TCGCTGTGAG
421 CCCAGCAATG AAGTTCACTG TGTTGTAGCA GACTGCGCAG TTCCTGAGTG TGTCAACCCA
481 GTCTATGAAC CAGAACAATG TTGTCCTGTC TGCAAAAATG GTCCAAACTG CTTTGCAGGA
541 ACGACGATAA TTCCAGCTGG CATTGAAGTG AAAGTGGACG AATGTAACAT CTGTCATTGT
601 CACAACGGGG ACTGGTGGAA GCCTGCTCAG TGTTCGAAAC GTGAATGCCA AGGCAAGCAG
661 ACTGTG
SEQ ID 47
(INSP124 cloned polypeptide sequence)
    1 MALHIHEACI LLLVIPGLVT SAAISHEDYP ADEGDQISSN DNLIFDDYRG KGCVDDSGFV
  61 YKLGERFFPG HSNCPCVCAL DGPVCDQPEC PKIHPKCTKV EHNGCCPECK EVKNFCEYHG
121 KNYKILEEFK PSPCEWCRCE PSNEVHCVVA DCAVPECVNP VYEPEQCCPV CKNGPNCFAG
181 TTIIPAGIEV KVDECNICHC HNGDWWKPAQ CSKRECQGKQ TV
SEQ ID 48
(INSP124 cloned mature nucleotide sequence 1)
    1 GCTATCAGTC ATGAAGACTA TCCTGCTGAT GAAGGTGACC AGATCTCCAG TAATGACAAT
  61 CTGATCTTTG ATGACTATCG AGGGAAAGGG TGTGTCGATG ACAGCGGCTT TGTATACAAG
121 TTGGGAGAAC GATTTTTCCC TGGGCATTCC AACTGTCCAT GTGTCTGTGC TCTAGATGGA
181 CCTGTTTGCG ACCAACCAGA ATGCCCTAAA ATTCACCCAA AGTGTACTAA AGTGGAACAC
241 AATGGATGCT GTCCTGAGTG CAAAGAAGTA AAAAACTTCT GTGAATATCA CGGGAAAAAT
301 TACAAAATCT TGGAGGAATT TAAGCCCTCT CCATGTGAAT GGTGTCGCTG TGAGCCCAGC
361 AATGAAGTTC ACTGTGTTGT AGCAGACTGC GCAGTTCCTG AGTGTGTCAA CCCAGTCTAT
421 GAACCAGAAC AATGTTGTCC TGTCTGCAAA AATGGTCCAA ACTGCTTTGC AGGAACGACG
481 ATAATTCCAG CTGGCATTGA AGTGAAAGTG GACGAATGTA ACATCTGTCA TTGTCACAAC
541 GGGGACTGGT GGAAGCCTGC TCAGTGTTCG AAACGTGAAT GCCAAGGCAA GCAGACTGTG
SEQ ID 49
(INSP124 cloned mature polypeptide sequence 1)
    1 AISHEDYFAD EGDQISSNDN LIFDDYRGKG CVDDSGFVYK LGERFFPGHS NCPCVCALDG
  61 PVCDQPECFK IHPKCTKVEH NGCCPECKEV KNFCEYHGKN YKILEEFKPS PCEWCRCEPS
121 NEVHCVVADC AVPECVNPVY EPEQCCPVCK NGPNCFAGTT IIPAGIEVKV DECNICNCHN
181 GDWWKPAQCS KRECQGKQTV
SEQ ID 50
(INSP124 cloned mature nucleotide sequence 2)
    1 GCTGCTATCA GTCATGAAGA CTATCCTGCT GATGAAGGTG ACCAGATCTC CAGTAATGAC
  61 AATCTGATCT TTGATGACTA TCGAGGGAAA GGGTGTGTCG ATGACAGCGG CTTTGTATAC
121 AAGTTGGGAG AAGGATTTTT CCCTGGGCAT TCCAACTGTC CATGTGTCTG TGCTCTAGAT
181 GGACCTGTTT GCGACCAACC AGAATGCCCT AAAATTCACC CAAAGTGTAC TAAAGTGGAA
241 CACAATGGAT GCTGTCCTGA GTGCAAAGAA GTAAAAAACT TCTGTGAATA TCACGGGAAA
301 AATTACAAAA TCTTGGAGGA ATTTAAGCCC TCTCCATGTG AATGGTGTCG CTGTGAGCCC
361 AGCAATGAAG TTCACTGTGT TGTAGCAGAC TGCGCAGTTC CTGAGTGTGT CAACCCAGTC
421 TATGAACCAG AACAATGTTG TCCTGTCTGC AAAAATGGTC CAAACTGCTT TGCAGGAACG
481 ACGATAATTC CAGCTGGCAT TGAAGTGAAA GTGGACGAAT GTAACATCTG TCATTGTCAC
541 AACGGGGACT GGTGGAAGCC TGCTCAGTGT TCGAAACGTG AATGCCAAGG CAAGCAGACT
601 GTG
SEQ ID 51
(INSP124 cloned mature polypeptide sequence 2)
    1 AAISHEDYPA DEGDQISSND NLIFDDYRGK GCVDDSGFVY KLGERFFPGH SNCPCVCALD
  61 GPVCDQPECP KIHPKCTKVE HNGCCPECKE VKNFCEYHGK NYKILEEFKP SFCEWCRCEP
121 SNEVHCVVAD CAVPECVNPV YEPEQCCPVC KNGPNCFAGT TIIPAGIEVK VDECNICHCH
181 NGDWWKPAQC SKRECQGKQT V
SEQ ID 52
(INSP124 cloned mature nucleotide sequence 3)
    1 GATGAAGGTG ACCAGATCTC CAGTAATGAC AATCTGATCT TTGATGACTA TCGAGGGAAA
  61 GGGTGTGTCG ATGACAGCGG CTTTGTATAC AAGTTGGGAG AACGATTTTT CCCTGGGCAT
121 TCCAACTGTC CATGTGTCTG TGCTCTAGAT GGACCTGTTT GCGACCAACC AGAATGCCCT
181 AAAATTCACC CAAAGTGTAC TAAAGTGGAA CACAATGGAT GCTGTCCTGA GTGCAAAGAA
241 GTAAAAAACT TCTGTGAATA TCACGGGAAA AATTACAAAA TCTTGGAGGA ATTTAAGCCC
301 TCTCCATGTG AATGGTGTCG CTGTGAGCCC AGCAATGAAG TTCACTGTGT TGTAGCAGAC
361 TGCGCAGTTC CTGAGTGTGT CAACCCAGTC TATGAACCAG AACAATGTTG TCCTGTCTGC
421 AAAAATGGTC CAAACTGCTT TGCAGGAACG ACGATAATTC CAGCTGGCAT TGAAGTGAAA
481 GTGGACGAAT GTAACATCTG TCATTGTCAC AACGGGGACT GGTGGAAGCC TGCTCAGTGT
541 TCGAAACGTG AATGCCAAGG CAAGCAGACT GTG
SEQ ID 53
(INSP124 cloned mature polypeptide sequence 3)
    1 DEGDQISSND NLIFDDYRGK GCVDDSGFVY KLGERFFPGH SNCPCVCALD GPVCDQPECP
  61 KIHPKCTKVE HNGCCPECKE VKNFCEYHGK NYKILEEFKP SPCEWCRCEP SNEVHCVVAD
121 CAVPECVNPV YEPEQCCPVC KNGPNCFAGT TIIPAGIEVK VDECNICHCH NGDWWKPAQC
181 SKRECQGKQT V
SEQ ID 54
(INSP125 cloned nucleotide sequence)
    1 ATGGCTCTTC ATATTCATGA AGCTTGCATA CTTCTGTTGG TCATCCCTGG ATTGGTCACC
  61 TCTGCTGCTA TCAGTCATGA AGACTATCCT GCTGATGAAG ATGGACCTGT TTGCGACCAA
121 CCAGAATGCC CTAAAATTCA CCCAAAGTGT ACTAAAGTGG AACACAATGG ATGCTGTCCT
181 GAGTGCAAAG AAGTAAAAAA CTTCTGTGAA TATCACGGGA AAAATTACAA AATCTTGGAG
241 GAATTTAAGC CCTCTCCATG TGAATGGTGT CGCTGTGAGC CCAGCAATGA AGTTCACTGT
301 GTTGTAGCAG ACTGCGCAGT TCCTGAGTGT GTCAACCCAG TCTATGAACC AGAACAATGT
361 TGTCCTGTCT GCAAAAATGG TCCAAACTGC TTTGCAGGAA CGACGATAAT TCCAGCTGGC
421 ATTGAAGTGA AAGTGGACGA ATGTAACATC TGTCATTGTC ACAACGGGGA CTGGTGGAAG
481 CCTGCTCAGT GTTCGAAACG TGAATGCCAA GGCAAGCAGA CTGTG
SEQ ID 55
(INSP125 cloned polypeptide sequence)
    1 MALHIHEACI LLLVIPGLVT SAAISHEDYP ADEDGPVCDQ PECPKIHPKC TKVEHNGCCP
  61 ECKEVKNFCE YHGKNYKILE EFKPSPCEWC RCEPSNEVHC VVADCAVPEC VNPVYEFEQC
121 CPVCKNGPNC FAGTTIIPAG IEVKVDECNI CHCHNGDWWK PAQCSKRECQ GKQTV
SEQ ID 56
(INSP125 cloned mature nucleotide sequence 1)
    1 GCTATCAGTC ATGAAGACTA TCCTGCTGAT GAAGATGGAC CTGTTTGCGA CCAACCAGAA
  61 TGCCCTAAAA TTCACCCAAA GTGTACTAAA GTGGAACACA ATGGATGCTG TCCTGAGTGC
121 AAAGAAGTAA AAAACTTCTG TGAATATCAC GGGAAAAATT ACAAAATCTT GGAGGAATTT
181 AAGCCCTCTC CATGTGAATG GTGTCGCTGT GAGCCCAGCA ATGAAGTTCA CTGTGTTGTA
241 GCAGACTGCG CAGTTCCTGA GTGTGTCAAC CCAGTCTATG AACCAGAACA ATGTTGTCCT
301 GTCTGCAAAA ATGGTCCAAA CTGCTTTGCA GGAACGACGA TAATTCCAGC TGGCATTGAA
361 GTGAAAGTGG ACGAATGTAA CATCTGTCAT TGTCACAACG GGGACTGGTG GAAGCCTGCT
421 CAGTGTTCGA AACGTGAATG CCAAGGCAAG CAGACTGTG
SEQ ID 57
(INSP125 cloned mature polypeptide sequence 1)
    1 AISHEDYPAD EDGPVCDQPE CPKIHPKCTK VEHNGCCPEC KEVKNFCEYH GKNYKILEEF
  61 KPSPCEWCRC EPSNEVHCVV ADCAVPECVN PVYEPEQCCP VCKNGPNCFA GTTIIPAGIE
121 VKVDECNICH CHNGDWWKPA QCSKRECQGK QTV
SEQ ID 58
(INSP125 cloned mature nucleotide sequence 2)
    1 GCTGCTATCA GTCATGAAGA CTATCCTGCT GATGAAGATG GACCTGTTTG CGACCAACCA
  61 GAATGCCCTA AAATTCACCC AAAGTGTACT AAAGTGGAAC ACAATGGATG CTGTCCTGAG
121 TGCAAAGAAG TAAAAAACTT CTGTGAATAT CACGGGAAAA ATTACAAAAT CTTGGAGGAA
181 TTTAAGCCCT CTCCATGTGA ATGGTGTCGC TGTGAGCCCA GGAATGAAGT TCACTGTGTT
241 GTAGCAGACT GCGCAGTTCC TGAGTGTGTC AACCCAGTCT ATGAACCAGA ACAATGTTGT
301 CCTGTCTGCA AAAATGGTCC AAACTGCTTT GCAGGAACGA CGATAATTCC AGCTGGCATT
361 GAAGTGAAAG TGGACGAATG TAACATCTGT CATTGTCACA ACGGGGACTG GTGGAAGCCT
421 GCTCAGTGTT CGAAACGTGA ATGCCAAGGC AAGCAGACTG TG
SEQ ID 59
(INSP125 cloned mature polypeptide sequence 2)
    1 AAISHEDYPA DEDGPVCDQP ECPKIHPICT KVEHNGCCPE CKEVKNFCEY HGKNYKILEE
  61 FKPSPCEWCR CEPSNEVHCV VADCAVPECV NFVYEPEQCC PVCKNGPNCF AGTTIIPAGI
121 EVKVDECNIC HCHNGDWWKP AQCSKRECQG KQTV
SEQ ID 60
(INSP125 cloned mature nucleotide sequence 3)
    1 GATGAAGATG GACCTGTTTG CGACCAACCA GAATGCCCTA AAATTCACCC AAAGTGTACT
  61 AAAGTGGAAC ACAATGGATG CTGTCCTGAG TGCAAAGAAG TAAAAAACTT CTGTGAATAT
121 CACGGGAAAA ATTACAAAAT CTTGGAGGAA TTTAAGCCCT CTCCATGTGA ATGGTGTCGC
181 TGTGAGCCCA GCAATGAAGT TCACTGTGTT GTAGCAGACT GCGCAGTTCC TGAGTGTGTC
241 AACCCAGTCT ATGAACCAGA ACAATGTTGT CCTGTCTGCA AAAATGGTCC AAACTGCTTT
301 GCAGGAACGA CGATAATTCC AGCTGGCATT GAAGTGAAAG TGGACGAATG TAACATCTGT
361 CATTGTCACA ACGGGGACTG GTGGAAGCCT GCTCAGTGTT CGAAACGTGA ATGCCAAGGC
421 AAGCAGAGTG TG
SEQ ID 61
(INSP125 cloned mature polypeptide sequence 3)
    1 DEOGPVCDQP ECPKIHPKCT KVEHNGCCPE CKEVKNFCEY HGKNYKILEE FKPSPCEWCR
  61 CEPSNEVHCV VADCAVPECV NPVYEPEQCC PVCKNGPNCF AGTTIIPAGI EVKVDECNIC
121 HCHNGDWWKP AQCSKRECQG KQTV
```

We claim:

1: A method of identifying a member of the SECFAM3 family comprising searching a database of translated nucleic acid sequences or amino acid sequences to identify an amino acid sequence that matches the following sequence profile:

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | M | −2 | −2 | −3 | −4 | −2 | −1 | −3 | −4 | −3 | 0 | 2 | −2 | 8 | 0 | −3 | −2 | −2 | −2 | −2 | 0 |
| 2 | A | 3 | −1 | −3 | −3 | −1 | −1 | −2 | −2 | −3 | 0 | 0 | 1 | 2 | 1 | −2 | −1 | −1 | −3 | −1 | 1 |
| 3 | L | 1 | −2 | −2 | −3 | −2 | −2 | −2 | −2 | 2 | 0 | 2 | −2 | 0 | −2 | 0 | 1 | −1 | −3 | −2 | 2 |
| 4 | H | −1 | −1 | 1 | −1 | −3 | −1 | −1 | 3 | 6 | −3 | −1 | −1 | −2 | −2 | −2 | 2 | −1 | −3 | −1 | −3 |
| 5 | I | 0 | −2 | −3 | −3 | −2 | 0 | 0 | −3 | −3 | 3 | 2 | −2 | 0 | −1 | −3 | −2 | −1 | −3 | −2 | 2 |
| 6 | H | 0 | −2 | −1 | −2 | −2 | −1 | −1 | −3 | 7 | 0 | 0 | −2 | −1 | −2 | 0 | 0 | −1 | −3 | 0 | 2 |
| 7 | E | 0 | −2 | −2 | −1 | −2 | −1 | 2 | −3 | −1 | 0 | 1 | −1 | 2 | 2 | −3 | 0 | −1 | −2 | 2 | 0 |
| 8 | A | 2 | −2 | −1 | −2 | 3 | −2 | −2 | 2 | −2 | −2 | −2 | −2 | −2 | −3 | −2 | 3 | 1 | −3 | −3 | −2 |
| 9 | C | 0 | −3 | −2 | −3 | 7 | −2 | −3 | −3 | −3 | 0 | −1 | −2 | 3 | 3 | −3 | 0 | −1 | −2 | −1 | 0 |
| 10 | I | −1 | −2 | −2 | −2 | −2 | −2 | 0 | 0 | −2 | 3 | 2 | −2 | 0 | 0 | −3 | 0 | 0 | −2 | 3 | 0 |
| 11 | L | −2 | 0 | −3 | −4 | −2 | −2 | −3 | −4 | −3 | 0 | 4 | −2 | 3 | 3 | −4 | −2 | −2 | −2 | 0 | 0 |
| 12 | L | 0 | 0 | −3 | −4 | −2 | −2 | −3 | −3 | −3 | 0 | 3 | −2 | 0 | −1 | 1 | −2 | −1 | −3 | −2 | 1 |
| 13 | L | −1 | −2 | −2 | −2 | −2 | −2 | 0 | −3 | −3 | 0 | 3 | −2 | 0 | 1 | −3 | 1 | 1 | −3 | −1 | 1 |
| 14 | V | 0 | 0 | −3 | −3 | 4 | 0 | −2 | −3 | −3 | 0 | 0 | −2 | 2 | −2 | −3 | −2 | −1 | −3 | −2 | 4 |
| 15 | I | −1 | 0 | −2 | −3 | 4 | −2 | −2 | −3 | 2 | 2 | 1 | −2 | 0 | −1 | −3 | 0 | 0 | 4 | −1 | 0 |
| 16 | P | 0 | 0 | −2 | −2 | −2 | −2 | −2 | −3 | 4 | −1 | 0 | −2 | −1 | 2 | 3 | 0 | −1 | −2 | 0 | 0 |
| 17 | G | 0 | −3 | −2 | −3 | −2 | −2 | −3 | 2 | −3 | 1 | 0 | −3 | 3 | 3 | −3 | −1 | −2 | −2 | 0 | 1 |
| 18 | L | 1 | −3 | −3 | −4 | −1 | −2 | −3 | −3 | −3 | 0 | 4 | −2 | 0 | −1 | −3 | −2 | −1 | −3 | −2 | 2 |
| 19 | V | 2 | −1 | −1 | −2 | −2 | 3 | −1 | −2 | −2 | 0 | −1 | −1 | −1 | −3 | −2 | 1 | 1 | −3 | −2 | 2 |
| 20 | T | −1 | −2 | −2 | −3 | 4 | −2 | −2 | −3 | −3 | 0 | 3 | −2 | 0 | −2 | −3 | 1 | 3 | −3 | −2 | 0 |
| 21 | S | 0 | −2 | −1 | −2 | 4 | −2 | −2 | 1 | −3 | −1 | 1 | −2 | 2 | −2 | 0 | 2 | −1 | −3 | −3 | −1 |
| 22 | A | 3 | −2 | −2 | −2 | −1 | −2 | −2 | −1 | −2 | −1 | 0 | −2 | −1 | 1 | 2 | 2 | −1 | −3 | −2 | 0 |
| 23 | A | 2 | −2 | −3 | −2 | 5 | 0 | 0 | −2 | −3 | −1 | 0 | −2 | −1 | −3 | 2 | −1 | −1 | −3 | −2 | 1 |
| 24 | I | 1 | 1 | −2 | −2 | −2 | −1 | −2 | −2 | −2 | 2 | 0 | −1 | −1 | −1 | −2 | 2 | −1 | −3 | 1 | 1 |
| 25 | S | −1 | −1 | 2 | −1 | −2 | 1 | −1 | −2 | 4 | 2 | −1 | −1 | −1 | −2 | −2 | 3 | −1 | −4 | −1 | −1 |
| 26 | H | 0 | −2 | 3 | −1 | −3 | −1 | −1 | −2 | 5 | −2 | −2 | −1 | −2 | 0 | 3 | 0 | −1 | −3 | −1 | 0 |
| 27 | E | −1 | −1 | 0 | 1 | −3 | 0 | 5 | −2 | −1 | −3 | −3 | 0 | −2 | −3 | −1 | 1 | 2 | −3 | −2 | −2 |
| 28 | D | 1 | −2 | 0 | 4 | −2 | −1 | 0 | −1 | −2 | −2 | 0 | −1 | −1 | −2 | −1 | 2 | 0 | −4 | −3 | −2 |
| 29 | Y | −2 | −1 | −2 | −2 | −3 | 2 | −1 | −3 | 0 | 0 | −1 | −1 | −1 | 0 | 3 | −1 | −2 | 0 | 5 | 0 |
| 30 | P | 0 | −1 | −1 | −1 | −2 | −1 | −1 | 0 | 5 | −3 | −3 | −1 | −2 | −3 | 4 | 0 | 0 | −3 | −1 | −2 |
| 31 | A | 3 | −2 | −2 | −2 | −1 | −1 | −2 | −1 | −3 | 0 | 2 | −1 | 0 | −2 | 0 | 0 | 0 | −3 | −2 | 0 |
| 32 | D | −2 | −1 | 0 | 5 | −3 | 0 | 2 | −2 | −1 | −2 | −3 | 2 | −2 | −3 | −1 | 0 | −1 | −4 | −3 | 0 |
| 33 | E | 0 | −1 | −2 | 0 | −2 | 0 | 3 | −3 | −1 | 0 | 0 | −1 | 0 | 3 | −2 | −1 | −1 | −2 | 0 | 0 |
| 34 | G | 1 | −2 | 0 | −1 | −2 | −1 | −2 | 4 | −2 | −2 | −2 | −2 | 0 | −2 | −2 | 1 | 1 | −2 | −3 | 0 |
| 35 | D | 0 | 2 | 0 | 2 | −2 | 0 | 0 | −2 | −1 | −2 | 0 | 0 | −1 | −3 | 2 | −1 | −1 | −3 | −2 | −2 |
| 36 | Q | 0 | 3 | 0 | −1 | −2 | 3 | 0 | −2 | −1 | −2 | −2 | 0 | −1 | −3 | −1 | 1 | 3 | −3 | −2 | −2 |
| 37 | I | 0 | −2 | −3 | −2 | −1 | −2 | −2 | −2 | −3 | 1 | 0 | −2 | 0 | −2 | 4 | −1 | −1 | −3 | −2 | 2 |
| 38 | S | −1 | −1 | 0 | 1 | −2 | 0 | 2 | −2 | −1 | 2 | −1 | 1 | −1 | −2 | −2 | 2 | −1 | −3 | −2 | 0 |
| 39 | S | 3 | −1 | −1 | −1 | −1 | −1 | −1 | −1 | −2 | −1 | 0 | −1 | −1 | −2 | 2 | 3 | 0 | −3 | −2 | −1 |
| 40 | N | 0 | −2 | 2 | −1 | −3 | −1 | 1 | 3 | −1 | 0 | 0 | −1 | −1 | −3 | 1 | −1 | −1 | −3 | −2 | 0 |
| 41 | D | 1 | −2 | −1 | 3 | −2 | 0 | 2 | −2 | −2 | −1 | 0 | −1 | −1 | 0 | −2 | 0 | −1 | −3 | −2 | −1 |
| 42 | N | −1 | −1 | 2 | 1 | −3 | 0 | 3 | 0 | −1 | −2 | 0 | 0 | −1 | −2 | −2 | 1 | −1 | −3 | −2 | −2 |
| 43 | L | 1 | −3 | −3 | −3 | 4 | −2 | −3 | −3 | −1 | 0 | 1 | −3 | 0 | 3 | −3 | −2 | −2 | 0 | 5 | 0 |
| 44 | I | 1 | −1 | −1 | 0 | −2 | 0 | 2 | −2 | −2 | 1 | −1 | 1 | −1 | −2 | −2 | 2 | −1 | −4 | −2 | 0 |
| 45 | F | −2 | −3 | −2 | 1 | −2 | −2 | −2 | −3 | −1 | 2 | 0 | −3 | 0 | 4 | −3 | −2 | −2 | −1 | 4 | 1 |
| 46 | D | 1 | −2 | −1 | 2 | −2 | −1 | 0 | 0 | −2 | −3 | −3 | −1 | −2 | −3 | 4 | 1 | −1 | −4 | −3 | −2 |
| 47 | D | 0 | −2 | 0 | 5 | −3 | −1 | 0 | 1 | −2 | −3 | −4 | −1 | −3 | −3 | 2 | 1 | −1 | −4 | −3 | −3 |
| 48 | Y | −2 | 3 | −1 | −2 | −3 | 2 | −1 | −3 | 0 | −2 | −2 | 0 | −1 | 0 | −3 | −2 | −2 | 0 | 6 | −2 |
| 49 | R | −2 | 4 | 0 | −2 | −4 | 2 | 0 | −2 | 5 | −4 | −3 | 0 | −2 | −3 | 2 | −1 | −2 | −3 | −1 | −3 |
| 50 | G | −1 | −2 | 0 | 2 | −3 | −1 | 1 | 4 | −2 | −2 | −3 | −1 | −2 | −3 | −2 | 1 | −1 | −3 | −3 | 0 |
| 51 | K | −1 | 2 | 1 | −1 | −3 | 0 | 0 | 1 | −1 | −3 | −3 | 4 | −2 | −3 | −2 | 1 | −1 | −3 | −3 | −3 |
| 52 | G | −1 | −2 | −1 | 2 | 4 | −2 | −1 | 3 | −2 | −2 | −3 | −2 | −2 | 0 | −2 | 0 | 2 | −2 | −2 | −2 |
| 53 | C | −1 | −4 | −4 | −4 | 10 | −4 | −4 | −4 | −3 | −1 | −1 | −4 | −1 | 2 | −4 | −2 | −2 | −2 | −1 | −1 |
| 54 | V | −1 | −2 | −1 | 0 | −2 | −1 | 1 | −2 | −2 | 0 | 0 | −1 | 3 | −2 | −2 | 1 | 0 | −3 | −2 | 3 |
| 55 | D | 0 | −2 | 0 | 6 | −3 | 0 | 1 | −1 | −1 | −3 | −4 | −1 | −3 | −3 | −1 | 0 | −1 | −4 | −3 | −3 |
| 56 | D | −2 | −1 | 3 | 4 | −4 | 0 | 2 | 2 | −1 | −4 | −4 | −1 | −3 | −4 | −2 | 0 | −1 | −4 | −3 | −3 |
| 57 | S | 0 | −1 | 2 | 2 | −3 | 1 | 0 | 2 | −1 | −3 | −3 | −1 | −2 | −3 | −2 | 3 | 0 | −3 | −3 | −3 |
| 58 | G | 0 | 1 | 0 | −1 | −3 | −1 | −2 | 5 | −2 | −4 | −4 | −1 | −3 | −3 | −2 | 1 | −2 | −3 | −3 | −3 |
| 59 | F | −1 | −4 | −4 | −4 | −2 | −3 | −3 | −4 | −2 | 3 | 0 | −3 | 0 | 5 | −4 | −2 | −1 | −1 | 0 | 3 |
| 60 | V | −1 | −3 | −3 | −2 | −2 | −1 | 1 | −3 | −1 | 0 | 0 | −2 | 0 | 3 | −3 | −2 | −1 | −1 | 4 | 3 |
| 61 | Y | −2 | −2 | −2 | −3 | −2 | −2 | −2 | −3 | 0 | −1 | −1 | −2 | −1 | 4 | −3 | 0 | −2 | 0 | 7 | −2 |
| 62 | K | 1 | −1 | −2 | −2 | −2 | −1 | −1 | 0 | −3 | 0 | −1 | 1 | −1 | −2 | 2 | −1 | −1 | −4 | −2 | 3 |
| 63 | L | −1 | −3 | −2 | −3 | −2 | −3 | −3 | 4 | −3 | 4 | 1 | −3 | 0 | −1 | −3 | −1 | −2 | −3 | −2 | 0 |
| 64 | G | −1 | −1 | 0 | 0 | −4 | 0 | 3 | 5 | −1 | −4 | −4 | −1 | −3 | −4 | −2 | 0 | −2 | −3 | −3 | −3 |
| 65 | E | −2 | −1 | −1 | 0 | −3 | 3 | 3 | −3 | −1 | −2 | −1 | 0 | 3 | −1 | −2 | −1 | −2 | 7 | −1 | −2 |
| 66 | R | −2 | 2 | −2 | −3 | −2 | 0 | −1 | −3 | 0 | 1 | −1 | 1 | −1 | 3 | −3 | −2 | −2 | −1 | 4 | 0 |
| 67 | F | −2 | −3 | −3 | −3 | −2 | −3 | −3 | −3 | −1 | −1 | −1 | −3 | −1 | 6 | −4 | −2 | 1 | 6 | 3 | −1 |
| 68 | F | 0 | 1 | −2 | −3 | −2 | −1 | −2 | −2 | −1 | −1 | 1 | −1 | 0 | 2 | −3 | 0 | 0 | −1 | 2 | −1 |
| 69 | P | −2 | −2 | −1 | 2 | −4 | 0 | 2 | −2 | −2 | −4 | −4 | −1 | −3 | −4 | 6 | −1 | −1 | −4 | −3 | −3 |
| 70 | G | 0 | −2 | 0 | −1 | −2 | −1 | −1 | 5 | −2 | −4 | −4 | −1 | −2 | −3 | −2 | 3 | −1 | −3 | −3 | −3 |
| 71 | H | −2 | −2 | 0 | 5 | −3 | −1 | 0 | −2 | 4 | −3 | −4 | −1 | −3 | −3 | 3 | 0 | −1 | −4 | −2 | −3 |
| 72 | S | −1 | −1 | 0 | −1 | −2 | −1 | −1 | −2 | 5 | −3 | −3 | −1 | −2 | −3 | 4 | 3 | 1 | −3 | −1 | −2 |
| 73 | N | 2 | −2 | 2 | −2 | 8 | −2 | −2 | −2 | −2 | −2 | −2 | −2 | −2 | −3 | −3 | 0 | 0 | −3 | −2 | −1 |
| 74 | C | −1 | −2 | −1 | −1 | 8 | −1 | 3 | −3 | −2 | −2 | −2 | −1 | −2 | −3 | −2 | 0 | 2 | −3 | −2 | −1 |
| 75 | P | −1 | 2 | −2 | −2 | −3 | 3 | 0 | −3 | −2 | −2 | 0 | 0 | −1 | −3 | 5 | −1 | −1 | −3 | −2 | −2 |
| 76 | C | 0 | −4 | −4 | −4 | 10 | −4 | −5 | −4 | −4 | −1 | −1 | −4 | −1 | −2 | −4 | −1 | −1 | −2 | −2 | −1 |
| 77 | V | −1 | −2 | −2 | −1 | −2 | −1 | 2 | −3 | −2 | 0 | 2 | −1 | 0 | −1 | −2 | −1 | 0 | −3 | −2 | 3 |
| 78 | C | 0 | −4 | −4 | −4 | 10 | −4 | −5 | −4 | −4 | −1 | −1 | −4 | −1 | −2 | −4 | −1 | −1 | −2 | −2 | −1 |
| 79 | A | 2 | −1 | −1 | −1 | −1 | 1 | −1 | −2 | −2 | −2 | −2 | −1 | −1 | −3 | −1 | 0 | 4 | −3 | −2 | −1 |
| 80 | L | 1 | −1 | −1 | 0 | −2 | 0 | 2 | −2 | −2 | −1 | 0 | −1 | −1 | −2 | −2 | 1 | 1 | −3 | −2 | −1 |
| 81 | D | −2 | −2 | 0 | 5 | −4 | 0 | 4 | −2 | −1 | −3 | −4 | −1 | −3 | −3 | −2 | 0 | 1 | −4 | −3 | −3 |
| 82 | G | 0 | −3 | 0 | −2 | −4 | −3 | −3 | 7 | −3 | −5 | −5 | −3 | −4 | −4 | −3 | 0 | −3 | −3 | −4 | −4 |
| 83 | P | −1 | −2 | −2 | −1 | −3 | −1 | −1 | −2 | −2 | −3 | −3 | −1 | −2 | −4 | 7 | 1 | −1 | −4 | −3 | −3 |
| 84 | V | 0 | −2 | −2 | −3 | −2 | 1 | −1 | −3 | −3 | 1 | 1 | −2 | 0 | −2 | −2 | 0 | −1 | −3 | −2 | 4 |
| 85 | C | 0 | −3 | −3 | −3 | 10 | −3 | −4 | −3 | −3 | −2 | −2 | −3 | −2 | −3 | −3 | 0 | −1 | −3 | −3 | −2 |
| 86 | D | 1 | −2 | −1 | 3 | 4 | −1 | −1 | −2 | −2 | −1 | −2 | −2 | 0 | 0 | −2 | 1 | 0 | −3 | −2 | 0 |
| 87 | Q | −1 | 3 | −1 | −2 | −3 | 4 | 0 | −3 | −1 | −2 | −2 | 2 | −1 | −3 | −2 | −1 | −1 | −3 | −2 | 0 |
| 88 | P | −1 | −2 | −1 | −2 | −2 | −2 | −2 | −3 | −3 | −2 | −2 | −2 | −2 | −4 | 6 | 0 | 4 | −4 | −3 | −1 |
| 89 | E | −2 | 2 | 0 | 2 | −4 | 0 | 5 | −2 | −1 | −4 | −3 | 1 | −2 | −4 | −2 | −1 | −2 | −4 | −3 | −3 |
| 90 | C | 0 | −4 | −4 | −4 | 10 | −4 | −5 | −4 | −4 | −2 | −2 | −4 | −2 | −3 | −4 | −2 | −2 | −3 | −3 | −2 |
| 91 | P | −1 | −2 | −2 | −2 | −3 | −2 | −2 | −3 | −3 | −1 | −2 | −2 | −2 | −3 | 6 | −1 | 2 | −4 | −3 | 0 |
| 92 | K | 0 | 3 | 0 | 0 | −3 | 0 | 3 | −2 | −1 | −3 | −3 | 3 | −2 | −3 | −2 | 0 | −1 | −3 | −2 | −2 |
| 93 | I | −1 | −3 | −3 | −4 | −1 | −2 | −3 | −4 | −3 | 3 | 3 | −2 | 0 | −1 | −3 | −2 | 1 | −3 | −2 | 1 |
| 94 | H | −1 | −1 | 0 | −1 | 4 | −1 | −1 | −2 | 7 | −3 | −3 | −1 | −2 | −2 | 2 | 1 | −1 | −3 | 0 | −3 |
| 95 | P | 0 | −2 | −2 | −1 | −3 | −1 | 1 | −2 | −2 | −3 | −3 | −1 | −2 | −4 | 7 | −1 | −1 | −4 | −3 | −2 |
| 96 | K | 1 | 3 | −1 | −2 | −2 | 0 | 0 | −2 | 0 | −2 | −2 | 2 | −1 | −1 | −2 | 1 | −1 | −2 | 2 | −2 |
| 97 | C | 0 | −3 | −3 | −3 | 10 | −3 | −4 | −3 | −3 | −2 | −2 | −3 | −2 | −3 | 2 | −1 | −1 | −3 | −3 | −2 |
| 98 | T | −1 | −3 | −2 | −3 | −1 | −2 | −3 | −4 | −3 | 4 | 0 | −3 | 1 | −1 | −3 | −1 | 3 | −3 | −2 | 2 |
| 99 | K | −2 | 1 | 0 | −1 | −4 | 0 | 2 | −2 | 7 | −3 | −3 | 2 | −2 | −2 | −2 | −1 | −2 | −3 | 0 | −3 |
| 100 | V | −1 | −3 | −3 | −3 | −1 | −3 | −3 | −4 | −3 | 3 | 0 | −3 | 0 | −1 | −3 | −2 | −1 | −3 | −1 | 5 |
| 101 | E | 1 | −1 | 0 | 3 | −2 | 0 | 2 | −1 | −1 | −3 | −3 | 1 | −2 | −3 | −1 | 2 | 0 | −4 | −3 | −2 |
| 102 | H | −2 | 2 | 1 | −1 | −3 | 0 | −1 | −2 | 7 | −3 | −2 | 0 | −2 | −1 | −2 | −1 | 1 | −2 | 3 | −2 |
| 103 | N | −1 | 0 | 3 | −1 | −2 | 0 | 0 | −1 | 4 | −2 | −2 | 1 | −2 | 0 | −2 | 1 | 0 | −1 | 4 | −2 |
| 104 | G | −1 | −1 | 0 | 2 | −3 | 4 | 2 | 2 | −1 | −4 | −3 | 0 | −2 | −3 | −2 | 0 | −1 | −3 | −2 | −3 |
| 105 | C | 0 | −3 | −3 | −3 | 10 | −3 | −5 | −3 | −3 | −1 | −1 | −3 | −1 | −2 | −3 | −1 | −1 | −2 | −2 | −1 |
| 106 | C | 0 | −3 | −3 | −3 | 10 | −3 | −5 | −3 | −3 | −1 | −1 | −3 | −1 | −2 | −3 | −1 | −1 | −2 | −2 | −1 |
| 107 | P | −1 | −3 | −3 | −2 | −3 | −2 | −2 | −3 | −3 | −1 | −2 | −2 | −2 | −4 | 7 | −2 | −1 | −4 | −3 | 0 |
| 108 | E | −1 | 2 | 0 | 2 | −3 | 4 | 2 | −2 | −1 | −2 | −1 | 0 | −1 | −3 | −2 | −1 | −1 | −3 | −2 | 0 |
| 109 | C | 0 | −3 | −3 | −3 | 10 | −3 | −5 | −3 | −3 | −1 | −1 | −3 | −1 | −2 | −3 | −1 | −1 | −2 | −2 | −1 |
| 110 | K | −1 | 0 | 0 | 0 | −3 | 0 | 3 | −2 | −1 | −1 | −2 | 3 | −1 | −3 | −2 | 1 | −1 | −3 | −2 | 1 |
| 111 | E | 2 | 2 | −1 | 0 | −3 | 0 | 4 | −2 | −1 | −3 | −2 | 2 | −2 | −3 | −2 | 0 | −1 | −3 | −2 | −2 |
| 112 | V | −1 | 1 | −1 | −1 | −3 | −1 | 1 | 1 | −2 | 1 | −1 | 1 | −1 | −2 | −2 | −1 | −1 | −3 | −2 | 2 |
| 113 | K | 0 | 0 | 0 | −1 | −3 | 0 | 0 | 4 | −2 | −4 | −3 | 4 | −2 | −3 | −2 | 1 | −1 | −3 | −3 | −3 |
| 114 | N | −2 | 0 | 6 | 0 | −3 | 0 | 0 | 0 | 0 | −3 | −3 | 2 | −2 | −3 | −2 | 0 | 0 | −4 | −2 | −3 |
| 115 | F | −1 | −3 | −3 | −3 | −2 | −2 | −2 | −3 | 0 | 0 | 0 | −2 | 0 | 4 | −3 | −2 | −1 | 0 | 6 | 1 |
| 116 | C | 0 | −3 | −3 | −3 | 10 | −3 | −5 | −3 | −3 | −1 | −1 | −3 | −1 | −2 | −3 | −1 | −1 | −2 | −2 | −1 |
| 117 | E | −1 | −1 | 0 | 3 | −3 | 0 | 4 | −2 | −1 | −2 | 0 | 0 | −2 | −3 | −2 | 0 | 1 | −3 | −3 | −2 |
| 118 | Y | −2 | −2 | −2 | −2 | −3 | −1 | 1 | −3 | 0 | −1 | −1 | −2 | −1 | 5 | −3 | −2 | −2 | 0 | 6 | −2 |
| 119 | H | −2 | 5 | 2 | −2 | −3 | 0 | −1 | −2 | 4 | −2 | −1 | 0 | 3 | −2 | −2 | −1 | −1 | −3 | −1 | −2 |
| 120 | G | −1 | −2 | 2 | −1 | −3 | −2 | −2 | 6 | −2 | −4 | −4 | −2 | −3 | −3 | −2 | 0 | −2 | −3 | −3 | −3 |
| 121 | K | −1 | 4 | −1 | −2 | −3 | 0 | 0 | −3 | −1 | −1 | −2 | 5 | −1 | −3 | −2 | −1 | −1 | −3 | −2 | 0 |
| 122 | N | −1 | −2 | 2 | −2 | −2 | −2 | −2 | −2 | −2 | 2 | 0 | −2 | 0 | −2 | −2 | 0 | 4 | −3 | −2 | 1 |
| 123 | Y | −2 | −3 | −3 | −3 | −2 | −2 | −3 | −3 | 0 | −1 | −1 | −3 | −1 | 4 | −4 | −2 | −2 | 1 | 8 | −1 |
| 124 | K | 0 | 1 | 0 | −1 | −3 | 2 | 2 | −2 | 4 | −3 | −2 | 2 | −1 | −1 | −2 | −1 | −2 | −2 | 3 | −2 |
| 125 | I | −1 | −2 | 2 | −2 | −2 | −2 | −2 | −2 | −2 | 2 | 1 | −2 | 0 | −1 | −2 | 0 | 1 | −3 | −2 | 2 |
| 126 | L | −1 | −2 | 2 | −2 | −2 | −2 | −2 | 3 | −2 | 0 | 3 | −2 | 0 | −1 | −3 | −1 | −1 | −3 | −2 | −1 |
| 127 | E | −1 | 0 | 0 | 1 | −4 | 3 | 6 | −2 | 0 | −3 | −3 | 0 | −2 | −3 | −1 | 0 | −1 | −3 | −2 | −2 |
| 128 | E | −1 | −1 | 2 | 0 | −3 | 0 | 4 | −2 | 0 | −3 | −3 | 0 | −2 | −1 | −2 | 0 | 1 | −2 | 3 | −2 |
| 129 | F | −2 | −3 | −3 | −3 | −2 | −3 | −3 | −3 | −1 | 0 | 0 | −3 | 0 | 7 | −5 | −2 | −2 | 0 | 2 | −1 |
| 130 | K | −1 | 1 | 2 | −1 | −3 | 2 | 0 | −2 | −1 | −1 | 0 | 2 | 1 | −2 | −2 | −1 | −1 | −3 | −2 | 0 |
| 131 | P | −1 | −3 | −3 | −3 | −2 | −2 | −2 | −3 | −3 | 1 | 2 | −2 | 0 | −2 | 3 | −2 | 0 | −3 | −2 | 3 |
| 132 | S | 0 | 0 | 1 | 2 | 2 | −1 | 0 | −1 | −1 | −3 | −3 | −1 | −2 | −3 | 1 | 4 | 1 | −4 | −3 | −2 |
| 133 | P | −1 | −2 | −2 | −1 | −3 | −1 | 1 | −3 | −2 | −2 | −3 | −1 | −2 | −4 | 7 | −1 | −1 | −4 | −3 | 0 |
| 134 | C | 0 | −3 | −3 | −3 | 10 | −3 | −4 | −3 | −3 | −1 | −1 | −3 | −1 | −2 | −3 | −1 | 0 | −2 | −2 | −1 |
| 135 | E | −1 | 1 | −1 | 0 | −3 | 0 | 5 | −3 | −1 | −1 | 1 | 0 | −1 | −2 | −2 | −1 | −1 | −3 | −2 | −1 |
| 136 | W | −1 | 2 | −1 | −2 | −3 | 2 | 0 | −2 | 3 | −2 | 0 | 1 | −1 | −2 | −2 | 1 | −1 | 4 | −1 | −2 |
| 137 | C | 0 | 0 | −3 | −3 | 10 | −3 | −4 | −3 | −3 | 0 | −1 | −3 | −1 | −2 | −3 | −1 | 0 | −3 | −2 | −1 |
| 138 | R | −1 | 4 | −1 | −2 | −2 | 0 | −1 | −3 | −1 | 2 | −1 | 2 | −1 | −1 | −2 | −1 | 1 | −2 | 1 | 0 |
| 139 | C | 0 | −4 | −4 | −4 | 10 | −4 | −5 | −4 | −4 | −1 | −1 | −4 | −1 | −2 | −4 | −1 | −1 | −2 | −2 | −1 |
| 140 | E | −1 | −1 | 0 | 2 | −3 | 0 | 5 | −3 | −1 | −2 | 0 | 0 | −1 | −3 | −2 | 0 | 1 | −3 | −2 | −2 |
| 141 | P | 1 | −2 | 1 | −2 | −2 | −1 | −1 | −2 | −2 | 0 | 0 | −1 | −1 | −2 | 2 | 1 | 1 | −3 | −2 | 0 |
| 142 | S | −1 | −1 | 4 | 2 | −3 | 1 | 0 | 2 | −1 | −3 | −3 | −1 | −2 | −3 | −2 | 2 | −1 | −4 | −3 | −3 |
| 143 | N | −1 | 1 | 2 | −1 | −3 | −1 | −1 | 4 | −1 | −4 | −4 | 1 | −2 | −3 | −2 | 1 | −1 | −3 | −3 | −3 |
| 144 | E | −1 | 0 | −1 | 0 | −3 | 0 | 4 | −3 | −1 | 1 | −1 | 0 | −1 | −2 | −2 | −1 | 1 | −3 | −2 | 1 |
| 145 | V | 3 | −2 | −3 | −3 | −1 | −2 | −2 | −2 | −3 | 0 | 0 | −2 | 0 | −2 | −2 | −1 | −1 | −3 | −2 | 3 |
| 146 | H | −2 | 1 | −2 | −3 | −2 | −1 | −2 | −3 | 3 | 0 | 2 | −1 | 0 | 2 | −3 | −2 | −2 | −1 | 4 | −1 |
| 147 | C | −1 | 0 | −3 | −3 | 10 | −3 | −3 | −3 | −3 | −2 | −2 | −2 | −2 | −3 | 1 | −1 | −1 | −3 | −3 | −2 |
| 148 | V | 0 | −2 | −1 | −2 | −1 | −1 | −2 | −2 | −3 | 0 | 1 | −1 | 0 | −2 | −2 | 2 | 3 | −3 | −2 | 2 |
| 149 | V | −1 | −3 | −3 | −3 | 1 | −2 | −2 | −3 | −3 | 2 | 0 | −2 | 0 | −2 | 2 | −2 | −1 | −4 | −2 | 5 |
| 150 | A | 3 | −2 | −2 | −2 | 2 | −1 | −1 | −1 | −2 | 0 | −1 | −1 | −1 | −2 | −2 | 2 | 0 | −3 | −2 | 2 |
| 151 | D | 1 | −2 | 0 | 5 | −2 | 1 | 0 | 0 | −2 | −3 | −3 | −1 | −2 | −3 | −2 | 1 | −1 | −4 | −3 | −2 |
| 152 | C | −1 | −3 | −3 | −3 | 10 | −3 | −4 | 0 | −3 | −2 | −2 | −3 | −1 | 1 | −4 | −1 | −2 | −2 | −1 | −2 |
| 153 | A | 2 | −2 | −2 | −2 | −2 | −1 | 0 | −2 | −2 | −1 | 0 | −1 | −1 | 1 | 4 | −1 | −1 | −3 | −1 | −1 |
| 154 | V | 2 | −1 | −2 | −1 | −2 | 4 | 1 | −2 | −2 | −1 | −2 | 0 | −1 | −3 | 3 | −1 | −1 | −3 | −2 | 0 |
| 155 | P | −1 | −2 | −1 | 1 | −2 | −2 | −1 | −2 | −2 | 1 | 0 | −2 | −1 | −2 | 4 | 0 | 2 | −4 | −3 | 0 |
| 156 | E | −2 | 1 | −1 | 0 | −4 | 0 | 3 | −3 | 2 | −3 | −3 | 1 | −2 | 2 | 3 | −1 | −2 | −3 | −1 | −3 |
| 157 | C | −1 | −3 | −3 | −3 | 9 | 1 | −3 | −3 | −3 | −2 | −1 | −3 | −1 | −2 | −3 | −2 | −2 | 5 | −2 | −2 |
| 158 | V | −1 | 0 | −2 | −3 | −2 | 0 | −2 | −3 | −3 | 2 | 0 | −2 | 0 | 1 | −2 | −2 | 1 | −2 | −1 | 4 |
| 159 | N | −2 | 1 | 4 | 4 | −3 | 0 | 0 | −1 | 0 | −3 | −3 | −1 | −2 | −1 | −2 | 0 | −1 | 3 | 3 | −3 |
| 160 | P | −1 | −2 | −2 | −1 | −3 | −1 | −1 | −2 | −2 | −1 | −3 | 1 | −2 | −4 | 7 | 0 | −1 | −4 | −3 | −2 |
| 161 | V | −1 | −3 | −3 | −3 | −2 | 0 | 0 | −4 | −3 | 4 | 0 | −2 | 0 | 2 | −3 | −2 | −1 | −2 | 0 | 3 |
| 162 | Y | −2 | 1 | −1 | −3 | −3 | −1 | −1 | −3 | 4 | −2 | 0 | −1 | −1 | 0 | −3 | 0 | −2 | 0 | 6 | −2 |
| 163 | E | −1 | −1 | −1 | 0 | −3 | 3 | 4 | −3 | −1 | −2 | −1 | 0 | −1 | −3 | 3 | 0 | 0 | −3 | −2 | −2 |
| 164 | P | −1 | −1 | −2 | −1 | −3 | 0 | 1 | −3 | −1 | −2 | −1 | 0 | −2 | −2 | 5 | −1 | −1 | −2 | 2 | −2 |
| 165 | E | −1 | −2 | 1 | 2 | −3 | −1 | 1 | 3 | −1 | −3 | −3 | −1 | −3 | −1 | −2 | 0 | −1 | −2 | 2 | −3 |
| 166 | Q | −2 | 0 | −1 | −1 | −3 | 4 | 2 | −3 | 3 | −3 | 0 | 2 | −1 | −2 | −2 | −1 | −2 | 5 | 0 | −2 |
| 167 | C | 0 | −3 | −3 | −4 | 9 | −3 | −4 | −4 | −3 | 0 | 0 | −3 | −1 | −2 | −3 | −2 | −1 | −2 | −2 | 0 |
| 168 | C | 0 | −3 | −3 | −3 | 9 | −3 | −3 | −3 | −3 | 0 | −1 | −3 | −1 | −2 | −3 | 0 | −1 | −3 | −2 | 1 |
| 169 | P | 0 | −2 | −3 | −2 | −3 | −1 | −2 | −2 | −3 | −2 | 0 | −1 | −1 | −3 | 7 | −1 | −1 | −4 | −3 | −1 |
| 170 | V | −1 | −2 | −2 | −2 | −2 | −1 | 1 | −3 | −2 | 3 | 0 | 1 | 0 | −2 | −2 | −2 | −1 | −3 | −2 | 4 |
| 171 | C | −1 | −2 | −3 | −3 | 9 | −2 | −3 | −3 | −3 | −1 | 0 | 1 | −1 | −2 | −3 | −1 | −1 | −3 | −2 | −1 |
| 172 | K | −1 | 1 | 0 | −1 | −3 | 0 | 0 | −2 | −1 | −3 | −2 | 6 | −1 | −3 | −1 | 0 | −1 | −3 | −2 | −2 |
| 173 | N | 2 | −1 | 3 | 2 | −2 | −1 | 0 | 2 | −1 | −3 | −3 | −1 | −2 | −3 | −2 | 1 | −1 | −4 | −3 | −2 |
| 174 | G | 0 | −1 | 0 | −1 | −3 | −2 | −2 | 5 | −2 | −4 | −3 | 0 | −3 | −3 | −2 | 0 | −2 | −2 | −3 | −3 |
| 175 | P | −1 | −2 | 1 | −1 | −3 | −1 | 0 | −2 | −2 | −3 | −3 | −1 | −2 | −4 | 6 | −1 | −1 | −4 | −3 | −2 |
| 176 | N | −1 | −1 | 5 | 0 | −3 | −1 | −1 | −1 | 0 | −3 | −3 | −1 | −2 | −2 | −2 | 1 | 0 | 6 | −1 | −3 |
| 177 | C | −1 | −2 | −2 | −2 | 8 | −2 | −2 | −3 | 3 | −2 | −2 | −2 | −2 | −2 | 1 | −1 | 0 | −3 | −1 | −2 |
| 178 | F | −2 | −2 | −1 | −2 | −3 | −2 | −2 | 1 | 4 | −2 | −2 | 0 | −1 | 4 | 0 | −1 | −2 | 0 | 3 | −2 |
| 179 | A | 2 | −2 | −2 | −3 | −1 | −2 | −2 | 2 | −3 | 0 | 1 | −2 | 0 | 0 | −2 | −1 | −1 | −3 | −1 | 1 |
| 180 | G | 0 | −1 | 0 | 0 | −2 | −1 | 1 | 3 | −2 | −2 | 0 | −1 | −1 | −2 | 1 | 1 | 1 | −3 | −2 | −1 |
| 181 | T | 0 | −2 | −1 | −1 | −1 | −1 | −1 | −2 | −2 | 1 | −1 | −1 | −1 | −2 | 1 | 0 | 5 | −3 | −2 | 0 |
| 182 | T | 1 | −1 | 0 | −1 | −1 | 2 | 0 | −1 | −2 | −1 | 0 | −1 | 0 | −2 | −1 | 1 | 3 | −3 | −2 | −1 |
| 183 | I | 1 | −3 | −2 | −3 | −1 | −2 | −2 | −2 | −3 | 3 | 0 | −2 | 0 | −1 | −2 | 0 | 0 | −3 | −1 | 3 |
| 184 | I | −1 | −3 | −3 | −3 | −2 | −3 | −3 | 1 | −3 | 5 | 0 | −3 | 0 | 2 | −3 | −2 | −1 | −2 | 0 | 1 |
| 185 | P | −1 | −2 | −2 | −1 | −3 | 0 | 1 | −2 | −2 | −2 | 0 | −1 | −1 | −3 | 6 | −1 | −1 | −4 | −3 | −2 |
| 186 | A | 4 | 1 | −2 | −2 | 0 | −1 | −1 | 0 | −2 | −1 | −1 | 0 | −1 | −2 | −1 | 0 | 0 | −3 | −2 | 0 |
| 187 | G | 0 | −2 | 0 | −1 | −3 | −2 | −2 | 6 | −2 | −2 | −3 | −2 | −2 | −3 | −2 | 0 | −2 | −2 | −3 | 0 |
| 188 | I | −1 | 2 | −1 | 0 | −3 | 0 | 3 | −3 | −1 | 2 | −1 | 0 | −1 | −2 | 1 | −1 | −1 | −3 | −2 | 0 |
| 189 | E | −1 | −1 | −1 | 0 | −3 | 0 | 4 | −2 | −1 | 0 | −2 | 0 | −1 | −3 | 3 | −1 | −1 | −3 | −2 | −1 |
| 190 | V | 0 | 0 | −1 | 1 | −2 | −1 | −1 | −2 | −2 | 0 | 0 | −1 | 0 | −2 | −2 | −1 | 2 | −3 | −2 | 3 |
| 191 | K | −1 | 0 | −1 | −1 | −3 | 0 | 1 | −2 | −1 | −2 | −2 | 4 | −1 | −2 | −2 | −1 | −1 | 7 | −1 | 0 |
| 192 | V | 0 | −1 | −1 | −2 | −2 | 1 | 0 | −3 | −2 | 0 | 0 | 1 | 0 | −2 | −2 | −1 | 2 | −3 | −1 | 3 |
| 193 | D | −2 | −2 | 0 | 6 | −3 | 0 | 1 | −1 | −1 | −3 | −4 | −1 | −3 | −3 | −1 | 0 | 1 | −4 | −3 | −3 |
| 194 | E | 1 | −1 | −1 | 1 | −2 | 0 | 4 | −2 | −1 | −1 | −2 | 0 | −1 | −2 | −1 | 0 | −1 | −3 | −2 | 1 |
| 195 | C | 0 | −3 | −3 | −3 | 10 | −3 | −4 | −3 | −3 | −1 | −1 | −3 | −1 | −2 | −3 | −1 | −1 | −2 | −2 | −1 |
| 196 | N | −1 | −1 | 3 | −1 | −2 | −1 | −1 | −1 | −1 | −1 | −1 | −1 | −1 | 2 | −2 | 0 | 4 | −2 | 0 | −1 |
| 197 | I | −1 | −1 | −2 | −3 | 5 | −2 | −2 | −3 | −3 | 4 | 0 | 1 | 0 | −1 | −3 | −1 | −1 | −3 | −1 | 1 |
| 198 | C | 0 | −3 | −2 | −3 | 9 | −3 | −3 | −3 | −3 | −1 | −1 | −3 | −1 | −2 | −3 | −1 | 1 | −2 | −2 | −1 |
| 199 | H | −2 | 2 | 0 | −2 | −3 | 0 | 0 | −2 | 7 | −3 | −2 | 0 | −2 | 0 | −2 | −1 | −2 | −1 | 3 | −3 |
| 200 | C | 0 | −3 | −3 | −3 | 10 | −3 | −4 | −3 | −3 | −1 | −1 | −3 | −1 | −2 | −3 | −1 | −1 | −2 | −2 | −1 |
| 201 | H | −1 | 0 | 0 | −1 | −2 | 0 | 0 | −2 | 7 | −2 | −2 | −1 | −2 | −1 | −2 | 0 | 3 | −2 | 0 | −2 |
| 202 | N | −2 | −1 | 4 | 3 | −3 | 0 | 0 | −1 | 0 | −2 | −2 | −1 | −2 | 0 | −2 | 0 | −1 | −1 | 4 | −2 |
| 203 | G | 0 | −1 | 0 | 0 | −3 | 0 | 3 | 5 | −1 | −4 | −4 | −1 | −3 | −3 | −2 | 0 | −2 | −2 | −3 | −3 |
| 204 | D | −1 | 0 | 0 | 4 | −3 | 3 | 3 | −2 | 0 | −3 | −3 | 0 | −2 | −3 | −1 | 0 | −1 | −3 | −2 | −2 |
| 205 | W | −1 | −2 | 0 | 3 | −3 | −1 | 0 | 3 | −2 | −3 | −3 | −2 | −2 | −1 | −2 | −1 | −2 | 8 | 0 | −3 |
| 206 | W | −2 | −3 | −2 | −3 | −2 | −2 | −3 | 3 | −2 | −3 | −3 | −3 | −1 | 0 | −3 | −2 | −2 | 11 | 0 | −3 |
| 207 | K | −1 | 3 | 0 | −2 | −3 | 0 | 0 | −2 | 0 | −2 | −2 | 3 | −1 | 0 | −2 | −1 | −1 | −1 | 4 | −2 |
| 208 | P | −1 | −1 | −1 | −1 | −2 | 3 | 0 | −3 | −1 | −1 | 1 | 0 | 0 | −2 | 4 | −1 | −1 | −3 | −2 | −1 |
| 209 | A | 5 | −1 | −2 | −2 | 0 | −1 | −1 | 0 | −2 | −1 | −1 | −1 | −1 | −2 | −1 | 0 | 0 | −3 | −2 | 0 |
| 210 | Q | −1 | 0 | −1 | −1 | −2 | 3 | 0 | −2 | −1 | −1 | 0 | 0 | 4 | −1 | −1 | 0 | 2 | −2 | −1 | 0 |
| 211 | C | 0 | −3 | −3 | −3 | 10 | −3 | −4 | −3 | −3 | −1 | −1 | −3 | −1 | −2 | −3 | −1 | −1 | −2 | −2 | −1 |
| 212 | S | 0 | −1 | 0 | 0 | −1 | 0 | 0 | −1 | −1 | −2 | −2 | 0 | −1 | −2 | −1 | 4 | 3 | −3 | −2 | −1 |
| 213 | K | −1 | 5 | 0 | −2 | −3 | 0 | 0 | −2 | 0 | −3 | −2 | 4 | −1 | −3 | −2 | −1 | −1 | −3 | −2 | −3 |
| 214 | R | −1 | 3 | 0 | −2 | −2 | 0 | −1 | −3 | 6 | −1 | 1 | 0 | 0 | −1 | −2 | −1 | −1 | −2 | 0 | −1 |
| 215 | E | −1 | 0 | 0 | 1 | −4 | 1 | 5 | −2 | 0 | −3 | −3 | 3 | −2 | −3 | −1 | 0 | −1 | −3 | −2 | −2 |
| 216 | C | 0 | −2 | 3 | −1 | 9 | −2 | −2 | −2 | −1 | −1 | −1 | −2 | −1 | −2 | −3 | 0 | −1 | −3 | −2 | −1 |
| 217 | Q | −1 | 2 | −1 | −1 | 6 | 3 | 0 | −2 | −1 | −2 | −2 | 1 | −1 | −3 | −2 | 0 | −1 | −2 | −2 | −2 |
| 218 | G | −1 | 0 | 3 | 0 | −3 | 3 | 0 | 3 | 0 | −3 | −3 | 0 | −1 | −3 | −2 | 0 | −1 | −3 | −2 | −3 |
| 219 | K | −1 | 0 | 0 | −2 | −2 | 0 | 0 | −2 | 5 | 0 | 0 | 3 | 3 | −1 | −2 | −1 | −1 | −2 | 0 | 0 |
| 220 | Q | −1 | 0 | 0 | 0 | −3 | 4 | 4 | −2 | 0 | −3 | −2 | 0 | −1 | −3 | −1 | 0 | −1 | −2 | −1 | −2 |
| 221 | T | 0 | −1 | 0 | −1 | −2 | −1 | −1 | 4 | −2 | −2 | −2 | −1 | −2 | −2 | −1 | 0 | 4 | −2 | −2 | −1 |
| 222 | V | 0 | −2 | −2 | −3 | −1 | −1 | −2 | −3 | −2 | 2 | 1 | −1 | 4 | 0 | −2 | −1 | 0 | −2 | −1 | 3 |

wherein, when said profile is input as a query sequence into the search program BLAST, using the default parameters specified by the NCBI (the National Center for Biotechnology Information) [Blosum 62 matrix; gap open penalty=11 and gap extension penalty=1], members of the SECFAM3 family are those which have an E value of 10⁻² or less.

2: The method of claim 1 wherein said E value is 10⁻⁵ or less.

3: The method of claim 1 wherein the database of translated nucleic acid sequences is derived from cDNA, EST, mRNA, whole or partial genome databases.

4: The method of claim 1 wherein the database is an EST database.

5: The method of claim 1 wherein the database is a human sequence database.

6-60. (canceled)

61: A composition of matter comprising:
a) an isolated polypeptide:
1) comprising an amino acid sequence that has an E value of 10⁻² or less when the below is input as a query sequence into the search program BLAST, using the default ers specified by the NCBI (the National Center for Biotechnology Information) [Blosum 62 matrix; gap open penalty=11 and gap extension penalty=1]:

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | M | −2 | −2 | −3 | −4 | −2 | −1 | −3 | −4 | −3 | 0 | 2 | −2 | 8 | 0 | −3 | −2 | −2 | −2 | −2 | 0 |
| 2 | A | 3 | −1 | −3 | −3 | −1 | −1 | −2 | −2 | −3 | 0 | 0 | 1 | 2 | 1 | −2 | −1 | −1 | −3 | −1 | 1 |
| 3 | L | 1 | −2 | −2 | −3 | −2 | −2 | −2 | −2 | 2 | 0 | 2 | −2 | 0 | −2 | 0 | 1 | −1 | −3 | −2 | 2 |
| 4 | H | −1 | −1 | 1 | −1 | −3 | −1 | −1 | 3 | 6 | −3 | −1 | −1 | −2 | −2 | −2 | 2 | −1 | −3 | −1 | −3 |
| 5 | I | 0 | −2 | −3 | −3 | −2 | 0 | 0 | −3 | −3 | 3 | 2 | −2 | 0 | −1 | −3 | −2 | −1 | −3 | −2 | 2 |
| 6 | H | 0 | −2 | −1 | −2 | −2 | −1 | −1 | −3 | 7 | 0 | 0 | −2 | −1 | −2 | 0 | 0 | −1 | −3 | 0 | 2 |
| 7 | E | 0 | −2 | −2 | −1 | −2 | −1 | 2 | −3 | −1 | 0 | 1 | −1 | 2 | 2 | −3 | 0 | −1 | −2 | 2 | 0 |
| 8 | A | 2 | −2 | −1 | −2 | 3 | −2 | −2 | 2 | −2 | −2 | −2 | −2 | −2 | −3 | −2 | 3 | 1 | −3 | −3 | −2 |
| 9 | C | 0 | −3 | −2 | −3 | 7 | −2 | −3 | −3 | −3 | 0 | −1 | −2 | 3 | 3 | −3 | 0 | −1 | −2 | −1 | 0 |
| 10 | I | −1 | −2 | −2 | −2 | −2 | −2 | 0 | 0 | −2 | 3 | 2 | −2 | 0 | 0 | −3 | 0 | 0 | −2 | 3 | 0 |
| 11 | L | −2 | 0 | −3 | −4 | −2 | −2 | −3 | −4 | −3 | 0 | 4 | −2 | 3 | 3 | −4 | −2 | −2 | −2 | 0 | 0 |
| 12 | L | 0 | 0 | −3 | −4 | −2 | −2 | −3 | −3 | −3 | 0 | 3 | −2 | 0 | −1 | 1 | −2 | −1 | −3 | −2 | 1 |
| 13 | L | −1 | −2 | −2 | −2 | −2 | −2 | 0 | −3 | −3 | 0 | 3 | −2 | 0 | 1 | −3 | 1 | 1 | −3 | −1 | 1 |
| 14 | V | 0 | 0 | −3 | −3 | 4 | 0 | −2 | −3 | −3 | 0 | 0 | −2 | 2 | −2 | −3 | −2 | −1 | −3 | −2 | 4 |
| 15 | I | −1 | 0 | −2 | −3 | 4 | −2 | −2 | −3 | 2 | 2 | 1 | −2 | 0 | −1 | −3 | 0 | 0 | 4 | −1 | 0 |
| 16 | P | 0 | 0 | −2 | −2 | −2 | −2 | −2 | −3 | 4 | −1 | 0 | −2 | −1 | 2 | 3 | 0 | −1 | −2 | 0 | 0 |
| 17 | G | 0 | −3 | −2 | −3 | −2 | −2 | −3 | 2 | −3 | 1 | 0 | −3 | 3 | 3 | −3 | −1 | −2 | −2 | 0 | 1 |
| 18 | L | 1 | −3 | −3 | −4 | −1 | −2 | −3 | −3 | −3 | 0 | 4 | −2 | 0 | −1 | −3 | −2 | −1 | −3 | −2 | 2 |
| 19 | V | 2 | −1 | −1 | −2 | −2 | 3 | −1 | −2 | −2 | 0 | −1 | −1 | −1 | −3 | −2 | 1 | 1 | −3 | −2 | 2 |
| 20 | T | −1 | −2 | −2 | −3 | 4 | −2 | −2 | −3 | −3 | 0 | 3 | −2 | 0 | −2 | −3 | 1 | 3 | −3 | −2 | 0 |
| 21 | S | 0 | −2 | −1 | −2 | 4 | −2 | −2 | 1 | −3 | −1 | 1 | −2 | 2 | −2 | 0 | 2 | −1 | −3 | −3 | −1 |
| 22 | A | 3 | −2 | −2 | −2 | −1 | −2 | −2 | −1 | −2 | −1 | 0 | −2 | −1 | 1 | 2 | 2 | −1 | −3 | −2 | 0 |
| 23 | A | 2 | −2 | −3 | −2 | 5 | 0 | 0 | −2 | −3 | −1 | 0 | −2 | −1 | −3 | 2 | −1 | −1 | −3 | −2 | 1 |
| 24 | I | 1 | 1 | −2 | −2 | −2 | −1 | −2 | −2 | −2 | 2 | 0 | −1 | −1 | −1 | −2 | 2 | −1 | −3 | 1 | 1 |
| 25 | S | −1 | −1 | 2 | −1 | −2 | 1 | −1 | −2 | 4 | 2 | −1 | −1 | −1 | −2 | −2 | 3 | −1 | −4 | −1 | −1 |
| 26 | H | 0 | −2 | 3 | −1 | −3 | −1 | −1 | −2 | 5 | −2 | −2 | −1 | −2 | 0 | 3 | 0 | −1 | −3 | −1 | 0 |
| 27 | E | −1 | −1 | 0 | 1 | −3 | 0 | 5 | −2 | −1 | −3 | −3 | 0 | −2 | −3 | −1 | 1 | 2 | −3 | −2 | −2 |
| 28 | D | 1 | −2 | 0 | 4 | −2 | −1 | 0 | −1 | −2 | −2 | 0 | −1 | −1 | −2 | −1 | 2 | 0 | −4 | −3 | −2 |
| 29 | Y | −2 | −1 | −2 | −2 | −3 | 2 | −1 | −3 | 0 | 0 | −1 | −1 | −1 | 0 | 3 | −1 | −2 | 0 | 5 | 0 |
| 30 | P | 0 | −1 | −1 | −1 | −2 | −1 | −1 | 0 | 5 | −3 | −3 | −1 | −2 | −3 | 4 | 0 | 0 | −3 | −1 | −2 |
| 31 | A | 3 | −2 | −2 | −2 | −1 | −1 | −2 | −1 | −3 | 0 | 2 | −1 | 0 | −2 | 0 | 0 | 0 | −3 | −2 | 0 |
| 32 | D | −2 | −1 | 0 | 5 | −3 | 0 | 2 | −2 | −1 | −2 | −3 | 2 | −2 | −3 | −1 | 0 | −1 | −4 | −3 | 0 |
| 33 | E | 0 | −1 | −2 | 0 | −2 | 0 | 3 | −3 | −1 | 0 | 0 | −1 | 0 | 3 | −2 | −1 | −1 | −2 | 0 | 0 |
| 34 | G | 1 | −2 | 0 | −1 | −2 | −1 | −2 | 4 | −2 | −2 | −2 | −2 | 0 | −2 | −2 | 1 | 1 | −2 | −3 | 0 |
| 35 | D | 0 | 2 | 0 | 2 | −2 | 0 | 0 | −2 | −1 | −2 | 0 | 0 | −1 | −3 | 2 | −1 | −1 | −3 | −2 | −2 |
| 36 | Q | 0 | 3 | 0 | −1 | −2 | 3 | 0 | −2 | −1 | −2 | −2 | 0 | −1 | −3 | −1 | 1 | 3 | −3 | −2 | −2 |
| 37 | I | 0 | −2 | −3 | −2 | −1 | −2 | −2 | −2 | −3 | 1 | 0 | −2 | 0 | −2 | 4 | −1 | −1 | −3 | −2 | 2 |
| 38 | S | −1 | −1 | 0 | 1 | −2 | 0 | 2 | −2 | −1 | 2 | −1 | 1 | −1 | −2 | −2 | 2 | −1 | −3 | −2 | 0 |
| 39 | S | 3 | −1 | −1 | −1 | −1 | −1 | −1 | −1 | −2 | −1 | 0 | −1 | −1 | −2 | 2 | 3 | 0 | −3 | −2 | −1 |
| 40 | N | 0 | −2 | 2 | −1 | −3 | −1 | 1 | 3 | −1 | 0 | 0 | −1 | −1 | −3 | 1 | −1 | −1 | −3 | −2 | 0 |
| 41 | D | 1 | −2 | −1 | 3 | −2 | 0 | 2 | −2 | −2 | −1 | 0 | −1 | −1 | 0 | −2 | 0 | −1 | −3 | −2 | −1 |
| 42 | N | −1 | −1 | 2 | 1 | −3 | 0 | 3 | 0 | −1 | −2 | 0 | 0 | −1 | −2 | −2 | 1 | −1 | −3 | −2 | −2 |
| 43 | L | 1 | −3 | −3 | −3 | 4 | −2 | −3 | −3 | −1 | 0 | 1 | −3 | 0 | 3 | −3 | −2 | −2 | 0 | 5 | 0 |
| 44 | I | 1 | −1 | −1 | 0 | −2 | 0 | 2 | −2 | −2 | 1 | −1 | 1 | −1 | −2 | −2 | 2 | −1 | −4 | −2 | 0 |
| 45 | F | −2 | −3 | −2 | 1 | −2 | −2 | −2 | −3 | −1 | 2 | 0 | −3 | 0 | 4 | −3 | −2 | −2 | −1 | 4 | 1 |
| 46 | D | 1 | −2 | −1 | 2 | −2 | −1 | 0 | 0 | −2 | −3 | −3 | −1 | −2 | −3 | 4 | 1 | −1 | −4 | −3 | −2 |
| 47 | D | 0 | −2 | 0 | 5 | −3 | −1 | 0 | 1 | −2 | −3 | −4 | −1 | −3 | −3 | 2 | 1 | −1 | −4 | −3 | −3 |
| 48 | Y | −2 | 3 | −1 | −2 | −3 | 2 | −1 | −3 | 0 | −2 | −2 | 0 | −1 | 0 | −3 | −2 | −2 | 0 | 6 | −2 |
| 49 | R | −2 | 4 | 0 | −2 | −4 | 2 | 0 | −2 | 5 | −4 | −3 | 0 | −2 | −3 | 2 | −1 | −2 | −3 | −1 | −3 |
| 50 | G | −1 | −2 | 0 | 2 | −3 | −1 | 1 | 4 | −2 | −2 | −3 | −1 | −2 | −3 | −2 | 1 | −1 | −3 | −3 | 0 |
| 51 | K | −1 | 2 | 1 | −1 | −3 | 0 | 0 | 1 | −1 | −3 | −3 | 4 | −2 | −3 | −2 | 1 | −1 | −3 | −3 | −3 |
| 52 | G | −1 | −2 | −1 | 2 | 4 | −2 | −1 | 3 | −2 | −2 | −3 | −2 | −2 | 0 | −2 | 0 | 2 | −2 | −2 | −2 |
| 53 | C | −1 | −4 | −4 | −4 | 10 | −4 | −4 | −4 | −3 | −1 | −1 | −4 | −1 | 2 | −4 | −2 | −2 | −2 | −1 | −1 |
| 54 | V | −1 | −2 | −1 | 0 | −2 | −1 | 1 | −2 | −2 | 0 | 0 | −1 | 3 | −2 | −2 | 1 | 0 | −3 | −2 | 3 |
| 55 | D | 0 | −2 | 0 | 6 | −3 | 0 | 1 | −1 | −1 | −3 | −4 | −1 | −3 | −3 | −1 | 0 | −1 | −4 | −3 | −3 |
| 56 | D | −2 | −1 | 3 | 4 | −4 | 0 | 2 | 2 | −1 | −4 | −4 | −1 | −3 | −4 | −2 | 0 | −1 | −4 | −3 | −3 |
| 57 | S | 0 | −1 | 2 | 2 | −3 | 1 | 0 | 2 | −1 | −3 | −3 | −1 | −2 | −3 | −2 | 3 | 0 | −3 | −3 | −3 |
| 58 | G | 0 | 1 | 0 | −1 | −3 | −1 | −2 | 5 | −2 | −4 | −4 | −1 | −3 | −3 | −2 | 1 | −2 | −3 | −3 | −3 |
| 59 | F | −1 | −4 | −4 | −4 | −2 | −3 | −3 | −4 | −2 | 3 | 0 | −3 | 0 | 5 | −4 | −2 | −1 | −1 | 0 | 3 |
| 60 | V | −1 | −3 | −3 | −2 | −2 | −1 | 1 | −3 | −1 | 0 | 0 | −2 | 0 | 3 | −3 | −2 | −1 | −1 | 4 | 3 |
| 61 | Y | −2 | −2 | −2 | −3 | −2 | −2 | −2 | −3 | 0 | −1 | −1 | −2 | −1 | 4 | −3 | 0 | −2 | 0 | 7 | −2 |
| 62 | K | 1 | −1 | −2 | −2 | −2 | −1 | −1 | 0 | −3 | 0 | −1 | 1 | −1 | −2 | 2 | −1 | −1 | −4 | −2 | 3 |
| 63 | L | −1 | −3 | −2 | −3 | −2 | −3 | −3 | 4 | −3 | 4 | 1 | −3 | 0 | −1 | −3 | −1 | −2 | −3 | −2 | 0 |
| 64 | G | −1 | −1 | 0 | 0 | −4 | 0 | 3 | 5 | −1 | −4 | −4 | −1 | −3 | −4 | −2 | 0 | −2 | −3 | −3 | −3 |
| 65 | E | −2 | −1 | −1 | 0 | −3 | 3 | 3 | −3 | −1 | −2 | −1 | 0 | 3 | −1 | −2 | −1 | −2 | 7 | −1 | −2 |
| 66 | R | −2 | 2 | −2 | −3 | −2 | 0 | −1 | −3 | 0 | 1 | −1 | 1 | −1 | 3 | −3 | −2 | −2 | −1 | 4 | 0 |
| 67 | F | −2 | −3 | −3 | −3 | −2 | −3 | −3 | −3 | −1 | −1 | −1 | −3 | −1 | 6 | −4 | −2 | 1 | 6 | 3 | −1 |
| 68 | F | 0 | 1 | −2 | −3 | −2 | −1 | −2 | −2 | −1 | −1 | 1 | −1 | 0 | 2 | −3 | 0 | 0 | −1 | 2 | −1 |
| 69 | P | −2 | −2 | −1 | 2 | −4 | 0 | 2 | −2 | −2 | −4 | −4 | −1 | −3 | −4 | 6 | −1 | −1 | −4 | −3 | −3 |
| 70 | G | 0 | −2 | 0 | −1 | −2 | −1 | −1 | 5 | −2 | −4 | −4 | −1 | −2 | −3 | −2 | 3 | −1 | −3 | −3 | −3 |
| 71 | H | −2 | −2 | 0 | 5 | −3 | −1 | 0 | −2 | 4 | −3 | −4 | −1 | −3 | −3 | 3 | 0 | −1 | −4 | −2 | −3 |
| 72 | S | −1 | −1 | 0 | −1 | −2 | −1 | −1 | −2 | 5 | −3 | −3 | −1 | −2 | −3 | 4 | 3 | 1 | −3 | −1 | −2 |
| 73 | N | 2 | −2 | 2 | −2 | 8 | −2 | −2 | −2 | −2 | −2 | −2 | −2 | −2 | −3 | −3 | 0 | 0 | −3 | −2 | −1 |
| 74 | C | −1 | −2 | −1 | −1 | 8 | −1 | 3 | −3 | −2 | −2 | −2 | −1 | −2 | −3 | −2 | 0 | 2 | −3 | −2 | −1 |
| 75 | P | −1 | 2 | −2 | −2 | −3 | 3 | 0 | −3 | −2 | −2 | 0 | 0 | −1 | −3 | 5 | −1 | −1 | −3 | −2 | −2 |
| 76 | C | 0 | −4 | −4 | −4 | 10 | −4 | −5 | −4 | −4 | −1 | −1 | −4 | −1 | −2 | −4 | −1 | −1 | −2 | −2 | −1 |
| 77 | V | −1 | −2 | −2 | −1 | −2 | −1 | 2 | −3 | −2 | 0 | 2 | −1 | 0 | −1 | −2 | −1 | 0 | −3 | −2 | 3 |
| 78 | C | 0 | −4 | −4 | −4 | 10 | −4 | −5 | −4 | −4 | −1 | −1 | −4 | −1 | −2 | −4 | −1 | −1 | −2 | −2 | −1 |
| 79 | A | 2 | −1 | −1 | −1 | −1 | 1 | −1 | −2 | −2 | −2 | −2 | −1 | −1 | −3 | −1 | 0 | 4 | −3 | −2 | −1 |
| 80 | L | 1 | −1 | −1 | 0 | −2 | 0 | 2 | −2 | −2 | −1 | 0 | −1 | −1 | −2 | −2 | 1 | 1 | −3 | −2 | −1 |
| 81 | D | −2 | −2 | 0 | 5 | −4 | 0 | 4 | −2 | −1 | −3 | −4 | −1 | −3 | −3 | −2 | 0 | 1 | −4 | −3 | −3 |
| 82 | G | 0 | −3 | 0 | −2 | −4 | −3 | −3 | 7 | −3 | −5 | −5 | −3 | −4 | −4 | −3 | 0 | −3 | −3 | −4 | −4 |
| 83 | P | −1 | −2 | −2 | −1 | −3 | −1 | −1 | −2 | −2 | −3 | −3 | −1 | −2 | −4 | 7 | 1 | −1 | −4 | −3 | −3 |
| 84 | V | 0 | −2 | −2 | −3 | −2 | 1 | −1 | −3 | −3 | 1 | 1 | −2 | 0 | −2 | −2 | 0 | −1 | −3 | −2 | 4 |
| 85 | C | 0 | −3 | −3 | −3 | 10 | −3 | −4 | −3 | −3 | −2 | −2 | −3 | −2 | −3 | −3 | 0 | −1 | −3 | −3 | −2 |
| 86 | D | 1 | −2 | −1 | 3 | 4 | −1 | −1 | −2 | −2 | −1 | −2 | −2 | 0 | 0 | −2 | 1 | 0 | −3 | −2 | 0 |
| 87 | Q | −1 | 3 | −1 | −2 | −3 | 4 | 0 | −3 | −1 | −2 | −2 | 2 | −1 | −3 | −2 | −1 | −1 | −3 | −2 | 0 |
| 88 | P | −1 | −2 | −1 | −2 | −2 | −2 | −2 | −3 | −3 | −2 | −2 | −2 | −2 | −4 | 6 | 0 | 4 | −4 | −3 | −1 |
| 89 | E | −2 | 2 | 0 | 2 | −4 | 0 | 5 | −2 | −1 | −4 | −3 | 1 | −2 | −4 | −2 | −1 | −2 | −4 | −3 | −3 |
| 90 | C | 0 | −4 | −4 | −4 | 10 | −4 | −5 | −4 | −4 | −2 | −2 | −4 | −2 | −3 | −4 | −2 | −2 | −3 | −3 | −2 |
| 91 | P | −1 | −2 | −2 | −2 | −3 | −2 | −2 | −3 | −3 | −1 | −2 | −2 | −2 | −3 | 6 | −1 | 2 | −4 | −3 | 0 |
| 92 | K | 0 | 3 | 0 | 0 | −3 | 0 | 3 | −2 | −1 | −3 | −3 | 3 | −2 | −3 | −2 | 0 | −1 | −3 | −2 | −2 |
| 93 | I | −1 | −3 | −3 | −4 | −1 | −2 | −3 | −4 | −3 | 3 | 3 | −2 | 0 | −1 | −3 | −2 | 1 | −3 | −2 | 1 |
| 94 | H | −1 | −1 | 0 | −1 | 4 | −1 | −1 | −2 | 7 | −3 | −3 | −1 | −2 | −2 | 2 | 1 | −1 | −3 | 0 | −3 |
| 95 | P | 0 | −2 | −2 | −1 | −3 | −1 | 1 | −2 | −2 | −3 | −3 | −1 | −2 | −4 | 7 | −1 | −1 | −4 | −3 | −2 |
| 96 | K | 1 | 3 | −1 | −2 | −2 | 0 | 0 | −2 | 0 | −2 | −2 | 2 | −1 | −1 | −2 | 1 | −1 | −2 | 2 | −2 |
| 97 | C | 0 | −3 | −3 | −3 | 10 | −3 | −4 | −3 | −3 | −2 | −2 | −3 | −2 | −3 | 2 | −1 | −1 | −3 | −3 | −2 |
| 98 | T | −1 | −3 | −2 | −3 | −1 | −2 | −3 | −4 | −3 | 4 | 0 | −3 | 1 | −1 | −3 | −1 | 3 | −3 | −2 | 2 |
| 99 | K | −2 | 1 | 0 | −1 | −4 | 0 | 2 | −2 | 7 | −3 | −3 | 2 | −2 | −2 | −2 | −1 | −2 | −3 | 0 | −3 |
| 100 | V | −1 | −3 | −3 | −3 | −1 | −3 | −3 | −4 | −3 | 3 | 0 | −3 | 0 | −1 | −3 | −2 | −1 | −3 | −1 | 5 |
| 101 | E | 1 | −1 | 0 | 3 | −2 | 0 | 2 | −1 | −1 | −3 | −3 | 1 | −2 | −3 | −1 | 2 | 0 | −4 | −3 | −2 |
| 102 | H | −2 | 2 | 1 | −1 | −3 | 0 | −1 | −2 | 7 | −3 | −2 | 0 | −2 | −1 | −2 | −1 | 1 | −2 | 3 | −2 |
| 103 | N | −1 | 0 | 3 | −1 | −2 | 0 | 0 | −1 | 4 | −2 | −2 | 1 | −2 | 0 | −2 | 1 | 0 | −1 | 4 | −2 |
| 104 | G | −1 | −1 | 0 | 2 | −3 | 4 | 2 | 2 | −1 | −4 | −3 | 0 | −2 | −3 | −2 | 0 | −1 | −3 | −2 | −3 |
| 105 | C | 0 | −3 | −3 | −3 | 10 | −3 | −5 | −3 | −3 | −1 | −1 | −3 | −1 | −2 | −3 | −1 | −1 | −2 | −2 | −1 |
| 106 | C | 0 | −3 | −3 | −3 | 10 | −3 | −5 | −3 | −3 | −1 | −1 | −3 | −1 | −2 | −3 | −1 | −1 | −2 | −2 | −1 |
| 107 | P | −1 | −3 | −3 | −2 | −3 | −2 | −2 | −3 | −3 | −1 | −2 | −2 | −2 | −4 | 7 | −2 | −1 | −4 | −3 | 0 |
| 108 | E | −1 | 2 | 0 | 2 | −3 | 4 | 2 | −2 | −1 | −2 | −1 | 0 | −1 | −3 | −2 | −1 | −1 | −3 | −2 | 0 |
| 109 | C | 0 | −3 | −3 | −3 | 10 | −3 | −5 | −3 | −3 | −1 | −1 | −3 | −1 | −2 | −3 | −1 | −1 | −2 | −2 | −1 |
| 110 | K | −1 | 0 | 0 | 0 | −3 | 0 | 3 | −2 | −1 | −1 | −2 | 3 | −1 | −3 | −2 | 1 | −1 | −3 | −2 | 1 |
| 111 | E | 2 | 2 | −1 | 0 | −3 | 0 | 4 | −2 | −1 | −3 | −2 | 2 | −2 | −3 | −2 | 0 | −1 | −3 | −2 | −2 |
| 112 | V | −1 | 1 | −1 | −1 | −3 | −1 | 1 | 1 | −2 | 1 | −1 | 1 | −1 | −2 | −2 | −1 | −1 | −3 | −2 | 2 |
| 113 | K | 0 | 0 | 0 | −1 | −3 | 0 | 0 | 4 | −2 | −4 | −3 | 4 | −2 | −3 | −2 | 1 | −1 | −3 | −3 | −3 |
| 114 | N | −2 | 0 | 6 | 0 | −3 | 0 | 0 | 0 | 0 | −3 | −3 | 2 | −2 | −3 | −2 | 0 | 0 | −4 | −2 | −3 |
| 115 | F | −1 | −3 | −3 | −3 | −2 | −2 | −2 | −3 | 0 | 0 | 0 | −2 | 0 | 4 | −3 | −2 | −1 | 0 | 6 | 1 |
| 116 | C | 0 | −3 | −3 | −3 | 10 | −3 | −5 | −3 | −3 | −1 | −1 | −3 | −1 | −2 | −3 | −1 | −1 | −2 | −2 | −1 |
| 117 | E | −1 | −1 | 0 | 3 | −3 | 0 | 4 | −2 | −1 | −2 | 0 | 0 | −2 | −3 | −2 | 0 | 1 | −3 | −3 | −2 |
| 118 | Y | −2 | −2 | −2 | −2 | −3 | −1 | 1 | −3 | 0 | −1 | −1 | −2 | −1 | 5 | −3 | −2 | −2 | 0 | 6 | −2 |
| 119 | H | −2 | 5 | 2 | −2 | −3 | 0 | −1 | −2 | 4 | −2 | −1 | 0 | 3 | −2 | −2 | −1 | −1 | −3 | −1 | −2 |
| 120 | G | −1 | −2 | 2 | −1 | −3 | −2 | −2 | 6 | −2 | −4 | −4 | −2 | −3 | −3 | −2 | 0 | −2 | −3 | −3 | −3 |
| 121 | K | −1 | 4 | −1 | −2 | −3 | 0 | 0 | −3 | −1 | −1 | −2 | 5 | −1 | −3 | −2 | −1 | −1 | −3 | −2 | 0 |
| 122 | N | −1 | −2 | 2 | −2 | −2 | −2 | −2 | −2 | −2 | 2 | 0 | −2 | 0 | −2 | −2 | 0 | 4 | −3 | −2 | 1 |
| 123 | Y | −2 | −3 | −3 | −3 | −2 | −2 | −3 | −3 | 0 | −1 | −1 | −3 | −1 | 4 | −4 | −2 | −2 | 1 | 8 | −1 |
| 124 | K | 0 | 1 | 0 | −1 | −3 | 2 | 2 | −2 | 4 | −3 | −2 | 2 | −1 | −1 | −2 | −1 | −2 | −2 | 3 | −2 |
| 125 | I | −1 | −2 | 2 | −2 | −2 | −2 | −2 | −2 | −2 | 2 | 1 | −2 | 0 | −1 | −2 | 0 | 1 | −3 | −2 | 2 |
| 126 | L | −1 | −2 | 2 | −2 | −2 | −2 | −2 | 3 | −2 | 0 | 3 | −2 | 0 | −1 | −3 | −1 | −1 | −3 | −2 | −1 |
| 127 | E | −1 | 0 | 0 | 1 | −4 | 3 | 6 | −2 | 0 | −3 | −3 | 0 | −2 | −3 | −1 | 0 | −1 | −3 | −2 | −2 |
| 128 | E | −1 | −1 | 2 | 0 | −3 | 0 | 4 | −2 | 0 | −3 | −3 | 0 | −2 | −1 | −2 | 0 | 1 | −2 | 3 | −2 |
| 129 | F | −2 | −3 | −3 | −3 | −2 | −3 | −3 | −3 | −1 | 0 | 0 | −3 | 0 | 7 | −5 | −2 | −2 | 0 | 2 | −1 |
| 130 | K | −1 | 1 | 2 | −1 | −3 | 2 | 0 | −2 | −1 | −1 | 0 | 2 | 1 | −2 | −2 | −1 | −1 | −3 | −2 | 0 |
| 131 | P | −1 | −3 | −3 | −3 | −2 | −2 | −2 | −3 | −3 | 1 | 2 | −2 | 0 | −2 | 3 | −2 | 0 | −3 | −2 | 3 |
| 132 | S | 0 | 0 | 1 | 2 | 2 | −1 | 0 | −1 | −1 | −3 | −3 | −1 | −2 | −3 | 1 | 4 | 1 | −4 | −3 | −2 |
| 133 | P | −1 | −2 | −2 | −1 | −3 | −1 | 1 | −3 | −2 | −2 | −3 | −1 | −2 | −4 | 7 | −1 | −1 | −4 | −3 | 0 |
| 134 | C | 0 | −3 | −3 | −3 | 10 | −3 | −4 | −3 | −3 | −1 | −1 | −3 | −1 | −2 | −3 | −1 | 0 | −2 | −2 | −1 |
| 135 | E | −1 | 1 | −1 | 0 | −3 | 0 | 5 | −3 | −1 | −1 | 1 | 0 | −1 | −2 | −2 | −1 | −1 | −3 | −2 | −1 |
| 136 | W | −1 | 2 | −1 | −2 | −3 | 2 | 0 | −2 | 3 | −2 | 0 | 1 | −1 | −2 | −2 | 1 | −1 | 4 | −1 | −2 |
| 137 | C | 0 | 0 | −3 | −3 | 10 | −3 | −4 | −3 | −3 | 0 | −1 | −3 | −1 | −2 | −3 | −1 | 0 | −3 | −2 | −1 |
| 138 | R | −1 | 4 | −1 | −2 | −2 | 0 | −1 | −3 | −1 | 2 | −1 | 2 | −1 | −1 | −2 | −1 | 1 | −2 | 1 | 0 |
| 139 | C | 0 | −4 | −4 | −4 | 10 | −4 | −5 | −4 | −4 | −1 | −1 | −4 | −1 | −2 | −4 | −1 | −1 | −2 | −2 | −1 |
| 140 | E | −1 | −1 | 0 | 2 | −3 | 0 | 5 | −3 | −1 | −2 | 0 | 0 | −1 | −3 | −2 | 0 | 1 | −3 | −2 | −2 |
| 141 | P | 1 | −2 | 1 | −2 | −2 | −1 | −1 | −2 | −2 | 0 | 0 | −1 | −1 | −2 | 2 | 1 | 1 | −3 | −2 | 0 |
| 142 | S | −1 | −1 | 4 | 2 | −3 | 1 | 0 | 2 | −1 | −3 | −3 | −1 | −2 | −3 | −2 | 2 | −1 | −4 | −3 | −3 |
| 143 | N | −1 | 1 | 2 | −1 | −3 | −1 | −1 | 4 | −1 | −4 | −4 | 1 | −2 | −3 | −2 | 1 | −1 | −3 | −3 | −3 |
| 144 | E | −1 | 0 | −1 | 0 | −3 | 0 | 4 | −3 | −1 | 1 | −1 | 0 | −1 | −2 | −2 | −1 | 1 | −3 | −2 | 1 |
| 145 | V | 3 | −2 | −3 | −3 | −1 | −2 | −2 | −2 | −3 | 0 | 0 | −2 | 0 | −2 | −2 | −1 | −1 | −3 | −2 | 3 |
| 146 | H | −2 | 1 | −2 | −3 | −2 | −1 | −2 | −3 | 3 | 0 | 2 | −1 | 0 | 2 | −3 | −2 | −2 | −1 | 4 | −1 |
| 147 | C | −1 | 0 | −3 | −3 | 10 | −3 | −3 | −3 | −3 | −2 | −2 | −2 | −2 | −3 | 1 | −1 | −1 | −3 | −3 | −2 |
| 148 | V | 0 | −2 | −1 | −2 | −1 | −1 | −2 | −2 | −3 | 0 | 1 | −1 | 0 | −2 | −2 | 2 | 3 | −3 | −2 | 2 |
| 149 | V | −1 | −3 | −3 | −3 | 1 | −2 | −2 | −3 | −3 | 2 | 0 | −2 | 0 | −2 | 2 | −2 | −1 | −4 | −2 | 5 |
| 150 | A | 3 | −2 | −2 | −2 | 2 | −1 | −1 | −1 | −2 | 0 | −1 | −1 | −1 | −2 | −2 | 2 | 0 | −3 | −2 | 2 |
| 151 | D | 1 | −2 | 0 | 5 | −2 | 1 | 0 | 0 | −2 | −3 | −3 | −1 | −2 | −3 | −2 | 1 | −1 | −4 | −3 | −2 |
| 152 | C | −1 | −3 | −3 | −3 | 10 | −3 | −4 | 0 | −3 | −2 | −2 | −3 | −1 | 1 | −4 | −1 | −2 | −2 | −1 | −2 |
| 153 | A | 2 | −2 | −2 | −2 | −2 | −1 | 0 | −2 | −2 | −1 | 0 | −1 | −1 | 1 | 4 | −1 | −1 | −3 | −1 | −1 |
| 154 | V | 2 | −1 | −2 | −1 | −2 | 4 | 1 | −2 | −2 | −1 | −2 | 0 | −1 | −3 | 3 | −1 | −1 | −3 | −2 | 0 |
| 155 | P | −1 | −2 | −1 | 1 | −2 | −2 | −1 | −2 | −2 | 1 | 0 | −2 | −1 | −2 | 4 | 0 | 2 | −4 | −3 | 0 |
| 156 | E | −2 | 1 | −1 | 0 | −4 | 0 | 3 | −3 | 2 | −3 | −3 | 1 | −2 | 2 | 3 | −1 | −2 | −3 | −1 | −3 |
| 157 | C | −1 | −3 | −3 | −3 | 9 | 1 | −3 | −3 | −3 | −2 | −1 | −3 | −1 | −2 | −3 | −2 | −2 | 5 | −2 | −2 |
| 158 | V | −1 | 0 | −2 | −3 | −2 | 0 | −2 | −3 | −3 | 2 | 0 | −2 | 0 | 1 | −2 | −2 | 1 | −2 | −1 | 4 |
| 159 | N | −2 | 1 | 4 | 4 | −3 | 0 | 0 | −1 | 0 | −3 | −3 | −1 | −2 | −1 | −2 | 0 | −1 | 3 | 3 | −3 |
| 160 | P | −1 | −2 | −2 | −1 | −3 | −1 | −1 | −2 | −2 | −1 | −3 | 1 | −2 | −4 | 7 | 0 | −1 | −4 | −3 | −2 |
| 161 | V | −1 | −3 | −3 | −3 | −2 | 0 | 0 | −4 | −3 | 4 | 0 | −2 | 0 | 2 | −3 | −2 | −1 | −2 | 0 | 3 |
| 162 | Y | −2 | 1 | −1 | −3 | −3 | −1 | −1 | −3 | 4 | −2 | 0 | −1 | −1 | 0 | −3 | 0 | −2 | 0 | 6 | −2 |
| 163 | E | −1 | −1 | −1 | 0 | −3 | 3 | 4 | −3 | −1 | −2 | −1 | 0 | −1 | −3 | 3 | 0 | 0 | −3 | −2 | −2 |
| 164 | P | −1 | −1 | −2 | −1 | −3 | 0 | 1 | −3 | −1 | −2 | −1 | 0 | −2 | −2 | 5 | −1 | −1 | −2 | 2 | −2 |
| 165 | E | −1 | −2 | 1 | 2 | −3 | −1 | 1 | 3 | −1 | −3 | −3 | −1 | −3 | −1 | −2 | 0 | −1 | −2 | 2 | −3 |
| 166 | Q | −2 | 0 | −1 | −1 | −3 | 4 | 2 | −3 | 3 | −3 | 0 | 2 | −1 | −2 | −2 | −1 | −2 | 5 | 0 | −2 |
| 167 | C | 0 | −3 | −3 | −4 | 9 | −3 | −4 | −4 | −3 | 0 | 0 | −3 | −1 | −2 | −3 | −2 | −1 | −2 | −2 | 0 |
| 168 | C | 0 | −3 | −3 | −3 | 9 | −3 | −3 | −3 | −3 | 0 | −1 | −3 | −1 | −2 | −3 | 0 | −1 | −3 | −2 | 1 |
| 169 | P | 0 | −2 | −3 | −2 | −3 | −1 | −2 | −2 | −3 | −2 | 0 | −1 | −1 | −3 | 7 | −1 | −1 | −4 | −3 | −1 |
| 170 | V | −1 | −2 | −2 | −2 | −2 | −1 | 1 | −3 | −2 | 3 | 0 | 1 | 0 | −2 | −2 | −2 | −1 | −3 | −2 | 4 |
| 171 | C | −1 | −2 | −3 | −3 | 9 | −2 | −3 | −3 | −3 | −1 | 0 | 1 | −1 | −2 | −3 | −1 | −1 | −3 | −2 | −1 |
| 172 | K | −1 | 1 | 0 | −1 | −3 | 0 | 0 | −2 | −1 | −3 | −2 | 6 | −1 | −3 | −1 | 0 | −1 | −3 | −2 | −2 |
| 173 | N | 2 | −1 | 3 | 2 | −2 | −1 | 0 | 2 | −1 | −3 | −3 | −1 | −2 | −3 | −2 | 1 | −1 | −4 | −3 | −2 |
| 174 | G | 0 | −1 | 0 | −1 | −3 | −2 | −2 | 5 | −2 | −4 | −3 | 0 | −3 | −3 | −2 | 0 | −2 | −2 | −3 | −3 |
| 175 | P | −1 | −2 | 1 | −1 | −3 | −1 | 0 | −2 | −2 | −3 | −3 | −1 | −2 | −4 | 6 | −1 | −1 | −4 | −3 | −2 |
| 176 | N | −1 | −1 | 5 | 0 | −3 | −1 | −1 | −1 | 0 | −3 | −3 | −1 | −2 | −2 | −2 | 1 | 0 | 6 | −1 | −3 |
| 177 | C | −1 | −2 | −2 | −2 | 8 | −2 | −2 | −3 | 3 | −2 | −2 | −2 | −2 | −2 | 1 | −1 | 0 | −3 | −1 | −2 |
| 178 | F | −2 | −2 | −1 | −2 | −3 | −2 | −2 | 1 | 4 | −2 | −2 | 0 | −1 | 4 | 0 | −1 | −2 | 0 | 3 | −2 |
| 179 | A | 2 | −2 | −2 | −3 | −1 | −2 | −2 | 2 | −3 | 0 | 1 | −2 | 0 | 0 | −2 | −1 | −1 | −3 | −1 | 1 |
| 180 | G | 0 | −1 | 0 | 0 | −2 | −1 | 1 | 3 | −2 | −2 | 0 | −1 | −1 | −2 | 1 | 1 | 1 | −3 | −2 | −1 |
| 181 | T | 0 | −2 | −1 | −1 | −1 | −1 | −1 | −2 | −2 | 1 | −1 | −1 | −1 | −2 | 1 | 0 | 5 | −3 | −2 | 0 |
| 182 | T | 1 | −1 | 0 | −1 | −1 | 2 | 0 | −1 | −2 | −1 | 0 | −1 | 0 | −2 | −1 | 1 | 3 | −3 | −2 | −1 |
| 183 | I | 1 | −3 | −2 | −3 | −1 | −2 | −2 | −2 | −3 | 3 | 0 | −2 | 0 | −1 | −2 | 0 | 0 | −3 | −1 | 3 |
| 184 | I | −1 | −3 | −3 | −3 | −2 | −3 | −3 | 1 | −3 | 5 | 0 | −3 | 0 | 2 | −3 | −2 | −1 | −2 | 0 | 1 |
| 185 | P | −1 | −2 | −2 | −1 | −3 | 0 | 1 | −2 | −2 | −2 | 0 | −1 | −1 | −3 | 6 | −1 | −1 | −4 | −3 | −2 |
| 186 | A | 4 | 1 | −2 | −2 | 0 | −1 | −1 | 0 | −2 | −1 | −1 | 0 | −1 | −2 | −1 | 0 | 0 | −3 | −2 | 0 |
| 187 | G | 0 | −2 | 0 | −1 | −3 | −2 | −2 | 6 | −2 | −2 | −3 | −2 | −2 | −3 | −2 | 0 | −2 | −2 | −3 | 0 |
| 188 | I | −1 | 2 | −1 | 0 | −3 | 0 | 3 | −3 | −1 | 2 | −1 | 0 | −1 | −2 | 1 | −1 | −1 | −3 | −2 | 0 |
| 189 | E | −1 | −1 | −1 | 0 | −3 | 0 | 4 | −2 | −1 | 0 | −2 | 0 | −1 | −3 | 3 | −1 | −1 | −3 | −2 | −1 |
| 190 | V | 0 | 0 | −1 | 1 | −2 | −1 | −1 | −2 | −2 | 0 | 0 | −1 | 0 | −2 | −2 | −1 | 2 | −3 | −2 | 3 |
| 191 | K | −1 | 0 | −1 | −1 | −3 | 0 | 1 | −2 | −1 | −2 | −2 | 4 | −1 | −2 | −2 | −1 | −1 | 7 | −1 | 0 |
| 192 | V | 0 | −1 | −1 | −2 | −2 | 1 | 0 | −3 | −2 | 0 | 0 | 1 | 0 | −2 | −2 | −1 | 2 | −3 | −1 | 3 |
| 193 | D | −2 | −2 | 0 | 6 | −3 | 0 | 1 | −1 | −1 | −3 | −4 | −1 | −3 | −3 | −1 | 0 | 1 | −4 | −3 | −3 |
| 194 | E | 1 | −1 | −1 | 1 | −2 | 0 | 4 | −2 | −1 | −1 | −2 | 0 | −1 | −2 | −1 | 0 | −1 | −3 | −2 | 1 |
| 195 | C | 0 | −3 | −3 | −3 | 10 | −3 | −4 | −3 | −3 | −1 | −1 | −3 | −1 | −2 | −3 | −1 | −1 | −2 | −2 | −1 |
| 196 | N | −1 | −1 | 3 | −1 | −2 | −1 | −1 | −1 | −1 | −1 | −1 | −1 | −1 | 2 | −2 | 0 | 4 | −2 | 0 | −1 |
| 197 | I | −1 | −1 | −2 | −3 | 5 | −2 | −2 | −3 | −3 | 4 | 0 | 1 | 0 | −1 | −3 | −1 | −1 | −3 | −1 | 1 |
| 198 | C | 0 | −3 | −2 | −3 | 9 | −3 | −3 | −3 | −3 | −1 | −1 | −3 | −1 | −2 | −3 | −1 | 1 | −2 | −2 | −1 |
| 199 | H | −2 | 2 | 0 | −2 | −3 | 0 | 0 | −2 | 7 | −3 | −2 | 0 | −2 | 0 | −2 | −1 | −2 | −1 | 3 | −3 |
| 200 | C | 0 | −3 | −3 | −3 | 10 | −3 | −4 | −3 | −3 | −1 | −1 | −3 | −1 | −2 | −3 | −1 | −1 | −2 | −2 | −1 |
| 201 | H | −1 | 0 | 0 | −1 | −2 | 0 | 0 | −2 | 7 | −2 | −2 | −1 | −2 | −1 | −2 | 0 | 3 | −2 | 0 | −2 |
| 202 | N | −2 | −1 | 4 | 3 | −3 | 0 | 0 | −1 | 0 | −2 | −2 | −1 | −2 | 0 | −2 | 0 | −1 | −1 | 4 | −2 |
| 203 | G | 0 | −1 | 0 | 0 | −3 | 0 | 3 | 5 | −1 | −4 | −4 | −1 | −3 | −3 | −2 | 0 | −2 | −2 | −3 | −3 |
| 204 | D | −1 | 0 | 0 | 4 | −3 | 3 | 3 | −2 | 0 | −3 | −3 | 0 | −2 | −3 | −1 | 0 | −1 | −3 | −2 | −2 |
| 205 | W | −1 | −2 | 0 | 3 | −3 | −1 | 0 | 3 | −2 | −3 | −3 | −2 | −2 | −1 | −2 | −1 | −2 | 8 | 0 | −3 |
| 206 | W | −2 | −3 | −2 | −3 | −2 | −2 | −3 | 3 | −2 | −3 | −3 | −3 | −1 | 0 | −3 | −2 | −2 | 11 | 0 | −3 |
| 207 | K | −1 | 3 | 0 | −2 | −3 | 0 | 0 | −2 | 0 | −2 | −2 | 3 | −1 | 0 | −2 | −1 | −1 | −1 | 4 | −2 |
| 208 | P | −1 | −1 | −1 | −1 | −2 | 3 | 0 | −3 | −1 | −1 | 1 | 0 | 0 | −2 | 4 | −1 | −1 | −3 | −2 | −1 |
| 209 | A | 5 | −1 | −2 | −2 | 0 | −1 | −1 | 0 | −2 | −1 | −1 | −1 | −1 | −2 | −1 | 0 | 0 | −3 | −2 | 0 |
| 210 | Q | −1 | 0 | −1 | −1 | −2 | 3 | 0 | −2 | −1 | −1 | 0 | 0 | 4 | −1 | −1 | 0 | 2 | −2 | −1 | 0 |
| 211 | C | 0 | −3 | −3 | −3 | 10 | −3 | −4 | −3 | −3 | −1 | −1 | −3 | −1 | −2 | −3 | −1 | −1 | −2 | −2 | −1 |
| 212 | S | 0 | −1 | 0 | 0 | −1 | 0 | 0 | −1 | −1 | −2 | −2 | 0 | −1 | −2 | −1 | 4 | 3 | −3 | −2 | −1 |
| 213 | K | −1 | 5 | 0 | −2 | −3 | 0 | 0 | −2 | 0 | −3 | −2 | 4 | −1 | −3 | −2 | −1 | −1 | −3 | −2 | −3 |
| 214 | R | −1 | 3 | 0 | −2 | −2 | 0 | −1 | −3 | 6 | −1 | 1 | 0 | 0 | −1 | −2 | −1 | −1 | −2 | 0 | −1 |
| 215 | E | −1 | 0 | 0 | 1 | −4 | 1 | 5 | −2 | 0 | −3 | −3 | 3 | −2 | −3 | −1 | 0 | −1 | −3 | −2 | −2 |
| 216 | C | 0 | −2 | 3 | −1 | 9 | −2 | −2 | −2 | −1 | −1 | −1 | −2 | −1 | −2 | −3 | 0 | −1 | −3 | −2 | −1 |
| 217 | Q | −1 | 2 | −1 | −1 | 6 | 3 | 0 | −2 | −1 | −2 | −2 | 1 | −1 | −3 | −2 | 0 | −1 | −2 | −2 | −2 |
| 218 | G | −1 | 0 | 3 | 0 | −3 | 3 | 0 | 3 | 0 | −3 | −3 | 0 | −1 | −3 | −2 | 0 | −1 | −3 | −2 | −3 |
| 219 | K | −1 | 0 | 0 | −2 | −2 | 0 | 0 | −2 | 5 | 0 | 0 | 3 | 3 | −1 | −2 | −1 | −1 | −2 | 0 | 0 |
| 220 | Q | −1 | 0 | 0 | 0 | −3 | 4 | 4 | −2 | 0 | −3 | −2 | 0 | −1 | −3 | −1 | 0 | −1 | −2 | −1 | −2 |
| 221 | T | 0 | −1 | 0 | −1 | −2 | −1 | −1 | 4 | −2 | −2 | −2 | −1 | −2 | −2 | −1 | 0 | 4 | −2 | −2 | −1 |
| 222 | V | 0 | −2 | −2 | −3 | −1 | −1 | −2 | −3 | −2 | 2 | 1 | −1 | 4 | 0 | −2 | −1 | 0 | −2 | −1 | 3; or |

2) comprising a fragment of the amino acid sequence of a1), wherein said fragment is a member of the vWFC domain containing protein family, or has an antigenic determinant in common with the amino acid sequence of a1); or
3) comprising a functional equivalent of the amino acid sequence of a1) or a2); or
4) consisting of the amino acid sequence of a1); or
5) of any of a1)-a4), wherein the polypeptide has a maximum threshold E value of 10⁻²; or
6) comprising an amino acid sequence satisfying the consensus amino acid sequence [GTDFC](0,1)-[CF](0,1)-[VMSED](0,1)-[DEA](0,1)-[DENG](0,1)-[SQNDG](0,1)-[SGR](0,1)-[FIV](0,1)-[VYFE](0,1)-[YFS](0,1)-[KVAGP](0,1)-[LIG](0,1)-[GE](0,1)-[EWQM](0,1)-[RKYFQVI](0,1)-[FYWT](0,1)-[FALYRTS](0,1)-[PED](0,1)-[GS](0,1)-[HPDS](0,1)-[STHP](0,1)-[CNAT](0,1)-[CTE](0,1)-[PQRL](0,1)-C(0,1)-[VELT](0,1)-C(0,1)-[TAQ](0,1)-[ELATSD](0,1)-[EDT]-G-[PS]-[VLAQS]-[CS]-[DAMSTFCV]-[QRKV]-R(0,1)-[PT]-[ERDK]-C-[PTV]-[KERSA]-[LIVT]-[HPSC]-[PAE]-[KRASY]-[CP]-[TIVM]-[HKRE]-[VI]-[DESAKP]-[HTNRYG]-[NSTYHK](0,1)-[PA](0,1)-[TG](0,1)-[QGDES]-C-C-[PV]-[EQRDLV]-C; or
7) comprising a fragment of the amino acid sequence of a6), wherein said fragment is a member of the vWFC domain containing protein family, or has an antigenic determinant in common with the polypeptides of a6); or
8) comprising a functional equivalent of a6) or a7); or
9) consisting of an amino acid sequence satisfying the consensus amino acid sequence[GTDFC](0,1)-[CF](0,1)-[VMSED](0,1)-[DEA](0,1)-[DENG](0,1)-[SQNDG](0,1)-[SGR](0,1)-[FIV](0,1)-[VYFE](0,1)-[YFS](0,1)-[KVAGP](0,1)-[LIG](0,1)-[GE](0,1)-[EWQM](0,1)-[RKYFQVI](0,1)-[FYWT](0,1)-[FALYRTS](0,1)-[PED](0,1)-[GS](0,1)-[HPDS](0,1)-[STHP](0,1)-[CNAT](0,1)-[CTE](0,1)-[PQRL](0,1)-C(0,1)-[VELT](0,1)-C(0,1)-[TAQ](0,1)-[ELATSD](0,1)-[EDT]-G-[PS]-[VLAQS]-[CS]-[DAMSTFCV]-[QRKV]-R(0,1)-[PT]-[ERDK]-C-[PTV]-[KERSA]-[LIVT]-[HPSC]-[PAE]-[KRASY]-[CP]-[TIVM]-[HKRE]-[VI]-[DESAKP]-[HTNRYG]-[NSTYHK](0,1)-[PA](0,1)-[TG](0,1)-[QGDES]-C-C-[PV]-[EQRDLV]-C; or
10) comprising an amino acid sequence selected from the group consisting of SEQ ID NO:2, SEQ ID NO:4, SEQ ID NO:39, SEQ ID NO:41, SEQ ID NO:43, and SEQ ID NO:45; or
11) comprising a fragment of the amino acid sequence of a10), wherein said fragment is a member of the vWFC domain containing protein family, or has an antigenic determinant in common with the amino acid sequence of a10); or
12) comprising a functional equivalent of a10) or a11); or
13) consisting of an amino acid sequence selected from the group consisting of SEQ ID NO:2, SEQ ID NO:4, SEQ ID NO:39, SEQ ID NO:41, SEQ ID NO:43, and SEQ ID NO:45; or
14) comprising a fragment of the amino acid sequence of a13), wherein said fragment is a member of the vWFC domain containing protein family, or has an antigenic determinant in common with the amino acid sequence of a13); or
15) comprising a functional equivalent of a13) or a14); or
16) comprising an amino acid sequence selected from the group consisting of SEQ ID NO:6, SEQ ID NO:8, SEQ ID NO:10, SEQ ID NO:12, SEQ ID NO:14, SEQ ID NO:16, SEQ ID NO:47, SEQ ID NO:49, SEQ ID NO:51, and SEQ ID NO:53; or
17) comprising a fragment of the amino acid sequence of a16), wherein said fragment is a member of the vWFC domain containing protein family, or has an antigenic determinant in common with the amino acid sequence of a16); or
18) comprising a functional equivalent of a16) or a17); or
19) consisting of an amino acid sequence selected from the group consisting of SEQ ID NO:6, SEQ ID NO:8, SEQ ID NO:10, SEQ ID NO:12, SEQ ID NO:14, SEQ ID NO:16, SEQ ID NO:47, SEQ ID NO:49, SEQ ID NO:51, and SEQ ID NO:53; or
20) comprising a fragment of the amino acid sequence of a19), wherein said fragment is a member of the vWFC domain containing protein family, or has an antigenic determinant in common with the amino acid sequence of a19); or
21) comprising a functional equivalent of a19) or a20); or
22) comprising the amino acid sequence selected from the group consisting of SEQ ID NO:18, SEQ ID NO:20, SEQ ID NO:22, SEQ ID NO:24, SEQ ID NO:26, SEQ ID NO:28, SEQ ID NO:30, SEQ ID NO:55, SEQ ID NO:57, SEQ ID NO:59, and SEQ ID NO:61; or
23) comprising a fragment of the amino acid sequence of a22), wherein said fragment is a member of the vWFC domain containing protein family, or has an antigenic determinant in common with the amino acid sequence of a22); or
24) comprising a functional equivalent of a22) or a23); or
25) consisting of an amino acid sequence selected from the group consisting of SEQ ID NO:18, SEQ ID NO:20, SEQ ID NO:22, SEQ ID NO:24, SEQ ID NO:26, SEQ ID NO:28, SEQ ID NO:30, SEQ ID NO:55, SEQ ID NO:57, SEQ ID NO:59, and SEQ ID NO:61; or
26) comprising a fragment of the amino acid sequence of a25), wherein said fragment is a member of the vWFC domain containing protein family, or has an antigenic determinant in common with the amino acid sequence of a25); or
27) comprising a functional equivalent of a25) or a26); or
28) comprising the functional equivalent of any of a3), a5), a8), a12), a15), a18), a21), a24), or a27), characterised in that it is homologous to an amino acid sequence selected from the group consisting of SEQ ID NO:2, SEQ ID NO:4, SEQ ID NO:6, SEQ ID NO: 8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, and SEQ ID NO:61, and is a member of the vWFC domain containing protein family; or
29) comprising the fragment or functional equivalent of any of a2), a3), a5), a7), a8), a11), a12), a14), a15), a17), a18), a20), a21), a23), a24), a26), a27), or a28), wherein the fragment or functional equivalent has greater than 80% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, and SEQ ID NO:49, or with an active fragment of any of the foregoing; or
30) comprising the functional equivalent of any of a3), a5), a8), a12), a15), a18), a21), a24), a27), a28, or a29), wherein the functional equivalent exhibits significant structural homology with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, and SEQ ID NO:61; or
31) comprising the fragment of any of a2), a5), a7), a11), a14), a17), a20), a23), a26), or a29), wherein the fragment has an antigenic determinant in common with the amino acid sequence of any one of a1), a4), a5,), a6), a9), a10), a13), a16), a19), a22), or a25), which consists of 7 or more amino acid residues from the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59 or SEQ ID NO:61; or
b) a purified nucleic acid molecule:
1) comprising a nucleic acid sequence encoding a polypeptide according to any one of a1)-a31); or
2) comprising a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, and SEQ ID NO:60, or a redundant equivalent or fragment of any of the foregoing; or
3) consisting of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27 SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, and SEQ ID NO:60, or a redundant equivalent or fragment of any of the foregoing; or
4) that hybridizes under high stringency conditions with a nucleic acid molecule according to any one of b1) to b3); or
c) a vector comprising a nucleic acid molecule according to any one of b1) to b4); or
d) a host cell transformed with a vector according to c); or
e) a ligand:
1) that binds specifically to the polypeptide of any of a1) to a31); or
2) which is an antibody that binds specifically to the polypeptide of any of a1) to a31); or
f) a compound:
1) that increases the level of expression or activity of a polypeptide according to any of a1) to a31); or
2) that decreases the level of expression or activity of a polypeptide according to any of a1) to a31); or
g) the compound of f1) or f2), wherein the compound binds to a polypeptide according to any of a1) to a31) without inducing any of the biological effects of the polypeptide; or
h) the compound of g), wherein the compound is a natural or modified substrate, ligand, enzyme, receptor or structural or functional mimetic; or
i) a pharmaceutical composition comprising any one of a) to h), and a pharmaceutically acceptable carrier; or
j) a vaccine composition comprising any one of a1) to a31) or b1) to b4); or
k) a kit for diagnosing disease, comprising a first container containing a nucleic acid probe that hybridizes under stringent conditions with a nucleic acid molecule of any one of b 1) to b4), a second container containing primers useful for amplifying the nucleic acid molecule, and instructions for using the probe and primers for facilitating the diagnosis of disease; or
l) a kit for diagnosing disease, comprising a first container containing a nucleic acid probe that hybridizes under stringent conditions with a nucleic acid molecule of any one of b1) to b4); a second container containing primers useful for amplifying the nucleic acid molecule; a third container holding an agent for digesting unhybridized RNA; and instructions for using the probe and primers for facilitating the diagnosis of disease; or
m) a kit comprising an array of nucleic acid molecules, at least one of which is a nucleic acid molecule according to any one of b1) to b4); or
n) a kit comprising one or more antibodies that bind to a polypeptide as recited in any one of a1) to a31); and a reagent useful for the detection of a binding reaction between the one or more antibodies and the polypeptide; or
o) a transgenic or knockout non-human animal that has been transformed to express higher, lower, or absent levels of a polypeptide according to any one of a1) to a31).

62: A method of using a composition of matter, comprising obtaining a composition of matter according to claim 61 and using said composition of matter in a method selected from: diagnosing a disease in a patient; treatment of a disease in a patient; monitoring the therapeutic treatment of a disease; identification of a compound that is effective in the treatment and/or diagnosis of a disease; and screening candidate compounds.

63: The method of claim 62, wherein said method of using a composition of matter comprises the method for treatment of a disease, comprising administering to the patient:
a) an isolated polypeptide:
1) comprising an amino acid sequence that has an E value of 10⁻² or less when the profile below is input as a query sequence into the search program BLAST, using the default parameters specified by the NCBI (the National Center for Biotechnology Information) [Blosum 62 matrix; gap open penalty=11 and gap extension penalty=1]:

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | M | −2 | −2 | −3 | −4 | −2 | −1 | −3 | −4 | −3 | 0 | 2 | −2 | 8 | 0 | −3 | −2 | −2 | −2 | −2 | 0 |
| 2 | A | 3 | −1 | −3 | −3 | −1 | −1 | −2 | −2 | −3 | 0 | 0 | 1 | 2 | 1 | −2 | −1 | −1 | −3 | −1 | 1 |
| 3 | L | 1 | −2 | −2 | −3 | −2 | −2 | −2 | −2 | 2 | 0 | 2 | −2 | 0 | −2 | 0 | 1 | −1 | −3 | −2 | 2 |
| 4 | H | −1 | −1 | 1 | −1 | −3 | −1 | −1 | 3 | 6 | −3 | −1 | −1 | −2 | −2 | −2 | 2 | −1 | −3 | −1 | −3 |
| 5 | I | 0 | −2 | −3 | −3 | −2 | 0 | 0 | −3 | −3 | 3 | 2 | −2 | 0 | −1 | −3 | −2 | −1 | −3 | −2 | 2 |
| 6 | H | 0 | −2 | −1 | −2 | −2 | −1 | −1 | −3 | 7 | 0 | 0 | −2 | −1 | −2 | 0 | 0 | −1 | −3 | 0 | 2 |
| 7 | E | 0 | −2 | −2 | −1 | −2 | −1 | 2 | −3 | −1 | 0 | 1 | −1 | 2 | 2 | −3 | 0 | −1 | −2 | 2 | 0 |
| 8 | A | 2 | −2 | −1 | −2 | 3 | −2 | −2 | 2 | −2 | −2 | −2 | −2 | −2 | −3 | −2 | 3 | 1 | −3 | −3 | −2 |
| 9 | C | 0 | −3 | −2 | −3 | 7 | −2 | −3 | −3 | −3 | 0 | −1 | −2 | 3 | 3 | −3 | 0 | −1 | −2 | −1 | 0 |
| 10 | I | −1 | −2 | −2 | −2 | −2 | −2 | 0 | 0 | −2 | 3 | 2 | −2 | 0 | 0 | −3 | 0 | 0 | −2 | 3 | 0 |
| 11 | L | −2 | 0 | −3 | −4 | −2 | −2 | −3 | −4 | −3 | 0 | 4 | −2 | 3 | 3 | −4 | −2 | −2 | −2 | 0 | 0 |
| 12 | L | 0 | 0 | −3 | −4 | −2 | −2 | −3 | −3 | −3 | 0 | 3 | −2 | 0 | −1 | 1 | −2 | −1 | −3 | −2 | 1 |
| 13 | L | −1 | −2 | −2 | −2 | −2 | −2 | 0 | −3 | −3 | 0 | 3 | −2 | 0 | 1 | −3 | 1 | 1 | −3 | −1 | 1 |
| 14 | V | 0 | 0 | −3 | −3 | 4 | 0 | −2 | −3 | −3 | 0 | 0 | −2 | 2 | −2 | −3 | −2 | −1 | −3 | −2 | 4 |
| 15 | I | −1 | 0 | −2 | −3 | 4 | −2 | −2 | −3 | 2 | 2 | 1 | −2 | 0 | −1 | −3 | 0 | 0 | 4 | −1 | 0 |
| 16 | P | 0 | 0 | −2 | −2 | −2 | −2 | −2 | −3 | 4 | −1 | 0 | −2 | −1 | 2 | 3 | 0 | −1 | −2 | 0 | 0 |
| 17 | G | 0 | −3 | −2 | −3 | −2 | −2 | −3 | 2 | −3 | 1 | 0 | −3 | 3 | 3 | −3 | −1 | −2 | −2 | 0 | 1 |
| 18 | L | 1 | −3 | −3 | −4 | −1 | −2 | −3 | −3 | −3 | 0 | 4 | −2 | 0 | −1 | −3 | −2 | −1 | −3 | −2 | 2 |
| 19 | V | 2 | −1 | −1 | −2 | −2 | 3 | −1 | −2 | −2 | 0 | −1 | −1 | −1 | −3 | −2 | 1 | 1 | −3 | −2 | 2 |
| 20 | T | −1 | −2 | −2 | −3 | 4 | −2 | −2 | −3 | −3 | 0 | 3 | −2 | 0 | −2 | −3 | 1 | 3 | −3 | −2 | 0 |
| 21 | S | 0 | −2 | −1 | −2 | 4 | −2 | −2 | 1 | −3 | −1 | 1 | −2 | 2 | −2 | 0 | 2 | −1 | −3 | −3 | −1 |
| 22 | A | 3 | −2 | −2 | −2 | −1 | −2 | −2 | −1 | −2 | −1 | 0 | −2 | −1 | 1 | 2 | 2 | −1 | −3 | −2 | 0 |
| 23 | A | 2 | −2 | −3 | −2 | 5 | 0 | 0 | −2 | −3 | −1 | 0 | −2 | −1 | −3 | 2 | −1 | −1 | −3 | −2 | 1 |
| 24 | I | 1 | 1 | −2 | −2 | −2 | −1 | −2 | −2 | −2 | 2 | 0 | −1 | −1 | −1 | −2 | 2 | −1 | −3 | 1 | 1 |
| 25 | S | −1 | −1 | 2 | −1 | −2 | 1 | −1 | −2 | 4 | 2 | −1 | −1 | −1 | −2 | −2 | 3 | −1 | −4 | −1 | −1 |
| 26 | H | 0 | −2 | 3 | −1 | −3 | −1 | −1 | −2 | 5 | −2 | −2 | −1 | −2 | 0 | 3 | 0 | −1 | −3 | −1 | 0 |
| 27 | E | −1 | −1 | 0 | 1 | −3 | 0 | 5 | −2 | −1 | −3 | −3 | 0 | −2 | −3 | −1 | 1 | 2 | −3 | −2 | −2 |
| 28 | D | 1 | −2 | 0 | 4 | −2 | −1 | 0 | −1 | −2 | −2 | 0 | −1 | −1 | −2 | −1 | 2 | 0 | −4 | −3 | −2 |
| 29 | Y | −2 | −1 | −2 | −2 | −3 | 2 | −1 | −3 | 0 | 0 | −1 | −1 | −1 | 0 | 3 | −1 | −2 | 0 | 5 | 0 |
| 30 | P | 0 | −1 | −1 | −1 | −2 | −1 | −1 | 0 | 5 | −3 | −3 | −1 | −2 | −3 | 4 | 0 | 0 | −3 | −1 | −2 |
| 31 | A | 3 | −2 | −2 | −2 | −1 | −1 | −2 | −1 | −3 | 0 | 2 | −1 | 0 | −2 | 0 | 0 | 0 | −3 | −2 | 0 |
| 32 | D | −2 | −1 | 0 | 5 | −3 | 0 | 2 | −2 | −1 | −2 | −3 | 2 | −2 | −3 | −1 | 0 | −1 | −4 | −3 | 0 |
| 33 | E | 0 | −1 | −2 | 0 | −2 | 0 | 3 | −3 | −1 | 0 | 0 | −1 | 0 | 3 | −2 | −1 | −1 | −2 | 0 | 0 |
| 34 | G | 1 | −2 | 0 | −1 | −2 | −1 | −2 | 4 | −2 | −2 | −2 | −2 | 0 | −2 | −2 | 1 | 1 | −2 | −3 | 0 |
| 35 | D | 0 | 2 | 0 | 2 | −2 | 0 | 0 | −2 | −1 | −2 | 0 | 0 | −1 | −3 | 2 | −1 | −1 | −3 | −2 | −2 |
| 36 | Q | 0 | 3 | 0 | −1 | −2 | 3 | 0 | −2 | −1 | −2 | −2 | 0 | −1 | −3 | −1 | 1 | 3 | −3 | −2 | −2 |
| 37 | I | 0 | −2 | −3 | −2 | −1 | −2 | −2 | −2 | −3 | 1 | 0 | −2 | 0 | −2 | 4 | −1 | −1 | −3 | −2 | 2 |
| 38 | S | −1 | −1 | 0 | 1 | −2 | 0 | 2 | −2 | −1 | 2 | −1 | 1 | −1 | −2 | −2 | 2 | −1 | −3 | −2 | 0 |
| 39 | S | 3 | −1 | −1 | −1 | −1 | −1 | −1 | −1 | −2 | −1 | 0 | −1 | −1 | −2 | 2 | 3 | 0 | −3 | −2 | −1 |
| 40 | N | 0 | −2 | 2 | −1 | −3 | −1 | 1 | 3 | −1 | 0 | 0 | −1 | −1 | −3 | 1 | −1 | −1 | −3 | −2 | 0 |
| 41 | D | 1 | −2 | −1 | 3 | −2 | 0 | 2 | −2 | −2 | −1 | 0 | −1 | −1 | 0 | −2 | 0 | −1 | −3 | −2 | −1 |
| 42 | N | −1 | −1 | 2 | 1 | −3 | 0 | 3 | 0 | −1 | −2 | 0 | 0 | −1 | −2 | −2 | 1 | −1 | −3 | −2 | −2 |
| 43 | L | 1 | −3 | −3 | −3 | 4 | −2 | −3 | −3 | −1 | 0 | 1 | −3 | 0 | 3 | −3 | −2 | −2 | 0 | 5 | 0 |
| 44 | I | 1 | −1 | −1 | 0 | −2 | 0 | 2 | −2 | −2 | 1 | −1 | 1 | −1 | −2 | −2 | 2 | −1 | −4 | −2 | 0 |
| 45 | F | −2 | −3 | −2 | 1 | −2 | −2 | −2 | −3 | −1 | 2 | 0 | −3 | 0 | 4 | −3 | −2 | −2 | −1 | 4 | 1 |
| 46 | D | 1 | −2 | −1 | 2 | −2 | −1 | 0 | 0 | −2 | −3 | −3 | −1 | −2 | −3 | 4 | 1 | −1 | −4 | −3 | −2 |
| 47 | D | 0 | −2 | 0 | 5 | −3 | −1 | 0 | 1 | −2 | −3 | −4 | −1 | −3 | −3 | 2 | 1 | −1 | −4 | −3 | −3 |
| 48 | Y | −2 | 3 | −1 | −2 | −3 | 2 | −1 | −3 | 0 | −2 | −2 | 0 | −1 | 0 | −3 | −2 | −2 | 0 | 6 | −2 |
| 49 | R | −2 | 4 | 0 | −2 | −4 | 2 | 0 | −2 | 5 | −4 | −3 | 0 | −2 | −3 | 2 | −1 | −2 | −3 | −1 | −3 |
| 50 | G | −1 | −2 | 0 | 2 | −3 | −1 | 1 | 4 | −2 | −2 | −3 | −1 | −2 | −3 | −2 | 1 | −1 | −3 | −3 | 0 |
| 51 | K | −1 | 2 | 1 | −1 | −3 | 0 | 0 | 1 | −1 | −3 | −3 | 4 | −2 | −3 | −2 | 1 | −1 | −3 | −3 | −3 |
| 52 | G | −1 | −2 | −1 | 2 | 4 | −2 | −1 | 3 | −2 | −2 | −3 | −2 | −2 | 0 | −2 | 0 | 2 | −2 | −2 | −2 |
| 53 | C | −1 | −4 | −4 | −4 | 10 | −4 | −4 | −4 | −3 | −1 | −1 | −4 | −1 | 2 | −4 | −2 | −2 | −2 | −1 | −1 |
| 54 | V | −1 | −2 | −1 | 0 | −2 | −1 | 1 | −2 | −2 | 0 | 0 | −1 | 3 | −2 | −2 | 1 | 0 | −3 | −2 | 3 |
| 55 | D | 0 | −2 | 0 | 6 | −3 | 0 | 1 | −1 | −1 | −3 | −4 | −1 | −3 | −3 | −1 | 0 | −1 | −4 | −3 | −3 |
| 56 | D | −2 | −1 | 3 | 4 | −4 | 0 | 2 | 2 | −1 | −4 | −4 | −1 | −3 | −4 | −2 | 0 | −1 | −4 | −3 | −3 |
| 57 | S | 0 | −1 | 2 | 2 | −3 | 1 | 0 | 2 | −1 | −3 | −3 | −1 | −2 | −3 | −2 | 3 | 0 | −3 | −3 | −3 |
| 58 | G | 0 | 1 | 0 | −1 | −3 | −1 | −2 | 5 | −2 | −4 | −4 | −1 | −3 | −3 | −2 | 1 | −2 | −3 | −3 | −3 |
| 59 | F | −1 | −4 | −4 | −4 | −2 | −3 | −3 | −4 | −2 | 3 | 0 | −3 | 0 | 5 | −4 | −2 | −1 | −1 | 0 | 3 |
| 60 | V | −1 | −3 | −3 | −2 | −2 | −1 | 1 | −3 | −1 | 0 | 0 | −2 | 0 | 3 | −3 | −2 | −1 | −1 | 4 | 3 |
| 61 | Y | −2 | −2 | −2 | −3 | −2 | −2 | −2 | −3 | 0 | −1 | −1 | −2 | −1 | 4 | −3 | 0 | −2 | 0 | 7 | −2 |
| 62 | K | 1 | −1 | −2 | −2 | −2 | −1 | −1 | 0 | −3 | 0 | −1 | 1 | −1 | −2 | 2 | −1 | −1 | −4 | −2 | 3 |
| 63 | L | −1 | −3 | −2 | −3 | −2 | −3 | −3 | 4 | −3 | 4 | 1 | −3 | 0 | −1 | −3 | −1 | −2 | −3 | −2 | 0 |
| 64 | G | −1 | −1 | 0 | 0 | −4 | 0 | 3 | 5 | −1 | −4 | −4 | −1 | −3 | −4 | −2 | 0 | −2 | −3 | −3 | −3 |
| 65 | E | −2 | −1 | −1 | 0 | −3 | 3 | 3 | −3 | −1 | −2 | −1 | 0 | 3 | −1 | −2 | −1 | −2 | 7 | −1 | −2 |
| 66 | R | −2 | 2 | −2 | −3 | −2 | 0 | −1 | −3 | 0 | 1 | −1 | 1 | −1 | 3 | −3 | −2 | −2 | −1 | 4 | 0 |
| 67 | F | −2 | −3 | −3 | −3 | −2 | −3 | −3 | −3 | −1 | −1 | −1 | −3 | −1 | 6 | −4 | −2 | 1 | 6 | 3 | −1 |
| 68 | F | 0 | 1 | −2 | −3 | −2 | −1 | −2 | −2 | −1 | −1 | 1 | −1 | 0 | 2 | −3 | 0 | 0 | −1 | 2 | −1 |
| 69 | P | −2 | −2 | −1 | 2 | −4 | 0 | 2 | −2 | −2 | −4 | −4 | −1 | −3 | −4 | 6 | −1 | −1 | −4 | −3 | −3 |
| 70 | G | 0 | −2 | 0 | −1 | −2 | −1 | −1 | 5 | −2 | −4 | −4 | −1 | −2 | −3 | −2 | 3 | −1 | −3 | −3 | −3 |
| 71 | H | −2 | −2 | 0 | 5 | −3 | −1 | 0 | −2 | 4 | −3 | −4 | −1 | −3 | −3 | 3 | 0 | −1 | −4 | −2 | −3 |
| 72 | S | −1 | −1 | 0 | −1 | −2 | −1 | −1 | −2 | 5 | −3 | −3 | −1 | −2 | −3 | 4 | 3 | 1 | −3 | −1 | −2 |
| 73 | N | 2 | −2 | 2 | −2 | 8 | −2 | −2 | −2 | −2 | −2 | −2 | −2 | −2 | −3 | −3 | 0 | 0 | −3 | −2 | −1 |
| 74 | C | −1 | −2 | −1 | −1 | 8 | −1 | 3 | −3 | −2 | −2 | −2 | −1 | −2 | −3 | −2 | 0 | 2 | −3 | −2 | −1 |
| 75 | P | −1 | 2 | −2 | −2 | −3 | 3 | 0 | −3 | −2 | −2 | 0 | 0 | −1 | −3 | 5 | −1 | −1 | −3 | −2 | −2 |
| 76 | C | 0 | −4 | −4 | −4 | 10 | −4 | −5 | −4 | −4 | −1 | −1 | −4 | −1 | −2 | −4 | −1 | −1 | −2 | −2 | −1 |
| 77 | V | −1 | −2 | −2 | −1 | −2 | −1 | 2 | −3 | −2 | 0 | 2 | −1 | 0 | −1 | −2 | −1 | 0 | −3 | −2 | 3 |
| 78 | C | 0 | −4 | −4 | −4 | 10 | −4 | −5 | −4 | −4 | −1 | −1 | −4 | −1 | −2 | −4 | −1 | −1 | −2 | −2 | −1 |
| 79 | A | 2 | −1 | −1 | −1 | −1 | 1 | −1 | −2 | −2 | −2 | −2 | −1 | −1 | −3 | −1 | 0 | 4 | −3 | −2 | −1 |
| 80 | L | 1 | −1 | 1 | 0 | −2 | 0 | 2 | −2 | −2 | −1 | 0 | −1 | −1 | −2 | −2 | 1 | 1 | −3 | −2 | −1 |
| 81 | D | −2 | −2 | 0 | 5 | −4 | 0 | 4 | −2 | −1 | −3 | −4 | −1 | −3 | −3 | −2 | 0 | 1 | −4 | −3 | −3 |
| 82 | G | 0 | −3 | 0 | −2 | −4 | −3 | −3 | 7 | −3 | −5 | −5 | −3 | −4 | −4 | −3 | 0 | −3 | −3 | −4 | −4 |
| 83 | P | −1 | −2 | −2 | −1 | −3 | −1 | −1 | −2 | −2 | −3 | −3 | −1 | −2 | −4 | 7 | 1 | −1 | −4 | −3 | −3 |
| 84 | V | 0 | −2 | −2 | −3 | −2 | 1 | −1 | −3 | −3 | 1 | 1 | −2 | 0 | −2 | −2 | 0 | −1 | −3 | −2 | 4 |
| 85 | C | 0 | −3 | −3 | −3 | 10 | −3 | −4 | −3 | −3 | −2 | −2 | −3 | −2 | −3 | −3 | 0 | −1 | −3 | −3 | −2 |
| 86 | D | 1 | −2 | −1 | 3 | 4 | −1 | −1 | −2 | −2 | −1 | −2 | −2 | 0 | 0 | −2 | 1 | 0 | −3 | −2 | 0 |
| 87 | Q | −1 | 3 | −1 | −2 | −3 | 4 | 0 | −3 | −1 | −2 | −2 | 2 | −1 | −3 | −2 | −1 | −1 | −3 | −2 | 0 |
| 88 | P | −1 | −2 | −1 | −2 | −2 | −2 | −2 | −3 | −3 | −2 | −2 | −2 | −2 | −4 | 6 | 0 | 4 | −4 | −3 | −1 |
| 89 | E | −2 | 2 | 0 | 2 | −4 | 0 | 5 | −2 | −1 | −4 | −3 | 1 | −2 | −4 | −2 | −1 | −2 | −4 | −3 | −3 |
| 90 | C | 0 | −4 | −4 | −4 | 10 | −4 | −5 | −4 | −4 | −2 | −2 | −4 | −2 | −3 | −4 | −2 | −2 | −3 | −3 | −2 |
| 91 | P | −1 | −2 | −2 | −2 | −3 | −2 | −2 | −3 | −3 | −1 | −2 | −2 | −2 | −3 | 6 | −1 | 2 | −4 | −3 | 0 |
| 92 | K | 0 | 3 | 0 | 0 | −3 | 0 | 3 | −2 | −1 | −3 | −3 | 3 | −2 | −3 | −2 | 0 | −1 | −3 | −2 | −2 |
| 93 | I | −1 | −3 | −3 | −4 | −1 | −2 | −3 | −4 | −3 | 3 | 3 | −2 | 0 | −1 | −3 | −2 | 1 | −3 | −2 | 1 |
| 94 | H | −1 | −1 | 0 | −1 | 4 | −1 | −1 | −2 | 7 | −3 | −3 | −1 | −2 | −2 | 2 | 1 | −1 | −3 | 0 | −3 |
| 95 | P | 0 | −2 | −2 | −1 | −3 | −1 | 1 | −2 | −2 | −3 | −3 | −1 | −2 | −4 | 7 | −1 | −1 | −4 | −3 | −2 |
| 96 | K | 1 | 3 | −1 | −2 | −2 | 0 | 0 | −2 | 0 | −2 | −2 | 2 | −1 | −1 | −2 | 1 | −1 | −2 | 2 | −2 |
| 97 | C | 0 | −3 | −3 | −3 | 10 | −3 | −4 | −3 | −3 | −2 | −2 | −3 | −2 | −3 | 2 | −1 | −1 | −3 | −3 | −2 |
| 98 | T | −1 | −3 | −2 | −3 | −1 | −2 | −3 | −4 | −3 | 4 | 0 | −3 | 1 | −1 | −3 | −1 | 3 | −3 | −2 | 2 |
| 99 | K | −2 | 1 | 0 | −1 | −4 | 0 | 2 | −2 | 7 | −3 | −3 | 2 | −2 | −2 | −2 | −1 | −2 | −3 | 0 | −3 |
| 100 | V | −1 | −3 | −3 | −3 | −1 | −3 | −3 | −4 | −3 | 3 | 0 | −3 | 0 | −1 | −3 | −2 | −1 | −3 | −1 | 5 |
| 101 | E | 1 | −1 | 0 | 3 | −2 | 0 | 2 | −1 | −1 | −3 | −3 | 1 | −2 | −3 | −1 | 2 | 0 | −4 | −3 | −2 |
| 102 | H | −2 | 2 | 1 | −1 | −3 | 0 | −1 | −2 | 7 | −3 | −2 | 0 | −2 | −1 | −2 | −1 | 1 | −2 | 3 | −2 |
| 103 | N | −1 | 0 | 3 | −1 | −2 | 0 | 0 | −1 | 4 | −2 | −2 | 1 | −2 | 0 | −2 | 1 | 0 | −1 | 4 | −2 |
| 104 | G | −1 | −1 | 0 | 2 | −3 | 4 | 2 | 2 | −1 | −4 | −3 | 0 | −2 | −3 | −2 | 0 | −1 | −3 | −2 | −3 |
| 105 | C | 0 | −3 | −3 | −3 | 10 | −3 | −5 | −3 | −3 | −1 | −1 | −3 | −1 | −2 | −3 | −1 | −1 | −2 | −2 | −1 |
| 106 | C | 0 | −3 | −3 | −3 | 10 | −3 | −5 | −3 | −3 | −1 | −1 | −3 | −1 | −2 | −3 | −1 | −1 | −2 | −2 | −1 |
| 107 | P | −1 | −3 | −3 | −2 | −3 | −2 | −2 | −3 | −3 | −1 | −2 | −2 | −2 | −4 | 7 | −2 | −1 | −4 | −3 | 0 |
| 108 | E | −1 | 2 | 0 | 2 | −3 | 4 | 2 | −2 | −1 | −2 | −1 | 0 | −1 | −3 | −2 | −1 | −1 | −3 | −2 | 0 |
| 109 | C | 0 | −3 | −3 | −3 | 10 | −3 | −5 | −3 | −3 | −1 | −1 | −3 | −1 | −2 | −3 | −1 | −1 | −2 | −2 | −1 |
| 110 | K | −1 | 0 | 0 | 0 | −3 | 0 | 3 | −2 | −1 | −1 | −2 | 3 | −1 | −3 | −2 | 1 | −1 | −3 | −2 | 1 |
| 111 | E | 2 | 2 | −1 | 0 | −3 | 0 | 4 | −2 | −1 | −3 | −2 | 2 | −2 | −3 | −2 | 0 | −1 | −3 | −2 | −2 |
| 112 | V | −1 | 1 | −1 | −1 | −3 | −1 | 1 | 1 | −2 | 1 | −1 | 1 | −1 | −2 | −2 | −1 | −1 | −3 | −2 | 2 |
| 113 | K | 0 | 0 | 0 | −1 | −3 | 0 | 0 | 4 | −2 | −4 | −3 | 4 | −2 | −3 | −2 | 1 | −1 | −3 | −3 | −3 |
| 114 | N | −2 | 0 | 6 | 0 | −3 | 0 | 0 | 0 | 0 | −3 | −3 | 2 | −2 | −3 | −2 | 0 | 0 | −4 | −2 | −3 |
| 115 | F | −1 | −3 | −3 | −3 | −2 | −2 | −2 | −3 | 0 | 0 | 0 | −2 | 0 | 4 | −3 | −2 | −1 | 0 | 6 | 1 |
| 116 | C | 0 | −3 | −3 | −3 | 10 | −3 | −5 | −3 | −3 | −1 | −1 | −3 | −1 | −2 | −3 | −1 | −1 | −2 | −2 | −1 |
| 117 | E | −1 | −1 | 0 | 3 | −3 | 0 | 4 | −2 | −1 | −2 | 0 | 0 | −2 | −3 | −2 | 0 | 1 | −3 | −3 | −2 |
| 118 | Y | −2 | −2 | −2 | −2 | −3 | −1 | 1 | −3 | 0 | −1 | −1 | −2 | −1 | 5 | −3 | −2 | −2 | 0 | 6 | −2 |
| 119 | H | −2 | 5 | 2 | −2 | −3 | 0 | −1 | −2 | 4 | −2 | −1 | 0 | 3 | −2 | −2 | −1 | −1 | −3 | −1 | −2 |
| 120 | G | −1 | −2 | 2 | −1 | −3 | −2 | −2 | 6 | −2 | −4 | −4 | −2 | −3 | −3 | −2 | 0 | −2 | −3 | −3 | −3 |
| 121 | K | −1 | 4 | −1 | −2 | −3 | 0 | 0 | −3 | −1 | −1 | −2 | 5 | −1 | −3 | −2 | −1 | −1 | −3 | −2 | 0 |
| 122 | N | −1 | −2 | 2 | −2 | −2 | −2 | −2 | −2 | −2 | 2 | 0 | −2 | 0 | −2 | −2 | 0 | 4 | −3 | −2 | 1 |
| 123 | Y | −2 | −3 | −3 | −3 | −2 | −2 | −3 | −3 | 0 | −1 | −1 | −3 | −1 | 4 | −4 | −2 | −2 | 1 | 8 | −1 |
| 124 | K | 0 | 1 | 0 | −1 | −3 | 2 | 2 | −2 | 4 | −3 | −2 | 2 | −1 | −1 | −2 | −1 | −2 | −2 | 3 | −2 |
| 125 | I | −1 | −2 | 2 | −2 | −2 | −2 | −2 | −2 | −2 | 2 | 1 | −2 | 0 | −1 | −2 | 0 | 1 | −3 | −2 | 2 |
| 126 | L | −1 | −2 | 2 | −2 | −2 | −2 | −2 | 3 | −2 | 0 | 3 | −2 | 0 | −1 | −3 | −1 | −1 | −3 | −2 | −1 |
| 127 | E | −1 | 0 | 0 | 1 | −4 | 3 | 6 | −2 | 0 | −3 | −3 | 0 | −2 | −3 | −1 | 0 | −1 | −3 | −2 | −2 |
| 128 | E | −1 | −1 | 2 | 0 | −3 | 0 | 4 | −2 | 0 | −3 | −3 | 0 | −2 | −1 | −2 | 0 | 1 | −2 | 3 | −2 |
| 129 | F | −2 | −3 | −3 | −3 | −2 | −3 | −3 | −3 | −1 | 0 | 0 | −3 | 0 | 7 | −5 | −2 | −2 | 0 | 2 | −1 |
| 130 | K | −1 | 1 | 2 | −1 | −3 | 2 | 0 | −2 | −1 | −1 | 0 | 2 | 1 | −2 | −2 | −1 | −1 | −3 | −2 | 0 |
| 131 | P | −1 | −3 | −3 | −3 | −2 | −2 | −2 | −3 | −3 | 1 | 2 | −2 | 0 | −2 | 3 | −2 | 0 | −3 | −2 | 3 |
| 132 | S | 0 | 0 | 1 | 2 | 2 | −1 | 0 | −1 | −1 | −3 | −3 | −1 | −2 | −3 | 1 | 4 | 1 | −4 | −3 | −2 |
| 133 | P | −1 | −2 | −2 | −1 | −3 | −1 | 1 | −3 | −2 | −2 | −3 | −1 | −2 | −4 | 7 | −1 | −1 | −4 | −3 | 0 |
| 134 | C | 0 | −3 | −3 | −3 | 10 | −3 | −4 | −3 | −3 | −1 | −1 | −3 | −1 | −2 | −3 | −1 | 0 | −2 | −2 | −1 |
| 135 | E | −1 | 1 | −1 | 0 | −3 | 0 | 5 | −3 | −1 | −1 | 1 | 0 | −1 | −2 | −2 | −1 | −1 | −3 | −2 | −1 |
| 136 | W | −1 | 2 | −1 | −2 | −3 | 2 | 0 | −2 | 3 | −2 | 0 | 1 | −1 | −2 | −2 | 1 | −1 | 4 | −1 | −2 |
| 137 | C | 0 | 0 | −3 | −3 | 10 | −3 | −4 | −3 | −3 | 0 | −1 | −3 | −1 | −2 | −3 | −1 | 0 | −3 | −2 | −1 |
| 138 | R | −1 | 4 | −1 | −2 | −2 | 0 | −1 | −3 | −1 | 2 | −1 | 2 | −1 | −1 | −2 | −1 | 1 | −2 | 1 | 0 |
| 139 | C | 0 | −4 | −4 | −4 | 10 | −4 | −5 | −4 | −4 | −1 | −1 | −4 | −1 | −2 | −4 | −1 | −1 | −2 | −2 | −1 |
| 140 | E | −1 | −1 | 0 | 2 | −3 | 0 | 5 | −3 | −1 | −2 | 0 | 0 | −1 | −3 | −2 | 0 | 1 | −3 | −2 | −2 |
| 141 | P | 1 | −2 | 1 | −2 | −2 | −1 | −1 | −2 | −2 | 0 | 0 | −1 | −1 | −2 | 2 | 1 | 1 | −3 | −2 | 0 |
| 142 | S | −1 | −1 | 4 | 2 | −3 | 1 | 0 | 2 | −1 | −3 | −3 | −1 | −2 | −3 | −2 | 2 | −1 | −4 | −3 | −3 |
| 143 | N | −1 | 1 | 2 | −1 | −3 | −1 | −1 | 4 | −1 | −4 | −4 | 1 | −2 | −3 | −2 | 1 | −1 | −3 | −3 | −3 |
| 144 | E | −1 | 0 | −1 | 0 | −3 | 0 | 4 | −3 | −1 | 1 | −1 | 0 | −1 | −2 | −2 | −1 | 1 | −3 | −2 | 1 |
| 145 | V | 3 | −2 | −3 | −3 | −1 | −2 | −2 | −2 | −3 | 0 | 0 | −2 | 0 | −2 | −2 | −1 | −1 | −3 | −2 | 3 |
| 146 | H | −2 | 1 | −2 | −3 | −2 | −1 | −2 | −3 | 3 | 0 | 2 | −1 | 0 | 2 | −3 | −2 | −2 | −1 | 4 | −1 |
| 147 | C | −1 | 0 | −3 | −3 | 10 | −3 | −3 | −3 | −3 | −2 | −2 | −2 | −2 | −3 | 1 | −1 | −1 | −3 | −3 | −2 |
| 148 | V | 0 | −2 | −1 | −2 | −1 | −1 | −2 | −2 | −3 | 0 | 1 | −1 | 0 | −2 | −2 | 2 | 3 | −3 | −2 | 2 |
| 149 | V | −1 | −3 | −3 | −3 | 1 | −2 | −2 | −3 | −3 | 2 | 0 | −2 | 0 | −2 | 2 | −2 | −1 | −4 | −2 | 5 |
| 150 | A | 3 | −2 | −2 | −2 | 2 | −1 | −1 | −1 | −2 | 0 | −1 | −1 | −1 | −2 | −2 | 2 | 0 | −3 | −2 | 2 |
| 151 | D | 1 | −2 | 0 | 5 | −2 | 1 | 0 | 0 | −2 | −3 | −3 | −1 | −2 | −3 | −2 | 1 | −1 | −4 | −3 | −2 |
| 152 | C | −1 | −3 | −3 | −3 | 10 | −3 | −4 | 0 | −3 | −2 | −2 | −3 | −1 | 1 | −4 | −1 | −2 | −2 | −1 | −2 |
| 153 | A | 2 | −2 | −2 | −2 | −2 | −1 | 0 | −2 | −2 | −1 | 0 | −1 | −1 | 1 | 4 | −1 | −1 | −3 | −1 | −1 |
| 154 | V | 2 | −1 | −2 | −1 | −2 | 4 | 1 | −2 | −2 | −1 | −2 | 0 | −1 | −3 | 3 | −1 | −1 | −3 | −2 | 0 |
| 155 | P | −1 | −2 | −1 | 1 | −2 | −2 | −1 | −2 | −2 | 1 | 0 | −2 | −1 | −2 | 4 | 0 | 2 | −4 | −3 | 0 |
| 156 | E | −2 | 1 | −1 | 0 | −4 | 0 | 3 | −3 | 2 | −3 | −3 | 1 | −2 | 2 | 3 | −1 | −2 | −3 | −1 | −3 |
| 157 | C | −1 | −3 | −3 | −3 | 9 | 1 | −3 | −3 | −3 | −2 | −1 | −3 | −1 | −2 | −3 | −2 | −2 | 5 | −2 | −2 |
| 158 | V | −1 | 0 | −2 | −3 | −2 | 0 | −2 | −3 | −3 | 2 | 0 | −2 | 0 | 1 | −2 | −2 | 1 | −2 | −1 | 4 |
| 159 | N | −2 | 1 | 4 | 4 | −3 | 0 | 0 | −1 | 0 | −3 | −3 | −1 | −2 | −1 | −2 | 0 | −1 | 3 | 3 | −3 |
| 160 | P | −1 | −2 | −2 | −1 | −3 | −1 | −1 | −2 | −2 | −1 | −3 | 1 | −2 | −4 | 7 | 0 | −1 | −4 | −3 | −2 |
| 161 | V | −1 | −3 | −3 | −3 | −2 | 0 | 0 | −4 | −3 | 4 | 0 | −2 | 0 | 2 | −3 | −2 | −1 | −2 | 0 | 3 |
| 162 | Y | −2 | 1 | −1 | −3 | −3 | −1 | −1 | −3 | 4 | −2 | 0 | −1 | −1 | 0 | −3 | 0 | −2 | 0 | 6 | −2 |
| 163 | E | −1 | −1 | −1 | 0 | −3 | 3 | 4 | −3 | −1 | −2 | −1 | 0 | −1 | −3 | 3 | 0 | 0 | −3 | −2 | −2 |
| 164 | P | −1 | −1 | −2 | −1 | −3 | 0 | 1 | −3 | −1 | −2 | −1 | 0 | −2 | −2 | 5 | −1 | −1 | −2 | 2 | −2 |
| 165 | E | −1 | −2 | 1 | 2 | −3 | −1 | 1 | 3 | −1 | −3 | −3 | −1 | −3 | −1 | −2 | 0 | −1 | −2 | 2 | −3 |
| 166 | Q | −2 | 0 | −1 | −1 | −3 | 4 | 2 | −3 | 3 | −3 | 0 | 2 | −1 | −2 | −2 | −1 | −2 | 5 | 0 | −2 |
| 167 | C | 0 | −3 | −3 | −4 | 9 | −3 | −4 | −4 | −3 | 0 | 0 | −3 | −1 | −2 | −3 | −2 | −1 | −2 | −2 | 0 |
| 168 | C | 0 | −3 | −3 | −3 | 9 | −3 | −3 | −3 | −3 | 0 | −1 | −3 | −1 | −2 | −3 | 0 | −1 | −3 | −2 | 1 |
| 169 | P | 0 | −2 | −3 | −2 | −3 | −1 | −2 | −2 | −3 | −2 | 0 | −1 | −1 | −3 | 7 | −1 | −1 | −4 | −3 | −1 |
| 170 | V | −1 | −2 | −2 | −2 | −2 | −1 | 1 | −3 | −2 | 3 | 0 | 1 | 0 | −2 | −2 | −2 | −1 | −3 | −2 | 4 |
| 171 | C | −1 | −2 | −3 | −3 | 9 | −2 | −3 | −3 | −3 | −1 | 0 | 1 | −1 | −2 | −3 | −1 | −1 | −3 | −2 | −1 |
| 172 | K | −1 | 1 | 0 | −1 | −3 | 0 | 0 | −2 | −1 | −3 | −2 | 6 | −1 | −3 | −1 | 0 | −1 | −3 | −2 | −2 |
| 173 | N | 2 | −1 | 3 | 2 | −2 | −1 | 0 | 2 | −1 | −3 | −3 | −1 | −2 | −3 | −2 | 1 | −1 | −4 | −3 | −2 |
| 174 | G | 0 | −1 | 0 | −1 | −3 | −2 | −2 | 5 | −2 | −4 | −3 | 0 | −3 | −3 | −2 | 0 | −2 | −2 | −3 | −3 |
| 175 | P | −1 | −2 | 1 | −1 | −3 | −1 | 0 | −2 | −2 | −3 | −3 | −1 | −2 | −4 | 6 | −1 | −1 | −4 | −3 | −2 |
| 176 | N | −1 | −1 | 5 | 0 | −3 | −1 | −1 | −1 | 0 | −3 | −3 | −1 | −2 | −2 | −2 | 1 | 0 | 6 | −1 | −3 |
| 177 | C | −1 | −2 | −2 | −2 | 8 | −2 | −2 | −3 | 3 | −2 | −2 | −2 | −2 | −2 | 1 | −1 | 0 | −3 | −1 | −2 |
| 178 | F | −2 | −2 | −1 | −2 | −3 | −2 | −2 | 1 | 4 | −2 | −2 | 0 | −1 | 4 | 0 | −1 | −2 | 0 | 3 | −2 |
| 179 | A | 2 | −2 | −2 | −3 | −1 | −2 | −2 | 2 | −3 | 0 | 1 | −2 | 0 | 0 | −2 | −1 | −1 | −3 | −1 | 1 |
| 180 | G | 0 | −1 | 0 | 0 | −2 | −1 | 1 | 3 | −2 | −2 | 0 | −1 | −1 | −2 | 1 | 1 | 1 | −3 | −2 | −1 |
| 181 | T | 0 | −2 | −1 | −1 | −1 | −1 | −1 | −2 | −2 | 1 | −1 | −1 | −1 | −2 | 1 | 0 | 5 | −3 | −2 | 0 |
| 182 | T | 1 | −1 | 0 | −1 | −1 | 2 | 0 | −1 | −2 | −1 | 0 | −1 | 0 | −2 | −1 | 1 | 3 | −3 | −2 | −1 |
| 183 | I | 1 | −3 | −2 | −3 | −1 | −2 | −2 | −2 | −3 | 3 | 0 | −2 | 0 | −1 | −2 | 0 | 0 | −3 | −1 | 3 |
| 184 | I | −1 | −3 | −3 | −3 | −2 | −3 | −3 | 1 | −3 | 5 | 0 | −3 | 0 | 2 | −3 | −2 | −1 | −2 | 0 | 1 |
| 185 | P | −1 | −2 | −2 | −1 | −3 | 0 | 1 | −2 | −2 | −2 | 0 | −1 | −1 | −3 | 6 | −1 | −1 | −4 | −3 | −2 |
| 186 | A | 4 | 1 | −2 | −2 | 0 | −1 | −1 | 0 | −2 | −1 | −1 | 0 | −1 | −2 | −1 | 0 | 0 | −3 | −2 | 0 |
| 187 | G | 0 | −2 | 0 | −1 | −3 | −2 | −2 | 6 | −2 | −2 | −3 | −2 | −2 | −3 | −2 | 0 | −2 | −2 | −3 | 0 |
| 188 | I | −1 | 2 | −1 | 0 | −3 | 0 | 3 | −3 | −1 | 2 | −1 | 0 | −1 | −2 | 1 | −1 | −1 | −3 | −2 | 0 |
| 189 | E | −1 | −1 | −1 | 0 | −3 | 0 | 4 | −2 | −1 | 0 | −2 | 0 | −1 | −3 | 3 | −1 | −1 | −3 | −2 | −1 |
| 190 | V | 0 | 0 | −1 | 1 | −2 | −1 | −1 | −2 | −2 | 0 | 0 | −1 | 0 | −2 | −2 | −1 | 2 | −3 | −2 | 3 |
| 191 | K | −1 | 0 | −1 | −1 | −3 | 0 | 1 | −2 | −1 | −2 | −2 | 4 | −1 | −2 | −2 | −1 | −1 | 7 | −1 | 0 |
| 192 | V | 0 | −1 | −1 | −2 | −2 | 1 | 0 | −3 | −2 | 0 | 0 | 1 | 0 | −2 | −2 | −1 | 2 | −3 | −1 | 3 |
| 193 | D | −2 | −2 | 0 | 6 | −3 | 0 | 1 | −1 | −1 | −3 | −4 | −1 | −3 | −3 | −1 | 0 | 1 | −4 | −3 | −3 |
| 194 | E | 1 | −1 | −1 | 1 | −2 | 0 | 4 | −2 | −1 | −1 | −2 | 0 | −1 | −2 | −1 | 0 | −1 | −3 | −2 | 1 |
| 195 | C | 0 | −3 | −3 | −3 | 10 | −3 | −4 | −3 | −3 | −1 | −1 | −3 | −1 | −2 | −3 | −1 | −1 | −2 | −2 | −1 |
| 196 | N | −1 | −1 | 3 | −1 | −2 | −1 | −1 | −1 | −1 | −1 | −1 | −1 | −1 | 2 | −2 | 0 | 4 | −2 | 0 | −1 |
| 197 | I | −1 | −1 | −2 | −3 | 5 | −2 | −2 | −3 | −3 | 4 | 0 | 1 | 0 | −1 | −3 | −1 | −1 | −3 | −1 | 1 |
| 198 | C | 0 | −3 | −2 | −3 | 9 | −3 | −3 | −3 | −3 | −1 | −1 | −3 | −1 | −2 | −3 | −1 | 1 | −2 | −2 | −1 |
| 199 | H | −2 | 2 | 0 | −2 | −3 | 0 | 0 | −2 | 7 | −3 | −2 | 0 | −2 | 0 | −2 | −1 | −2 | −1 | 3 | −3 |
| 200 | C | 0 | −3 | −3 | −3 | 10 | −3 | −4 | −3 | −3 | −1 | −1 | −3 | −1 | −2 | −3 | −1 | −1 | −2 | −2 | −1 |
| 201 | H | −1 | 0 | 0 | −1 | −2 | 0 | 0 | −2 | 7 | −2 | −2 | −1 | −2 | −1 | −2 | 0 | 3 | −2 | 0 | −2 |
| 202 | N | −2 | −1 | 4 | 3 | −3 | 0 | 0 | −1 | 0 | −2 | −2 | −1 | −2 | 0 | −2 | 0 | −1 | −1 | 4 | −2 |
| 203 | G | 0 | −1 | 0 | 0 | −3 | 0 | 3 | 5 | −1 | −4 | −4 | −1 | −3 | −3 | −2 | 0 | −2 | −2 | −3 | −3 |
| 204 | D | −1 | 0 | 0 | 4 | −3 | 3 | 3 | −2 | 0 | −3 | −3 | 0 | −2 | −3 | −1 | 0 | −1 | −3 | −2 | −2 |
| 205 | W | −1 | −2 | 0 | 3 | −3 | −1 | 0 | 3 | −2 | −3 | −3 | −2 | −2 | −1 | −2 | −1 | −2 | 8 | 0 | −3 |
| 206 | W | −2 | −3 | −2 | −3 | −2 | −2 | −3 | 3 | −2 | −3 | −3 | −3 | −1 | 0 | −3 | −2 | −2 | 11 | 0 | −3 |
| 207 | K | −1 | 3 | 0 | −2 | −3 | 0 | 0 | −2 | 0 | −2 | −2 | 3 | −1 | 0 | −2 | −1 | −1 | −1 | 4 | −2 |
| 208 | P | −1 | −1 | −1 | −1 | −2 | 3 | 0 | −3 | −1 | −1 | 1 | 0 | 0 | −2 | 4 | −1 | −1 | −3 | −2 | −1 |
| 209 | A | 5 | −1 | −2 | −2 | 0 | −1 | −1 | 0 | −2 | −1 | −1 | −1 | −1 | −2 | −1 | 0 | 0 | −3 | −2 | 0 |
| 210 | Q | −1 | 0 | −1 | −1 | −2 | 3 | 0 | −2 | −1 | −1 | 0 | 0 | 4 | −1 | −1 | 0 | 2 | −2 | −1 | 0 |
| 211 | C | 0 | −3 | −3 | −3 | 10 | −3 | −4 | −3 | −3 | −1 | −1 | −3 | −1 | −2 | −3 | −1 | −1 | −2 | −2 | −1 |
| 212 | S | 0 | −1 | 0 | 0 | −1 | 0 | 0 | −1 | −1 | −2 | −2 | 0 | −1 | −2 | −1 | 4 | 3 | −3 | −2 | −1 |
| 213 | K | −1 | 5 | 0 | −2 | −3 | 0 | 0 | −2 | 0 | −3 | −2 | 4 | −1 | −3 | −2 | −1 | −1 | −3 | −2 | −3 |
| 214 | R | −1 | 3 | 0 | −2 | −2 | 0 | −1 | −3 | 6 | −1 | 1 | 0 | 0 | −1 | −2 | −1 | −1 | −2 | 0 | −1 |
| 215 | E | −1 | 0 | 0 | 1 | −4 | 1 | 5 | −2 | 0 | −3 | −3 | 3 | −2 | −3 | −1 | 0 | −1 | −3 | −2 | −2 |
| 216 | C | 0 | −2 | 3 | −1 | 9 | −2 | −2 | −2 | −1 | −1 | −1 | −2 | −1 | −2 | −3 | 0 | −1 | −3 | −2 | −1 |
| 217 | Q | −1 | 2 | −1 | −1 | 6 | 3 | 0 | −2 | −1 | −2 | −2 | 1 | −1 | −3 | −2 | 0 | −1 | −2 | −2 | −2 |
| 218 | G | −1 | 0 | 3 | 0 | −3 | 3 | 0 | 3 | 0 | −3 | −3 | 0 | −1 | −3 | −2 | 0 | −1 | −3 | −2 | −3 |
| 219 | K | −1 | 0 | 0 | −2 | −2 | 0 | 0 | −2 | 5 | 0 | 0 | 3 | 3 | −1 | −2 | −1 | −1 | −2 | 0 | 0 |
| 220 | Q | −1 | 0 | 0 | 0 | −3 | 4 | 4 | −2 | 0 | −3 | −2 | 0 | −1 | −3 | −1 | 0 | −1 | −2 | −1 | −2 |
| 221 | T | 0 | −1 | 0 | −1 | −2 | −1 | −1 | 4 | −2 | −2 | −2 | −1 | −2 | −2 | −1 | 0 | 4 | −2 | −2 | −1 |
| 222 | V | 0 | −2 | −2 | −3 | −1 | −1 | −2 | −3 | −2 | 2 | 1 | −1 | 4 | 0 | −2 | −1 | 0 | −2 | −1 | 3; Or |

2) comprising a fragment of the amino acid sequence of a1), wherein said fragment is a member of the vWFC domain containing protein family, or has an antigenic determinant in common with the amino acid sequence of a1); or
3) comprising a functional equivalent of the amino acid sequence of a1) or a2); or
4) consisting of the amino acid sequence of a1); or
5) of any of a1)-a4), wherein the polypeptide has a maximum threshold E value of 10⁻²; or
6) comprising an amino acid sequence satisfying the consensus amino acid sequence [GTDFC](0,1)-[CF](0,1)-[VMSED](0,1)-[DEA](0,1)-[DENG](0,1)-[SQNDG](0,1)-[SGR](0,1)-[FIV](0,1)-[VYFE](0,1)-[YFS](0,1)-[KVAGP](0,1)-[LIG](0,1)-[GE](0,1)-[EWQM](0,1)-[RKYFQVI](0,1)-[FYWT](0,1)-[FALYRTS](0,1)-[PED](0,1)-[GS](0,1)-[HPDS](0,1)-[STHP](0,1)-[CNAT](0,1)-[CTE](0,1)-[PQRL](0,1)-C(0,1)-[VELT](0,1)-C(0,1)-[TAQ](0,1)-[ELATSD](0,1)-[EDT]-G-[PS]-[VLAQS]-[CS]-[DAMSTFCV]-[QRKV]-R(0,1)-[PT]-[ERDK]-C-[PTV]-[KERSA]-[LIVT]-[HPSC]-[PAE]-[KRASY]-[CP]-[TIVM]-[HKRE]-[VI]-[DESAKP]-[HTNRYG]-[NSTYHK](0,1)-[PA](0,1)-[TG](0,1)-[QGDES]-C-C-[PV]-[EQRDLV]-C; or
7) comprising a fragment of the amino acid sequence of a6), wherein said fragment is a member of the vWFC domain containing protein family, or has an antigenic determinant in common with the polypeptides of a6); or
8) comprising a functional equivalent of a6) or a7); or
9) consisting of an amino acid sequence satisfying the consensus amino acid sequence[GTDFC](0,1)-[CF](0,1)-[VMSED](0,1)-[DEA](0,1)-[DENG](0,1)-[SQNDG](0,1)-[SGR](0,1)-[FIV](0,1)-[VYFE](0,1)-[YFS](0,1)-[KVAGP](0,1)-[LIG](0,1)-[GE](0,1)-[EWQM](0,1)-[RKYFQVI](0,1)-[FYWT](0,1)-[FALYRTS](0,1)-[PED](0,1)-[GS](0,1)-[HPDS](0,1)-[STHP](0,1)-[CNAT](0,1)-[CTE](0,1)-[PQRL](0,1)-C(0,1)-[VELT](0,1)-C(0,1)-[TAQ](0,1)-[ELATSD](0,1)-[EDT]-G-[PS]-[VLAQS]-[CS]-[DAMSTFCV]-[QRKV]-R(0,1)-[PT]-[ERDK]-C-[PTV]-[KERSA]-[LIVT]-[HPSC]-[PAE]-[KRASY]-[CP]-[TIVM]-[HKRE]-[VI]-[DESAKP]-[HTNRYG]-[NSTYHK](0,1)-[PA](0,1)-[TG](0,1)-[QGDES]-C-C-[PV]-[EQRDLV]-C; or
10) comprising an amino acid sequence selected from the group consisting of SEQ ID NO:2, SEQ ID NO:4, SEQ ID NO:39, SEQ ID NO:41, SEQ ID NO:43, and SEQ ID NO:45; or
11) comprising a fragment of the amino acid sequence of a10), wherein said fragment is a member of the vWFC domain containing protein family, or has an antigenic determinant in common with the amino acid sequence of a10); or
12) comprising a functional equivalent of a10) or a11); or
13) consisting of an amino acid sequence selected from the group consisting of SEQ ID NO:2, SEQ ID NO:4, SEQ ID NO:39, SEQ ID NO:41, SEQ ID NO:43, and SEQ ID NO:45; or
14) comprising a fragment of the amino acid sequence of a13), wherein said fragment is a member of the vWFC domain containing protein family, or has an antigenic determinant in common with the amino acid sequence of a13); or
15) comprising a functional equivalent of a13) or a14); or
16) comprising an amino acid sequence selected from the group consisting of SEQ ID NO:6, SEQ ID NO:8, SEQ ID NO:10, SEQ ID NO:12, SEQ ID NO:14, SEQ ID NO:16, SEQ ID NO:47, SEQ ID NO:49, SEQ ID NO:51, and SEQ ID NO:53; or
17) comprising a fragment of the amino acid sequence of a16), wherein said fragment is a member of the vWFC domain containing protein family, or has an antigenic determinant in common with the amino acid sequence of a16); or
18) comprising a functional equivalent of a16) or a17); or 19) consisting of an amino acid sequence selected from the group consisting of SEQ ID NO:6, SEQ ID NO:8, SEQ ID NO:10, SEQ ID NO:12, SEQ ID NO:14, SEQ ID NO:16, SEQ ID NO:47, SEQ ID NO:49, SEQ ID NO:51, and SEQ ID NO:53; or
20) comprising a fragment of the amino acid sequence of a19), wherein said fragment is a member of the vWFC domain containing protein family, or has an antigenic determinant in common with the amino acid sequence of a19); or
21) comprising a functional equivalent of a19) or a20); or
22) comprising the amino acid sequence selected from the group consisting of SEQ ID NO:18, SEQ ID NO:20, SEQ ID NO:22, SEQ ID NO:24, SEQ ID NO:26, SEQ ID NO:28, SEQ ID NO:30, SEQ ID NO:55, SEQ ID NO:57, SEQ ID NO:59, and SEQ ID NO:61; or
23) comprising a fragment of the amino acid sequence of a22), wherein said fragment is a member of the vWFC domain containing protein family, or has an antigenic determinant in common with the amino acid sequence of a22); or
24) comprising a functional equivalent of a22) or a23); or
25) consisting of an amino acid sequence selected from the group consisting of SEQ ID NO:18, SEQ ID NO:20, SEQ ID NO:22, SEQ ID NO:24, SEQ ID NO:26, SEQ ID NO:28, SEQ ID NO:30, SEQ ID NO:55, SEQ ID NO:57, SEQ ID NO:59, and SEQ ID NO:61; or
26) comprising a fragment of the amino acid sequence of a25), wherein said fragment is a member of the vWFC domain containing protein family, or has an antigenic determinant in common with the amino acid sequence of a25); or
27) comprising a functional equivalent of a25) or a26); or
28) comprising the functional equivalent of any of a3), a5), a8), a12), a15), a18), a21), a24), or a27), characterised in that it is homologous to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, and SEQ ID NO:61, and is a member of the vWFC domain containing protein family; or
29) comprising the fragment or functional equivalent of any of a2), a3), a5), a7), a8), a11), a12), a14), a15), a17), a18), a20), a21), a23), a24), a26), a27), or a28), wherein the fragment or functional equivalent has greater than 80% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, and SEQ ID NO:49, or with an active fragment of any of the foregoing; or
30) comprising the functional equivalent of any of a3), a5), a8), a12), a15), a18), a21), a24), a27), a28, or a29), wherein the functional equivalent exhibits significant structural homology with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, and SEQ ID NO:61; or
31) comprising the fragment of any of a2), a5), a7), a11), a14), a17), a20), a23), a26), or a29), wherein the fragment has an antigenic determinant in common with the amino acid sequence of any one of a1), a4), a5,), a6), a9), a10), a13), a16), a19), a22), or a25), which consists of 7 or more amino acid residues from the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59 or SEQ ID NO:61; or
b) a purified nucleic acid molecule:
1) comprising a nucleic acid sequence encoding a polypeptide according to any one of a1)-a31); or
2) comprising a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, and SEQ ID NO:60, or a redundant equivalent or fragment of any of the foregoing; or
3) consisting of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27 SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, and SEQ ID NO:60, or a redundant equivalent or fragment of any of the foregoing; or
4) that hybridizes under high stringency conditions with a nucleic acid molecule according to any one of b1) to b3); or
c) a vector comprising a nucleic acid molecule according to any one of b1) to b4); or
d) a host cell transformed with a vector according to c); or
e) a ligand:
1) that binds specifically to the polypeptide of any of a1) to a31); or
2) which is an antibody that binds specifically to the polypeptide of any of a1) to a31); or
f) a compound:
1) that increases the level of expression or activity of a polypeptide according to any of a1) to a31); or
2) that decreases the level of expression or activity of a polypeptide according to any of a1) to a31); or
g) the compound of f1) or f2), wherein the compound binds to a polypeptide according to any of a1) to a31) without inducing any of the biological effects of the polypeptide; or
h) the compound of g), wherein the compound is a natural or modified substrate, ligand, enzyme, receptor or structural or functional mimetic; or
i) a pharmaceutical composition comprising any one of a) to h), and a pharmaceutically acceptable carrier.

64: The method of claim 63, wherein the disease includes one or more of among cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders such as those relating to cartilage and bone skeletal development, including osteoarthritis; metabolic disorders including diabetes mellitus, osteoporosis, and obesity, AIDS and renal disease; infections including viral infection, bacterial infection, fungal infection and parasitic infection.

65: The method of claim 63, wherein the disease is one in which lymphocyte antigens are implicated.

66: The method of claim 63, wherein the disease is one for which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

67: The method of claim 63, wherein the disease is one for which expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.

68: The method of claim 62, wherein said method of using a composition of matter comprises the method for diagnosing a disease in a patient, comprising assessing the level of expression of a natural gene encoding a polypeptide, or assessing the activity of the polypeptide, in tissue from said patient; and comparing said level of expression or activity to a control level, wherein a level that is different to said control level is indicative of disease, and wherein the polypeptide:
a) comprises an amino acid sequence that has an E value of 10⁻² or less when the profile below is input as a query sequence into the search program BLAST, using the default parameters specified by the NCBI (the National Center for Biotechnology Information) [Blosum 62 matrix; gap open penalty=11 and gap extension penalty=1]:

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | M | −2 | −2 | −3 | −4 | −2 | −1 | −3 | −4 | −3 | 0 | 2 | −2 | 8 | 0 | −3 | −2 | −2 | −2 | −2 | 0 |
| 2 | A | 3 | −1 | −3 | −3 | −1 | −1 | −2 | −2 | −3 | 0 | 0 | 1 | 2 | 1 | −2 | −1 | −1 | −3 | −1 | 1 |
| 3 | L | 1 | −2 | −2 | −3 | −2 | −2 | −2 | −2 | 2 | 0 | 2 | −2 | 0 | −2 | 0 | 1 | −1 | −3 | −2 | 2 |
| 4 | H | −1 | −1 | 1 | −1 | −3 | −1 | −1 | 3 | 6 | −3 | −1 | −1 | −2 | −2 | −2 | 2 | −1 | −3 | −1 | −3 |
| 5 | I | 0 | −2 | −3 | −3 | −2 | 0 | 0 | −3 | −3 | 3 | 2 | −2 | 0 | −1 | −3 | −2 | −1 | −3 | −2 | 2 |
| 6 | H | 0 | −2 | −1 | −2 | −2 | −1 | −1 | −3 | 7 | 0 | 0 | −2 | −1 | −2 | 0 | 0 | −1 | −3 | 0 | 2 |
| 7 | E | 0 | −2 | −2 | −1 | −2 | −1 | 2 | −3 | −1 | 0 | 1 | −1 | 2 | 2 | −3 | 0 | −1 | −2 | 2 | 0 |
| 8 | A | 2 | −2 | −1 | −2 | 3 | −2 | −2 | 2 | −2 | −2 | −2 | −2 | −2 | −3 | −2 | 3 | 1 | −3 | −3 | −2 |
| 9 | C | 0 | −3 | −2 | −3 | 7 | −2 | −3 | −3 | −3 | 0 | −1 | −2 | 3 | 3 | −3 | 0 | −1 | −2 | −1 | 0 |
| 10 | I | −1 | −2 | −2 | −2 | −2 | −2 | 0 | 0 | −2 | 3 | 2 | −2 | 0 | 0 | −3 | 0 | 0 | −2 | 3 | 0 |
| 11 | L | −2 | 0 | −3 | −4 | −2 | −2 | −3 | −4 | −3 | 0 | 4 | −2 | 3 | 3 | −4 | −2 | −2 | −2 | 0 | 0 |
| 12 | L | 0 | 0 | −3 | −4 | −2 | −2 | −3 | −3 | −3 | 0 | 3 | −2 | 0 | −1 | 1 | −2 | −1 | −3 | −2 | 1 |
| 13 | L | −1 | −2 | −2 | −2 | −2 | −2 | 0 | −3 | −3 | 0 | 3 | −2 | 0 | 1 | −3 | 1 | 1 | −3 | −1 | 1 |
| 14 | V | 0 | 0 | −3 | −3 | 4 | 0 | −2 | −3 | −3 | 0 | 0 | −2 | 2 | −2 | −3 | −2 | −1 | −3 | −2 | 4 |
| 15 | I | −1 | 0 | −2 | −3 | 4 | −2 | −2 | −3 | 2 | 2 | 1 | −2 | 0 | −1 | −3 | 0 | 0 | 4 | −1 | 0 |
| 16 | P | 0 | 0 | −2 | −2 | −2 | −2 | −2 | −3 | 4 | −1 | 0 | −2 | −1 | 2 | 3 | 0 | −1 | −2 | 0 | 0 |
| 17 | G | 0 | −3 | −2 | −3 | −2 | −2 | −3 | 2 | −3 | 1 | 0 | −3 | 3 | 3 | −3 | −1 | −2 | −2 | 0 | 1 |
| 18 | L | 1 | −3 | −3 | −4 | −1 | −2 | −3 | −3 | −3 | 0 | 4 | −2 | 0 | −1 | −3 | −2 | −1 | −3 | −2 | 2 |
| 19 | V | 2 | −1 | −1 | −2 | −2 | 3 | −1 | −2 | −2 | 0 | −1 | −1 | −1 | −3 | −2 | 1 | 1 | −3 | −2 | 2 |
| 20 | T | −1 | −2 | −2 | −3 | 4 | −2 | −2 | −3 | −3 | 0 | 3 | −2 | 0 | −2 | −3 | 1 | 3 | −3 | −2 | 0 |
| 21 | S | 0 | −2 | −1 | −2 | 4 | −2 | −2 | 1 | −3 | −1 | 1 | −2 | 2 | −2 | 0 | 2 | −1 | −3 | −3 | −1 |
| 22 | A | 3 | −2 | −2 | −2 | −1 | −2 | −2 | −1 | −2 | −1 | 0 | −2 | −1 | 1 | 2 | 2 | −1 | −3 | −2 | 0 |
| 23 | A | 2 | −2 | −3 | −2 | 5 | 0 | 0 | −2 | −3 | −1 | 0 | −2 | −1 | −3 | 2 | −1 | −1 | −3 | −2 | 1 |
| 24 | I | 1 | 1 | −2 | −2 | −2 | −1 | −2 | −2 | −2 | 2 | 0 | −1 | −1 | −1 | −2 | 2 | −1 | −3 | 1 | 1 |
| 25 | S | −1 | −1 | 2 | −1 | −2 | 1 | −1 | −2 | 4 | 2 | −1 | −1 | −1 | −2 | −2 | 3 | −1 | −4 | −1 | −1 |
| 26 | H | 0 | −2 | 3 | −1 | −3 | −1 | −1 | −2 | 5 | −2 | −2 | −1 | −2 | 0 | 3 | 0 | −1 | −3 | −1 | 0 |
| 27 | E | −1 | −1 | 0 | 1 | −3 | 0 | 5 | −2 | −1 | −3 | −3 | 0 | −2 | −3 | −1 | 1 | 2 | −3 | −2 | −2 |
| 28 | D | 1 | −2 | 0 | 4 | −2 | −1 | 0 | −1 | −2 | −2 | 0 | −1 | −1 | −2 | −1 | 2 | 0 | −4 | −3 | −2 |
| 29 | Y | −2 | −1 | −2 | −2 | −3 | 2 | −1 | −3 | 0 | 0 | −1 | −1 | −1 | 0 | 3 | −1 | −2 | 0 | 5 | 0 |
| 30 | P | 0 | −1 | −1 | −1 | −2 | −1 | −1 | 0 | 5 | −3 | −3 | −1 | −2 | −3 | 4 | 0 | 0 | −3 | −1 | −2 |
| 31 | A | 3 | −2 | −2 | −2 | −1 | −1 | −2 | −1 | −3 | 0 | 2 | −1 | 0 | −2 | 0 | 0 | 0 | −3 | −2 | 0 |
| 32 | D | −2 | −1 | 0 | 5 | −3 | 0 | 2 | −2 | −1 | −2 | −3 | 2 | −2 | −3 | −1 | 0 | −1 | −4 | −3 | 0 |
| 33 | E | 0 | −1 | −2 | 0 | −2 | 0 | 3 | −3 | −1 | 0 | 0 | −1 | 0 | 3 | −2 | −1 | −1 | −2 | 0 | 0 |
| 34 | G | 1 | −2 | 0 | −1 | −2 | −1 | −2 | 4 | −2 | −2 | −2 | −2 | 0 | −2 | −2 | 1 | 1 | −2 | −3 | 0 |
| 35 | D | 0 | 2 | 0 | 2 | −2 | 0 | 0 | −2 | −1 | −2 | 0 | 0 | −1 | −3 | 2 | −1 | −1 | −3 | −2 | −2 |
| 36 | Q | 0 | 3 | 0 | −1 | −2 | 3 | 0 | −2 | −1 | −2 | −2 | 0 | −1 | −3 | −1 | 1 | 3 | −3 | −2 | −2 |
| 37 | I | 0 | −2 | −3 | −2 | −1 | −2 | −2 | −2 | −3 | 1 | 0 | −2 | 0 | −2 | 4 | −1 | −1 | −3 | −2 | 2 |
| 38 | S | −1 | −1 | 0 | 1 | −2 | 0 | 2 | −2 | −1 | 2 | −1 | 1 | −1 | −2 | −2 | 2 | −1 | −3 | −2 | 0 |
| 39 | S | 3 | −1 | −1 | −1 | −1 | −1 | −1 | −1 | −2 | −1 | 0 | −1 | −1 | −2 | 2 | 3 | 0 | −3 | −2 | −1 |
| 40 | N | 0 | −2 | 2 | −1 | −3 | −1 | 1 | 3 | −1 | 0 | 0 | −1 | −1 | −3 | 1 | −1 | −1 | −3 | −2 | 0 |
| 41 | D | 1 | −2 | −1 | 3 | −2 | 0 | 2 | −2 | −2 | −1 | 0 | −1 | −1 | 0 | −2 | 0 | −1 | −3 | −2 | −1 |
| 42 | N | −1 | −1 | 2 | 1 | −3 | 0 | 3 | 0 | −1 | −2 | 0 | 0 | −1 | −2 | −2 | 1 | −1 | −3 | −2 | −2 |
| 43 | L | 1 | −3 | −3 | −3 | 4 | −2 | −3 | −3 | −1 | 0 | 1 | −3 | 0 | 3 | −3 | −2 | −2 | 0 | 5 | 0 |
| 44 | I | 1 | −1 | −1 | 0 | −2 | 0 | 2 | −2 | −2 | 1 | −1 | 1 | −1 | −2 | −2 | 2 | −1 | −4 | −2 | 0 |
| 45 | F | −2 | −3 | −2 | 1 | −2 | −2 | −2 | −3 | −1 | 2 | 0 | −3 | 0 | 4 | −3 | −2 | −2 | −1 | 4 | 1 |
| 46 | D | 1 | −2 | −1 | 2 | −2 | −1 | 0 | 0 | −2 | −3 | −3 | −1 | −2 | −3 | 4 | 1 | −1 | −4 | −3 | −2 |
| 47 | D | 0 | −2 | 0 | 5 | −3 | −1 | 0 | 1 | −2 | −3 | −4 | −1 | −3 | −3 | 2 | 1 | −1 | −4 | −3 | −3 |
| 48 | Y | −2 | 3 | −1 | −2 | −3 | 2 | −1 | −3 | 0 | −2 | −2 | 0 | −1 | 0 | −3 | −2 | −2 | 0 | 6 | −2 |
| 49 | R | −2 | 4 | 0 | −2 | −4 | 2 | 0 | −2 | 5 | −4 | −3 | 0 | −2 | −3 | 2 | −1 | −2 | −3 | −1 | −3 |
| 50 | G | −1 | −2 | 0 | 2 | −3 | −1 | 1 | 4 | −2 | −2 | −3 | −1 | −2 | −3 | −2 | 1 | −1 | −3 | −3 | 0 |
| 51 | K | −1 | 2 | 1 | −1 | −3 | 0 | 0 | 1 | −1 | −3 | −3 | 4 | −2 | −3 | −2 | 1 | −1 | −3 | −3 | −3 |
| 52 | G | −1 | −2 | −1 | 2 | 4 | −2 | −1 | 3 | −2 | −2 | −3 | −2 | −2 | 0 | −2 | 0 | 2 | −2 | −2 | −2 |
| 53 | C | −1 | −4 | −4 | −4 | 10 | −4 | −4 | −4 | −3 | −1 | −1 | −4 | −1 | 2 | −4 | −2 | −2 | −2 | −1 | −1 |
| 54 | V | −1 | −2 | −1 | 0 | −2 | −1 | 1 | −2 | −2 | 0 | 0 | −1 | 3 | −2 | −2 | 1 | 0 | −3 | −2 | 3 |
| 55 | D | 0 | −2 | 0 | 6 | −3 | 0 | 1 | −1 | −1 | −3 | −4 | −1 | −3 | −3 | −1 | 0 | −1 | −4 | −3 | −3 |
| 56 | D | −2 | −1 | 3 | 4 | −4 | 0 | 2 | 2 | −1 | −4 | −4 | −1 | −3 | −4 | −2 | 0 | −1 | −4 | −3 | −3 |
| 57 | S | 0 | −1 | 2 | 2 | −3 | 1 | 0 | 2 | −1 | −3 | −3 | −1 | −2 | −3 | −2 | 3 | 0 | −3 | −3 | −3 |
| 58 | G | 0 | 1 | 0 | −1 | −3 | −1 | −2 | 5 | −2 | −4 | −4 | −1 | −3 | −3 | −2 | 1 | −2 | −3 | −3 | −3 |
| 59 | F | −1 | −4 | −4 | −4 | −2 | −3 | −3 | −4 | −2 | 3 | 0 | −3 | 0 | 5 | −4 | −2 | −1 | −1 | 0 | 3 |
| 60 | V | −1 | −3 | −3 | −2 | −2 | −1 | 1 | −3 | −1 | 0 | 0 | −2 | 0 | 3 | −3 | −2 | −1 | −1 | 4 | 3 |
| 61 | Y | −2 | −2 | −2 | −3 | −2 | −2 | −2 | −3 | 0 | −1 | −1 | −2 | −1 | 4 | −3 | 0 | −2 | 0 | 7 | −2 |
| 62 | K | 1 | −1 | −2 | −2 | −2 | −1 | −1 | 0 | −3 | 0 | −1 | 1 | −1 | −2 | 2 | −1 | −1 | −4 | −2 | 3 |
| 63 | L | −1 | −3 | −2 | −3 | −2 | −3 | −3 | 4 | −3 | 4 | 1 | −3 | 0 | −1 | −3 | −1 | −2 | −3 | −2 | 0 |
| 64 | G | −1 | −1 | 0 | 0 | −4 | 0 | 3 | 5 | −1 | −4 | −4 | −1 | −3 | −4 | −2 | 0 | −2 | −3 | −3 | −3 |
| 65 | E | −2 | −1 | −1 | 0 | −3 | 3 | 3 | −3 | −1 | −2 | −1 | 0 | 3 | −1 | −2 | −1 | −2 | 7 | −1 | −2 |
| 66 | R | −2 | 2 | −2 | −3 | −2 | 0 | −1 | −3 | 0 | 1 | −1 | 1 | −1 | 3 | −3 | −2 | −2 | −1 | 4 | 0 |
| 67 | F | −2 | −3 | −3 | −3 | −2 | −3 | −3 | −3 | −1 | −1 | −1 | −3 | −1 | 6 | −4 | −2 | 1 | 6 | 3 | −1 |
| 68 | F | 0 | 1 | −2 | −3 | −2 | −1 | −2 | −2 | −1 | −1 | 1 | −1 | 0 | 2 | −3 | 0 | 0 | −1 | 2 | −1 |
| 69 | P | −2 | −2 | −1 | 2 | −4 | 0 | 2 | −2 | −2 | −4 | −4 | −1 | −3 | −4 | 6 | −1 | −1 | −4 | −3 | −3 |
| 70 | G | 0 | −2 | 0 | −1 | −2 | −1 | −1 | 5 | −2 | −4 | −4 | −1 | −2 | −3 | −2 | 3 | −1 | −3 | −3 | −3 |
| 71 | H | −2 | −2 | 0 | 5 | −3 | −1 | 0 | −2 | 4 | −3 | −4 | −1 | −3 | −3 | 3 | 0 | −1 | −4 | −2 | −3 |
| 72 | S | −1 | −1 | 0 | −1 | −2 | −1 | −1 | −2 | 5 | −3 | −3 | −1 | −2 | −3 | 4 | 3 | 1 | −3 | −1 | −2 |
| 73 | N | 2 | −2 | 2 | −2 | 8 | −2 | −2 | −2 | −2 | −2 | −2 | −2 | −2 | −3 | −3 | 0 | 0 | −3 | −2 | −1 |
| 74 | C | −1 | −2 | −1 | −1 | 8 | −1 | 3 | −3 | −2 | −2 | −2 | −1 | −2 | −3 | −2 | 0 | 2 | −3 | −2 | −1 |
| 75 | P | −1 | 2 | −2 | −2 | −3 | 3 | 0 | −3 | −2 | −2 | 0 | 0 | −1 | −3 | 5 | −1 | −1 | −3 | −2 | −2 |
| 76 | C | 0 | −4 | −4 | −4 | 10 | −4 | −5 | −4 | −4 | −1 | −1 | −4 | −1 | −2 | −4 | −1 | −1 | −2 | −2 | −1 |
| 77 | V | −1 | −2 | −2 | −1 | −2 | −1 | 2 | −3 | −2 | 0 | 2 | −1 | 0 | −1 | −2 | −1 | 0 | −3 | −2 | 3 |
| 78 | C | 0 | −4 | −4 | −4 | 10 | −4 | −5 | −4 | −4 | −1 | −1 | −4 | −1 | −2 | −4 | −1 | −1 | −2 | −2 | −1 |
| 79 | A | 2 | −1 | −1 | −1 | −1 | 1 | −1 | −2 | −2 | −2 | −2 | −1 | −1 | −3 | −1 | 0 | 4 | −3 | −2 | −1 |
| 80 | L | 1 | −1 | −1 | 0 | −2 | 0 | 2 | −2 | −2 | −1 | 0 | −1 | −1 | −2 | −2 | 1 | 1 | −3 | −2 | −1 |
| 81 | D | −2 | −2 | 0 | 5 | −4 | 0 | 4 | −2 | −1 | −3 | −4 | −1 | −3 | −3 | −2 | 0 | 1 | −4 | −3 | −3 |
| 82 | G | 0 | −3 | 0 | −2 | −4 | −3 | −3 | 7 | −3 | −5 | −5 | −3 | −4 | −4 | −3 | 0 | −3 | −3 | −4 | −4 |
| 83 | P | −1 | −2 | −2 | −1 | −3 | −1 | −1 | −2 | −2 | −3 | −3 | −1 | −2 | −4 | 7 | 1 | −1 | −4 | −3 | −3 |
| 84 | V | 0 | −2 | −2 | −3 | −2 | 1 | −1 | −3 | −3 | 1 | 1 | −2 | 0 | −2 | −2 | 0 | −1 | −3 | −2 | 4 |
| 85 | C | 0 | −3 | −3 | −3 | 10 | −3 | −4 | −3 | −3 | −2 | −2 | −3 | −2 | −3 | −3 | 0 | −1 | −3 | −3 | −2 |
| 86 | D | 1 | −2 | −1 | 3 | 4 | −1 | −1 | −2 | −2 | −1 | −2 | −2 | 0 | 0 | −2 | 1 | 0 | −3 | −2 | 0 |
| 87 | Q | −1 | 3 | −1 | −2 | −3 | 4 | 0 | −3 | −1 | −2 | −2 | 2 | −1 | −3 | −2 | −1 | −1 | −3 | −2 | 0 |
| 88 | P | −1 | −2 | −1 | −2 | −2 | −2 | −2 | −3 | −3 | −2 | −2 | −2 | −2 | −4 | 6 | 0 | 4 | −4 | −3 | −1 |
| 89 | E | −2 | 2 | 0 | 2 | −4 | 0 | 5 | −2 | −1 | −4 | −3 | 1 | −2 | −4 | −2 | −1 | −2 | −4 | −3 | −3 |
| 90 | C | 0 | −4 | −4 | −4 | 10 | −4 | −5 | −4 | −4 | −2 | −2 | −4 | −2 | −3 | −4 | −2 | −2 | −3 | −3 | −2 |
| 91 | P | −1 | −2 | −2 | −2 | −3 | −2 | −2 | −3 | −3 | −1 | −2 | −2 | −2 | −3 | 6 | −1 | 2 | −4 | −3 | 0 |
| 92 | K | 0 | 3 | 0 | 0 | −3 | 0 | 3 | −2 | −1 | −3 | −3 | 3 | −2 | −3 | −2 | 0 | −1 | −3 | −2 | −2 |
| 93 | I | −1 | −3 | −3 | −4 | −1 | −2 | −3 | −4 | −3 | 3 | 3 | −2 | 0 | −1 | −3 | −2 | 1 | −3 | −2 | 1 |
| 94 | H | −1 | −1 | 0 | −1 | 4 | −1 | −1 | −2 | 7 | −3 | −3 | −1 | −2 | −2 | 2 | 1 | −1 | −3 | 0 | −3 |
| 95 | P | 0 | −2 | −2 | −1 | −3 | −1 | 1 | −2 | −2 | −3 | −3 | −1 | −2 | −4 | 7 | −1 | −1 | −4 | −3 | −2 |
| 96 | K | 1 | 3 | −1 | −2 | −2 | 0 | 0 | −2 | 0 | −2 | −2 | 2 | −1 | −1 | −2 | 1 | −1 | −2 | 2 | −2 |
| 97 | C | 0 | −3 | −3 | −3 | 10 | −3 | −4 | −3 | −3 | −2 | −2 | −3 | −2 | −3 | 2 | −1 | −1 | −3 | −3 | −2 |
| 98 | T | −1 | −3 | −2 | −3 | −1 | −2 | −3 | −4 | −3 | 4 | 0 | −3 | 1 | −1 | −3 | −1 | 3 | −3 | −2 | 2 |
| 99 | K | −2 | 1 | 0 | −1 | −4 | 0 | 2 | −2 | 7 | −3 | −3 | 2 | −2 | −2 | −2 | −1 | −2 | −3 | 0 | −3 |
| 100 | V | −1 | −3 | −3 | −3 | −1 | −3 | −3 | −4 | −3 | 3 | 0 | −3 | 0 | −1 | −3 | −2 | −1 | −3 | −1 | 5 |
| 101 | E | 1 | −1 | 0 | 3 | −2 | 0 | 2 | −1 | −1 | −3 | −3 | 1 | −2 | −3 | −1 | 2 | 0 | −4 | −3 | −2 |
| 102 | H | −2 | 2 | 1 | −1 | −3 | 0 | −1 | −2 | 7 | −3 | −2 | 0 | −2 | −1 | −2 | −1 | 1 | −2 | 3 | −2 |
| 103 | N | −1 | 0 | 3 | −1 | −2 | 0 | 0 | −1 | 4 | −2 | −2 | 1 | −2 | 0 | −2 | 1 | 0 | −1 | 4 | −2 |
| 104 | G | −1 | −1 | 0 | 2 | −3 | 4 | 2 | 2 | −1 | −4 | −3 | 0 | −2 | −3 | −2 | 0 | −1 | −3 | −2 | −3 |
| 105 | C | 0 | −3 | −3 | −3 | 10 | −3 | −5 | −3 | −3 | −1 | −1 | −3 | −1 | −2 | −3 | −1 | −1 | −2 | −2 | −1 |
| 106 | C | 0 | −3 | −3 | −3 | 10 | −3 | −5 | −3 | −3 | −1 | −1 | −3 | −1 | −2 | −3 | −1 | −1 | −2 | −2 | −1 |
| 107 | P | −1 | −3 | −3 | −2 | −3 | −2 | −2 | −3 | −3 | −1 | −2 | −2 | −2 | −4 | 7 | −2 | −1 | −4 | −3 | 0 |
| 108 | E | −1 | 2 | 0 | 2 | −3 | 4 | 2 | −2 | −1 | −2 | −1 | 0 | −1 | −3 | −2 | −1 | −1 | −3 | −2 | 0 |
| 109 | C | 0 | −3 | −3 | −3 | 10 | −3 | −5 | −3 | −3 | −1 | −1 | −3 | −1 | −2 | −3 | −1 | −1 | −2 | −2 | −1 |
| 110 | K | −1 | 0 | 0 | 0 | −3 | 0 | 3 | −2 | −1 | −1 | −2 | 3 | −1 | −3 | −2 | 1 | −1 | −3 | −2 | 1 |
| 111 | E | 2 | 2 | −1 | 0 | −3 | 0 | 4 | −2 | −1 | −3 | −2 | 2 | −2 | −3 | −2 | 0 | −1 | −3 | −2 | −2 |
| 112 | V | −1 | 1 | −1 | −1 | −3 | −1 | 1 | 1 | −2 | 1 | −1 | 1 | −1 | −2 | −2 | −1 | −1 | −3 | −2 | 2 |
| 113 | K | 0 | 0 | 0 | −1 | −3 | 0 | 0 | 4 | −2 | −4 | −3 | 4 | −2 | −3 | −2 | 1 | −1 | −3 | −3 | −3 |
| 114 | N | −2 | 0 | 6 | 0 | −3 | 0 | 0 | 0 | 0 | −3 | −3 | 2 | −2 | −3 | −2 | 0 | 0 | −4 | −2 | −3 |
| 115 | F | −1 | −3 | −3 | −3 | −2 | −2 | −2 | −3 | 0 | 0 | 0 | −2 | 0 | 4 | −3 | −2 | −1 | 0 | 6 | 1 |
| 116 | C | 0 | −3 | −3 | −3 | 10 | −3 | −5 | −3 | −3 | −1 | −1 | −3 | −1 | −2 | −3 | −1 | −1 | −2 | −2 | −1 |
| 117 | E | −1 | −1 | 0 | 3 | −3 | 0 | 4 | −2 | −1 | −2 | 0 | 0 | −2 | −3 | −2 | 0 | 1 | −3 | −3 | −2 |
| 118 | Y | −2 | −2 | −2 | −2 | −3 | −1 | 1 | −3 | 0 | −1 | −1 | −2 | −1 | 5 | −3 | −2 | −2 | 0 | 6 | −2 |
| 119 | H | −2 | 5 | 2 | −2 | −3 | 0 | −1 | −2 | 4 | −2 | −1 | 0 | 3 | −2 | −2 | −1 | −1 | −3 | −1 | −2 |
| 120 | G | −1 | −2 | 2 | −1 | −3 | −2 | −2 | 6 | −2 | −4 | −4 | −2 | −3 | −3 | −2 | 0 | −2 | −3 | −3 | −3 |
| 121 | K | −1 | 4 | −1 | −2 | −3 | 0 | 0 | −3 | −1 | −1 | −2 | 5 | −1 | −3 | −2 | −1 | −1 | −3 | −2 | 0 |
| 122 | N | −1 | −2 | 2 | −2 | −2 | −2 | −2 | −2 | −2 | 2 | 0 | −2 | 0 | −2 | −2 | 0 | 4 | −3 | −2 | 1 |
| 123 | Y | −2 | −3 | −3 | −3 | −2 | −2 | −3 | −3 | 0 | −1 | −1 | −3 | −1 | 4 | −4 | −2 | −2 | 1 | 8 | −1 |
| 124 | K | 0 | 1 | 0 | −1 | −3 | 2 | 2 | −2 | 4 | −3 | −2 | 2 | −1 | −1 | −2 | −1 | −2 | −2 | 3 | −2 |
| 125 | I | −1 | −2 | 2 | −2 | −2 | −2 | −2 | −2 | −2 | 2 | 1 | −2 | 0 | −1 | −2 | 0 | 1 | −3 | −2 | 2 |
| 126 | L | −1 | −2 | 2 | −2 | −2 | −2 | −2 | 3 | −2 | 0 | 3 | −2 | 0 | −1 | −3 | −1 | −1 | −3 | −2 | −1 |
| 127 | E | −1 | 0 | 0 | 1 | −4 | 3 | 6 | −2 | 0 | −3 | −3 | 0 | −2 | −3 | −1 | 0 | −1 | −3 | −2 | −2 |
| 128 | E | −1 | −1 | 2 | 0 | −3 | 0 | 4 | −2 | 0 | −3 | −3 | 0 | −2 | −1 | −2 | 0 | 1 | −2 | 3 | −2 |
| 129 | F | −2 | −3 | −3 | −3 | −2 | −3 | −3 | −3 | −1 | 0 | 0 | −3 | 0 | 7 | −5 | −2 | −2 | 0 | 2 | −1 |
| 130 | K | −1 | 1 | 2 | −1 | −3 | 2 | 0 | −2 | −1 | −1 | 0 | 2 | 1 | −2 | −2 | −1 | −1 | −3 | −2 | 0 |
| 131 | P | −1 | −3 | −3 | −3 | −2 | −2 | −2 | −3 | −3 | 1 | 2 | −2 | 0 | −2 | 3 | −2 | 0 | −3 | −2 | 3 |
| 132 | S | 0 | 0 | 1 | 2 | 2 | −1 | 0 | −1 | −1 | −3 | −3 | −1 | −2 | −3 | 1 | 4 | 1 | −4 | −3 | −2 |
| 133 | P | −1 | −2 | −2 | −1 | −3 | −1 | 1 | −3 | −2 | −2 | −3 | −1 | −2 | −4 | 7 | −1 | −1 | −4 | −3 | 0 |
| 134 | C | 0 | −3 | −3 | −3 | 10 | −3 | −4 | −3 | −3 | −1 | −1 | −3 | −1 | −2 | −3 | −1 | 0 | −2 | −2 | −1 |
| 135 | E | −1 | 1 | −1 | 0 | −3 | 0 | 5 | −3 | −1 | −1 | 1 | 0 | −1 | −2 | −2 | −1 | −1 | −3 | −2 | −1 |
| 136 | W | −1 | 2 | −1 | −2 | −3 | 2 | 0 | −2 | 3 | −2 | 0 | 1 | −1 | −2 | −2 | 1 | −1 | 4 | −1 | −2 |
| 137 | C | 0 | 0 | −3 | −3 | 10 | −3 | −4 | −3 | −3 | 0 | −1 | −3 | −1 | −2 | −3 | −1 | 0 | −3 | −2 | −1 |
| 138 | R | −1 | 4 | −1 | −2 | −2 | 0 | −1 | −3 | −1 | 2 | −1 | 2 | −1 | −1 | −2 | −1 | 1 | −2 | 1 | 0 |
| 139 | C | 0 | −4 | −4 | −4 | 10 | −4 | −5 | −4 | −4 | −1 | −1 | −4 | −1 | −2 | −4 | −1 | −1 | −2 | −2 | −1 |
| 140 | E | −1 | −1 | 0 | 2 | −3 | 0 | 5 | −3 | −1 | −2 | 0 | 0 | −1 | −3 | −2 | 0 | 1 | −3 | −2 | −2 |
| 141 | P | 1 | −2 | 1 | −2 | −2 | −1 | −1 | −2 | −2 | 0 | 0 | −1 | −1 | −2 | 2 | 1 | 1 | −3 | −2 | 0 |
| 142 | S | −1 | −1 | 4 | 2 | −3 | 1 | 0 | 2 | −1 | −3 | −3 | −1 | −2 | −3 | −2 | 2 | −1 | −4 | −3 | −3 |
| 143 | N | −1 | 1 | 2 | −1 | −3 | −1 | −1 | 4 | −1 | −4 | −4 | 1 | −2 | −3 | −2 | 1 | −1 | −3 | −3 | −3 |
| 144 | E | −1 | 0 | −1 | 0 | −3 | 0 | 4 | −3 | −1 | 1 | −1 | 0 | −1 | −2 | −2 | −1 | 1 | −3 | −2 | 1 |
| 145 | V | 3 | −2 | −3 | −3 | −1 | −2 | −2 | −2 | −3 | 0 | 0 | −2 | 0 | −2 | −2 | −1 | −1 | −3 | −2 | 3 |
| 146 | H | −2 | 1 | −2 | −3 | −2 | −1 | −2 | −3 | 3 | 0 | 2 | −1 | 0 | 2 | −3 | −2 | −2 | −1 | 4 | −1 |
| 147 | C | −1 | 0 | −3 | −3 | 10 | −3 | −3 | −3 | −3 | −2 | −2 | −2 | −2 | −3 | 1 | −1 | −1 | −3 | −3 | −2 |
| 148 | V | 0 | −2 | −1 | −2 | −1 | −1 | −2 | −2 | −3 | 0 | 1 | −1 | 0 | −2 | −2 | 2 | 3 | −3 | −2 | 2 |
| 149 | V | −1 | −3 | −3 | −3 | 1 | −2 | −2 | −3 | −3 | 2 | 0 | −2 | 0 | −2 | 2 | −2 | −1 | −4 | −2 | 5 |
| 150 | A | 3 | −2 | −2 | −2 | 2 | −1 | −1 | −1 | −2 | 0 | −1 | −1 | −1 | −2 | −2 | 2 | 0 | −3 | −2 | 2 |
| 151 | D | 1 | −2 | 0 | 5 | −2 | 1 | 0 | 0 | −2 | −3 | −3 | −1 | −2 | −3 | −2 | 1 | −1 | −4 | −3 | −2 |
| 152 | C | −1 | −3 | −3 | −3 | 10 | −3 | −4 | 0 | −3 | −2 | −2 | −3 | −1 | 1 | −4 | −1 | −2 | −2 | −1 | −2 |
| 153 | A | 2 | −2 | −2 | −2 | −2 | −1 | 0 | −2 | −2 | −1 | 0 | −1 | −1 | 1 | 4 | −1 | −1 | −3 | −1 | −1 |
| 154 | V | 2 | −1 | −2 | −1 | −2 | 4 | 1 | −2 | −2 | −1 | −2 | 0 | −1 | −3 | 3 | −1 | −1 | −3 | −2 | 0 |
| 155 | P | −1 | −2 | −1 | 1 | −2 | −2 | −1 | −2 | −2 | 1 | 0 | −2 | −1 | −2 | 4 | 0 | 2 | −4 | −3 | 0 |
| 156 | E | −2 | 1 | −1 | 0 | −4 | 0 | 3 | −3 | 2 | −3 | −3 | 1 | −2 | 2 | 3 | −1 | −2 | −3 | −1 | −3 |
| 157 | C | −1 | −3 | −3 | −3 | 9 | 1 | −3 | −3 | −3 | −2 | −1 | −3 | −1 | −2 | −3 | −2 | −2 | 5 | −2 | −2 |
| 158 | V | −1 | 0 | −2 | −3 | −2 | 0 | −2 | −3 | −3 | 2 | 0 | −2 | 0 | 1 | −2 | −2 | 1 | −2 | −1 | 4 |
| 159 | N | −2 | 1 | 4 | 4 | −3 | 0 | 0 | −1 | 0 | −3 | −3 | −1 | −2 | −1 | −2 | 0 | −1 | 3 | 3 | −3 |
| 160 | P | −1 | −2 | −2 | −1 | −3 | −1 | −1 | −2 | −2 | −1 | −3 | 1 | −2 | −4 | 7 | 0 | −1 | −4 | −3 | −2 |
| 161 | V | −1 | −3 | −3 | −3 | −2 | 0 | 0 | −4 | −3 | 4 | 0 | −2 | 0 | 2 | −3 | −2 | −1 | −2 | 0 | 3 |
| 162 | Y | −2 | 1 | −1 | −3 | −3 | −1 | −1 | −3 | 4 | −2 | 0 | −1 | −1 | 0 | −3 | 0 | −2 | 0 | 6 | −2 |
| 163 | E | −1 | −1 | −1 | 0 | −3 | 3 | 4 | −3 | −1 | −2 | −1 | 0 | −1 | −3 | 3 | 0 | 0 | −3 | −2 | −2 |
| 164 | P | −1 | −1 | −2 | −1 | −3 | 0 | 1 | −3 | −1 | −2 | −1 | 0 | −2 | −2 | 5 | −1 | −1 | −2 | 2 | −2 |
| 165 | E | −1 | −2 | 1 | 2 | −3 | −1 | 1 | 3 | −1 | −3 | −3 | −1 | −3 | −1 | −2 | 0 | −1 | −2 | 2 | −3 |
| 166 | Q | −2 | 0 | −1 | −1 | −3 | 4 | 2 | −3 | 3 | −3 | 0 | 2 | −1 | −2 | −2 | −1 | −2 | 5 | 0 | −2 |
| 167 | C | 0 | −3 | −3 | −4 | 9 | −3 | −4 | −4 | −3 | 0 | 0 | −3 | −1 | −2 | −3 | −2 | −1 | −2 | −2 | 0 |
| 168 | C | 0 | −3 | −3 | −3 | 9 | −3 | −3 | −3 | −3 | 0 | −1 | −3 | −1 | −2 | −3 | 0 | −1 | −3 | −2 | 1 |
| 169 | P | 0 | −2 | −3 | −2 | −3 | −1 | −2 | −2 | −3 | −2 | 0 | −1 | −1 | −3 | 7 | −1 | −1 | −4 | −3 | −1 |
| 170 | V | −1 | −2 | −2 | −2 | −2 | −1 | 1 | −3 | −2 | 3 | 0 | 1 | 0 | −2 | −2 | −2 | −1 | −3 | −2 | 4 |
| 171 | C | −1 | −2 | −3 | −3 | 9 | −2 | −3 | −3 | −3 | −1 | 0 | 1 | −1 | −2 | −3 | −1 | −1 | −3 | −2 | −1 |
| 172 | K | −1 | 1 | 0 | −1 | −3 | 0 | 0 | −2 | −1 | −3 | −2 | 6 | −1 | −3 | −1 | 0 | −1 | −3 | −2 | −2 |
| 173 | N | 2 | −1 | 3 | 2 | −2 | −1 | 0 | 2 | −1 | −3 | −3 | −1 | −2 | −3 | −2 | 1 | −1 | −4 | −3 | −2 |
| 174 | G | 0 | −1 | 0 | −1 | −3 | −2 | −2 | 5 | −2 | −4 | −3 | 0 | −3 | −3 | −2 | 0 | −2 | −2 | −3 | −3 |
| 175 | P | −1 | −2 | 1 | −1 | −3 | −1 | 0 | −2 | −2 | −3 | −3 | −1 | −2 | −4 | 6 | −1 | −1 | −4 | −3 | −2 |
| 176 | N | −1 | −1 | 5 | 0 | −3 | −1 | −1 | −1 | 0 | −3 | −3 | −1 | −2 | −2 | −2 | 1 | 0 | 6 | −1 | −3 |
| 177 | C | −1 | −2 | −2 | −2 | 8 | −2 | −2 | −3 | 3 | −2 | −2 | −2 | −2 | −2 | 1 | −1 | 0 | −3 | −1 | −2 |
| 178 | F | −2 | −2 | −1 | −2 | −3 | −2 | −2 | 1 | 4 | −2 | −2 | 0 | −1 | 4 | 0 | −1 | −2 | 0 | 3 | −2 |
| 179 | A | 2 | −2 | −2 | −3 | −1 | −2 | −2 | 2 | −3 | 0 | 1 | −2 | 0 | 0 | −2 | −1 | −1 | −3 | −1 | 1 |
| 180 | G | 0 | −1 | 0 | 0 | −2 | −1 | 1 | 3 | −2 | −2 | 0 | −1 | −1 | −2 | 1 | 1 | 1 | −3 | −2 | −1 |
| 181 | T | 0 | −2 | −1 | −1 | −1 | −1 | −1 | −2 | −2 | 1 | −1 | −1 | −1 | −2 | 1 | 0 | 5 | −3 | −2 | 0 |
| 182 | T | 1 | −1 | 0 | −1 | −1 | 2 | 0 | −1 | −2 | −1 | 0 | −1 | 0 | −2 | −1 | 1 | 3 | −3 | −2 | −1 |
| 183 | I | 1 | −3 | −2 | −3 | −1 | −2 | −2 | −2 | −3 | 3 | 0 | −2 | 0 | −1 | −2 | 0 | 0 | −3 | −1 | 3 |
| 184 | I | −1 | −3 | −3 | −3 | −2 | −3 | −3 | 1 | −3 | 5 | 0 | −3 | 0 | 2 | −3 | −2 | −1 | −2 | 0 | 1 |
| 185 | P | −1 | −2 | −2 | −1 | −3 | 0 | 1 | −2 | −2 | −2 | 0 | −1 | −1 | −3 | 6 | −1 | −1 | −4 | −3 | −2 |
| 186 | A | 4 | 1 | −2 | −2 | 0 | −1 | −1 | 0 | −2 | −1 | −1 | 0 | −1 | −2 | −1 | 0 | 0 | −3 | −2 | 0 |
| 187 | G | 0 | −2 | 0 | −1 | −3 | −2 | −2 | 6 | −2 | −2 | −3 | −2 | −2 | −3 | −2 | 0 | −2 | −2 | −3 | 0 |
| 188 | I | −1 | 2 | −1 | 0 | −3 | 0 | 3 | −3 | −1 | 2 | −1 | 0 | −1 | −2 | 1 | −1 | −1 | −3 | −2 | 0 |
| 189 | E | −1 | −1 | −1 | 0 | −3 | 0 | 4 | −2 | −1 | 0 | −2 | 0 | −1 | −3 | 3 | −1 | −1 | −3 | −2 | −1 |
| 190 | V | 0 | 0 | −1 | 1 | −2 | −1 | −1 | −2 | −2 | 0 | 0 | −1 | 0 | −2 | −2 | −1 | 2 | −3 | −2 | 3 |
| 191 | K | −1 | 0 | −1 | −1 | −3 | 0 | 1 | −2 | −1 | −2 | −2 | 4 | −1 | −2 | −2 | −1 | −1 | 7 | −1 | 0 |
| 192 | V | 0 | −1 | −1 | −2 | −2 | 1 | 0 | −3 | −2 | 0 | 0 | 1 | 0 | −2 | −2 | −1 | 2 | −3 | −1 | 3 |
| 193 | D | −2 | −2 | 0 | 6 | −3 | 0 | 1 | −1 | −1 | −3 | −4 | −1 | −3 | −3 | −1 | 0 | 1 | −4 | −3 | −3 |
| 194 | E | 1 | −1 | −1 | 1 | −2 | 0 | 4 | −2 | −1 | −1 | −2 | 0 | −1 | −2 | −1 | 0 | −1 | −3 | −2 | 1 |
| 195 | C | 0 | −3 | −3 | −3 | 10 | −3 | −4 | −3 | −3 | −1 | −1 | −3 | −1 | −2 | −3 | −1 | −1 | −2 | −2 | −1 |
| 196 | N | −1 | −1 | 3 | −1 | −2 | −1 | −1 | −1 | −1 | −1 | −1 | −1 | −1 | 2 | −2 | 0 | 4 | −2 | 0 | −1 |
| 197 | I | −1 | −1 | −2 | −3 | 5 | −2 | −2 | −3 | −3 | 4 | 0 | 1 | 0 | −1 | −3 | −1 | −1 | −3 | −1 | 1 |
| 198 | C | 0 | −3 | −2 | −3 | 9 | −3 | −3 | −3 | −3 | −1 | −1 | −3 | −1 | −2 | −3 | −1 | 1 | −2 | −2 | −1 |
| 199 | H | −2 | 2 | 0 | −2 | −3 | 0 | 0 | −2 | 7 | −3 | −2 | 0 | −2 | 0 | −2 | −1 | −2 | −1 | 3 | −3 |
| 200 | C | 0 | −3 | −3 | −3 | 10 | −3 | −4 | −3 | −3 | −1 | −1 | −3 | −1 | −2 | −3 | −1 | −1 | −2 | −2 | −1 |
| 201 | H | −1 | 0 | 0 | −1 | −2 | 0 | 0 | −2 | 7 | −2 | −2 | −1 | −2 | −1 | −2 | 0 | 3 | −2 | 0 | −2 |
| 202 | N | −2 | −1 | 4 | 3 | −3 | 0 | 0 | −1 | 0 | −2 | −2 | −1 | −2 | 0 | −2 | 0 | −1 | −1 | 4 | −2 |
| 203 | G | 0 | −1 | 0 | 0 | −3 | 0 | 3 | 5 | −1 | −4 | −4 | −1 | −3 | −3 | −2 | 0 | −2 | −2 | −3 | −3 |
| 204 | D | −1 | 0 | 0 | 4 | −3 | 3 | 3 | −2 | 0 | −3 | −3 | 0 | −2 | −3 | −1 | 0 | −1 | −3 | −2 | −2 |
| 205 | W | −1 | −2 | 0 | 3 | −3 | −1 | 0 | 3 | −2 | −3 | −3 | −2 | −2 | −1 | −2 | −1 | −2 | 8 | 0 | −3 |
| 206 | W | −2 | −3 | −2 | −3 | −2 | −2 | −3 | 3 | −2 | −3 | −3 | −3 | −1 | 0 | −3 | −2 | −2 | 11 | 0 | −3 |
| 207 | K | −1 | 3 | 0 | −2 | −3 | 0 | 0 | −2 | 0 | −2 | −2 | 3 | −1 | 0 | −2 | −1 | −1 | −1 | 4 | −2 |
| 208 | P | −1 | −1 | −1 | −1 | −2 | 3 | 0 | −3 | −1 | −1 | 1 | 0 | 0 | −2 | 4 | −1 | −1 | −3 | −2 | −1 |
| 209 | A | 5 | −1 | −2 | −2 | 0 | −1 | −1 | 0 | −2 | −1 | −1 | −1 | −1 | −2 | −1 | 0 | 0 | −3 | −2 | 0 |
| 210 | Q | −1 | 0 | −1 | −1 | −2 | 3 | 0 | −2 | −1 | −1 | 0 | 0 | 4 | −1 | −1 | 0 | 2 | −2 | −1 | 0 |
| 211 | C | 0 | −3 | −3 | −3 | 10 | −3 | −4 | −3 | −3 | −1 | −1 | −3 | −1 | −2 | −3 | −1 | −1 | −2 | −2 | −1 |
| 212 | S | 0 | −1 | 0 | 0 | −1 | 0 | 0 | −1 | −1 | −2 | −2 | 0 | −1 | −2 | −1 | 4 | 3 | −3 | −2 | −1 |
| 213 | K | −1 | 5 | 0 | −2 | −3 | 0 | 0 | −2 | 0 | −3 | −2 | 4 | −1 | −3 | −2 | −1 | −1 | −3 | −2 | −3 |
| 214 | R | −1 | 3 | 0 | −2 | −2 | 0 | −1 | −3 | 6 | −1 | 1 | 0 | 0 | −1 | −2 | −1 | −1 | −2 | 0 | −1 |
| 215 | E | −1 | 0 | 0 | 1 | −4 | 1 | 5 | −2 | 0 | −3 | −3 | 3 | −2 | −3 | −1 | 0 | −1 | −3 | −2 | −2 |
| 216 | C | 0 | −2 | 3 | −1 | 9 | −2 | −2 | −2 | −1 | −1 | −1 | −2 | −1 | −2 | −3 | 0 | −1 | −3 | −2 | −1 |
| 217 | Q | −1 | 2 | −1 | −1 | 6 | 3 | 0 | −2 | −1 | −2 | −2 | 1 | −1 | −3 | −2 | 0 | −1 | −2 | −2 | −2 |
| 218 | G | −1 | 0 | 3 | 0 | −3 | 3 | 0 | 3 | 0 | −3 | −3 | 0 | −1 | −3 | −2 | 0 | −1 | −3 | −2 | −3 |
| 219 | K | −1 | 0 | 0 | −2 | −2 | 0 | 0 | −2 | 5 | 0 | 0 | 3 | 3 | −1 | −2 | −1 | −1 | −2 | 0 | 0 |
| 220 | Q | −1 | 0 | 0 | 0 | −3 | 4 | 4 | −2 | 0 | −3 | −2 | 0 | −1 | −3 | −1 | 0 | −1 | −2 | −1 | −2 |
| 221 | T | 0 | −1 | 0 | −1 | −2 | −1 | −1 | 4 | −2 | −2 | −2 | −1 | −2 | −2 | −1 | 0 | 4 | −2 | −2 | −1 |
| 222 | V | 0 | −2 | −2 | −3 | −1 | −1 | −2 | −3 | −2 | 2 | 1 | −1 | 4 | 0 | −2 | −1 | 0 | −2 | −1 | 3; or |

b) comprises a fragment of the amino acid sequence of a), wherein said fragment is a member of the vWFC domain containing protein family, or has an antigenic determinant in common with the amino acid sequence of a); or
c) comprises a functional equivalent of the amino acid sequence of a) or b); or
d) consists of the amino acid sequence of a); or
e) is any of a)-d), wherein the polypeptide has a maximum threshold E value of 10⁻²; or
f) comprises an amino acid sequence satisfying the consensus amino acid sequence [GTDFC](0,1)-[CF](0,1)-[VMSED](0,1)-[DEA](0,1)-[DENG](0,1)-[SQNDG](0,1)-[SGR](0,1)-[FIV](0,1)-[VYFE](0,1)-[YFS](0,1)-[KVAGP](0,1)-[LIG](0,1)-[GE](0,1)-[EWQM](0,1)-[RKYFQVI](0,1)-[FYWT](0,1)-[FALYRTS](0,1)-[PED](0,1)-[GS](0,1)-[HPDS](0,1)-[STHP](0,1)-[CNAT](0,1)-[CTE](0,1)-[PQRL](0,1)-C(0,1)-[VELT](0,1)-C(0,1)-[TAQ](0,1)-[ELATSD](0,1)-[EDT]-G-[PS]-[VLAQS]-[CS]-[DAMSTFCV]-[QRKV]-R(0,1)-[PT]-[ERDK]-C-[PTV]-[KERSA]-[LIVT]-[HPSC]-[PAE]-[KRASY]-[CP]-[TIVM]-[HKRE-]-[DESAKP]-[HTNRYG]-[NSTYHK](0,1)-[PA](0,1)-[TG](0,1)-[QGDES]-C-C-[PV]-[EQRDLV]-C; or
g) comprises a fragment of the amino acid sequence of f), wherein said fragment is a member of the vWFC domain containing protein family, or has an antigenic determinant in common with the polypeptides of f); or
h) comprises a functional equivalent of f) or g); or
i) consists of an amino acid sequence satisfying the consensus amino acid sequence[GTDFC](0,1)-[CF](0,1)-[VMSED](0,1)-[DEA](0,1)-[DENG](0,1)-[SQNDG](0,1)-[SGR](0,1)-[FIV](0,1)-[VYFE](0,1)-[YFS](0,1)-[KVAGP](0,1)-[LIG](0,1)-[GE](0,1)-[EWQM](0,1)-[RKYFQVI](0,1)-[FYWT](0,1)-[FALYRTS](0,1)-[PED](0,1)-[GS](0,1)-[HPDS](0,1)-[STHP](0,1)-[CNAT](0,1)-[CTE](0,1)-[PQRL](0,1)-C(0,1)-[VELT](0,1)-C(0,1)-[TAQ](0,1)-[ELATSD](0,1)-[EDT]-G-[PS]-[VLAQS]-[CS]-[DAMSTFCV]-[QRKV]-R(0,1)-[PT]-[ERDK]-C-[PTV]-[KERSA]-[LIVT]-[HPSC]-[PAE]-[KRASY]-[CP]-[TIVM]-[HKRE]-[VI]-[DESAKP]-[HTNRYG]-[NSTYHK](0,1)-[PA](0,1)-[TG](0,1)-[QGDES]-C-C-[PV]-[EQRDLV]-C; or
j) comprises an amino acid sequence selected from the group consisting of SEQ ID NO:2, SEQ ID NO:4, SEQ ID NO:39, SEQ ID NO:41, SEQ ID NO:43, and SEQ ID NO:45; or
k) comprises a fragment of the amino acid sequence of j), wherein said fragment is a member of the vWFC domain containing protein family, or has an antigenic determinant in common with the amino acid sequence of j); or
l) comprises a functional equivalent of j) or k); or
m) consists of an amino acid sequence selected from the group consisting of SEQ ID NO:2, SEQ ID NO:4, SEQ ID NO:39, SEQ ID NO:41, SEQ ID NO:43, and SEQ ID NO:45; or
n) comprises a fragment of the amino acid sequence of m), wherein said fragment is a member of the vWFC domain containing protein family, or has an antigenic determinant in common with the amino acid sequence of m); or
o) comprising a functional equivalent of m) or n); or
p) comprises an amino acid sequence selected from the group consisting of SEQ ID NO:6, SEQ ID NO:8, SEQ ID NO:10, SEQ ID NO:12, SEQ ID NO:14, SEQ ID NO:16, SEQ ID NO:47, SEQ ID NO:49, SEQ ID NO:51, and SEQ ID NO:53; or
q) comprises a fragment of the amino acid sequence of p), wherein said fragment is a member of the vWFC domain containing protein family, or has an antigenic determinant in common with the amino acid sequence of p); or
r) comprises a functional equivalent of p) or q); or
s) consists of an amino acid sequence selected from the group consisting of SEQ ID NO:6, SEQ ID NO:8, SEQ ID NO:10, SEQ ID NO:12, SEQ ID NO:14, SEQ ID NO:16, SEQ ID NO:47, SEQ ID NO:49, SEQ ID NO:51, and SEQ ID NO:53; or
t) comprises a fragment of the amino acid sequence of s), wherein said fragment is a member of the vWFC domain containing protein family, or has an antigenic determinant in common with the amino acid sequence of s); or
u) comprises a functional equivalent of s) or t); or
v) comprises the amino acid sequence selected from the group consisting of SEQ ID NO:18, SEQ ID NO:20, SEQ ID NO:22, SEQ ID NO:24, SEQ ID NO:26, SEQ ID NO:28, SEQ ID NO:30, SEQ ID NO:55, SEQ ID NO:57, SEQ ID NO:59, and SEQ ID NO:61; or
w) comprises a fragment of the amino acid sequence of w), wherein said fragment is a member of the vWFC domain containing protein family, or has an antigenic determinant in common with the amino acid sequence of w); or
x) comprises a functional equivalent of v) or w); or
y) consists of an amino acid sequence selected from the group consisting of SEQ ID NO:18, SEQ ID NO:20, SEQ ID NO:22, SEQ ID NO:24, SEQ ID NO:26, SEQ ID NO:28, SEQ ID NO:30, SEQ ID NO:55, SEQ ID NO:57, SEQ ID NO:59, and SEQ ID NO:61; or
z) comprises a fragment of the amino acid sequence of y), wherein said fragment is a member of the vWFC domain containing protein family, or has an antigenic determinant in common with the amino acid sequence of y); or
aa) comprises a functional equivalent of y) or z); or
bb) comprises the functional equivalent of any of c), e), h), l), o), r), u), x), or aa), characterised in that it is homologous to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, and SEQ ID NO:61, and is a member of the vWFC domain containing protein family; or
cc) comprises the fragment or functional equivalent of any of b), c), d), g), h), k), l), n), o), q), r), t), u), w), x), z), aa), or bb), wherein the fragment or functional equivalent has greater than 80% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, and SEQ ID NO:49, or with an active fragment of any of the foregoing; or
dd) comprises the functional equivalent of any of c), e), h), l), o), r), u), x), aa), bb, or cc), wherein the functional equivalent exhibits significant structural homology with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, and SEQ ID NO:61; or
ee) comprises the fragment of any of b), e), g), k), n), q), t), w), z), or cc), wherein the fragment has an antigenic determinant in common with the amino acid sequence of any one of a), d), e,), f), i), j), m), p), s), v), or y), which consists of 7 or more amino acid residues from the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59 or SEQ ID NO:61.

69: The method of claim 68, which is carried out in vitro.

70: The method of claim 68, comprising:
a) contacting a ligand with a biological sample under conditions suitable for the formation of a ligand-polypeptide complex; and
b) detecting said complex, wherein the ligand binds specifically to the polypeptide of any of a) to cc) of claim 68, or wherein the ligand is an antibody that binds specifically to the polypeptide of any of a) to ee) of claim 68.

71: The method of claim 68, comprising:
a) contacting a sample of tissue from the patient with a nucleic acid probe under stringent conditions that allow the formation of a hybrid complex between a nucleic acid molecule and the probe;
b) contacting a control sample with said probe under the same conditions used in step a); and
c) detecting the presence of hybrid complexes in said samples; wherein detection of levels of the hybrid complex in the patient sample that differ from levels of the hybrid complex in the control sample is indicative of disease, wherein the nucleic acid molecule:
1) comprises a nucleic acid sequence encoding a polypeptide according to any one of a)-ee) of claim 68; or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, and SEQ ID NO:60, or a redundant equivalent or fragment of any of the foregoing; or
3) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27 SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, and SEQ ID NO:60, or a redundant equivalent or fragment of any of the foregoing; or
4) hybridizes under high stringency conditions with a nucleic acid molecule according to any one of c1) to c3).

72: The method of claim 68, comprising:
a) contacting a sample of nucleic acid from tissue of the patient with a nucleic acid primer under stringent conditions that allow the formation of a hybrid complex between a nucleic acid molecule and the primer;
b) contacting a control sample with said primer under the same conditions used in step a);
c) amplifying the sampled nucleic acid; and
d) detecting the level of amplified nucleic acid from both patient and control samples; wherein detection of levels of the amplified nucleic acid in the patient sample that differ significantly from levels of the amplified nucleic acid in the control sample is indicative of disease, wherein the nucleic acid molecule:
1) comprises a nucleic acid sequence encoding a polypeptide according to any one of a)-ee) of claim 68; or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, and SEQ ID NO:60, or a redundant equivalent or fragment of any of the foregoing; or
3) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27 SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, and SEQ ID NO:60, or a redundant equivalent or fragment of any of the foregoing; or
4) hybridizes under high stringency conditions with a nucleic acid molecule according to any one of d1) to d3).

73: The method of claim 68, comprising:
a) obtaining a tissue sample from a patient being tested for disease;
b) isolating a nucleic acid molecule from said tissue sample; and
c) diagnosing the patient for disease by detecting the presence of a mutation which is associated with disease in the nucleic acid molecule as an indication of the disease, wherein the nucleic acid molecule:
1) comprises a nucleic acid sequence encoding a polypeptide according to any one of a)-ee) of claim 68; or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, and SEQ ID NO:60, or a redundant equivalent or fragment of any of the foregoing; or
3) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27 SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, and SEQ ID NO:60, or a redundant equivalent or fragment of any of the foregoing; or
4) hybridizes under high stringency conditions with a nucleic acid molecule according to any one of c1) to c3).

74: The method of claim 73, further comprising amplifying the nucleic acid molecule to form an amplified product and detecting the presence or absence of a mutation in the amplified product.

75: The method of claim 73, wherein the presence or absence of the mutation in the patient is detected by contacting said nucleic acid molecule with a nucleic acid probe that hybridizes to said nucleic acid molecule under stringent conditions to form a hybrid double-stranded molecule, the hybrid double-stranded molecule having an unhybridized portion of the nucleic acid probe strand at any portion corresponding to a mutation associated with disease; and detecting the presence or absence of an unhybridized portion of the probe strand as an indication of the presence or absence of a disease-associated mutation.

76: The method of claim 68, wherein said disease includes one or more of among cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders such as those relating to cartilage and bone skeletal development, including osteoarthritis; metabolic disorders including diabetes mellitus, osteoporosis, and obesity, AIDS and renal disease; infections including viral infection, bacterial infection, fungal infection and parasitic infection.

77: The method of claim 71, wherein said disease is a disease in which lymphocyte antigens are implicated.

78: The method of claim 62, wherein said method of using a composition of matter comprises the method of monitoring the therapeutic treatment of a disease, comprising monitoring over a period of time the level of expression or activity of a polypeptide, or the level of expression of a nucleic acid molecule, in tissue from said patient, wherein altering said level of expression or activity over the period of time towards a control level is indicative of regression of said disease, wherein
a) the polypeptide:
1) comprises an amino acid sequence that has an E value of 10⁻² or less when the profile below is input as a query sequence into the search program BLAST, using the default parameters specified by the NCBI (the National Center for Biotechnology Information) [Blosum 62 matrix; gap open penalty=11 and gap extension penalty=1]:

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | M | −2 | −2 | −3 | −4 | −2 | −1 | −3 | −4 | −3 | 0 | 2 | −2 | 8 | 0 | −3 | −2 | −2 | −2 | −2 | 0 |
| 2 | A | 3 | −1 | −3 | −3 | −1 | −1 | −2 | −2 | −3 | 0 | 0 | 1 | 2 | 1 | −2 | −1 | −1 | −3 | −1 | 1 |
| 3 | L | 1 | −2 | −2 | −3 | −2 | −2 | −2 | −2 | 2 | 0 | 2 | −2 | 0 | −2 | 0 | 1 | −1 | −3 | −2 | 2 |
| 4 | H | −1 | −1 | 1 | −1 | −3 | −1 | −1 | 3 | 6 | −3 | −1 | −1 | −2 | −2 | −2 | 2 | −1 | −3 | −1 | −3 |
| 5 | I | 0 | −2 | −3 | −3 | −2 | 0 | 0 | −3 | −3 | 3 | 2 | −2 | 0 | −1 | −3 | −2 | −1 | −3 | −2 | 2 |
| 6 | H | 0 | −2 | −1 | −2 | −2 | −1 | −1 | −3 | 7 | 0 | 0 | −2 | −1 | −2 | 0 | 0 | −1 | −3 | 0 | 2 |
| 7 | E | 0 | −2 | −2 | −1 | −2 | −1 | 2 | −3 | −1 | 0 | 1 | −1 | 2 | 2 | −3 | 0 | −1 | −2 | 2 | 0 |
| 8 | A | 2 | −2 | −1 | −2 | 3 | −2 | −2 | 2 | −2 | −2 | −2 | −2 | −2 | −3 | −2 | 3 | 1 | −3 | −3 | −2 |
| 9 | C | 0 | −3 | −2 | −3 | 7 | −2 | −3 | −3 | −3 | 0 | −1 | −2 | 3 | 3 | −3 | 0 | −1 | −2 | −1 | 0 |
| 10 | I | −1 | −2 | −2 | −2 | −2 | −2 | 0 | 0 | −2 | 3 | 2 | −2 | 0 | 0 | −3 | 0 | 0 | −2 | 3 | 0 |
| 11 | L | −2 | 0 | −3 | −4 | −2 | −2 | −3 | −4 | −3 | 0 | 4 | −2 | 3 | 3 | −4 | −2 | −2 | −2 | 0 | 0 |
| 12 | L | 0 | 0 | −3 | −4 | −2 | −2 | −3 | −3 | −3 | 0 | 3 | −2 | 0 | −1 | 1 | −2 | −1 | −3 | −2 | 1 |
| 13 | L | −1 | −2 | −2 | −2 | −2 | −2 | 0 | −3 | −3 | 0 | 3 | −2 | 0 | 1 | −3 | −1 | 1 | −3 | −1 | 1 |
| 14 | V | 0 | 0 | −3 | −3 | 4 | 0 | −2 | −3 | −3 | 0 | 0 | −2 | 2 | −2 | −3 | −2 | −1 | −3 | −2 | 4 |
| 15 | I | −1 | 0 | −2 | −3 | 4 | −2 | −2 | −3 | 2 | 2 | 1 | −2 | 0 | −1 | −3 | 0 | 0 | 4 | −1 | 0 |
| 16 | P | 0 | 0 | −2 | −2 | −2 | −2 | −2 | −3 | 4 | −1 | 0 | −2 | −1 | 2 | 3 | 0 | −1 | −2 | 0 | 0 |
| 17 | G | 0 | −3 | −2 | −3 | −2 | −2 | −3 | 2 | −3 | 1 | 0 | −3 | 3 | 3 | −3 | −1 | −2 | −2 | 0 | 1 |
| 18 | L | 1 | −3 | −3 | −4 | −1 | −2 | −3 | −3 | −3 | 0 | 4 | −2 | 0 | −1 | −3 | −2 | −1 | −3 | −2 | 2 |
| 19 | V | 2 | −1 | −1 | −2 | −2 | 3 | −1 | −2 | −2 | 0 | −1 | −1 | −1 | −3 | −2 | 1 | 1 | −3 | −2 | 2 |
| 20 | T | −1 | −2 | −2 | −3 | 4 | −2 | −2 | −3 | −3 | 0 | 3 | −2 | 0 | −2 | −3 | 1 | 3 | −3 | −2 | 0 |
| 21 | S | 0 | −2 | −1 | −2 | 4 | −2 | −2 | 1 | −3 | −1 | 1 | −2 | 2 | −2 | 0 | 2 | −1 | −3 | −3 | −1 |
| 22 | A | 3 | −2 | −2 | −2 | −1 | −2 | −2 | −1 | −2 | −1 | 0 | −2 | −1 | 1 | 2 | 2 | −1 | −3 | −2 | 0 |
| 23 | A | 2 | −2 | −3 | −2 | 5 | 0 | 0 | −2 | −3 | −1 | 0 | −2 | −1 | −3 | 2 | −1 | −1 | −3 | −2 | 1 |
| 24 | I | 1 | 1 | −2 | −2 | −2 | −1 | −2 | −2 | −2 | 2 | 0 | −1 | −1 | −1 | −2 | 2 | −1 | −3 | 1 | 1 |
| 25 | S | −1 | −1 | 2 | −1 | −2 | 1 | −1 | −2 | 4 | 2 | −1 | −1 | −1 | −2 | −2 | 3 | −1 | −4 | −1 | −1 |
| 26 | H | 0 | −2 | 3 | −1 | −3 | −1 | −1 | −2 | 5 | −2 | −2 | −1 | −2 | 0 | 3 | 0 | −1 | −3 | −1 | 0 |
| 27 | E | −1 | −1 | 0 | 1 | −3 | 0 | 5 | −2 | −1 | −3 | −3 | 0 | −2 | −3 | −1 | 1 | 2 | −3 | −2 | −2 |
| 28 | D | 1 | −2 | 0 | 4 | −2 | −1 | 0 | −1 | −2 | −2 | 0 | −1 | −1 | −2 | −1 | 2 | 0 | −4 | −3 | −2 |
| 29 | Y | −2 | −1 | −2 | −2 | −3 | 2 | −1 | −3 | 0 | 0 | −1 | −1 | −1 | 0 | 3 | −1 | −2 | 0 | 5 | 0 |
| 30 | P | 0 | −1 | −1 | −1 | −2 | −1 | −1 | 0 | 5 | −3 | −3 | −1 | −2 | −3 | 4 | 0 | 0 | −3 | −1 | −2 |
| 31 | A | 3 | −2 | −2 | −2 | −1 | −1 | −2 | −1 | −3 | 0 | 2 | −1 | 0 | −2 | 0 | 0 | 0 | −3 | −2 | 0 |
| 32 | D | −2 | −1 | 0 | 5 | −3 | 0 | 2 | −2 | −1 | −2 | −3 | 2 | −2 | −3 | −1 | 0 | −1 | −4 | −3 | 0 |
| 33 | E | 0 | −1 | −2 | 0 | −2 | 0 | 3 | −3 | −1 | 0 | 0 | −1 | 0 | 3 | −2 | −1 | −1 | −2 | 0 | 0 |
| 34 | G | 1 | −2 | 0 | −1 | −2 | −1 | −2 | 4 | −2 | −2 | −2 | −2 | 0 | −2 | −2 | 1 | 1 | −2 | −3 | 0 |
| 35 | D | 0 | 2 | 0 | 2 | −2 | 0 | 0 | −2 | −1 | −2 | 0 | 0 | −1 | −3 | 2 | −1 | −1 | −3 | −2 | −2 |
| 36 | Q | 0 | 3 | 0 | −1 | −2 | 3 | 0 | −2 | −1 | −2 | −2 | 0 | −1 | −3 | −1 | 1 | 3 | −3 | −2 | −2 |
| 37 | I | 0 | −2 | −3 | −2 | −1 | −2 | −2 | −2 | −3 | 1 | 0 | −2 | 0 | −2 | 4 | −1 | −1 | −3 | −2 | 2 |
| 38 | S | −1 | −1 | 0 | 1 | −2 | 0 | 2 | −2 | −1 | 2 | −1 | 1 | −1 | −2 | −2 | 2 | −1 | −3 | −2 | 0 |
| 39 | S | 3 | −1 | −1 | −1 | −1 | −1 | −1 | −1 | −2 | −1 | 0 | −1 | −1 | −2 | 2 | 3 | 0 | −3 | −2 | −1 |
| 40 | N | 0 | −2 | 2 | −1 | −3 | −1 | 1 | 3 | −1 | 0 | 0 | −1 | −1 | −3 | 1 | −1 | −1 | −3 | −2 | 0 |
| 41 | D | 1 | −2 | −1 | 3 | −2 | 0 | 2 | −2 | −2 | −1 | 0 | −1 | −1 | 0 | −2 | 0 | −1 | −3 | −2 | −1 |
| 42 | N | −1 | −1 | 2 | 1 | −3 | 0 | 3 | 0 | −1 | −2 | 0 | 0 | −1 | −2 | −2 | 1 | −1 | −3 | −2 | −2 |
| 43 | L | 1 | −3 | −3 | −3 | 4 | −2 | −3 | −3 | −1 | 0 | 1 | −3 | 0 | 3 | −3 | −2 | −2 | 0 | 5 | 0 |
| 44 | I | 1 | −1 | −1 | 0 | −2 | 0 | 2 | −2 | −2 | 1 | −1 | 1 | −1 | −2 | −2 | 2 | −1 | −4 | −2 | 0 |
| 45 | F | −2 | −3 | −2 | 1 | −2 | −2 | −2 | −3 | −1 | 2 | 0 | −3 | 0 | 4 | −3 | −2 | −2 | −1 | 4 | 1 |
| 46 | D | 1 | −2 | −1 | 2 | −2 | −1 | 0 | 0 | −2 | −3 | −3 | −1 | −2 | −3 | 4 | 1 | −1 | −4 | −3 | −2 |
| 47 | D | 0 | −2 | 0 | 5 | −3 | −1 | 0 | 1 | −2 | −3 | −4 | −1 | −3 | −3 | 2 | 1 | −1 | −4 | −3 | −3 |
| 48 | Y | −2 | 3 | −1 | −2 | −3 | 2 | −1 | −3 | 0 | −2 | −2 | 0 | −1 | 0 | −3 | −2 | −2 | 0 | 6 | −2 |
| 49 | R | −2 | 4 | 0 | −2 | −4 | 2 | 0 | −2 | 5 | −4 | −3 | 0 | −2 | −3 | 2 | −1 | −2 | −3 | −1 | −3 |
| 50 | G | −1 | −2 | 0 | 2 | −3 | −1 | 1 | 4 | −2 | −2 | −3 | −1 | −2 | −3 | −2 | 1 | −1 | −3 | −3 | 0 |
| 51 | K | −1 | 2 | 1 | −1 | −3 | 0 | 0 | 1 | −1 | −3 | −3 | 4 | −2 | −3 | −2 | 1 | −1 | −3 | −3 | −3 |
| 52 | G | −1 | −2 | −1 | 2 | 4 | −2 | −1 | 3 | −2 | −2 | −3 | −2 | −2 | 0 | −2 | 0 | 2 | −2 | −2 | −2 |
| 53 | C | −1 | −4 | −4 | −4 | 10 | −4 | −4 | −4 | −3 | −1 | −1 | −4 | −1 | 2 | −4 | −2 | −2 | −2 | −1 | −1 |
| 54 | V | −1 | −2 | −1 | 0 | −2 | −1 | 1 | −2 | −2 | 0 | 0 | −1 | 3 | −2 | −2 | 1 | 0 | −3 | −2 | 3 |
| 55 | D | 0 | −2 | 0 | 6 | −3 | 0 | 1 | −1 | −1 | −3 | −4 | −1 | −3 | −3 | −1 | 0 | −1 | −4 | −3 | −3 |
| 56 | D | −2 | −1 | 3 | 4 | −4 | 0 | 2 | 2 | −1 | −4 | −4 | −1 | −3 | −4 | −2 | 0 | −1 | −4 | −3 | −3 |
| 57 | S | 0 | −1 | 2 | 2 | −3 | 1 | 0 | 2 | −1 | −3 | −3 | −1 | −2 | −3 | −2 | 3 | 0 | −3 | −3 | −3 |
| 58 | G | 0 | 1 | 0 | −1 | −3 | −1 | −2 | 5 | −2 | −4 | −4 | −1 | −3 | −3 | −2 | 1 | −2 | −3 | −3 | −3 |
| 59 | F | −1 | −4 | −4 | −4 | −2 | −3 | −3 | −4 | −2 | 3 | 0 | −3 | 0 | 5 | −4 | −2 | −1 | −1 | 0 | 3 |
| 60 | V | −1 | −3 | −3 | −2 | −2 | −1 | 1 | −3 | −1 | 0 | 0 | −2 | 0 | 3 | −3 | −2 | −1 | −1 | 4 | 3 |
| 61 | Y | −2 | −2 | −2 | −3 | −2 | −2 | −2 | −3 | 0 | −1 | −1 | −2 | −1 | 4 | −3 | 0 | −2 | 0 | 7 | −2 |
| 62 | K | 1 | −1 | −2 | −2 | −2 | −1 | −1 | 0 | −3 | 0 | −1 | 1 | −1 | −2 | 2 | −1 | −1 | −4 | −2 | 3 |
| 63 | L | −1 | −3 | −2 | −3 | −2 | −3 | −3 | 4 | −3 | 4 | 1 | −3 | 0 | −1 | −3 | −1 | −2 | −3 | −2 | 0 |
| 64 | G | −1 | −1 | 0 | 0 | −4 | 0 | 3 | 5 | −1 | −4 | −4 | −1 | −3 | −4 | −2 | 0 | −2 | −3 | −3 | −3 |
| 65 | E | −2 | −1 | −1 | 0 | −3 | 3 | 3 | −3 | −1 | −2 | −1 | 0 | 3 | −1 | −2 | −1 | −2 | 7 | −1 | −2 |
| 66 | R | −2 | 2 | −2 | −3 | −2 | 0 | −1 | −3 | 0 | 1 | −1 | 1 | −1 | 3 | −3 | −2 | −2 | −1 | 4 | 0 |
| 67 | F | −2 | −3 | −3 | −3 | −2 | −3 | −3 | −3 | −1 | −1 | −1 | −3 | −1 | 6 | −4 | −2 | 1 | 6 | 3 | −1 |
| 68 | F | 0 | 1 | −2 | −3 | −2 | −1 | −2 | −2 | −1 | −1 | 1 | −1 | 0 | 2 | −3 | 0 | 0 | −1 | 2 | −1 |
| 69 | P | −2 | −2 | −1 | 2 | −4 | 0 | 2 | −2 | −2 | −4 | −4 | −1 | −3 | −4 | 6 | −1 | −1 | −4 | −3 | −3 |
| 70 | G | 0 | −2 | 0 | −1 | −2 | −1 | −1 | 5 | −2 | −4 | −4 | −1 | −2 | −3 | −2 | 3 | −1 | −3 | −3 | −3 |
| 71 | H | −2 | −2 | 0 | 5 | −3 | −1 | 0 | −2 | 4 | −3 | −4 | −1 | −3 | −3 | 3 | 0 | −1 | −4 | −2 | −3 |
| 72 | S | −1 | −1 | 0 | −1 | −2 | −1 | −1 | −2 | 5 | −3 | −3 | −1 | −2 | −3 | 4 | 3 | 1 | −3 | −1 | −2 |
| 73 | N | 2 | −2 | 2 | −2 | 8 | −2 | −2 | −2 | −2 | −2 | −2 | −2 | −2 | −3 | −3 | 0 | 0 | −3 | −2 | −1 |
| 74 | C | −1 | −2 | −1 | −1 | 8 | −1 | 3 | −3 | −2 | −2 | −2 | −1 | −2 | −3 | −2 | 0 | 2 | −3 | −2 | −1 |
| 75 | P | −1 | 2 | −2 | −2 | −3 | 3 | 0 | −3 | −2 | −2 | 0 | 0 | −1 | −3 | 5 | −1 | −1 | −3 | −2 | −2 |
| 76 | C | 0 | −4 | −4 | −4 | 10 | −4 | −5 | −4 | −4 | −1 | −1 | −4 | −1 | −2 | −4 | −1 | −1 | −2 | −2 | −1 |
| 77 | V | −1 | −2 | −2 | −1 | −2 | −1 | 2 | −3 | −2 | 0 | 2 | −1 | 0 | −1 | −2 | −1 | 0 | −3 | −2 | 3 |
| 78 | C | 0 | −4 | −4 | −4 | 10 | −4 | −5 | −4 | −4 | −1 | −1 | −4 | −1 | −2 | −4 | −1 | −1 | −2 | −2 | −1 |
| 79 | A | 2 | −1 | −1 | −1 | −1 | 1 | −1 | −2 | −2 | −2 | −2 | −1 | −1 | −3 | −1 | 0 | 4 | −3 | −2 | −1 |
| 80 | L | 1 | −1 | −1 | 0 | −2 | 0 | 2 | −2 | −2 | −1 | 0 | −1 | −1 | −2 | −2 | 1 | 1 | −3 | −2 | −1 |
| 81 | D | −2 | −2 | 0 | 5 | −4 | 0 | 4 | −2 | −1 | −3 | −4 | −1 | −3 | −3 | −2 | 0 | 1 | −4 | −3 | −3 |
| 82 | G | 0 | −3 | 0 | −2 | −4 | −3 | −3 | 7 | −3 | −5 | −5 | −3 | −4 | −4 | −3 | 0 | −3 | −3 | −4 | −4 |
| 83 | P | −1 | −2 | −2 | −1 | −3 | −1 | −1 | −2 | −2 | −3 | −3 | −1 | −2 | −4 | 7 | 1 | −1 | −4 | −3 | −3 |
| 84 | V | 0 | −2 | −2 | −3 | −2 | 1 | −1 | −3 | −3 | 1 | 1 | −2 | 0 | −2 | −2 | 0 | −1 | −3 | −2 | 4 |
| 85 | C | 0 | −3 | −3 | −3 | 10 | −3 | −4 | −3 | −3 | −2 | −2 | −3 | −2 | −3 | −3 | 0 | −1 | −3 | −3 | −2 |
| 86 | D | 1 | −2 | −1 | 3 | 4 | −1 | −1 | −2 | −2 | −1 | −2 | −2 | 0 | 0 | −2 | 1 | 0 | −3 | −2 | 0 |
| 87 | Q | −1 | 3 | −1 | −2 | −3 | 4 | 0 | −3 | −1 | −2 | −2 | 2 | −1 | −3 | −2 | −1 | −1 | −3 | −2 | 0 |
| 88 | P | −1 | −2 | −1 | −2 | −2 | −2 | −2 | −3 | −3 | −2 | −2 | −2 | −2 | −4 | 6 | 0 | 4 | −4 | −3 | −1 |
| 89 | E | −2 | 2 | 0 | 2 | −4 | 0 | 5 | −2 | −1 | −4 | −3 | 1 | −2 | −4 | −2 | −1 | −2 | −4 | −3 | −3 |
| 90 | C | 0 | −4 | −4 | −4 | 10 | −4 | −5 | −4 | −4 | −2 | −2 | −4 | −2 | −3 | −4 | −2 | −2 | −3 | −3 | −2 |
| 91 | P | −1 | −2 | −2 | −2 | −3 | −2 | −2 | −3 | −3 | −1 | −2 | −2 | −2 | −3 | 6 | −1 | 2 | −4 | −3 | 0 |
| 92 | K | 0 | 3 | 0 | 0 | −3 | 0 | 3 | −2 | −1 | −3 | −3 | 3 | −2 | −3 | −2 | 0 | −1 | −3 | −2 | −2 |
| 93 | I | −1 | −3 | −3 | −4 | −1 | −2 | −3 | −4 | −3 | 3 | 3 | −2 | 0 | −1 | −3 | −2 | 1 | −3 | −2 | 1 |
| 94 | H | −1 | −1 | 0 | −1 | 4 | −1 | −1 | −2 | 7 | −3 | −3 | −1 | −2 | −2 | 2 | 1 | −1 | −3 | 0 | −3 |
| 95 | P | 0 | −2 | −2 | −1 | −3 | −1 | 1 | −2 | −2 | −3 | −3 | −1 | −2 | −4 | 7 | −1 | −1 | −4 | −3 | −2 |
| 96 | K | 1 | 3 | −1 | −2 | −2 | 0 | 0 | −2 | 0 | −2 | −2 | 2 | −1 | −1 | −2 | 1 | −1 | −2 | 2 | −2 |
| 97 | C | 0 | −3 | −3 | −3 | 10 | −3 | −4 | −3 | −3 | −2 | −2 | −3 | −2 | −3 | 2 | −1 | −1 | −3 | −3 | −2 |
| 98 | T | −1 | −3 | −2 | −3 | −1 | −2 | −3 | −4 | −3 | 4 | 0 | −3 | 1 | −1 | −3 | −1 | 3 | −3 | −2 | 2 |
| 99 | K | −2 | 1 | 0 | −1 | −4 | 0 | 2 | −2 | 7 | −3 | −3 | 2 | −2 | −2 | −2 | −1 | −2 | −3 | 0 | −3 |
| 100 | V | −1 | −3 | −3 | −3 | −1 | −3 | −3 | −4 | −3 | 3 | 0 | −3 | 0 | −1 | −3 | −2 | −1 | −3 | −1 | 5 |
| 101 | E | 1 | −1 | 0 | 3 | −2 | 0 | 2 | −1 | −1 | −3 | −3 | 1 | −2 | −3 | −1 | 2 | 0 | −4 | −3 | −2 |
| 102 | H | −2 | 2 | 1 | −1 | −3 | 0 | −1 | −2 | 7 | −3 | −2 | 0 | −2 | −1 | −2 | −1 | 1 | −2 | 3 | −2 |
| 103 | N | −1 | 0 | 3 | −1 | −2 | 0 | 0 | −1 | 4 | −2 | −2 | 1 | −2 | 0 | −2 | 1 | 0 | −1 | 4 | −2 |
| 104 | G | −1 | −1 | 0 | 2 | −3 | 4 | 2 | 2 | −1 | −4 | −3 | 0 | −2 | −3 | −2 | 0 | −1 | −3 | −2 | −3 |
| 105 | C | 0 | −3 | −3 | −3 | 10 | −3 | −5 | −3 | −3 | −1 | −1 | −3 | −1 | −2 | −3 | −1 | −1 | −2 | −2 | −1 |
| 106 | C | 0 | −3 | −3 | −3 | 10 | −3 | −5 | −3 | −3 | −1 | −1 | −3 | −1 | −2 | −3 | −1 | −1 | −2 | −2 | −1 |
| 107 | P | −1 | −3 | −3 | −2 | −3 | −2 | −2 | −3 | −3 | −1 | −2 | −2 | −2 | −4 | 7 | −2 | −1 | −4 | −3 | 0 |
| 108 | E | −1 | 2 | 0 | 2 | −3 | 4 | 2 | −2 | −1 | −2 | −1 | 0 | −1 | −3 | −2 | −1 | −1 | −3 | −2 | 0 |
| 109 | C | 0 | −3 | −3 | −3 | 10 | −3 | −5 | −3 | −3 | −1 | −1 | −3 | −1 | −2 | −3 | −1 | −1 | −2 | −2 | −1 |
| 110 | K | −1 | 0 | 0 | 0 | −3 | 0 | 3 | −2 | −1 | −1 | −2 | 3 | −1 | −3 | −2 | 1 | −1 | −3 | −2 | 1 |
| 111 | E | 2 | 2 | −1 | 0 | −3 | 0 | 4 | −2 | −1 | −3 | −2 | 2 | −2 | −3 | −2 | 0 | −1 | −3 | −2 | −2 |
| 112 | V | −1 | 1 | −1 | −1 | −3 | −1 | 1 | 1 | −2 | 1 | −1 | 1 | −1 | −2 | −2 | −1 | −1 | −3 | −2 | 2 |
| 113 | K | 0 | 0 | 0 | −1 | −3 | 0 | 0 | 4 | −2 | −4 | −3 | 4 | −2 | −3 | −2 | 1 | −1 | −3 | −3 | −3 |
| 114 | N | −2 | 0 | 6 | 0 | −3 | 0 | 0 | 0 | 0 | −3 | −3 | 2 | −2 | −3 | −2 | 0 | 0 | −4 | −2 | −3 |
| 115 | F | −1 | −3 | −3 | −3 | −2 | −2 | −2 | −3 | 0 | 0 | 0 | −2 | 0 | 4 | −3 | −2 | −1 | 0 | 6 | 1 |
| 116 | C | 0 | −3 | −3 | −3 | 10 | −3 | −5 | −3 | −3 | −1 | −1 | −3 | −1 | −2 | −3 | −1 | −1 | −2 | −2 | −1 |
| 117 | E | −1 | −1 | 0 | 3 | −3 | 0 | 4 | −2 | −1 | −2 | 0 | 0 | −2 | −3 | −2 | 0 | 1 | −3 | −3 | −2 |
| 118 | Y | −2 | −2 | −2 | −2 | −3 | −1 | 1 | −3 | 0 | −1 | −1 | −2 | −1 | 5 | −3 | −2 | −2 | 0 | 6 | −2 |
| 119 | H | −2 | 5 | 2 | −2 | −3 | 0 | −1 | −2 | 4 | −2 | −1 | 0 | 3 | −2 | −2 | −1 | −1 | −3 | −1 | −2 |
| 120 | G | −1 | −2 | 2 | −1 | −3 | −2 | −2 | 6 | −2 | −4 | −4 | −2 | −3 | −3 | −2 | 0 | −2 | −3 | −3 | −3 |
| 121 | K | −1 | 4 | −1 | −2 | −3 | 0 | 0 | −3 | −1 | −1 | −2 | 5 | −1 | −3 | −2 | −1 | −1 | −3 | −2 | 0 |
| 122 | N | −1 | −2 | 2 | −2 | −2 | −2 | −2 | −2 | −2 | 2 | 0 | −2 | 0 | −2 | −2 | 0 | 4 | −3 | −2 | 1 |
| 123 | Y | −2 | −3 | −3 | −3 | −2 | −2 | −3 | −3 | 0 | −1 | −1 | −3 | −1 | 4 | −4 | −2 | −2 | 1 | 8 | −1 |
| 124 | K | 0 | 1 | 0 | −1 | −3 | 2 | 2 | −2 | 4 | −3 | −2 | 2 | −1 | −1 | −2 | −1 | −2 | −2 | 3 | −2 |
| 125 | I | −1 | −2 | 2 | −2 | −2 | −2 | −2 | −2 | −2 | 2 | 1 | −2 | 0 | −1 | −2 | 0 | 1 | −3 | −2 | 2 |
| 126 | L | −1 | −2 | 2 | −2 | −2 | −2 | −2 | 3 | −2 | 0 | 3 | −2 | 0 | −1 | −3 | −1 | −1 | −3 | −2 | −1 |
| 127 | E | −1 | 0 | 0 | 1 | −4 | 3 | 6 | −2 | 0 | −3 | −3 | 0 | −2 | −3 | −1 | 0 | −1 | −3 | −2 | −2 |
| 128 | E | −1 | −1 | 2 | 0 | −3 | 0 | 4 | −2 | 0 | −3 | −3 | 0 | −2 | −1 | −2 | 0 | 1 | −2 | 3 | −2 |
| 129 | F | −2 | −3 | −3 | −3 | −2 | −3 | −3 | −3 | −1 | 0 | 0 | −3 | 0 | 7 | −5 | −2 | −2 | 0 | 2 | −1 |
| 130 | K | −1 | 1 | 2 | −1 | −3 | 2 | 0 | −2 | −1 | −1 | 0 | 2 | 1 | −2 | −2 | −1 | −1 | −3 | −2 | 0 |
| 131 | P | −1 | −3 | −3 | −3 | −2 | −2 | −2 | −3 | −3 | 1 | 2 | −2 | 0 | −2 | 3 | −2 | 0 | −3 | −2 | 3 |
| 132 | S | 0 | 0 | 1 | 2 | 2 | −1 | 0 | −1 | −1 | −3 | −3 | −1 | −2 | −3 | 1 | 4 | 1 | −4 | −3 | −2 |
| 133 | P | −1 | −2 | −2 | −1 | −3 | −1 | 1 | −3 | −2 | −2 | −3 | −1 | −2 | −4 | 7 | −1 | −1 | −4 | −3 | 0 |
| 134 | C | 0 | −3 | −3 | −3 | 10 | −3 | −4 | −3 | −3 | −1 | −1 | −3 | −1 | −2 | −3 | −1 | 0 | −2 | −2 | −1 |
| 135 | E | −1 | 1 | −1 | 0 | −3 | 0 | 5 | −3 | −1 | −1 | 1 | 0 | −1 | −2 | −2 | −1 | −1 | −3 | −2 | −1 |
| 136 | W | −1 | 2 | −1 | −2 | −3 | 2 | 0 | −2 | 3 | −2 | 0 | 1 | −1 | −2 | −2 | 1 | −1 | 4 | −1 | −2 |
| 137 | C | 0 | 0 | −3 | −3 | 10 | −3 | −4 | −3 | −3 | 0 | −1 | −3 | −1 | −2 | −3 | −1 | 0 | −3 | −2 | −1 |
| 138 | R | −1 | 4 | −1 | −2 | −2 | 0 | −1 | −3 | −1 | 2 | −1 | 2 | −1 | −1 | −2 | −1 | 1 | −2 | 1 | 0 |
| 139 | C | 0 | −4 | −4 | −4 | 10 | −4 | −5 | −4 | −4 | −1 | −1 | −4 | −1 | −2 | −4 | −1 | −1 | −2 | −2 | −1 |
| 140 | E | −1 | −1 | 0 | 2 | −3 | 0 | 5 | −3 | −1 | −2 | 0 | 0 | −1 | −3 | −2 | 0 | 1 | −3 | −2 | −2 |
| 141 | P | 1 | −2 | 1 | −2 | −2 | −1 | −1 | −2 | −2 | 0 | 0 | −1 | −1 | −2 | 2 | 1 | 1 | −3 | −2 | 0 |
| 142 | S | −1 | −1 | 4 | 2 | −3 | 1 | 0 | 2 | −1 | −3 | −3 | −1 | −2 | −3 | −2 | 2 | −1 | −4 | −3 | −3 |
| 143 | N | −1 | 1 | 2 | −1 | −3 | −1 | −1 | 4 | −1 | −4 | −4 | 1 | −2 | −3 | −2 | 1 | −1 | −3 | −3 | −3 |
| 144 | E | −1 | 0 | −1 | 0 | −3 | 0 | 4 | −3 | −1 | 1 | −1 | 0 | −1 | −2 | −2 | −1 | 1 | −3 | −2 | 1 |
| 145 | V | 3 | −2 | −3 | −3 | −1 | −2 | −2 | −2 | −3 | 0 | 0 | −2 | 0 | −2 | −2 | −1 | −1 | −3 | −2 | 3 |
| 146 | H | −2 | 1 | −2 | −3 | −2 | −1 | −2 | −3 | 3 | 0 | 2 | −1 | 0 | 2 | −3 | −2 | −2 | −1 | 4 | −1 |
| 147 | C | −1 | 0 | −3 | −3 | 10 | −3 | −3 | −3 | −3 | −2 | −2 | −2 | −2 | −3 | 1 | −1 | −1 | −3 | −3 | −2 |
| 148 | V | 0 | −2 | −1 | −2 | −1 | −1 | −2 | −2 | −3 | 0 | 1 | −1 | 0 | −2 | −2 | 2 | 3 | −3 | −2 | 2 |
| 149 | V | −1 | −3 | −3 | −3 | 1 | −2 | −2 | −3 | −3 | 2 | 0 | −2 | 0 | −2 | 2 | −2 | −1 | −4 | −2 | 5 |
| 150 | A | 3 | −2 | −2 | −2 | 2 | −1 | −1 | −1 | −2 | 0 | −1 | −1 | −1 | −2 | −2 | 2 | 0 | −3 | −2 | 2 |
| 151 | D | 1 | −2 | 0 | 5 | −2 | 1 | 0 | 0 | −2 | −3 | −3 | −1 | −2 | −3 | −2 | 1 | −1 | −4 | −3 | −2 |
| 152 | C | −1 | −3 | −3 | −3 | 10 | −3 | −4 | 0 | −3 | −2 | −2 | −3 | −1 | 1 | −4 | −1 | −2 | −2 | −1 | −2 |
| 153 | A | 2 | −2 | −2 | −2 | −2 | −1 | 0 | −2 | −2 | −1 | 0 | −1 | −1 | 1 | 4 | −1 | −1 | −3 | −1 | −1 |
| 154 | V | 2 | −1 | −2 | −1 | −2 | 4 | 1 | −2 | −2 | −1 | −2 | 0 | −1 | −3 | 3 | −1 | −1 | −3 | −2 | 0 |
| 155 | P | −1 | −2 | −1 | 1 | −2 | −2 | −1 | −2 | −2 | 1 | 0 | −2 | −1 | −2 | 4 | 0 | 2 | −4 | −3 | 0 |
| 156 | E | −2 | 1 | −1 | 0 | −4 | 0 | 3 | −3 | 2 | −3 | −3 | 1 | −2 | 2 | 3 | −1 | −2 | −3 | −1 | −3 |
| 157 | C | −1 | −3 | −3 | −3 | 9 | 1 | −3 | −3 | −3 | −2 | −1 | −3 | −1 | −2 | −3 | −2 | −2 | 5 | −2 | −2 |
| 158 | V | −1 | 0 | −2 | −3 | −2 | 0 | −2 | −3 | −3 | 2 | 0 | −2 | 0 | 1 | −2 | −2 | 1 | −2 | −1 | 4 |
| 159 | N | −2 | 1 | 4 | 4 | −3 | 0 | 0 | −1 | 0 | −3 | −3 | −1 | −2 | −1 | −2 | 0 | −1 | 3 | 3 | −3 |
| 160 | P | −1 | −2 | −2 | −1 | −3 | −1 | −1 | −2 | −2 | −1 | −3 | 1 | −2 | −4 | 7 | 0 | −1 | −4 | −3 | −2 |
| 161 | V | −1 | −3 | −3 | −3 | −2 | 0 | 0 | −4 | −3 | 4 | 0 | −2 | 0 | 2 | −3 | −2 | −1 | −2 | 0 | 3 |
| 162 | Y | −2 | 1 | −1 | −3 | −3 | −1 | −1 | −3 | 4 | −2 | 0 | −1 | −1 | 0 | −3 | 0 | −2 | 0 | 6 | −2 |
| 163 | E | −1 | −1 | −1 | 0 | −3 | 3 | 4 | −3 | −1 | −2 | −1 | 0 | −1 | −3 | 3 | 0 | 0 | −3 | −2 | −2 |
| 164 | P | −1 | −1 | −2 | −1 | −3 | 0 | 1 | −3 | −1 | −2 | −1 | 0 | −2 | −2 | 5 | −1 | −1 | −2 | 2 | −2 |
| 165 | E | −1 | −2 | 1 | 2 | −3 | −1 | 1 | 3 | −1 | −3 | −3 | −1 | −3 | −1 | −2 | 0 | −1 | −2 | 2 | −3 |
| 166 | Q | −2 | 0 | −1 | −1 | −3 | 4 | 2 | −3 | 3 | −3 | 0 | 2 | −1 | −2 | −2 | −1 | −2 | 5 | 0 | −2 |
| 167 | C | 0 | −3 | −3 | −4 | 9 | −3 | −4 | −4 | −3 | 0 | 0 | −3 | −1 | −2 | −3 | −2 | −1 | −2 | −2 | 0 |
| 168 | C | 0 | −3 | −3 | −3 | 9 | −3 | −3 | −3 | −3 | 0 | −1 | −3 | −1 | −2 | −3 | 0 | −1 | −3 | −2 | 1 |
| 169 | P | 0 | −2 | −3 | −2 | −3 | −1 | −2 | −2 | −3 | −2 | 0 | −1 | −1 | −3 | 7 | −1 | −1 | −4 | −3 | −1 |
| 170 | V | −1 | −2 | −2 | −2 | −2 | −1 | 1 | −3 | −2 | 3 | 0 | 1 | 0 | −2 | −2 | −2 | −1 | −3 | −2 | 4 |
| 171 | C | −1 | −2 | −3 | −3 | 9 | −2 | −3 | −3 | −3 | −1 | 0 | 1 | −1 | −2 | −3 | −1 | −1 | −3 | −2 | −1 |
| 172 | K | −1 | 1 | 0 | −1 | −3 | 0 | 0 | −2 | −1 | −3 | −2 | 6 | −1 | −3 | −1 | 0 | −1 | −3 | −2 | −2 |
| 173 | N | 2 | −1 | 3 | 2 | −2 | −1 | 0 | 2 | −1 | −3 | −3 | −1 | −2 | −3 | −2 | 1 | −1 | −4 | −3 | −2 |
| 174 | G | 0 | −1 | 0 | −1 | −3 | −2 | −2 | 5 | −2 | −4 | −3 | 0 | −3 | −3 | −2 | 0 | −2 | −2 | −3 | −3 |
| 175 | P | −1 | −2 | 1 | −1 | −3 | −1 | 0 | −2 | −2 | −3 | −3 | −1 | −2 | −4 | 6 | −1 | −1 | −4 | −3 | −2 |
| 176 | N | −1 | −1 | 5 | 0 | −3 | −1 | −1 | −1 | 0 | −3 | −3 | −1 | −2 | −2 | −2 | 1 | 0 | 6 | −1 | −3 |
| 177 | C | −1 | −2 | −2 | −2 | 8 | −2 | −2 | −3 | 3 | −2 | −2 | −2 | −2 | −2 | 1 | −1 | 0 | −3 | −1 | −2 |
| 178 | F | −2 | −2 | −1 | −2 | −3 | −2 | −2 | 1 | 4 | −2 | −2 | 0 | −1 | 4 | 0 | −1 | −2 | 0 | 3 | −2 |
| 179 | A | 2 | −2 | −2 | −3 | −1 | −2 | −2 | 2 | −3 | 0 | 1 | −2 | 0 | 0 | −2 | −1 | −1 | −3 | −1 | 1 |
| 180 | G | 0 | −1 | 0 | 0 | −2 | −1 | 1 | 3 | −2 | −2 | 0 | −1 | −1 | −2 | 1 | 1 | 1 | −3 | −2 | −1 |
| 181 | T | 0 | −2 | −1 | −1 | −1 | −1 | −1 | −2 | −2 | 1 | −1 | −1 | −1 | −2 | 1 | 0 | 5 | −3 | −2 | 0 |
| 182 | T | 1 | −1 | 0 | −1 | −1 | 2 | 0 | −1 | −2 | −1 | 0 | −1 | 0 | −2 | −1 | 1 | 3 | −3 | −2 | −1 |
| 183 | I | 1 | −3 | −2 | −3 | −1 | −2 | −2 | −2 | −3 | 3 | 0 | −2 | 0 | −1 | −2 | 0 | 0 | −3 | −1 | 3 |
| 184 | I | −1 | −3 | −3 | −3 | −2 | −3 | −3 | 1 | −3 | 5 | 0 | −3 | 0 | 2 | −3 | −2 | −1 | −2 | 0 | 1 |
| 185 | P | −1 | −2 | −2 | −1 | −3 | 0 | 1 | −2 | −2 | −2 | 0 | −1 | −1 | −3 | 6 | −1 | −1 | −4 | −3 | −2 |
| 186 | A | 4 | 1 | −2 | −2 | 0 | −1 | −1 | 0 | −2 | −1 | −1 | 0 | −1 | −2 | −1 | 0 | 0 | −3 | −2 | 0 |
| 187 | G | 0 | −2 | 0 | −1 | −3 | −2 | −2 | 6 | −2 | −2 | −3 | −2 | −2 | −3 | −2 | 0 | −2 | −2 | −3 | 0 |
| 188 | I | −1 | 2 | −1 | 0 | −3 | 0 | 3 | −3 | −1 | 2 | −1 | 0 | −1 | −2 | 1 | −1 | −1 | −3 | −2 | 0 |
| 189 | E | −1 | −1 | −1 | 0 | −3 | 0 | 4 | −2 | −1 | 0 | −2 | 0 | −1 | −3 | 3 | −1 | −1 | −3 | −2 | −1 |
| 190 | V | 0 | 0 | −1 | 1 | −2 | −1 | −1 | −2 | −2 | 0 | 0 | −1 | 0 | −2 | −2 | −1 | 2 | −3 | −2 | 3 |
| 191 | K | −1 | 0 | −1 | −1 | −3 | 0 | 1 | −2 | −1 | −2 | −2 | 4 | −1 | −2 | −2 | −1 | −1 | 7 | −1 | 0 |
| 192 | V | 0 | −1 | −1 | −2 | −2 | 1 | 0 | −3 | −2 | 0 | 0 | 1 | 0 | −2 | −2 | −1 | 2 | −3 | −1 | 3 |
| 193 | D | −2 | −2 | 0 | 6 | −3 | 0 | 1 | −1 | −1 | −3 | −4 | −1 | −3 | −3 | −1 | 0 | 1 | −4 | −3 | −3 |
| 194 | E | 1 | −1 | −1 | 1 | −2 | 0 | 4 | −2 | −1 | −1 | −2 | 0 | −1 | −2 | −1 | 0 | −1 | −3 | −2 | 1 |
| 195 | C | 0 | −3 | −3 | −3 | 10 | −3 | −4 | −3 | −3 | −1 | −1 | −3 | −1 | −2 | −3 | −1 | −1 | −2 | −2 | −1 |
| 196 | N | −1 | −1 | 3 | −1 | −2 | −1 | −1 | −1 | −1 | −1 | −1 | −1 | −1 | 2 | −2 | 0 | 4 | −2 | 0 | −1 |
| 197 | I | −1 | −1 | −2 | −3 | 5 | −2 | −2 | −3 | −3 | 4 | 0 | 1 | 0 | −1 | −3 | −1 | −1 | −3 | −1 | 1 |
| 198 | C | 0 | −3 | −2 | −3 | 9 | −3 | −3 | −3 | −3 | −1 | −1 | −3 | −1 | −2 | −3 | −1 | 1 | −2 | −2 | −1 |
| 199 | H | −2 | 2 | 0 | −2 | −3 | 0 | 0 | −2 | 7 | −3 | −2 | 0 | −2 | 0 | −2 | −1 | −2 | −1 | 3 | −3 |
| 200 | C | 0 | −3 | −3 | −3 | 10 | −3 | −4 | −3 | −3 | −1 | −1 | −3 | −1 | −2 | −3 | −1 | −1 | −2 | −2 | −1 |
| 201 | H | −1 | 0 | 0 | −1 | −2 | 0 | 0 | −2 | 7 | −2 | −2 | −1 | −2 | −1 | −2 | 0 | 3 | −2 | 0 | −2 |
| 202 | N | −2 | −1 | 4 | 3 | −3 | 0 | 0 | −1 | 0 | −2 | −2 | −1 | −2 | 0 | −2 | 0 | −1 | −1 | 4 | −2 |
| 203 | G | 0 | −1 | 0 | 0 | −3 | 0 | 3 | 5 | −1 | −4 | −4 | −1 | −3 | −3 | −2 | 0 | −2 | −2 | −3 | −3 |
| 204 | D | −1 | 0 | 0 | 4 | −3 | 3 | 3 | −2 | 0 | −3 | −3 | 0 | −2 | −3 | −1 | 0 | −1 | −3 | −2 | −2 |
| 205 | W | −1 | −2 | 0 | 3 | −3 | −1 | 0 | 3 | −2 | −3 | −3 | −2 | −2 | −1 | −2 | −1 | −2 | 8 | 0 | −3 |
| 206 | W | −2 | −3 | −2 | −3 | −2 | −2 | −3 | 3 | −2 | −3 | −3 | −3 | −1 | 0 | −3 | −2 | −2 | 11 | 0 | −3 |
| 207 | K | −1 | 3 | 0 | −2 | −3 | 0 | 0 | −2 | 0 | −2 | −2 | 3 | −1 | 0 | −2 | −1 | −1 | −1 | 4 | −2 |
| 208 | P | −1 | −1 | −1 | −1 | −2 | 3 | 0 | −3 | −1 | −1 | 1 | 0 | 0 | −2 | 4 | −1 | −1 | −3 | −2 | −1 |
| 209 | A | 5 | −1 | −2 | −2 | 0 | −1 | −1 | 0 | −2 | −1 | −1 | −1 | −1 | −2 | −1 | 0 | 0 | −3 | −2 | 0 |
| 210 | Q | −1 | 0 | −1 | −1 | −2 | 3 | 0 | −2 | −1 | −1 | 0 | 0 | 4 | −1 | −1 | 0 | 2 | −2 | −1 | 0 |
| 211 | C | 0 | −3 | −3 | −3 | 10 | −3 | −4 | −3 | −3 | −1 | −1 | −3 | −1 | −2 | −3 | −1 | −1 | −2 | −2 | −1 |
| 212 | S | 0 | −1 | 0 | 0 | −1 | 0 | 0 | −1 | −1 | −2 | −2 | 0 | −1 | −2 | −1 | 4 | 3 | −3 | −2 | −1 |
| 213 | K | −1 | 5 | 0 | −2 | −3 | 0 | 0 | −2 | 0 | −3 | −2 | 4 | −1 | −3 | −2 | −1 | −1 | −3 | −2 | −3 |
| 214 | R | −1 | 3 | 0 | −2 | −2 | 0 | −1 | −3 | 6 | −1 | 1 | 0 | 0 | −1 | −2 | −1 | −1 | −2 | 0 | −1 |
| 215 | E | −1 | 0 | 0 | 1 | −4 | 1 | 5 | −2 | 0 | −3 | −3 | 3 | −2 | −3 | −1 | 0 | −1 | −3 | −2 | −2 |
| 216 | C | 0 | −2 | 3 | −1 | 9 | −2 | −2 | −2 | −1 | −1 | −1 | −2 | −1 | −2 | −3 | 0 | −1 | −3 | −2 | −1 |
| 217 | Q | −1 | 2 | −1 | −1 | 6 | 3 | 0 | −2 | −1 | −2 | −2 | 1 | −1 | −3 | −2 | 0 | −1 | −2 | −2 | −2 |
| 218 | G | −1 | 0 | 3 | 0 | −3 | 3 | 0 | 3 | 0 | −3 | −3 | 0 | −1 | −3 | −2 | 0 | −1 | −3 | −2 | −3 |
| 219 | K | −1 | 0 | 0 | −2 | −2 | 0 | 0 | −2 | 5 | 0 | 0 | 3 | 3 | −1 | −2 | −1 | −1 | −2 | 0 | 0 |
| 220 | Q | −1 | 0 | 0 | 0 | −3 | 4 | 4 | −2 | 0 | −3 | −2 | 0 | −1 | −3 | −1 | 0 | −1 | −2 | −1 | −2 |
| 221 | T | 0 | −1 | 0 | −1 | −2 | −1 | −1 | 4 | −2 | −2 | −2 | −1 | −2 | −2 | −1 | 0 | 4 | −2 | −2 | −1 |
| 222 | V | 0 | −2 | −2 | −3 | −1 | −1 | −2 | −3 | −2 | 2 | 1 | −1 | 4 | 0 | −2 | −1 | 0 | −2 | −1 | 3; or |

2) comprises a fragment of the amino acid sequence of a1), wherein said fragment is a member of the vWFC domain containing protein family, or has an antigenic determinant in common with the amino acid sequence of a1); or
3) comprises a functional equivalent of the amino acid sequence of a1) or a2); or
4) consists of the amino acid sequence of a1); or
5) is any of a1)-a4), wherein the polypeptide has a maximum threshold E value of 10⁻²; or
6) comprises an amino acid sequence satisfying the consensus amino acid sequence[GTDFC](0,1)-[CF](0,1)-[VMSED](0,1)-[DEA](0,1)-[DENG](0,1)-[SQNDG](0,1)-[SGR](0,1)-[FIV](0,1)-[VYFE](0,1)-[YFS](0,1)-[KVAGP](0,1)-[LIG](0,1)-[GE](0,1)-[EWQM](0,1)-[RKYFQVI](0,1)-[FYWT](0,1)-[FALYRTS](0,1)-[PED](0,1)-[GS](0,1)-[HPDS](0,1)-[STHP](0,1)-[CNAT](0,1)-[CTE](0,1)-[PQRL](0,1)-C(0,1)-[VELT](0,1)-C(0,1)-[TAQ](0,1)-[ELATSD](0,1)-[EDT]-G-[PS]-[VLAQS]-[CS]-[DAMSTFCV]-[QRKV]-R(0,1)-[PT]-[ERDK]-C-[PTV]-[KERSA]-[LIVT]-[HPSC]-[PAE]-[KRASY]-[CP]-[TIVM]-[HKRE][VI]-[DESAKP]-[HTNRYG]-[NSTYHK](0,1)-[PA](0,1)-[TG](0,1)-[QGDES]-C-C-[PV][EQRDLV]-C; or
7) comprises a fragment of the amino acid sequence of a6), wherein said fragment is a member of the vWFC domain containing protein family, or has an antigenic determinant in common with the polypeptides of a6); or
8) comprises a functional equivalent of a6) or a7); or
9) consists of an amino acid sequence satisfying the consensus amino acid sequence[GTDFC](0,1)-[CF](0,1)-[VMSED](0,1)-[DEA](0,1)-[DENG](0,1)-[SQNDG](0,1)-[SGR](0,1)-[FIV](0,1)-[VYFE](0,1)-[YFS](0,1)-[KVAGP](0,1)-[LIG](0,1)-[GE](0,1)-[EWQM](0,1)-[RKYFQVI](0,1)-[FYWT](0,1)-[FALYRTS](0,1)-[PED](0,1)-[GS](0,1)-[HPDS](0,1)-[STHP](0,1)-[CNAT](0,1)-[CTE](0,1)-[PQRL](0,1)-C(0,1)-[VELT](0,1)-C(0,1)-[TAQ](0,1)-[ELATSD](0,1)-[EDT]-G-[PS]-[VLAQS]-[CS]-[DAMSTFCV]-[QRKV]-R(0,1)-[PT]-[ERDK]-C-[PTV]-[KERSA]-[LIVT]-[HPSC]-[PAE]-[KRASY]-[CP]-[TIVM]-[HKRE]-[VI]-[DESAKP]-[HTNRYG]-[NSTYHK](0,1)-[PA](0,1)-[TG](0,1)-[QGDES]-C-C-[PV]-[EQRDLV]-C; or
10) comprises an amino acid sequence selected from the group consisting of SEQ ID NO:2, SEQ ID NO:4, SEQ ID NO:39, SEQ ID NO:41, SEQ ID NO:43, and SEQ ID NO:45; or
11) comprises a fragment of the amino acid sequence of a10), wherein said fragment is a member of the vWFC domain containing protein family, or has an antigenic determinant in common with the amino acid sequence of a10); or
12) comprises a functional equivalent of a10) or a11); or
13) consists of an amino acid sequence selected from the group consisting of SEQ ID NO:2, SEQ ID NO:4, SEQ ID NO:39, SEQ ID NO:41, SEQ ID NO:43, and SEQ ID NO:45; or
14) comprises a fragment of the amino acid sequence of a13), wherein said fragment is a member of the vWFC domain containing protein family, or has an antigenic determinant in common with the amino acid sequence of a13); or
15) comprises a functional equivalent of a13) or a14); or
16) comprises an amino acid sequence selected from the group consisting of SEQ ID NO:6, SEQ ID NO:8, SEQ ID NO:10, SEQ ID NO:12, SEQ ID NO:14, SEQ ID NO:16, SEQ ID NO:47, SEQ ID NO:49, SEQ ID NO:51, and SEQ ID NO:53; or
17) comprises a fragment of the amino acid sequence of a16), wherein said fragment is a member of the vWFC domain containing protein family, or has an antigenic determinant in common with the amino acid sequence of a16); or
18) comprises a functional equivalent of a16) or a17); or
19) consists of an amino acid sequence selected from the group consisting of SEQ ID NO:6, SEQ ID NO:8, SEQ ID NO:10, SEQ ID NO:12, SEQ ID NO:14, SEQ ID NO:16, SEQ ID NO:47, SEQ ID NO:49, SEQ ID NO:51, and SEQ ID NO:53; or
20) comprises a fragment of the amino acid sequence of a19), wherein said fragment is a member of the vWFC domain containing protein family, or has an antigenic determinant in common with the amino acid sequence of a19); or
21) comprises a functional equivalent of a19) or a20); or
22) comprises the amino acid sequence selected from the group consisting of SEQ ID NO:18, SEQ ID NO:20, SEQ ID NO:22, SEQ ID NO:24, SEQ ID NO:26, SEQ ID NO:28, SEQ ID NO:30, SEQ ID NO:55, SEQ ID NO:57, SEQ ID NO:59, and SEQ ID NO:61; or
23) comprises a fragment of the amino acid sequence of a22), wherein said fragment is a member of the vWFC domain containing protein family, or has an antigenic determinant in common with the amino acid sequence of a22); or
24) comprises a functional equivalent of a22) or a23); or
25) consists of an amino acid sequence selected from the group consisting of SEQ ID NO:18, SEQ ID NO:20, SEQ ID NO:22, SEQ ID NO:24, SEQ ID NO:26, SEQ ID NO:28, SEQ ID NO:30, SEQ ID NO:55, SEQ ID NO:57, SEQ ID NO:59, and SEQ ID NO:61; or
26) comprises a fragment of the amino acid sequence of a25), wherein said fragment is a member of the vWFC domain containing protein family, or has an antigenic determinant in common with the amino acid sequence of a25); or
27) comprises a functional equivalent of a25) or a26); or
28) comprises the functional equivalent of any of a3), a5), a8), a12), a15), a18), a21), a24), or a27), characterised in that it is homologous to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, and SEQ ID NO:61, and is a member of the vWFC domain containing protein family; or
29) comprises the fragment or functional equivalent of any of a2), a3), a5), a7), a8), a11), a12), a14), a15), a17), a18), a20), a21), a23), a24), a26), a27), or a28), wherein the fragment or functional equivalent has greater than 80% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, and SEQ ID NO:49, or with an active fragment of any of the foregoing; or
30) comprises the functional equivalent of any of a3), a5), a8), a12), a15), a18), a21), a24), a27), a28, or a29), wherein the functional equivalent exhibits significant structural homology with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, and SEQ ID NO:61; or
31) comprises the fragment of any of a2), a5), a7), a11), a14), a17), a20), a23), a26), or a29), wherein the fragment has an antigenic determinant in common with the amino acid sequence of any one of a1), a4), a5,), a6), a9), a10), a13), a16), a19), a22), or a25), which consists of 7 or more amino acid residues from the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59 or SEQ ID NO:61; and wherein
b) the purified nucleic acid molecule:
1) comprises a nucleic acid sequence encoding a polypeptide according to any one of a1)-a31); or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, and SEQ ID NO:60, or a redundant equivalent or fragment of any of the foregoing; or
3) consists of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27 SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, and SEQ ID NO:60, or a redundant equivalent or fragment of any of the foregoing; or
4) hybridizes under high stringency conditions with a nucleic acid molecule according to any one of b1) to b3).

79: The method of claim 62, wherein said method of using a composition of matter comprises the method for identification of a compound that is effective in the treatment and/or diagnosis of a disease, comprising contacting a polypeptide or a nucleic acid molecule of with one or more compounds suspected of possessing binding affinity for said polypeptide or nucleic acid molecule, and selecting a compound that binds specifically to said nucleic acid molecule or polypeptide, wherein
a) said polypeptide:
1) comprises an amino acid sequence that has an E value of 10⁻² or less when the profile below is input as a query sequence into the search program BLAST, using the default parameters specified by the NCBI (the National Center for Biotechnology Information) [Blosum 62 matrix; gap open penalty=11 and gap extension penalty=1]:

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | M | −2 | −2 | −3 | −4 | −2 | −1 | −3 | −4 | −3 | 0 | 2 | −2 | 8 | 0 | −3 | −2 | −2 | −2 | −2 | 0 |
| 2 | A | 3 | −1 | −3 | −3 | −1 | −1 | −2 | −2 | −3 | 0 | 0 | 1 | 2 | 1 | −2 | −1 | −1 | −3 | −1 | 1 |
| 3 | L | 1 | −2 | −2 | −3 | −2 | −2 | −2 | −2 | 2 | 0 | 2 | −2 | 0 | −2 | 0 | 1 | −1 | −3 | −2 | 2 |
| 4 | H | −1 | −1 | 1 | −1 | −3 | −1 | −1 | 3 | 6 | −3 | −1 | −1 | −2 | −2 | −2 | 2 | −1 | −3 | −1 | −3 |
| 5 | I | 0 | −2 | −3 | −3 | −2 | 0 | 0 | −3 | −3 | 3 | 2 | −2 | 0 | −1 | −3 | −2 | −1 | −3 | −2 | 2 |
| 6 | H | 0 | −2 | −1 | −2 | −2 | −1 | −1 | −3 | 7 | 0 | 0 | −2 | −1 | −2 | 0 | 0 | −1 | −3 | 0 | 2 |
| 7 | E | 0 | −2 | −2 | −1 | −2 | −1 | 2 | −3 | −1 | 0 | 1 | −1 | 2 | 2 | −3 | 0 | −1 | −2 | 2 | 0 |
| 8 | A | 2 | −2 | −1 | −2 | 3 | −2 | −2 | 2 | −2 | −2 | −2 | −2 | −2 | −3 | −2 | 3 | 1 | −3 | −3 | −2 |
| 9 | C | 0 | −3 | −2 | −3 | 7 | −2 | −3 | −3 | −3 | 0 | −1 | −2 | 3 | 3 | −3 | 0 | −1 | −2 | −1 | 0 |
| 10 | I | −1 | −2 | −2 | −2 | −2 | −2 | 0 | 0 | −2 | 3 | 2 | −2 | 0 | 0 | −3 | 0 | 0 | −2 | 3 | 0 |
| 11 | L | −2 | 0 | −3 | −4 | −2 | −2 | −3 | −4 | −3 | 0 | 4 | −2 | 3 | 3 | −4 | −2 | −2 | −2 | 0 | 0 |
| 12 | L | 0 | 0 | −3 | −4 | −2 | −2 | −3 | −3 | −3 | 0 | 3 | −2 | 0 | −1 | 1 | −2 | −1 | −3 | −2 | 1 |
| 13 | L | −1 | −2 | −2 | −2 | −2 | −2 | 0 | −3 | −3 | 0 | 3 | −2 | 0 | 1 | −3 | 1 | 1 | −3 | −1 | 1 |
| 14 | V | 0 | 0 | −3 | −3 | 4 | 0 | −2 | −3 | −3 | 0 | 0 | −2 | 2 | −2 | −3 | −2 | −1 | −3 | −2 | 4 |
| 15 | I | −1 | 0 | −2 | −3 | 4 | −2 | −2 | −3 | 2 | 2 | 1 | −2 | 0 | −1 | −3 | 0 | 0 | 4 | −1 | 0 |
| 16 | P | 0 | 0 | −2 | −2 | −2 | −2 | −2 | −3 | 4 | −1 | 0 | −2 | −1 | 2 | 3 | 0 | −1 | −2 | 0 | 0 |
| 17 | G | 0 | −3 | −2 | −3 | −2 | −2 | −3 | 2 | −3 | 1 | 0 | −3 | 3 | 3 | −3 | −1 | −2 | −2 | 0 | 1 |
| 18 | L | 1 | −3 | −3 | −4 | −1 | −2 | −3 | −3 | −3 | 0 | 4 | −2 | 0 | −1 | −3 | −2 | −1 | −3 | −2 | 2 |
| 19 | V | 2 | −1 | −1 | −2 | −2 | 3 | −1 | −2 | −2 | 0 | −1 | −1 | −1 | −3 | −2 | 1 | 1 | −3 | −2 | 2 |
| 20 | T | −1 | −2 | −2 | −3 | 4 | −2 | −2 | −3 | −3 | 0 | 3 | −2 | 0 | −2 | −3 | 1 | 3 | −3 | −2 | 0 |
| 21 | S | 0 | −2 | −1 | −2 | 4 | −2 | −2 | 1 | −3 | −1 | 1 | −2 | 2 | −2 | 0 | 2 | −1 | −3 | −3 | −1 |
| 22 | A | 3 | −2 | −2 | −2 | −1 | −2 | −2 | −1 | −2 | −1 | 0 | −2 | −1 | 1 | 2 | 2 | −1 | −3 | −2 | 0 |
| 23 | A | 2 | −2 | −3 | −2 | 5 | 0 | 0 | −2 | −3 | −1 | 0 | −2 | −1 | −3 | 2 | −1 | −1 | −3 | −2 | 1 |
| 24 | I | 1 | 1 | −2 | −2 | −2 | −1 | −2 | −2 | −2 | 2 | 0 | −1 | −1 | −1 | −2 | 2 | −1 | −3 | 1 | 1 |
| 25 | S | −1 | −1 | 2 | −1 | −2 | 1 | −1 | −2 | 4 | 2 | −1 | −1 | −1 | −2 | −2 | 3 | −1 | −4 | −1 | −1 |
| 26 | H | 0 | −2 | 3 | −1 | −3 | −1 | −1 | −2 | 5 | −2 | −2 | −1 | −2 | 0 | 3 | 0 | −1 | −3 | −1 | 0 |
| 27 | E | −1 | −1 | 0 | 1 | −3 | 0 | 5 | −2 | −1 | −3 | −3 | 0 | −2 | −3 | −1 | 1 | 2 | −3 | −2 | −2 |
| 28 | D | 1 | −2 | 0 | 4 | −2 | −1 | 0 | −1 | −2 | −2 | 0 | −1 | −1 | −2 | −1 | 2 | 0 | −4 | −3 | −2 |
| 29 | Y | −2 | −1 | −2 | −2 | −3 | 2 | −1 | −3 | 0 | 0 | −1 | −1 | −1 | 0 | 3 | −1 | −2 | 0 | 5 | 0 |
| 30 | P | 0 | −1 | −1 | −1 | −2 | −1 | −1 | 0 | 5 | −3 | −3 | −1 | −2 | −3 | 4 | 0 | 0 | −3 | −1 | −2 |
| 31 | A | 3 | −2 | −2 | −2 | −1 | −1 | −2 | −1 | −3 | 0 | 2 | −1 | 0 | −2 | 0 | 0 | 0 | −3 | −2 | 0 |
| 32 | D | −2 | −1 | 0 | 5 | −3 | 0 | 2 | −2 | −1 | −2 | −3 | 2 | −2 | −3 | −1 | 0 | −1 | −4 | −3 | 0 |
| 33 | E | 0 | −1 | −2 | 0 | −2 | 0 | 3 | −3 | −1 | 0 | 0 | −1 | 0 | 3 | −2 | −1 | −1 | −2 | 0 | 0 |
| 34 | G | 1 | −2 | 0 | −1 | −2 | −1 | −2 | 4 | −2 | −2 | −2 | −2 | 0 | −2 | −2 | 1 | 1 | −2 | −3 | 0 |
| 35 | D | 0 | 2 | 0 | 2 | −2 | 0 | 0 | −2 | −1 | −2 | 0 | 0 | −1 | −3 | 2 | −1 | −1 | −3 | −2 | −2 |
| 36 | Q | 0 | 3 | 0 | −1 | −2 | 3 | 0 | −2 | −1 | −2 | −2 | 0 | −1 | −3 | −1 | 1 | 3 | −3 | −2 | −2 |
| 37 | I | 0 | −2 | −3 | −2 | −1 | −2 | −2 | −2 | −3 | 1 | 0 | −2 | 0 | −2 | 4 | −1 | −1 | −3 | −2 | 2 |
| 38 | S | −1 | −1 | 0 | 1 | −2 | 0 | 2 | −2 | −1 | 2 | −1 | 1 | −1 | −2 | −2 | 2 | −1 | −3 | −2 | 0 |
| 39 | S | 3 | −1 | −1 | −1 | −1 | −1 | −1 | −1 | −2 | −1 | 0 | −1 | −1 | −2 | 2 | 3 | 0 | −3 | −2 | −1 |
| 40 | N | 0 | −2 | 2 | −1 | −3 | −1 | 1 | 3 | −1 | 0 | 0 | −1 | −1 | −3 | 1 | −1 | −1 | −3 | −2 | 0 |
| 41 | D | 1 | −2 | −1 | 3 | −2 | 0 | 2 | −2 | −2 | −1 | 0 | −1 | −1 | 0 | −2 | 0 | −1 | −3 | −2 | −1 |
| 42 | N | −1 | −1 | 2 | 1 | −3 | 0 | 3 | 0 | −1 | −2 | 0 | 0 | −1 | −2 | −2 | 1 | −1 | −3 | −2 | −2 |
| 43 | L | 1 | −3 | −3 | −3 | 4 | −2 | −3 | −3 | −1 | 0 | 1 | −3 | 0 | 3 | −3 | −2 | −2 | 0 | 5 | 0 |
| 44 | I | 1 | −1 | −1 | 0 | −2 | 0 | 2 | −2 | −2 | 1 | −1 | 1 | −1 | −2 | −2 | 2 | −1 | −4 | −2 | 0 |
| 45 | F | −2 | −3 | −2 | 1 | −2 | −2 | −2 | −3 | −1 | 2 | 0 | −3 | 0 | 4 | −3 | −2 | −2 | −1 | 4 | 1 |
| 46 | D | 1 | −2 | −1 | 2 | −2 | −1 | 0 | 0 | −2 | −3 | −3 | −1 | −2 | −3 | 4 | 1 | −1 | −4 | −3 | −2 |
| 47 | D | 0 | −2 | 0 | 5 | −3 | −1 | 0 | 1 | −2 | −3 | −4 | −1 | −3 | −3 | 2 | 1 | −1 | −4 | −3 | −3 |
| 48 | Y | −2 | 3 | −1 | −2 | −3 | 2 | −1 | −3 | 0 | −2 | −2 | 0 | −1 | 0 | −3 | −2 | −2 | 0 | 6 | −2 |
| 49 | R | −2 | 4 | 0 | −2 | −4 | 2 | 0 | −2 | 5 | −4 | −3 | 0 | −2 | −3 | 2 | −1 | −2 | −3 | −1 | −3 |
| 50 | G | −1 | −2 | 0 | 2 | −3 | −1 | 1 | 4 | −2 | −2 | −3 | −1 | −2 | −3 | −2 | 1 | −1 | −3 | −3 | 0 |
| 51 | K | −1 | 2 | 1 | −1 | −3 | 0 | 0 | 1 | −1 | −3 | −3 | 4 | −2 | −3 | −2 | 1 | −1 | −3 | −3 | −3 |
| 52 | G | −1 | −2 | −1 | 2 | 4 | −2 | −1 | 3 | −2 | −2 | −3 | −2 | −2 | 0 | −2 | 0 | 2 | −2 | −2 | −2 |
| 53 | C | −1 | −4 | −4 | −4 | 10 | −4 | −4 | −4 | −3 | −1 | −1 | −4 | −1 | 2 | −4 | −2 | −2 | −2 | −1 | −1 |
| 54 | V | −1 | −2 | −1 | 0 | −2 | −1 | 1 | −2 | −2 | 0 | 0 | −1 | 3 | −2 | −2 | 1 | 0 | −3 | −2 | 3 |
| 55 | D | 0 | −2 | 0 | 6 | −3 | 0 | 1 | −1 | −1 | −3 | −4 | −1 | −3 | −3 | −1 | 0 | −1 | −4 | −3 | −3 |
| 56 | D | −2 | −1 | 3 | 4 | −4 | 0 | 2 | 2 | −1 | −4 | −4 | −1 | −3 | −4 | −2 | 0 | −1 | −4 | −3 | −3 |
| 57 | S | 0 | −1 | 2 | 2 | −3 | 1 | 0 | 2 | −1 | −3 | −3 | −1 | −2 | −3 | −2 | 3 | 0 | −3 | −3 | −3 |
| 58 | G | 0 | 1 | 0 | −1 | −3 | −1 | −2 | 5 | −2 | −4 | −4 | −1 | −3 | −3 | −2 | 1 | −2 | −3 | −3 | −3 |
| 59 | F | −1 | −4 | −4 | −4 | −2 | −3 | −3 | −4 | −2 | 3 | 0 | −3 | 0 | 5 | −4 | −2 | −1 | −1 | 0 | 3 |
| 60 | V | −1 | −3 | −3 | −2 | −2 | −1 | 1 | −3 | −1 | 0 | 0 | −2 | 0 | 3 | −3 | −2 | −1 | −1 | 4 | 3 |
| 61 | Y | −2 | −2 | −2 | −3 | −2 | −2 | −2 | −3 | 0 | −1 | −1 | −2 | −1 | 4 | −3 | 0 | −2 | 0 | 7 | −2 |
| 62 | K | 1 | −1 | −2 | −2 | −2 | −1 | −1 | 0 | −3 | 0 | −1 | 1 | −1 | −2 | 2 | −1 | −1 | −4 | −2 | 3 |
| 63 | L | −1 | −3 | −2 | −3 | −2 | −3 | −3 | 4 | −3 | 4 | 1 | −3 | 0 | −1 | −3 | −1 | −2 | −3 | −2 | 0 |
| 64 | G | −1 | −1 | 0 | 0 | −4 | 0 | 3 | 5 | −1 | −4 | −4 | −1 | −3 | −4 | −2 | 0 | −2 | −3 | −3 | −3 |
| 65 | E | −2 | −1 | −1 | 0 | −3 | 3 | 3 | −3 | −1 | −2 | −1 | 0 | 3 | −1 | −2 | −1 | −2 | 7 | −1 | −2 |
| 66 | R | −2 | 2 | −2 | −3 | −2 | 0 | −1 | −3 | 0 | 1 | −1 | 1 | −1 | 3 | −3 | −2 | −2 | −1 | 4 | 0 |
| 67 | F | −2 | −3 | −3 | −3 | −2 | −3 | −3 | −3 | −1 | −1 | −1 | −3 | −1 | 6 | −4 | −2 | 1 | 6 | 3 | −1 |
| 68 | F | 0 | 1 | −2 | −3 | −2 | −1 | −2 | −2 | −1 | −1 | 1 | −1 | 0 | 2 | −3 | 0 | 0 | −1 | 2 | −1 |
| 69 | P | −2 | −2 | −1 | 2 | −4 | 0 | 2 | −2 | −2 | −4 | −4 | −1 | −3 | −4 | 6 | −1 | −1 | −4 | −3 | −3 |
| 70 | G | 0 | −2 | 0 | −1 | −2 | −1 | −1 | 5 | −2 | −4 | −4 | −1 | −2 | −3 | −2 | 3 | −1 | −3 | −3 | −3 |
| 71 | H | −2 | −2 | 0 | 5 | −3 | −1 | 0 | −2 | 4 | −3 | −4 | −1 | −3 | −3 | 3 | 0 | −1 | −4 | −2 | −3 |
| 72 | S | −1 | −1 | 0 | −1 | −2 | −1 | −1 | −2 | 5 | −3 | −3 | −1 | −2 | −3 | 4 | 3 | 1 | −3 | −1 | −2 |
| 73 | N | 2 | −2 | 2 | −2 | 8 | −2 | −2 | −2 | −2 | −2 | −2 | −2 | −2 | −3 | −3 | 0 | 0 | −3 | −2 | −1 |
| 74 | C | −1 | −2 | −1 | −1 | 8 | −1 | 3 | −3 | −2 | −2 | −2 | −1 | −2 | −3 | −2 | 0 | 2 | −3 | −2 | −1 |
| 75 | P | −1 | 2 | −2 | −2 | −3 | 3 | 0 | −3 | −2 | −2 | 0 | 0 | −1 | −3 | 5 | −1 | −1 | −3 | −2 | −2 |
| 76 | C | 0 | −4 | −4 | −4 | 10 | −4 | −5 | −4 | −4 | −1 | −1 | −4 | −1 | −2 | −4 | −1 | −1 | −2 | −2 | −1 |
| 77 | V | −1 | −2 | −2 | −1 | −2 | −1 | 2 | −3 | −2 | 0 | 2 | −1 | 0 | −1 | −2 | −1 | 0 | −3 | −2 | 3 |
| 78 | C | 0 | −4 | −4 | −4 | 10 | −4 | −5 | −4 | −4 | −1 | −1 | −4 | −1 | −2 | −4 | −1 | −1 | −2 | −2 | −1 |
| 79 | A | 2 | −1 | −1 | −1 | −1 | 1 | −1 | −2 | −2 | −2 | −2 | −1 | −1 | −3 | −1 | 0 | 4 | −3 | −2 | −1 |
| 80 | L | 1 | −1 | −1 | 0 | −2 | 0 | 2 | −2 | −2 | −1 | 0 | −1 | −1 | −2 | −2 | 1 | 1 | −3 | −2 | −1 |
| 81 | D | −2 | −2 | 0 | 5 | −4 | 0 | 4 | −2 | −1 | −3 | −4 | −1 | −3 | −3 | −2 | 0 | 1 | −4 | −3 | −3 |
| 82 | G | 0 | −3 | 0 | −2 | −4 | −3 | −3 | 7 | −3 | −5 | −5 | −3 | −4 | −4 | −3 | 0 | −3 | −3 | −4 | −4 |
| 83 | P | −1 | −2 | −2 | −1 | −3 | −1 | −1 | −2 | −2 | −3 | −3 | −1 | −2 | −4 | 7 | 1 | −1 | −4 | −3 | −3 |
| 84 | V | 0 | −2 | −2 | −3 | −2 | 1 | −1 | −3 | −3 | 1 | 1 | −2 | 0 | −2 | −2 | 0 | −1 | −3 | −2 | 4 |
| 85 | C | 0 | −3 | −3 | −3 | 10 | −3 | −4 | −3 | −3 | −2 | −2 | −3 | −2 | −3 | −3 | 0 | −1 | −3 | −3 | −2 |
| 86 | D | 1 | −2 | −1 | 3 | 4 | −1 | −1 | −2 | −2 | −1 | −2 | −2 | 0 | 0 | −2 | 1 | 0 | −3 | −2 | 0 |
| 87 | Q | −1 | 3 | −1 | −2 | −3 | 4 | 0 | −3 | −1 | −2 | −2 | 2 | −1 | −3 | −2 | −1 | −1 | −3 | −2 | 0 |
| 88 | P | −1 | −2 | −1 | −2 | −2 | −2 | −2 | −3 | −3 | −2 | −2 | −2 | −2 | −4 | 6 | 0 | 4 | −4 | −3 | −1 |
| 89 | E | −2 | 2 | 0 | 2 | −4 | 0 | 5 | −2 | −1 | −4 | −3 | 1 | −2 | −4 | −2 | −1 | −2 | −4 | −3 | −3 |
| 90 | C | 0 | −4 | −4 | −4 | 10 | −4 | −5 | −4 | −4 | −2 | −2 | −4 | −2 | −3 | −4 | −2 | −2 | −3 | −3 | −2 |
| 91 | P | −1 | −2 | −2 | −2 | −3 | −2 | −2 | −3 | −3 | −1 | −2 | −2 | −2 | −3 | 6 | −1 | 2 | −4 | −3 | 0 |
| 92 | K | 0 | 3 | 0 | 0 | −3 | 0 | 3 | −2 | −1 | −3 | −3 | 3 | −2 | −3 | −2 | 0 | −1 | −3 | −2 | −2 |
| 93 | I | −1 | −3 | −3 | −4 | −1 | −2 | −3 | −4 | −3 | 3 | 3 | −2 | 0 | −1 | −3 | −2 | 1 | −3 | −2 | 1 |
| 94 | H | −1 | −1 | 0 | −1 | 4 | −1 | −1 | −2 | 7 | −3 | −3 | −1 | −2 | −2 | 2 | 1 | −1 | −3 | 0 | −3 |
| 95 | P | 0 | −2 | −2 | −1 | −3 | −1 | 1 | −2 | −2 | −3 | −3 | −1 | −2 | −4 | 7 | −1 | −1 | −4 | −3 | −2 |
| 96 | K | 1 | 3 | −1 | −2 | −2 | 0 | 0 | −2 | 0 | −2 | −2 | 2 | −1 | −1 | −2 | 1 | −1 | −2 | 2 | −2 |
| 97 | C | 0 | −3 | −3 | −3 | 10 | −3 | −4 | −3 | −3 | −2 | −2 | −3 | −2 | −3 | 2 | −1 | −1 | −3 | −3 | −2 |
| 98 | T | −1 | −3 | −2 | −3 | −1 | −2 | −3 | −4 | −3 | 4 | 0 | −3 | 1 | −1 | −3 | −1 | 3 | −3 | −2 | 2 |
| 99 | K | −2 | 1 | 0 | −1 | −4 | 0 | 2 | −2 | 7 | −3 | −3 | 2 | −2 | −2 | −2 | −1 | −2 | −3 | 0 | −3 |
| 100 | V | −1 | −3 | −3 | −3 | −1 | −3 | −3 | −4 | −3 | 3 | 0 | −3 | 0 | −1 | −3 | −2 | −1 | −3 | −1 | 5 |
| 101 | E | 1 | −1 | 0 | 3 | −2 | 0 | 2 | −1 | −1 | −3 | −3 | 1 | −2 | −3 | −1 | 2 | 0 | −4 | −3 | −2 |
| 102 | H | −2 | 2 | 1 | −1 | −3 | 0 | −1 | −2 | 7 | −3 | −2 | 0 | −2 | −1 | −2 | −1 | 1 | −2 | 3 | −2 |
| 103 | N | −1 | 0 | 3 | −1 | −2 | 0 | 0 | −1 | 4 | −2 | −2 | 1 | −2 | 0 | −2 | 1 | 0 | −1 | 4 | −2 |
| 104 | G | −1 | −1 | 0 | 2 | −3 | 4 | 2 | 2 | −1 | −4 | −3 | 0 | −2 | −3 | −2 | 0 | −1 | −3 | −2 | −3 |
| 105 | C | 0 | −3 | −3 | −3 | 10 | −3 | −5 | −3 | −3 | −1 | −1 | −3 | −1 | −2 | −3 | −1 | −1 | −2 | −2 | −1 |
| 106 | C | 0 | −3 | −3 | −3 | 10 | −3 | −5 | −3 | −3 | −1 | −1 | −3 | −1 | −2 | −3 | −1 | −1 | −2 | −2 | −1 |
| 107 | P | −1 | −3 | −3 | −2 | −3 | −2 | −2 | −3 | −3 | −1 | −2 | −2 | −2 | −4 | 7 | −2 | −1 | −4 | −3 | 0 |
| 108 | E | −1 | 2 | 0 | 2 | −3 | 4 | 2 | −2 | −1 | −2 | −1 | 0 | −1 | −3 | −2 | −1 | −1 | −3 | −2 | 0 |
| 109 | C | 0 | −3 | −3 | −3 | 10 | −3 | −5 | −3 | −3 | −1 | −1 | −3 | −1 | −2 | −3 | −1 | −1 | −2 | −2 | −1 |
| 110 | K | −1 | 0 | 0 | 0 | −3 | 0 | 3 | −2 | −1 | −1 | −2 | 3 | −1 | −3 | −2 | 1 | −1 | −3 | −2 | 1 |
| 111 | E | 2 | 2 | −1 | 0 | −3 | 0 | 4 | −2 | −1 | −3 | −2 | 2 | −2 | −3 | −2 | 0 | −1 | −3 | −2 | −2 |
| 112 | V | −1 | 1 | −1 | −1 | −3 | −1 | 1 | 1 | −2 | 1 | −1 | 1 | −1 | −2 | −2 | −1 | −1 | −3 | −2 | 2 |
| 113 | K | 0 | 0 | 0 | −1 | −3 | 0 | 0 | 4 | −2 | −4 | −3 | 4 | −2 | −3 | −2 | 1 | −1 | −3 | −3 | −3 |
| 114 | N | −2 | 0 | 6 | 0 | −3 | 0 | 0 | 0 | 0 | −3 | −3 | 2 | −2 | −3 | −2 | 0 | 0 | −4 | −2 | −3 |
| 115 | F | −1 | −3 | −3 | −3 | −2 | −2 | −2 | −3 | 0 | 0 | 0 | −2 | 0 | 4 | −3 | −2 | −1 | 0 | 6 | 1 |
| 116 | C | 0 | −3 | −3 | −3 | 10 | −3 | −5 | −3 | −3 | −1 | −1 | −3 | −1 | −2 | −3 | −1 | −1 | −2 | −2 | −1 |
| 117 | E | −1 | −1 | 0 | 3 | −3 | 0 | 4 | −2 | −1 | −2 | 0 | 0 | −2 | −3 | −2 | 0 | 1 | −3 | −3 | −2 |
| 118 | Y | −2 | −2 | −2 | −2 | −3 | −1 | 1 | −3 | 0 | −1 | −1 | −2 | −1 | 5 | −3 | −2 | −2 | 0 | 6 | −2 |
| 119 | H | −2 | 5 | 2 | −2 | −3 | 0 | −1 | −2 | 4 | −2 | −1 | 0 | 3 | −2 | −2 | −1 | −1 | −3 | −1 | −2 |
| 120 | G | −1 | −2 | 2 | −1 | −3 | −2 | −2 | 6 | −2 | −4 | −4 | −2 | −3 | −3 | −2 | 0 | −2 | −3 | −3 | −3 |
| 121 | K | −1 | 4 | −1 | −2 | −3 | 0 | 0 | −3 | −1 | −1 | −2 | 5 | −1 | −3 | −2 | −1 | −1 | −3 | −2 | 0 |
| 122 | N | −1 | −2 | 2 | −2 | −2 | −2 | −2 | −2 | −2 | 2 | 0 | −2 | 0 | −2 | −2 | 0 | 4 | −3 | −2 | 1 |
| 123 | Y | −2 | −3 | −3 | −3 | −2 | −2 | −3 | −3 | 0 | −1 | −1 | −3 | −1 | 4 | −4 | −2 | −2 | 1 | 8 | −1 |
| 124 | K | 0 | 1 | 0 | −1 | −3 | 2 | 2 | −2 | 4 | −3 | −2 | 2 | −1 | −1 | −2 | −1 | −2 | −2 | 3 | −2 |
| 125 | I | −1 | −2 | 2 | −2 | −2 | −2 | −2 | −2 | −2 | 2 | 1 | −2 | 0 | −1 | −2 | 0 | 1 | −3 | −2 | 2 |
| 126 | L | −1 | −2 | 2 | −2 | −2 | −2 | −2 | 3 | −2 | 0 | 3 | −2 | 0 | −1 | −3 | −1 | −1 | −3 | −2 | −1 |
| 127 | E | −1 | 0 | 0 | 1 | −4 | 3 | 6 | −2 | 0 | −3 | −3 | 0 | −2 | −3 | −1 | 0 | −1 | −3 | −2 | −2 |
| 128 | E | −1 | −1 | 2 | 0 | −3 | 0 | 4 | −2 | 0 | −3 | −3 | 0 | −2 | −1 | −2 | 0 | 1 | −2 | 3 | −2 |
| 129 | F | −2 | −3 | −3 | −3 | −2 | −3 | −3 | −3 | −1 | 0 | 0 | −3 | 0 | 7 | −5 | −2 | −2 | 0 | 2 | −1 |
| 130 | K | −1 | 1 | 2 | −1 | −3 | 2 | 0 | −2 | −1 | −1 | 0 | 2 | 1 | −2 | −2 | −1 | −1 | −3 | −2 | 0 |
| 131 | P | −1 | −3 | −3 | −3 | −2 | −2 | −2 | −3 | −3 | 1 | 2 | −2 | 0 | −2 | 3 | −2 | 0 | −3 | −2 | 3 |
| 132 | S | 0 | 0 | 1 | 2 | 2 | −1 | 0 | −1 | −1 | −3 | −3 | −1 | −2 | −3 | 1 | 4 | 1 | −4 | −3 | −2 |
| 133 | P | −1 | −2 | −2 | −1 | −3 | −1 | 1 | −3 | −2 | −2 | −3 | −1 | −2 | −4 | 7 | −1 | −1 | −4 | −3 | 0 |
| 134 | C | 0 | −3 | −3 | −3 | 10 | −3 | −4 | −3 | −3 | −1 | −1 | −3 | −1 | −2 | −3 | −1 | 0 | −2 | −2 | −1 |
| 135 | E | −1 | 1 | −1 | 0 | −3 | 0 | 5 | −3 | −1 | −1 | 1 | 0 | −1 | −2 | −2 | −1 | −1 | −3 | −2 | −1 |
| 136 | W | −1 | 2 | −1 | −2 | −3 | 2 | 0 | −2 | 3 | −2 | 0 | 1 | −1 | −2 | −2 | 1 | −1 | 4 | −1 | −2 |
| 137 | C | 0 | 0 | −3 | −3 | 10 | −3 | −4 | −3 | −3 | 0 | −1 | −3 | −1 | −2 | −3 | −1 | 0 | −3 | −2 | −1 |
| 138 | R | −1 | 4 | −1 | −2 | −2 | 0 | −1 | −3 | −1 | 2 | −1 | 2 | −1 | −1 | −2 | −1 | 1 | −2 | 1 | 0 |
| 139 | C | 0 | −4 | −4 | −4 | 10 | −4 | −5 | −4 | −4 | −1 | −1 | −4 | −1 | −2 | −4 | −1 | −1 | −2 | −2 | −1 |
| 140 | E | −1 | −1 | 0 | 2 | −3 | 0 | 5 | −3 | −1 | −2 | 0 | 0 | −1 | −3 | −2 | 0 | 1 | −3 | −2 | −2 |
| 141 | P | 1 | −2 | 1 | −2 | −2 | −1 | −1 | −2 | −2 | 0 | 0 | −1 | −1 | −2 | 2 | 1 | 1 | −3 | −2 | 0 |
| 142 | S | −1 | −1 | 4 | 2 | −3 | 1 | 0 | 2 | −1 | −3 | −3 | −1 | −2 | −3 | −2 | 2 | −1 | −4 | −3 | −3 |
| 143 | N | −1 | 1 | 2 | −1 | −3 | −1 | −1 | 4 | −1 | −4 | −4 | 1 | −2 | −3 | −2 | 1 | −1 | −3 | −3 | −3 |
| 144 | E | −1 | 0 | −1 | 0 | −3 | 0 | 4 | −3 | −1 | 1 | −1 | 0 | −1 | −2 | −2 | −1 | 1 | −3 | −2 | 1 |
| 145 | V | 3 | −2 | −3 | −3 | −1 | −2 | −2 | −2 | −3 | 0 | 0 | −2 | 0 | −2 | −2 | −1 | −1 | −3 | −2 | 3 |
| 146 | H | −2 | 1 | −2 | −3 | −2 | −1 | −2 | −3 | 3 | 0 | 2 | −1 | 0 | 2 | −3 | −2 | −2 | −1 | 4 | −1 |
| 147 | C | −1 | 0 | −3 | −3 | 10 | −3 | −3 | −3 | −3 | −2 | −2 | −2 | −2 | −3 | 1 | −1 | −1 | −3 | −3 | −2 |
| 148 | V | 0 | −2 | −1 | −2 | −1 | −1 | −2 | −2 | −3 | 0 | 1 | −1 | 0 | −2 | −2 | 2 | 3 | −3 | −2 | 2 |
| 149 | V | −1 | −3 | −3 | −3 | 1 | −2 | −2 | −3 | −3 | 2 | 0 | −2 | 0 | −2 | 2 | −2 | −1 | −4 | −2 | 5 |
| 150 | A | 3 | −2 | −2 | −2 | 2 | −1 | −1 | −1 | −2 | 0 | −1 | −1 | −1 | −2 | −2 | 2 | 0 | −3 | −2 | 2 |
| 151 | D | 1 | −2 | 0 | 5 | −2 | 1 | 0 | 0 | −2 | −3 | −3 | −1 | −2 | −3 | −2 | 1 | −1 | −4 | −3 | −2 |
| 152 | C | −1 | −3 | −3 | −3 | 10 | −3 | −4 | 0 | −3 | −2 | −2 | −3 | −1 | 1 | −4 | −1 | −2 | −2 | −1 | −2 |
| 153 | A | 2 | −2 | −2 | −2 | −2 | −1 | 0 | −2 | −2 | −1 | 0 | −1 | −1 | 1 | 4 | −1 | −1 | −3 | −1 | −1 |
| 154 | V | 2 | −1 | −2 | −1 | −2 | 4 | 1 | −2 | −2 | −1 | −2 | 0 | −1 | −3 | 3 | −1 | −1 | −3 | −2 | 0 |
| 155 | P | −1 | −2 | −1 | 1 | −2 | −2 | −1 | −2 | −2 | 1 | 0 | −2 | −1 | −2 | 4 | 0 | 2 | −4 | −3 | 0 |
| 156 | E | −2 | 1 | −1 | 0 | −4 | 0 | 3 | −3 | 2 | −3 | −3 | 1 | −2 | 2 | 3 | −1 | −2 | −3 | −1 | −3 |
| 157 | C | −1 | −3 | −3 | −3 | 9 | 1 | −3 | −3 | −3 | −2 | −1 | −3 | −1 | −2 | −3 | −2 | −2 | 5 | −2 | −2 |
| 158 | V | −1 | 0 | −2 | −3 | −2 | 0 | −2 | −3 | −3 | 2 | 0 | −2 | 0 | 1 | −2 | −2 | 1 | −2 | −1 | 4 |
| 159 | N | −2 | 1 | 4 | 4 | −3 | 0 | 0 | −1 | 0 | −3 | −3 | −1 | −2 | −1 | −2 | 0 | −1 | 3 | 3 | −3 |
| 160 | P | −1 | −2 | −2 | −1 | −3 | −1 | −1 | −2 | −2 | −1 | −3 | 1 | −2 | −4 | 7 | 0 | −1 | −4 | −3 | −2 |
| 161 | V | −1 | −3 | −3 | −3 | −2 | 0 | 0 | −4 | −3 | 4 | 0 | −2 | 0 | 2 | −3 | −2 | −1 | −2 | 0 | 3 |
| 162 | Y | −2 | 1 | −1 | −3 | −3 | −1 | −1 | −3 | 4 | −2 | 0 | −1 | −1 | 0 | −3 | 0 | −2 | 0 | 6 | −2 |
| 163 | E | −1 | −1 | −1 | 0 | −3 | 3 | 4 | −3 | −1 | −2 | −1 | 0 | −1 | −3 | 3 | 0 | 0 | −3 | −2 | −2 |
| 164 | P | −1 | −1 | −2 | −1 | −3 | 0 | 1 | −3 | −1 | −2 | −1 | 0 | −2 | −2 | 5 | −1 | −1 | −2 | 2 | −2 |
| 165 | E | −1 | −2 | 1 | 2 | −3 | −1 | 1 | 3 | −1 | −3 | −3 | −1 | −3 | −1 | −2 | 0 | −1 | −2 | 2 | −3 |
| 166 | Q | −2 | 0 | −1 | −1 | −3 | 4 | 2 | −3 | 3 | −3 | 0 | 2 | −1 | −2 | −2 | −1 | −2 | 5 | 0 | −2 |
| 167 | C | 0 | −3 | −3 | −4 | 9 | −3 | −4 | −4 | −3 | 0 | 0 | −3 | −1 | −2 | −3 | −2 | −1 | −2 | −2 | 0 |
| 168 | C | 0 | −3 | −3 | −3 | 9 | −3 | −3 | −3 | −3 | 0 | −1 | −3 | −1 | −2 | −3 | 0 | −1 | −3 | −2 | 1 |
| 169 | P | 0 | −2 | −3 | −2 | −3 | −1 | −2 | −2 | −3 | −2 | 0 | −1 | −1 | −3 | 7 | −1 | −1 | −4 | −3 | −1 |
| 170 | V | −1 | −2 | −2 | −2 | −2 | −1 | 1 | −3 | −2 | 3 | 0 | 1 | 0 | −2 | −2 | −2 | −1 | −3 | −2 | 4 |
| 171 | C | −1 | −2 | −3 | −3 | 9 | −2 | −3 | −3 | −3 | −1 | 0 | 1 | −1 | −2 | −3 | −1 | −1 | −3 | −2 | −1 |
| 172 | K | −1 | 1 | 0 | −1 | −3 | 0 | 0 | −2 | −1 | −3 | −2 | 6 | −1 | −3 | −1 | 0 | −1 | −3 | −2 | −2 |
| 173 | N | 2 | −1 | 3 | 2 | −2 | −1 | 0 | 2 | −1 | −3 | −3 | −1 | −2 | −3 | −2 | 1 | −1 | −4 | −3 | −2 |
| 174 | G | 0 | −1 | 0 | −1 | −3 | −2 | −2 | 5 | −2 | −4 | −3 | 0 | −3 | −3 | −2 | 0 | −2 | −2 | −3 | −3 |
| 175 | P | −1 | −2 | 1 | −1 | −3 | −1 | 0 | −2 | −2 | −3 | −3 | −1 | −2 | −4 | 6 | −1 | −1 | −4 | −3 | −2 |
| 176 | N | −1 | −1 | 5 | 0 | −3 | −1 | −1 | −1 | 0 | −3 | −3 | −1 | −2 | −2 | −2 | 1 | 0 | 6 | −1 | −3 |
| 177 | C | −1 | −2 | −2 | −2 | 8 | −2 | −2 | −3 | 3 | −2 | −2 | −2 | −2 | −2 | 1 | −1 | 0 | −3 | −1 | −2 |
| 178 | F | −2 | −2 | −1 | −2 | −3 | −2 | −2 | 1 | 4 | −2 | −2 | 0 | −1 | 4 | 0 | −1 | −2 | 0 | 3 | −2 |
| 179 | A | 2 | −2 | −2 | −3 | −1 | −2 | −2 | 2 | −3 | 0 | 1 | −2 | 0 | 0 | −2 | −1 | −1 | −3 | −1 | 1 |
| 180 | G | 0 | −1 | 0 | 0 | −2 | −1 | 1 | 3 | −2 | −2 | 0 | −1 | −1 | −2 | 1 | 1 | 1 | −3 | −2 | −1 |
| 181 | T | 0 | −2 | −1 | −1 | −1 | −1 | −1 | −2 | −2 | 1 | −1 | −1 | −1 | −2 | 1 | 0 | 5 | −3 | −2 | 0 |
| 182 | T | 1 | −1 | 0 | −1 | −1 | 2 | 0 | −1 | −2 | −1 | 0 | −1 | 0 | −2 | −1 | 1 | 3 | −3 | −2 | −1 |
| 183 | I | 1 | −3 | −2 | −3 | −1 | −2 | −2 | −2 | −3 | 3 | 0 | −2 | 0 | −1 | −2 | 0 | 0 | −3 | −1 | 3 |
| 184 | I | −1 | −3 | −3 | −3 | −2 | −3 | −3 | 1 | −3 | 5 | 0 | −3 | 0 | 2 | −3 | −2 | −1 | −2 | 0 | 1 |
| 185 | P | −1 | −2 | −2 | −1 | −3 | 0 | 1 | −2 | −2 | −2 | 0 | −1 | −1 | −3 | 6 | −1 | −1 | −4 | −3 | −2 |
| 186 | A | 4 | 1 | −2 | −2 | 0 | −1 | −1 | 0 | −2 | −1 | −1 | 0 | −1 | −2 | −1 | 0 | 0 | −3 | −2 | 0 |
| 187 | G | 0 | −2 | 0 | −1 | −3 | −2 | −2 | 6 | −2 | −2 | −3 | −2 | −2 | −3 | −2 | 0 | −2 | −2 | −3 | 0 |
| 188 | I | −1 | 2 | −1 | 0 | −3 | 0 | 3 | −3 | −1 | 2 | −1 | 0 | −1 | −2 | 1 | −1 | −1 | −3 | −2 | 0 |
| 189 | E | −1 | −1 | −1 | 0 | −3 | 0 | 4 | −2 | −1 | 0 | −2 | 0 | −1 | −3 | 3 | −1 | −1 | −3 | −2 | −1 |
| 190 | V | 0 | 0 | −1 | 1 | −2 | −1 | −1 | −2 | −2 | 0 | 0 | −1 | 0 | −2 | −2 | −1 | 2 | −3 | −2 | 3 |
| 191 | K | −1 | 0 | −1 | −1 | −3 | 0 | 1 | −2 | −1 | −2 | −2 | 4 | −1 | −2 | −2 | −1 | −1 | 7 | −1 | 0 |
| 192 | V | 0 | −1 | −1 | −2 | −2 | 1 | 0 | −3 | −2 | 0 | 0 | 1 | 0 | −2 | −2 | −1 | 2 | −3 | −1 | 3 |
| 193 | D | −2 | −2 | 0 | 6 | −3 | 0 | 1 | −1 | −1 | −3 | −4 | −1 | −3 | −3 | −1 | 0 | 1 | −4 | −3 | −3 |
| 194 | E | 1 | −1 | −1 | 1 | −2 | 0 | 4 | −2 | −1 | −1 | −2 | 0 | −1 | −2 | −1 | 0 | −1 | −3 | −2 | 1 |
| 195 | C | 0 | −3 | −3 | −3 | 10 | −3 | −4 | −3 | −3 | −1 | −1 | −3 | −1 | −2 | −3 | −1 | −1 | −2 | −2 | −1 |
| 196 | N | −1 | −1 | 3 | −1 | −2 | −1 | −1 | −1 | −1 | −1 | −1 | −1 | −1 | 2 | −2 | 0 | 4 | −2 | 0 | −1 |
| 197 | I | −1 | −1 | −2 | −3 | 5 | −2 | −2 | −3 | −3 | 4 | 0 | 1 | 0 | −1 | −3 | −1 | −1 | −3 | −1 | 1 |
| 198 | C | 0 | −3 | −2 | −3 | 9 | −3 | −3 | −3 | −3 | −1 | −1 | −3 | −1 | −2 | −3 | −1 | 1 | −2 | −2 | −1 |
| 199 | H | −2 | 2 | 0 | −2 | −3 | 0 | 0 | −2 | 7 | −3 | −2 | 0 | −2 | 0 | −2 | −1 | −2 | −1 | 3 | −3 |
| 200 | C | 0 | −3 | −3 | −3 | 10 | −3 | −4 | −3 | −3 | −1 | −1 | −3 | −1 | −2 | −3 | −1 | −1 | −2 | −2 | −1 |
| 201 | H | −1 | 0 | 0 | −1 | −2 | 0 | 0 | −2 | 7 | −2 | −2 | −1 | −2 | −1 | −2 | 0 | 3 | −2 | 0 | −2 |
| 202 | N | −2 | −1 | 4 | 3 | −3 | 0 | 0 | −1 | 0 | −2 | −2 | −1 | −2 | 0 | −2 | 0 | −1 | −1 | 4 | −2 |
| 203 | G | 0 | −1 | 0 | 0 | −3 | 0 | 3 | 5 | −1 | −4 | −4 | −1 | −3 | −3 | −2 | 0 | −2 | −2 | −3 | −3 |
| 204 | D | −1 | 0 | 0 | 4 | −3 | 3 | 3 | −2 | 0 | −3 | −3 | 0 | −2 | −3 | −1 | 0 | −1 | −3 | −2 | −2 |
| 205 | W | −1 | −2 | 0 | 3 | −3 | −1 | 0 | 3 | −2 | −3 | −3 | −2 | −2 | −1 | −2 | −1 | −2 | 8 | 0 | −3 |
| 206 | W | −2 | −3 | −2 | −3 | −2 | −2 | −3 | 3 | −2 | −3 | −3 | −3 | −1 | 0 | −3 | −2 | −2 | 11 | 0 | −3 |
| 207 | K | −1 | 3 | 0 | −2 | −3 | 0 | 0 | −2 | 0 | −2 | −2 | 3 | −1 | 0 | −2 | −1 | −1 | −1 | 4 | −2 |
| 208 | P | −1 | −1 | −1 | −1 | −2 | 3 | 0 | −3 | −1 | −1 | 1 | 0 | 0 | −2 | 4 | −1 | −1 | −3 | −2 | −1 |
| 209 | A | 5 | −1 | −2 | −2 | 0 | −1 | −1 | 0 | −2 | −1 | −1 | −1 | −1 | −2 | −1 | 0 | 0 | −3 | −2 | 0 |
| 210 | Q | −1 | 0 | −1 | −1 | −2 | 3 | 0 | −2 | −1 | −1 | 0 | 0 | 4 | −1 | −1 | 0 | 2 | −2 | −1 | 0 |
| 211 | C | 0 | −3 | −3 | −3 | 10 | −3 | −4 | −3 | −3 | −1 | −1 | −3 | −1 | −2 | −3 | −1 | −1 | −2 | −2 | −1 |
| 212 | S | 0 | −1 | 0 | 0 | −1 | 0 | 0 | −1 | −1 | −2 | −2 | 0 | −1 | −2 | −1 | 4 | 3 | −3 | −2 | −1 |
| 213 | K | −1 | 5 | 0 | −2 | −3 | 0 | 0 | −2 | 0 | −3 | −2 | 4 | −1 | −3 | −2 | −1 | −1 | −3 | −2 | −3 |
| 214 | R | −1 | 3 | 0 | −2 | −2 | 0 | −1 | −3 | 6 | −1 | 1 | 0 | 0 | −1 | −2 | −1 | −1 | −2 | 0 | −1 |
| 215 | E | −1 | 0 | 0 | 1 | −4 | 1 | 5 | −2 | 0 | −3 | −3 | 3 | −2 | −3 | −1 | 0 | −1 | −3 | −2 | −2 |
| 216 | C | 0 | −2 | 3 | −1 | 9 | −2 | −2 | −2 | −1 | −1 | −1 | −2 | −1 | −2 | −3 | 0 | −1 | −3 | −2 | −1 |
| 217 | Q | −1 | 2 | −1 | −1 | 6 | 3 | 0 | −2 | −1 | −2 | −2 | 1 | −1 | −3 | −2 | 0 | −1 | −2 | −2 | −2 |
| 218 | G | −1 | 0 | 3 | 0 | −3 | 3 | 0 | 3 | 0 | −3 | −3 | 0 | −1 | −3 | −2 | 0 | −1 | −3 | −2 | −3 |
| 219 | K | −1 | 0 | 0 | −2 | −2 | 0 | 0 | −2 | 5 | 0 | 0 | 3 | 3 | −1 | −2 | −1 | −1 | −2 | 0 | 0 |
| 220 | Q | −1 | 0 | 0 | 0 | −3 | 4 | 4 | −2 | 0 | −3 | −2 | 0 | −1 | −3 | −1 | 0 | −1 | −2 | −1 | −2 |
| 221 | T | 0 | −1 | 0 | −1 | −2 | −1 | −1 | 4 | −2 | −2 | −2 | −1 | −2 | −2 | −1 | 0 | 4 | −2 | −2 | −1 |
| 222 | V | 0 | −2 | −2 | −3 | −1 | −1 | −2 | −3 | −2 | 2 | 1 | −1 | 4 | 0 | −2 | −1 | 0 | −2 | −1 | 3; or |

2) comprises a fragment of the amino acid sequence of a1), wherein said fragment is a member of the vWFC domain containing protein family, or has an antigenic determinant in common with the amino acid sequence of a1); or
3) comprises a functional equivalent of the amino acid sequence of a1) or a2); or
4) consists of the amino acid sequence of a1); or
5) is any of a1)-a4), wherein the polypeptide has a maximum threshold E value of 10⁻²; or
6) comprises an amino acid sequence satisfying the consensus amino acid sequence [GTDFC](0,1)-[CF](0,1)-[VMSED](0,1)-[DEA](0,1)-[DENG](0,1)-[SQNDG](0,1)-[SGR](0,1)-[FIV](0,1)-[VYFE](0,1)-[YFS](0,1)-[KVAGP](0,1)-[LIG](0,1)-[GE](0,1)-[EWQM](0,1)-[RKYFQVI](0,1)-[FYWT](0,1)-[FALYRTS](0,1)-[PED](0,1)-[GS](0,1)-[HPDS](0,1)-[STHP](0,1)-[CNAT](0,1)-[CTE](0,1)-[PQRL](0,1)-C(0,1)-[VELT](0,1)-C(0,1)-[TAQ](0,1)-[ELATSD](0,1)-[EDT]-G-[PS]-[VLAQS]-[CS]-[DAMSTFCV]-[QRKV]-R(0,1)-[PT]-[ERDK]-C-[PTV]-[KERSA]-[LIVT]-[HPSC]-[PAE]-[KRASY]-[CP]-[TIVM]-[HKRE]-[VI]-[DESAKP]-[HTNRYG]-[NSTYHK](0,1)-[PA](0,1)-[TG](0,1)-[QGDES]-C-C-[PV]-[EQRDLV]-C; or
7) comprises a fragment of the amino acid sequence of a6), wherein said fragment is a member of the vWFC domain containing protein family, or has an antigenic determinant in common with the polypeptides of a6); or
8) comprises a functional equivalent of a6) or a7); or
9) consists of an amino acid sequence satisfying the consensus amino acid sequence[GTDFC](0,1)-[CF](0,1)-[VMSED](0,1)-[DEA](0,1)-[DENG](0,1)-[SQNDG](0,1)-[SGR](0,1)-[FIV](0,1)-[VYFE](0,1)-[YFS](0,1)-[KVAGP](0,1)-[LIG](0,1)-[GE](0,1)-[EWQM](0,1)-[RKYFQVI](0,1)-[FYWT](0,1)-[FALYRTS](0,1)-[PED](0,1)-[GS](0,1)-[HPDS](0,1)-[STHP](0,1)-[CNAT](0,1)-[CTE](0,1)-[PQRL](0,1)-C(0,1)-[VELT](0,1)-C(0,1)-[TAQ](0,1)-[ELATSD](0,1)-[EDT]-G-[PS]-[VLAQS]-[CS]-[DAMSTFCV]-[QRKV]-R(0,1)-[PT]-[ERDK]-C-[PTV]-[KERSA]-[LIVT]-[HPSC]-[PAE]-[KRASY]-[CP]-[TIVM]-[HKRE]-[VI]-[DESAKP]-[HTNRYG]-[NSTYHK](0,1)-[PA](0,1)-[TG](0,1)-[QGDES]-C-C-[PV]-[EQRDLV]-C; or
10) comprises an amino acid sequence selected from the group consisting of SEQ ID NO:2, SEQ ID NO:4, SEQ ID NO:39, SEQ ID NO:41, SEQ ID NO:43, and SEQ ID NO:45; or
11) comprises a fragment of the amino acid sequence of a10), wherein said fragment is a member of the vWFC domain containing protein family, or has an antigenic determinant in common with the amino acid sequence of a10); or
12) comprises a functional equivalent of a10) or a11); or
13) consists of an amino acid sequence selected from the group consisting of SEQ ID NO:2, SEQ ID NO:4, SEQ ID NO:39, SEQ ID NO:41, SEQ ID NO:43, and SEQ ID NO:45; or
14) comprises a fragment of the amino acid sequence of a13), wherein said fragment is a member of the vWFC domain containing protein family, or has an antigenic determinant in common with the amino acid sequence of a13); or
15) comprises a functional equivalent of a13) or a14); or
16) comprises an amino acid sequence selected from the group consisting of SEQ ID NO:6, SEQ ID NO:8, SEQ ID NO:10, SEQ ID NO:12, SEQ ID NO:14, SEQ ID NO:16, SEQ ID NO:47, SEQ ID NO:49, SEQ ID NO:51, and SEQ ID NO:53; or
17) comprises a fragment of the amino acid sequence of a16), wherein said fragment is a member of the vWFC domain containing protein family, or has an antigenic determinant in common with the amino acid sequence of a16); or
18) comprises a functional equivalent of a16) or a17); or
19) consists of an amino acid sequence selected from the group consisting of SEQ ID NO:6, SEQ ID NO:8, SEQ ID NO:10, SEQ ID NO:12, SEQ ID NO:14, SEQ ID NO:16, SEQ ID NO:47, SEQ ID NO:49, SEQ ID NO:51, and SEQ ID NO:53; or
20) comprises a fragment of the amino acid sequence of a19), wherein said fragment is a member of the vWFC domain containing protein family, or has an antigenic determinant in common with the amino acid sequence of a19); or
21) comprises a functional equivalent of a19) or a20); or
22) comprises the amino acid sequence selected from the group consisting of SEQ ID NO:18, SEQ ID NO:20, SEQ ID NO:22, SEQ ID NO:24, SEQ ID NO:26, SEQ ID NO:28, SEQ ID NO:30, SEQ ID NO:55, SEQ ID NO:57, SEQ ID NO:59, and SEQ ID NO:61; or
23) comprises a fragment of the amino acid sequence of a22), wherein said fragment is a member of the vWFC domain containing protein family, or has an antigenic determinant in common with the amino acid sequence of a22); or
24) comprises a functional equivalent of a22) or a23); or
25) consists of an amino acid sequence selected from the group consisting of SEQ ID NO:18, SEQ ID NO:20, SEQ ID NO:22, SEQ ID NO:24, SEQ ID NO:26, SEQ ID NO:28, SEQ ID NO:30, SEQ ID NO:55, SEQ ID NO:57, SEQ ID NO:59, and SEQ ID NO:61; or
26) comprises a fragment of the amino acid sequence of a25), wherein said fragment is a member of the vWFC domain containing protein family, or has an antigenic determinant in common with the amino acid sequence of a25); or
27) comprises a functional equivalent of a25) or a26); or
28) comprises the functional equivalent of any of a3), a5), a8), a12), a15), a18), a21), a24), or a27), characterised in that it is homologous to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, and SEQ ID NO:61, and is a member of the vWFC domain containing protein family; or
29) comprises the fragment or functional equivalent of any of a2), a3), a5), a7), a8), a11), a12), a14), a15), a17), a18), a20), a21), a23), a24), a26), a27), or a28), wherein the fragment or functional equivalent has greater than 80% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, and SEQ ID NO:49, or with an active fragment of any of the foregoing; or
30) comprises the functional equivalent of any of a3), a5), a8), a12), a15), a18), a21), a24), a27), a28, or a29), wherein the functional equivalent exhibits significant structural homology with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, and SEQ ID NO:61; or
31) comprises the fragment of any of a2), a5), a7), a11), a14), a17), a20), a23), a26), or a29), wherein the fragment has an antigenic determinant in common with the amino acid sequence of any one of a1), a4), a5,), a6), a9), a10), a13), a16), a19), a22), or a25), which consists of 7 or more amino acid residues from the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59 or SEQ ID NO:61; and wherein
b) said purified nucleic acid molecule:
1) comprises a nucleic acid sequence encoding a polypeptide according to any one of a1)-a31); or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, and SEQ ID NO:60, or a redundant equivalent or fragment of any of the foregoing; or
3) consists of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27 SEQ ID NO:29, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, and SEQ ID NO:60, or a redundant equivalent or fragment of any of the foregoing; or
4) hybridizes under high stringency conditions with a nucleic acid molecule according to any one of b1) to b3).

80: The method of claim 62, wherein said method of using a composition of matter comprises the method for screening candidate compounds, comprising contacting a non-human transgenic animal with a candidate compound and determining the effect of the compound on the disease of the transgenic animal, wherein the transgenic animal has been transformed to express higher, lower, or absent levels of a polypeptide, wherein the polypeptide:
a) comprises an amino acid sequence that has an E value of 10⁻² or less when the profile below is input as a query sequence into the search program BLAST, using the default parameters specified by the NCBI (the National Center for Biotechnology Information) [Blosum 62 matrix; gap open penalty=11 and gap extension penalty=1]:

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | M | −2 | −2 | −3 | −4 | −2 | −1 | −3 | −4 | −3 | 0 | 2 | −2 | 8 | 0 | −3 | −2 | −2 | −2 | −2 | 0 |
| 2 | A | 3 | −1 | −3 | −3 | −1 | −1 | −2 | −2 | −3 | 0 | 0 | 1 | 2 | 1 | −2 | −1 | −1 | −3 | −1 | 1 |
| 3 | L | 1 | −2 | −2 | −3 | −2 | −2 | −2 | −2 | 2 | 0 | 2 | −2 | 0 | −2 | 0 | 1 | −1 | −3 | −2 | 2 |
| 4 | H | −1 | −1 | 1 | −1 | −3 | −1 | −1 | 3 | 6 | −3 | −1 | −1 | −2 | −2 | −2 | 2 | −1 | −3 | −1 | −3 |
| 5 | I | 0 | −2 | −3 | −3 | −2 | 0 | 0 | −3 | −3 | 3 | 2 | −2 | 0 | −1 | −3 | −2 | −1 | −3 | −2 | 2 |
| 6 | H | 0 | −2 | −1 | −2 | −2 | −1 | −1 | −3 | 7 | 0 | 0 | −2 | −1 | −2 | 0 | 0 | −1 | −3 | 0 | 2 |
| 7 | E | 0 | −2 | −2 | −1 | −2 | −1 | 2 | −3 | −1 | 0 | 1 | −1 | 2 | 2 | −3 | 0 | −1 | −2 | 2 | 0 |
| 8 | A | 2 | −2 | −1 | −2 | 3 | −2 | −2 | 2 | −2 | −2 | −2 | −2 | −2 | −3 | −2 | 3 | 1 | −3 | −3 | −2 |
| 9 | C | 0 | −3 | −2 | −3 | 7 | −2 | −3 | −3 | −3 | 0 | −1 | −2 | 3 | 3 | −3 | 0 | −1 | −2 | −1 | 0 |
| 10 | I | −1 | −2 | −2 | −2 | −2 | −2 | 0 | 0 | −2 | 3 | 2 | −2 | 0 | 0 | −3 | 0 | 0 | −2 | 3 | 0 |
| 11 | L | −2 | 0 | −3 | −4 | −2 | −2 | −3 | −4 | −3 | 0 | 4 | −2 | 3 | 3 | −4 | −2 | −2 | −2 | 0 | 0 |
| 12 | L | 0 | 0 | −3 | −4 | −2 | −2 | −3 | −3 | −3 | 0 | 3 | −2 | 0 | −1 | 1 | −2 | −1 | −3 | −2 | 1 |
| 13 | L | −1 | −2 | −2 | −2 | −2 | −2 | 0 | −3 | −3 | 0 | 3 | −2 | 0 | 1 | −3 | 1 | 1 | −3 | −1 | 1 |
| 14 | V | 0 | 0 | −3 | −3 | 4 | 0 | −2 | −3 | −3 | 0 | 0 | −2 | 2 | −2 | −3 | −2 | −1 | −3 | −2 | 4 |
| 15 | I | −1 | 0 | −2 | −3 | 4 | −2 | −2 | −3 | 2 | 2 | 1 | −2 | 0 | −1 | −3 | 0 | 0 | 4 | −1 | 0 |
| 16 | P | 0 | 0 | −2 | −2 | −2 | −2 | −2 | −3 | 4 | −1 | 0 | −2 | −1 | 2 | 3 | 0 | −1 | −2 | 0 | 0 |
| 17 | G | 0 | −3 | −2 | −3 | −2 | −2 | −3 | 2 | −3 | 1 | 0 | −3 | 3 | 3 | −3 | −1 | −2 | −2 | 0 | 1 |
| 18 | L | 1 | −3 | −3 | −4 | −1 | −2 | −3 | −3 | −3 | 0 | 4 | −2 | 0 | −1 | −3 | −2 | −1 | −3 | −2 | 2 |
| 19 | V | 2 | −1 | −1 | −2 | −2 | 3 | −1 | −2 | −2 | 0 | −1 | −1 | −1 | −3 | −2 | 1 | 1 | −3 | −2 | 2 |
| 20 | T | −1 | −2 | −2 | −3 | 4 | −2 | −2 | −3 | −3 | 0 | 3 | −2 | 0 | −2 | −3 | 1 | 3 | −3 | −2 | 0 |
| 21 | S | 0 | −2 | −1 | −2 | 4 | −2 | −2 | 1 | −3 | −1 | 1 | −2 | 2 | −2 | 0 | 2 | −1 | −3 | −3 | −1 |
| 22 | A | 3 | −2 | −2 | −2 | −1 | −2 | −2 | −1 | −2 | −1 | 0 | −2 | −1 | 1 | 2 | 2 | −1 | −3 | −2 | 0 |
| 23 | A | 2 | −2 | −3 | −2 | 5 | 0 | 0 | −2 | −3 | −1 | 0 | −2 | −1 | −3 | 2 | −1 | −1 | −3 | −2 | 1 |
| 24 | I | 1 | 1 | −2 | −2 | −2 | −1 | −2 | −2 | −2 | 2 | 0 | −1 | −1 | −1 | −2 | 2 | −1 | −3 | 1 | 1 |
| 25 | S | −1 | −1 | 2 | −1 | −2 | 1 | −1 | −2 | 4 | 2 | −1 | −1 | −1 | −2 | −2 | 3 | −1 | −4 | −1 | −1 |
| 26 | H | 0 | −2 | 3 | −1 | −3 | −1 | −1 | −2 | 5 | −2 | −2 | −1 | −2 | 0 | 3 | 0 | −1 | −3 | −1 | 0 |
| 27 | E | −1 | −1 | 0 | 1 | −3 | 0 | 5 | −2 | −1 | −3 | −3 | 0 | −2 | −3 | −1 | 1 | 2 | −3 | −2 | −2 |
| 28 | D | 1 | −2 | 0 | 4 | −2 | −1 | 0 | −1 | −2 | −2 | 0 | −1 | −1 | −2 | −1 | 2 | 0 | −4 | −3 | −2 |
| 29 | Y | −2 | −1 | −2 | −2 | −3 | 2 | −1 | −3 | 0 | 0 | −1 | −1 | −1 | 0 | 3 | −1 | −2 | 0 | 5 | 0 |
| 30 | P | 0 | −1 | −1 | −1 | −2 | −1 | −1 | 0 | 5 | −3 | −3 | −1 | −2 | −3 | 4 | 0 | 0 | −3 | −1 | −2 |
| 31 | A | 3 | −2 | −2 | −2 | −1 | −1 | −2 | −1 | −3 | 0 | 2 | −1 | 0 | −2 | 0 | 0 | 0 | −3 | −2 | 0 |
| 32 | D | −2 | −1 | 0 | 5 | −3 | 0 | 2 | −2 | −1 | −2 | −3 | 2 | −2 | −3 | −1 | 0 | −1 | −4 | −3 | 0 |
| 33 | E | 0 | −1 | −2 | 0 | −2 | 0 | 3 | −3 | −1 | 0 | 0 | −1 | 0 | 3 | −2 | −1 | −1 | −2 | 0 | 0 |
| 34 | G | 1 | −2 | 0 | −1 | −2 | −1 | −2 | 4 | −2 | −2 | −2 | −2 | 0 | −2 | −2 | 1 | 1 | −2 | −3 | 0 |
| 35 | D | 0 | 2 | 0 | 2 | −2 | 0 | 0 | −2 | −1 | −2 | 0 | 0 | −1 | −3 | 2 | −1 | −1 | −3 | −2 | −2 |
| 36 | Q | 0 | 3 | 0 | −1 | −2 | 3 | 0 | −2 | −1 | −2 | −2 | 0 | −1 | −3 | −1 | 1 | 3 | −3 | −2 | −2 |
| 37 | I | 0 | −2 | −3 | −2 | −1 | −2 | −2 | −2 | −3 | 1 | 0 | −2 | 0 | −2 | 4 | −1 | −1 | −3 | −2 | 2 |
| 38 | S | −1 | −1 | 0 | 1 | −2 | 0 | 2 | −2 | −1 | 2 | −1 | 1 | −1 | −2 | −2 | 2 | −1 | −3 | −2 | 0 |
| 39 | S | 3 | −1 | −1 | −1 | −1 | −1 | −1 | −1 | −2 | −1 | 0 | −1 | −1 | −2 | 2 | 3 | 0 | −3 | −2 | −1 |
| 40 | N | 0 | −2 | 2 | −1 | −3 | −1 | 1 | 3 | −1 | 0 | 0 | −1 | −1 | −3 | 1 | −1 | −1 | −3 | −2 | 0 |
| 41 | D | 1 | −2 | −1 | 3 | −2 | 0 | 2 | −2 | −2 | −1 | 0 | −1 | −1 | 0 | −2 | 0 | −1 | −3 | −2 | −1 |
| 42 | N | −1 | −1 | 2 | 1 | −3 | 0 | 3 | 0 | −1 | −2 | 0 | 0 | −1 | −2 | −2 | 1 | −1 | −3 | −2 | −2 |
| 43 | L | 1 | −3 | −3 | −3 | 4 | −2 | −3 | −3 | −1 | 0 | 1 | −3 | 0 | 3 | −3 | −2 | −2 | 0 | 5 | 0 |
| 44 | I | 1 | −1 | −1 | 0 | −2 | 0 | 2 | −2 | −2 | 1 | −1 | 1 | −1 | −2 | −2 | 2 | −1 | −4 | −2 | 0 |
| 45 | F | −2 | −3 | −2 | 1 | −2 | −2 | −2 | −3 | −1 | 2 | 0 | −3 | 0 | 4 | −3 | −2 | −2 | −1 | 4 | 1 |
| 46 | D | 1 | −2 | −1 | 2 | −2 | −1 | 0 | 0 | −2 | −3 | −3 | −1 | −2 | −3 | 4 | 1 | −1 | −4 | −3 | −2 |
| 47 | D | 0 | −2 | 0 | 5 | −3 | −1 | 0 | 1 | −2 | −3 | −4 | −1 | −3 | −3 | 2 | 1 | −1 | −4 | −3 | −3 |
| 48 | Y | −2 | 3 | −1 | −2 | −3 | 2 | −1 | −3 | 0 | −2 | −2 | 0 | −1 | 0 | −3 | −2 | −2 | 0 | 6 | −2 |
| 49 | R | −2 | 4 | 0 | −2 | −4 | 2 | 0 | −2 | 5 | −4 | −3 | 0 | −2 | −3 | 2 | −1 | −2 | −3 | −1 | −3 |
| 50 | G | −1 | −2 | 0 | 2 | −3 | −1 | 1 | 4 | −2 | −2 | −3 | −1 | −2 | −3 | −2 | 1 | −1 | −3 | −3 | 0 |
| 51 | K | −1 | 2 | 1 | −1 | −3 | 0 | 0 | 1 | −1 | −3 | −3 | 4 | −2 | −3 | −2 | 1 | −1 | −3 | −3 | −3 |
| 52 | G | −1 | −2 | −1 | 2 | 4 | −2 | −1 | 3 | −2 | −2 | −3 | −2 | −2 | 0 | −2 | 0 | 2 | −2 | −2 | −2 |
| 53 | C | −1 | −4 | −4 | −4 | 10 | −4 | −4 | −4 | −3 | −1 | −1 | −4 | −1 | 2 | −4 | −2 | −2 | −2 | −1 | −1 |
| 54 | V | −1 | −2 | −1 | 0 | −2 | −1 | 1 | −2 | −2 | 0 | 0 | −1 | 3 | −2 | −2 | 1 | 0 | −3 | −2 | 3 |
| 55 | D | 0 | −2 | 0 | 6 | −3 | 0 | 1 | −1 | −1 | −3 | −4 | −1 | −3 | −3 | −1 | 0 | −1 | −4 | −3 | −3 |
| 56 | D | −2 | −1 | 3 | 4 | −4 | 0 | 2 | 2 | −1 | −4 | −4 | −1 | −3 | −4 | −2 | 0 | −1 | −4 | −3 | −3 |
| 57 | S | 0 | −1 | 2 | 2 | −3 | 1 | 0 | 2 | −1 | −3 | −3 | −1 | −2 | −3 | −2 | 3 | 0 | −3 | −3 | −3 |
| 58 | G | 0 | 1 | 0 | −1 | −3 | −1 | −2 | 5 | −2 | −4 | −4 | −1 | −3 | −3 | −2 | 1 | −2 | −3 | −3 | −3 |
| 59 | F | −1 | −4 | −4 | −4 | −2 | −3 | −3 | −4 | −2 | 3 | 0 | −3 | 0 | 5 | −4 | −2 | −1 | −1 | 0 | 3 |
| 60 | V | −1 | −3 | −3 | −2 | −2 | −1 | 1 | −3 | −1 | 0 | 0 | −2 | 0 | 3 | −3 | −2 | −1 | −1 | 4 | 3 |
| 61 | Y | −2 | −2 | −2 | −3 | −2 | −2 | −2 | −3 | 0 | −1 | −1 | −2 | −1 | 4 | −3 | 0 | −2 | 0 | 7 | −2 |
| 62 | K | 1 | −1 | −2 | −2 | −2 | −1 | −1 | 0 | −3 | 0 | −1 | 1 | −1 | −2 | 2 | −1 | −1 | −4 | −2 | 3 |
| 63 | L | −1 | −3 | −2 | −3 | −2 | −3 | −3 | 4 | −3 | 4 | 1 | −3 | 0 | −1 | −3 | −1 | −2 | −3 | −2 | 0 |
| 64 | G | −1 | −1 | 0 | 0 | −4 | 0 | 3 | 5 | −1 | −4 | −4 | −1 | −3 | −4 | −2 | 0 | −2 | −3 | −3 | −3 |
| 65 | E | −2 | −1 | −1 | 0 | −3 | 3 | 3 | −3 | −1 | −2 | −1 | 0 | 3 | −1 | −2 | −1 | −2 | 7 | −1 | −2 |
| 66 | R | −2 | 2 | −2 | −3 | −2 | 0 | −1 | −3 | 0 | 1 | −1 | 1 | −1 | 3 | −3 | −2 | −2 | −1 | 4 | 0 |
| 67 | F | −2 | −3 | −3 | −3 | −2 | −3 | −3 | −3 | −1 | −1 | −1 | −3 | −1 | 6 | −4 | −2 | 1 | 6 | 3 | −1 |
| 68 | F | 0 | 1 | −2 | −3 | −2 | −1 | −2 | −2 | −1 | −1 | 1 | −1 | 0 | 2 | −3 | 0 | 0 | −1 | 2 | −1 |
| 69 | P | −2 | −2 | −1 | 2 | −4 | 0 | 2 | −2 | −2 | −4 | −4 | −1 | −3 | −4 | 6 | −1 | −1 | −4 | −3 | −3 |
| 70 | G | 0 | −2 | 0 | −1 | −2 | −1 | −1 | 5 | −2 | −4 | −4 | −1 | −2 | −3 | −2 | 3 | −1 | −3 | −3 | −3 |
| 71 | H | −2 | −2 | 0 | 5 | −3 | −1 | 0 | −2 | 4 | −3 | −4 | −1 | −3 | −3 | 3 | 0 | −1 | −4 | −2 | −3 |
| 72 | S | −1 | −1 | 0 | −1 | −2 | −1 | −1 | −2 | 5 | −3 | −3 | −1 | −2 | −3 | 4 | 3 | 1 | −3 | −1 | −2 |
| 73 | N | 2 | −2 | 2 | −2 | 8 | −2 | −2 | −2 | −2 | −2 | −2 | −2 | −2 | −3 | −3 | 0 | 0 | −3 | −2 | −1 |
| 74 | C | −1 | −2 | −1 | −1 | 8 | −1 | 3 | −3 | −2 | −2 | −2 | −1 | −2 | −3 | −2 | 0 | 2 | −3 | −2 | −1 |
| 75 | P | −1 | 2 | −2 | −2 | −3 | 3 | 0 | −3 | −2 | −2 | 0 | 0 | −1 | −3 | 5 | −1 | −1 | −3 | −2 | −2 |
| 76 | C | 0 | −4 | −4 | −4 | 10 | −4 | −5 | −4 | −4 | −1 | −1 | −4 | −1 | −2 | −4 | −1 | −1 | −2 | −2 | −1 |
| 77 | V | −1 | −2 | −2 | −1 | −2 | −1 | 2 | −3 | −2 | 0 | 2 | −1 | 0 | −1 | −2 | −1 | 0 | −3 | −2 | 3 |
| 78 | C | 0 | −4 | −4 | −4 | 10 | −4 | −5 | −4 | −4 | −1 | −1 | −4 | −1 | −2 | −4 | −1 | −1 | −2 | −2 | −1 |
| 79 | A | 2 | −1 | −1 | −1 | −1 | 1 | −1 | −2 | −2 | −2 | −2 | −1 | −1 | −3 | −1 | 0 | 4 | −3 | −2 | −1 |
| 80 | L | 1 | −1 | −1 | 0 | −2 | 0 | 2 | −2 | −2 | −1 | 0 | −1 | −1 | −2 | −2 | 1 | 1 | −3 | −2 | −1 |
| 81 | D | −2 | −2 | 0 | 5 | −4 | 0 | 4 | −2 | −1 | −3 | −4 | −1 | −3 | −3 | −2 | 0 | 1 | −4 | −3 | −3 |
| 82 | G | 0 | −3 | 0 | −2 | −4 | −3 | −3 | 7 | −3 | −5 | −5 | −3 | −4 | −4 | −3 | 0 | −3 | −3 | −4 | −4 |
| 83 | P | −1 | −2 | −2 | −1 | −3 | −1 | −1 | −2 | −2 | −3 | −3 | −1 | −2 | −4 | 7 | 1 | −1 | −4 | −3 | −3 |
| 84 | V | 0 | −2 | −2 | −3 | −2 | 1 | −1 | −3 | −3 | 1 | 1 | −2 | 0 | −2 | −2 | 0 | −1 | −3 | −2 | 4 |
| 85 | C | 0 | −3 | −3 | −3 | 10 | −3 | −4 | −3 | −3 | −2 | −2 | −3 | −2 | −3 | −3 | 0 | −1 | −3 | −3 | −2 |
| 86 | D | 1 | −2 | −1 | 3 | 4 | −1 | −1 | −2 | −2 | −1 | −2 | −2 | 0 | 0 | −2 | 1 | 0 | −3 | −2 | 0 |
| 87 | Q | −1 | 3 | −1 | −2 | −3 | 4 | 0 | −3 | −1 | −2 | −2 | 2 | −1 | −3 | −2 | −1 | −1 | −3 | −2 | 0 |
| 88 | P | −1 | −2 | −1 | −2 | −2 | −2 | −2 | −3 | −3 | −2 | −2 | −2 | −2 | −4 | 6 | 0 | 4 | −4 | −3 | −1 |
| 89 | E | −2 | 2 | 0 | 2 | −4 | 0 | 5 | −2 | −1 | −4 | −3 | 1 | −2 | −4 | −2 | −1 | −2 | −4 | −3 | −3 |
| 90 | C | 0 | −4 | −4 | −4 | 10 | −4 | −5 | −4 | −4 | −2 | −2 | −4 | −2 | −3 | −4 | −2 | −2 | −3 | −3 | −2 |
| 91 | P | −1 | −2 | −2 | −2 | −3 | −2 | −2 | −3 | −3 | −1 | −2 | −2 | −2 | −3 | 6 | −1 | 2 | −4 | −3 | 0 |
| 92 | K | 0 | 3 | 0 | 0 | −3 | 0 | 3 | −2 | −1 | −3 | −3 | 3 | −2 | −3 | −2 | 0 | −1 | −3 | −2 | −2 |
| 93 | I | −1 | −3 | −3 | −4 | −1 | −2 | −3 | −4 | −3 | 3 | 3 | −2 | 0 | −1 | −3 | −2 | 1 | −3 | −2 | 1 |
| 94 | H | −1 | −1 | 0 | −1 | 4 | −1 | −1 | −2 | 7 | −3 | −3 | −1 | −2 | −2 | 2 | 1 | −1 | −3 | 0 | −3 |
| 95 | P | 0 | −2 | −2 | −1 | −3 | −1 | 1 | −2 | −2 | −3 | −3 | −1 | −2 | −4 | 7 | −1 | −1 | −4 | −3 | −2 |
| 96 | K | 1 | 3 | −1 | −2 | −2 | 0 | 0 | −2 | 0 | −2 | −2 | 2 | −1 | −1 | −2 | 1 | −1 | −2 | 2 | −2 |
| 97 | C | 0 | −3 | −3 | −3 | 10 | −3 | −4 | −3 | −3 | −2 | −2 | −3 | −2 | −3 | 2 | −1 | −1 | −3 | −3 | −2 |
| 98 | T | −1 | −3 | −2 | −3 | −1 | −2 | −3 | −4 | −3 | 4 | 0 | −3 | 1 | −1 | −3 | −1 | 3 | −3 | −2 | 2 |
| 99 | K | −2 | 1 | 0 | −1 | −4 | 0 | 2 | −2 | 7 | −3 | −3 | 2 | −2 | −2 | −2 | −1 | −2 | −3 | 0 | −3 |
| 100 | V | −1 | −3 | −3 | −3 | −1 | −3 | −3 | −4 | −3 | 3 | 0 | −3 | 0 | −1 | −3 | −2 | −1 | −3 | −1 | 5 |
| 101 | E | 1 | −1 | 0 | 3 | −2 | 0 | 2 | −1 | −1 | −3 | −3 | 1 | −2 | −3 | −1 | 2 | 0 | −4 | −3 | −2 |
| 102 | H | −2 | 2 | 1 | −1 | −3 | 0 | −1 | −2 | 7 | −3 | −2 | 0 | −2 | −1 | −2 | −1 | 1 | −2 | 3 | −2 |
| 103 | N | −1 | 0 | 3 | −1 | −2 | 0 | 0 | −1 | 4 | −2 | −2 | 1 | −2 | 0 | −2 | 1 | 0 | −1 | 4 | −2 |
| 104 | G | −1 | −1 | 0 | 2 | −3 | 4 | 2 | 2 | −1 | −4 | −3 | 0 | −2 | −3 | −2 | 0 | −1 | −3 | −2 | −3 |
| 105 | C | 0 | −3 | −3 | −3 | 10 | −3 | −5 | −3 | −3 | −1 | −1 | −3 | −1 | −2 | −3 | −1 | −1 | −2 | −2 | −1 |
| 106 | C | 0 | −3 | −3 | −3 | 10 | −3 | −5 | −3 | −3 | −1 | −1 | −3 | −1 | −2 | −3 | −1 | −1 | −2 | −2 | −1 |
| 107 | P | −1 | −3 | −3 | −2 | −3 | −2 | −2 | −3 | −3 | −1 | −2 | −2 | −2 | −4 | 7 | −2 | −1 | −4 | −3 | 0 |
| 108 | E | −1 | 2 | 0 | 2 | −3 | 4 | 2 | −2 | −1 | −2 | −1 | 0 | −1 | −3 | −2 | −1 | −1 | −3 | −2 | 0 |
| 109 | C | 0 | −3 | −3 | −3 | 10 | −3 | −5 | −3 | −3 | −1 | −1 | −3 | −1 | −2 | −3 | −1 | −1 | −2 | −2 | −1 |
| 110 | K | −1 | 0 | 0 | 0 | −3 | 0 | 3 | −2 | −1 | −1 | −2 | 3 | −1 | −3 | −2 | 1 | −1 | −3 | −2 | 1 |
| 111 | E | 2 | 2 | −1 | 0 | −3 | 0 | 4 | −2 | −1 | −3 | −2 | 2 | −2 | −3 | −2 | 0 | −1 | −3 | −2 | −2 |
| 112 | V | −1 | 1 | −1 | −1 | −3 | −1 | 1 | 1 | −2 | 1 | −1 | 1 | −1 | −2 | −2 | −1 | −1 | −3 | −2 | 2 |
| 113 | K | 0 | 0 | 0 | −1 | −3 | 0 | 0 | 4 | −2 | −4 | −3 | 4 | −2 | −3 | −2 | 1 | −1 | −3 | −3 | −3 |
| 114 | N | −2 | 0 | 6 | 0 | −3 | 0 | 0 | 0 | 0 | −3 | −3 | 2 | −2 | −3 | −2 | 0 | 0 | −4 | −2 | −3 |
| 115 | F | −1 | −3 | −3 | −3 | −2 | −2 | −2 | −3 | 0 | 0 | 0 | −2 | 0 | 4 | −3 | −2 | −1 | 0 | 6 | 1 |
| 116 | C | 0 | −3 | −3 | −3 | 10 | −3 | −5 | −3 | −3 | −1 | −1 | −3 | −1 | −2 | −3 | −1 | −1 | −2 | −2 | −1 |
| 117 | E | −1 | −1 | 0 | 3 | −3 | 0 | 4 | −2 | −1 | −2 | 0 | 0 | −2 | −3 | −2 | 0 | 1 | −3 | −3 | −2 |
| 118 | Y | −2 | −2 | −2 | −2 | −3 | −1 | 1 | −3 | 0 | −1 | −1 | −2 | −1 | 5 | −3 | −2 | −2 | 0 | 6 | −2 |
| 119 | H | −2 | 5 | 2 | −2 | −3 | 0 | −1 | −2 | 4 | −2 | −1 | 0 | 3 | −2 | −2 | −1 | −1 | −3 | −1 | −2 |
| 120 | G | −1 | −2 | 2 | −1 | −3 | −2 | −2 | 6 | −2 | −4 | −4 | −2 | −3 | −3 | −2 | 0 | −2 | −3 | −3 | −3 |
| 121 | K | −1 | 4 | −1 | −2 | −3 | 0 | 0 | −3 | −1 | −1 | −2 | 5 | −1 | −3 | −2 | −1 | −1 | −3 | −2 | 0 |
| 122 | N | −1 | −2 | 2 | −2 | −2 | −2 | −2 | −2 | −2 | 2 | 0 | −2 | 0 | −2 | −2 | 0 | 4 | −3 | −2 | 1 |
| 123 | Y | −2 | −3 | −3 | −3 | −2 | −2 | −3 | −3 | 0 | −1 | −1 | −3 | −1 | 4 | −4 | −2 | −2 | 1 | 8 | −1 |
| 124 | K | 0 | 1 | 0 | −1 | −3 | 2 | 2 | −2 | 4 | −3 | −2 | 2 | −1 | −1 | −2 | −1 | −2 | −2 | 3 | −2 |
| 125 | I | −1 | −2 | 2 | −2 | −2 | −2 | −2 | −2 | −2 | 2 | 1 | −2 | 0 | −1 | −2 | 0 | 1 | −3 | −2 | 2 |
| 126 | L | −1 | −2 | 2 | −2 | −2 | −2 | −2 | 3 | −2 | 0 | 3 | −2 | 0 | −1 | −3 | −1 | −1 | −3 | −2 | −1 |
| 127 | E | −1 | 0 | 0 | 1 | −4 | 3 | 6 | −2 | 0 | −3 | −3 | 0 | −2 | −3 | −1 | 0 | −1 | −3 | −2 | −2 |
| 128 | E | −1 | −1 | 2 | 0 | −3 | 0 | 4 | −2 | 0 | −3 | −3 | 0 | −2 | −1 | −2 | 0 | 1 | −2 | 3 | −2 |
| 129 | F | −2 | −3 | −3 | −3 | −2 | −3 | −3 | −3 | −1 | 0 | 0 | −3 | 0 | 7 | −5 | −2 | −2 | 0 | 2 | −1 |
| 130 | K | −1 | 1 | 2 | −1 | −3 | 2 | 0 | −2 | −1 | −1 | 0 | 2 | 1 | −2 | −2 | −1 | −1 | −3 | −2 | 0 |
| 131 | P | −1 | −3 | −3 | −3 | −2 | −2 | −2 | −3 | −3 | 1 | 2 | −2 | 0 | −2 | 3 | −2 | 0 | −3 | −2 | 3 |
| 132 | S | 0 | 0 | 1 | 2 | 2 | −1 | 0 | −1 | −1 | −3 | −3 | −1 | −2 | −3 | 1 | 4 | 1 | −4 | −3 | −2 |
| 133 | P | −1 | −2 | −2 | −1 | −3 | −1 | 1 | −3 | −2 | −2 | −3 | −1 | −2 | −4 | 7 | −1 | −1 | −4 | −3 | 0 |
| 134 | C | 0 | −3 | −3 | −3 | 10 | −3 | −4 | −3 | −3 | −1 | −1 | −3 | −1 | −2 | −3 | −1 | 0 | −2 | −2 | −1 |
| 135 | E | −1 | 1 | −1 | 0 | −3 | 0 | 5 | −3 | −1 | −1 | 1 | 0 | −1 | −2 | −2 | −1 | −1 | −3 | −2 | −1 |
| 136 | W | −1 | 2 | −1 | −2 | −3 | 2 | 0 | −2 | 3 | −2 | 0 | 1 | −1 | −2 | −2 | 1 | −1 | 4 | −1 | −2 |
| 137 | C | 0 | 0 | −3 | −3 | 10 | −3 | −4 | −3 | −3 | 0 | −1 | −3 | −1 | −2 | −3 | −1 | 0 | −3 | −2 | −1 |
| 138 | R | −1 | 4 | −1 | −2 | −2 | 0 | −1 | −3 | −1 | 2 | −1 | 2 | −1 | −1 | −2 | −1 | 1 | −2 | 1 | 0 |
| 139 | C | 0 | −4 | −4 | −4 | 10 | −4 | −5 | −4 | −4 | −1 | −1 | −4 | −1 | −2 | −4 | −1 | −1 | −2 | −2 | −1 |
| 140 | E | −1 | −1 | 0 | 2 | −3 | 0 | 5 | −3 | −1 | −2 | 0 | 0 | −1 | −3 | −2 | 0 | 1 | −3 | −2 | −2 |
| 141 | P | 1 | −2 | 1 | −2 | −2 | −1 | −1 | −2 | −2 | 0 | 0 | −1 | −1 | −2 | 2 | 1 | 1 | −3 | −2 | 0 |
| 142 | S | −1 | −1 | 4 | 2 | −3 | 1 | 0 | 2 | −1 | −3 | −3 | −1 | −2 | −3 | −2 | 2 | −1 | −4 | −3 | −3 |
| 143 | N | −1 | 1 | 2 | −1 | −3 | −1 | −1 | 4 | −1 | −4 | −4 | 1 | −2 | −3 | −2 | 1 | −1 | −3 | −3 | −3 |
| 144 | E | −1 | 0 | −1 | 0 | −3 | 0 | 4 | −3 | −1 | 1 | −1 | 0 | −1 | −2 | −2 | −1 | 1 | −3 | −2 | 1 |
| 145 | V | 3 | −2 | −3 | −3 | −1 | −2 | −2 | −2 | −3 | 0 | 0 | −2 | 0 | −2 | −2 | −1 | −1 | −3 | −2 | 3 |
| 146 | H | −2 | 1 | −2 | −3 | −2 | −1 | −2 | −3 | 3 | 0 | 2 | −1 | 0 | 2 | −3 | −2 | −2 | −1 | 4 | −1 |
| 147 | C | −1 | 0 | −3 | −3 | 10 | −3 | −3 | −3 | −3 | −2 | −2 | −2 | −2 | −3 | 1 | −1 | −1 | −3 | −3 | −2 |
| 148 | V | 0 | −2 | −1 | −2 | −1 | −1 | −2 | −2 | −3 | 0 | 1 | −1 | 0 | −2 | −2 | 2 | 3 | −3 | −2 | 2 |
| 149 | V | −1 | −3 | −3 | −3 | 1 | −2 | −2 | −3 | −3 | 2 | 0 | −2 | 0 | −2 | 2 | −2 | −1 | −4 | −2 | 5 |
| 150 | A | 3 | −2 | −2 | −2 | 2 | −1 | −1 | −1 | −2 | 0 | −1 | −1 | −1 | −2 | −2 | 2 | 0 | −3 | −2 | 2 |
| 151 | D | 1 | −2 | 0 | 5 | −2 | 1 | 0 | 0 | −2 | −3 | −3 | −1 | −2 | −3 | −2 | 1 | −1 | −4 | −3 | −2 |
| 152 | C | −1 | −3 | −3 | −3 | 10 | −3 | −4 | 0 | −3 | −2 | −2 | −3 | −1 | 1 | −4 | −1 | −2 | −2 | −1 | −2 |
| 153 | A | 2 | −2 | −2 | −2 | −2 | −1 | 0 | −2 | −2 | −1 | 0 | −1 | −1 | 1 | 4 | −1 | −1 | −3 | −1 | −1 |
| 154 | V | 2 | −1 | −2 | −1 | −2 | 4 | 1 | −2 | −2 | −1 | −2 | 0 | −1 | −3 | 3 | −1 | −1 | −3 | −2 | 0 |
| 155 | P | −1 | −2 | −1 | 1 | −2 | −2 | −1 | −2 | −2 | 1 | 0 | −2 | −1 | −2 | 4 | 0 | 2 | −4 | −3 | 0 |
| 156 | E | −2 | 1 | −1 | 0 | −4 | 0 | 3 | −3 | 2 | −3 | −3 | 1 | −2 | 2 | 3 | −1 | −2 | −3 | −1 | −3 |
| 157 | C | −1 | −3 | −3 | −3 | 9 | 1 | −3 | −3 | −3 | −2 | −1 | −3 | −1 | −2 | −3 | −2 | −2 | 5 | −2 | −2 |
| 158 | V | −1 | 0 | −2 | −3 | −2 | 0 | −2 | −3 | −3 | 2 | 0 | −2 | 0 | 1 | −2 | −2 | 1 | −2 | −1 | 4 |
| 159 | N | −2 | 1 | 4 | 4 | −3 | 0 | 0 | −1 | 0 | −3 | −3 | −1 | −2 | −1 | −2 | 0 | −1 | 3 | 3 | −3 |
| 160 | P | −1 | −2 | −2 | −1 | −3 | −1 | −1 | −2 | −2 | −1 | −3 | 1 | −2 | −4 | 7 | 0 | −1 | −4 | −3 | −2 |
| 161 | V | −1 | −3 | −3 | −3 | −2 | 0 | 0 | −4 | −3 | 4 | 0 | −2 | 0 | 2 | −3 | −2 | −1 | −2 | 0 | 3 |
| 162 | Y | −2 | 1 | −1 | −3 | −3 | −1 | −1 | −3 | 4 | −2 | 0 | −1 | −1 | 0 | −3 | 0 | −2 | 0 | 6 | −2 |
| 163 | E | −1 | −1 | −1 | 0 | −3 | 3 | 4 | −3 | −1 | −2 | −1 | 0 | −1 | −3 | 3 | 0 | 0 | −3 | −2 | −2 |
| 164 | P | −1 | −1 | −2 | −1 | −3 | 0 | 1 | −3 | −1 | −2 | −1 | 0 | −2 | −2 | 5 | −1 | −1 | −2 | 2 | −2 |
| 165 | E | −1 | −2 | 1 | 2 | −3 | −1 | 1 | 3 | −1 | −3 | −3 | −1 | −3 | −1 | −2 | 0 | −1 | −2 | 2 | −3 |
| 166 | Q | −2 | 0 | −1 | −1 | −3 | 4 | 2 | −3 | 3 | −3 | 0 | 2 | −1 | −2 | −2 | −1 | −2 | 5 | 0 | −2 |
| 167 | C | 0 | −3 | −3 | −4 | 9 | −3 | −4 | −4 | −3 | 0 | 0 | −3 | −1 | −2 | −3 | −2 | −1 | −2 | −2 | 0 |
| 168 | C | 0 | −3 | −3 | −3 | 9 | −3 | −3 | −3 | −3 | 0 | −1 | −3 | −1 | −2 | −3 | 0 | −1 | −3 | −2 | 1 |
| 169 | P | 0 | −2 | −3 | −2 | −3 | −1 | −2 | −2 | −3 | −2 | 0 | −1 | −1 | −3 | 7 | −1 | −1 | −4 | −3 | −1 |
| 170 | V | −1 | −2 | −2 | −2 | −2 | −1 | 1 | −3 | −2 | 3 | 0 | 1 | 0 | −2 | −2 | −2 | −1 | −3 | −2 | 4 |
| 171 | C | −1 | −2 | −3 | −3 | 9 | −2 | −3 | −3 | −3 | −1 | 0 | 1 | −1 | −2 | −3 | −1 | −1 | −3 | −2 | −1 |
| 172 | K | −1 | 1 | 0 | −1 | −3 | 0 | 0 | −2 | −1 | −3 | −2 | 6 | −1 | −3 | −1 | 0 | −1 | −3 | −2 | −2 |
| 173 | N | 2 | −1 | 3 | 2 | −2 | −1 | 0 | 2 | −1 | −3 | −3 | −1 | −2 | −3 | −2 | 1 | −1 | −4 | −3 | −2 |
| 174 | G | 0 | −1 | 0 | −1 | −3 | −2 | −2 | 5 | −2 | −4 | −3 | 0 | −3 | −3 | −2 | 0 | −2 | −2 | −3 | −3 |
| 175 | P | −1 | −2 | 1 | −1 | −3 | −1 | 0 | −2 | −2 | −3 | −3 | −1 | −2 | −4 | 6 | −1 | −1 | −4 | −3 | −2 |
| 176 | N | −1 | −1 | 5 | 0 | −3 | −1 | −1 | −1 | 0 | −3 | −3 | −1 | −2 | −2 | −2 | 1 | 0 | 6 | −1 | −3 |
| 177 | C | −1 | −2 | −2 | −2 | 8 | −2 | −2 | −3 | 3 | −2 | −2 | −2 | −2 | −2 | 1 | −1 | 0 | −3 | −1 | −2 |
| 178 | F | −2 | −2 | −1 | −2 | −3 | −2 | −2 | 1 | 4 | −2 | −2 | 0 | −1 | 4 | 0 | −1 | −2 | 0 | 3 | −2 |
| 179 | A | 2 | −2 | −2 | −3 | −1 | −2 | −2 | 2 | −3 | 0 | 1 | −2 | 0 | 0 | −2 | −1 | −1 | −3 | −1 | 1 |
| 180 | G | 0 | −1 | 0 | 0 | −2 | −1 | 1 | 3 | −2 | −2 | 0 | −1 | −1 | −2 | 1 | 1 | 1 | −3 | −2 | 1 |
| 181 | T | 0 | −2 | −1 | −1 | −1 | −1 | −1 | −2 | −2 | 1 | −1 | −1 | −1 | −2 | 1 | 0 | 5 | −3 | −2 | 0 |
| 182 | T | 1 | −1 | 0 | −1 | −1 | 2 | 0 | −1 | −2 | −1 | 0 | −1 | 0 | −2 | −1 | 1 | 3 | −3 | −2 | 1 |
| 183 | I | 1 | −3 | −2 | −3 | −1 | −2 | −2 | −2 | −3 | 3 | 0 | −2 | 0 | −1 | −2 | 0 | 0 | −3 | −1 | 3 |
| 184 | I | −1 | −3 | −3 | −3 | −2 | −3 | −3 | 1 | −3 | 5 | 0 | −3 | 0 | 2 | −3 | −2 | −1 | −2 | 0 | 1 |
| 185 | P | −1 | −2 | −2 | −1 | −3 | 0 | 1 | −2 | −2 | −2 | 0 | −1 | −1 | −3 | 6 | −1 | −1 | −4 | −3 | −2 |
| 186 | A | 4 | 1 | −2 | −2 | 0 | −1 | −1 | 0 | −2 | −1 | −1 | 0 | −1 | −2 | −1 | 0 | 0 | −3 | −2 | 0 |
| 187 | G | 0 | −2 | 0 | −1 | −3 | −2 | −2 | 6 | −2 | −2 | −3 | −2 | −2 | −3 | −2 | 0 | −2 | −2 | −3 | 0 |
| 188 | I | −1 | 2 | −1 | 0 | −3 | 0 | 3 | −3 | −1 | 2 | −1 | 0 | −1 | −2 | 1 | −1 | −1 | −3 | −2 | 0 |
| 189 | E | −1 | −1 | −1 | 0 | −3 | 0 | 4 | −2 | −1 | 0 | −2 | 0 | −1 | −3 | 3 | −1 | −1 | −3 | −2 | −1 |
| 190 | V | 0 | 0 | −1 | 1 | −2 | −1 | −1 | −2 | −2 | 0 | 0 | −1 | 0 | −2 | −2 | −1 | 2 | −3 | −2 | 3 |
| 191 | K | −1 | 0 | −1 | −1 | −3 | 0 | 1 | −2 | −1 | −2 | −2 | 4 | −1 | −2 | −2 | −1 | −1 | 7 | −1 | 0 |
| 192 | V | 0 | −1 | −1 | −2 | −2 | 1 | 0 | −3 | −2 | 0 | 0 | 1 | 0 | −2 | −2 | −1 | 2 | −3 | −1 | 3 |
| 193 | D | −2 | −2 | 0 | 6 | −3 | 0 | 1 | −1 | −1 | −3 | −4 | −1 | −3 | −3 | −1 | 0 | 1 | −4 | −3 | −3 |
| 194 | E | 1 | −1 | −1 | 1 | −2 | 0 | 4 | −2 | −1 | −1 | −2 | 0 | −1 | −2 | −1 | 0 | −1 | −3 | −2 | 1 |
| 195 | C | 0 | −3 | −3 | −3 | 10 | −3 | −4 | −3 | −3 | −1 | −1 | −3 | −1 | −2 | −3 | −1 | −1 | −2 | −2 | −1 |
| 196 | N | −1 | −1 | 3 | −1 | −2 | −1 | −1 | −1 | −1 | −1 | −1 | −1 | −1 | 2 | −2 | 0 | 4 | −2 | 0 | −1 |
| 197 | I | −1 | −1 | −2 | −3 | 5 | −2 | −2 | −3 | −3 | 4 | 0 | 1 | 0 | −1 | −3 | −1 | −1 | −3 | −1 | 1 |
| 198 | C | 0 | −3 | −2 | −3 | 9 | −3 | −3 | −3 | −3 | −1 | −1 | −3 | −1 | −2 | −3 | −1 | 1 | −2 | −2 | −1 |
| 199 | H | −2 | 2 | 0 | −2 | −3 | 0 | 0 | −2 | 7 | −3 | −2 | 0 | −2 | 0 | −2 | −1 | −2 | −1 | 3 | −3 |
| 200 | C | 0 | −3 | −3 | −3 | 10 | −3 | −4 | −3 | −3 | −1 | −1 | −3 | −1 | −2 | −3 | −1 | −1 | −2 | −2 | −1 |
| 201 | H | −1 | 0 | 0 | −1 | −2 | 0 | 0 | −2 | 7 | −2 | −2 | −1 | −2 | −1 | −2 | 0 | 3 | −2 | 0 | −2 |
| 202 | N | −2 | −1 | 4 | 3 | −3 | 0 | 0 | −1 | 0 | −2 | −2 | −1 | −2 | 0 | −2 | 0 | −1 | −1 | 4 | −2 |
| 203 | G | 0 | −1 | 0 | 0 | −3 | 0 | 3 | 5 | −1 | −4 | −4 | −1 | −3 | −3 | −2 | 0 | −2 | −2 | −3 | −3 |
| 204 | D | −1 | 0 | 0 | 4 | −3 | 3 | 3 | −2 | 0 | −3 | −3 | 0 | −2 | −3 | −1 | 0 | −1 | −3 | −2 | −2 |
| 205 | W | −1 | −2 | 0 | 3 | −3 | −1 | 0 | 3 | −2 | −3 | −3 | −2 | −2 | −1 | −2 | −1 | −2 | 8 | 0 | −3 |
| 206 | W | −2 | −3 | −2 | −3 | −2 | −2 | −3 | 3 | −2 | −3 | −3 | −3 | −1 | 0 | −3 | −2 | −2 | 11 | 0 | −3 |
| 207 | K | −1 | 3 | 0 | −2 | −3 | 0 | 0 | −2 | 0 | −2 | −2 | 3 | −1 | 0 | −2 | −1 | −1 | −1 | 4 | −2 |
| 208 | P | −1 | −1 | −1 | −1 | −2 | 3 | 0 | −3 | −1 | −1 | 1 | 0 | 0 | −2 | 4 | −1 | −1 | −3 | −2 | −1 |
| 209 | A | 5 | −1 | −2 | −2 | 0 | −1 | −1 | 0 | −2 | −1 | −1 | −1 | −1 | −2 | −1 | 0 | 0 | −3 | −2 | 0 |
| 210 | Q | −1 | 0 | −1 | −1 | −2 | 3 | 0 | −2 | −1 | −1 | 0 | 0 | 4 | −1 | −1 | 0 | 2 | −2 | −1 | 0 |
| 211 | C | 0 | −3 | −3 | −3 | 10 | −3 | −4 | −3 | −3 | −1 | −1 | −3 | −1 | −2 | −3 | −1 | −1 | −2 | −2 | −1 |
| 212 | S | 0 | −1 | 0 | 0 | −1 | 0 | 0 | −1 | −1 | −2 | −2 | 0 | −1 | −2 | −1 | 4 | 3 | −3 | −2 | −1 |
| 213 | K | −1 | 5 | 0 | −2 | −3 | 0 | 0 | −2 | 0 | −3 | −2 | 4 | −1 | −3 | −2 | −1 | −1 | −3 | −2 | −3 |
| 214 | R | −1 | 3 | 0 | −2 | −2 | 0 | −1 | −3 | 6 | −1 | 1 | 0 | 0 | −1 | −2 | −1 | −1 | −2 | 0 | −1 |
| 215 | E | −1 | 0 | 0 | 1 | −4 | 1 | 5 | −2 | 0 | −3 | −3 | 3 | −2 | −3 | −1 | 0 | −1 | −3 | −2 | −2 |
| 216 | C | 0 | −2 | 3 | −1 | 9 | −2 | −2 | −2 | −1 | −1 | −1 | −2 | −1 | −2 | −3 | 0 | −1 | −3 | −2 | −1 |
| 217 | Q | −1 | 2 | −1 | −1 | 6 | 3 | 0 | −2 | −1 | −2 | −2 | 1 | −1 | −3 | −2 | 0 | −1 | −2 | −2 | −2 |
| 218 | G | −1 | 0 | 3 | 0 | −3 | 3 | 0 | 3 | 0 | −3 | −3 | 0 | −1 | −3 | −2 | 0 | −1 | −3 | −2 | −3 |
| 219 | K | −1 | 0 | 0 | −2 | −2 | 0 | 0 | −2 | 5 | 0 | 0 | 3 | 3 | −1 | −2 | −1 | −1 | −2 | 0 | 0 |
| 220 | Q | −1 | 0 | 0 | 0 | −3 | 4 | 4 | −2 | 0 | −3 | −2 | 0 | −1 | −3 | −1 | 0 | −1 | −2 | −1 | −2 |
| 221 | T | 0 | −1 | 0 | −1 | −2 | −1 | −1 | 4 | −2 | −2 | −2 | −1 | −2 | −2 | −1 | 0 | 4 | −2 | −2 | −1 |
| 222 | V | 0 | −2 | −2 | −3 | −1 | −1 | −2 | −3 | −2 | 2 | 1 | −1 | 4 | 0 | −2 | −1 | 0 | −2 | −1 | 3; or |

b) comprises a fragment of the amino acid sequence of a), wherein said fragment is a member of the vWFC domain containing protein family, or has an antigenic determinant in common with the amino acid sequence of a); or
c) comprises a functional equivalent of the amino acid sequence of a) or b); or
d) consists of the amino acid sequence of a); or
e) is any of a)-d), wherein the polypeptide has a maximum threshold E value of 10⁻²; or
f) comprises an amino acid sequence satisfying the consensus amino acid sequence [GTDFC](0,1)-[CF](0,1)-[VMSED](0,1)-[DEA](0,1)-[DENG](0,1)-[SQNDG](0,1)-[SGR](0,1)-[FIV](0,1)-[VYFE](0,1)-[YFS](0,1)-[KVAGP](0,1)-[LIG](0,1)-[GE](0,1)-[EWQM](0,1)-[RKYFQVI](0,1)-[FYWT](0,1)-[FALYRTS](0,1)-[PED](0,1)-[GS](0,1)-[HPDS](0,1)-[STHP](0,1)-[CNAT](0,1)-[CTE](0,1)-[PQRL](0,1)-C(0,1)-[VELT](0,1)-C(0,1)-[TAQ](0,1)-[ELATSD](0,1)-[EDT]-G-[PS]-[VLAQS]-[CS]-[DAMSTFCV]-[QRKV]-R(0,1)-[PT]-[ERDK]-C-[PTV]-[KERSA]-[LIVT]-[HPSC]-[PAE]-[KRASY]-[CP]-[TIVM]-[HKRE]-[VI]-[DESAKP]-[HTNRYG]-[NSTYHK](0,1)-[PA](0,1)-[TG](0,1)-[QGDES]-C-C-[PV]-[EQRDLV]-C; or
g) comprises a fragment of the amino acid sequence of f), wherein said fragment is a member of the vWFC domain containing protein family, or has an antigenic determinant in common with the polypeptides of f); or
h) comprises a functional equivalent of f) or g); or
i) consists of an amino acid sequence satisfying the consensus amino acid sequence[GTDFC](0,1)-[CF](0,1)-[VMSED](0,1)-[DEA](0,1)-[DENG](0,1)-[SQNDG](0,1)-[SGR](0,1)-[FIV](0,1)-[VYFE](0,1)-[YFS](0,1)-[KVAGP](0,1)-[LIG](0,1)-[GE](0,1)-[EWQM](0,1)-[RKYFQVI](0,1)-[FYWT](0,1)-[FALYRTS](0,1)-[PED](0,1)-[GS](0,1)-[HPDS](0,1)-[STHP](0,1)-[CNAT](0,1)-[CTE](0,1)-[PQRL](0,1)-C(0,1)-[VELT](0,1)-C(0,1)-[TAQ](0,1)-[ELATSD](0,1)-[EDT]-G-[PS]-[VLAQS]-[CS]-[DAMSTFCV]-[QRKV]-R(0,1)-[PT]-[ERDK]-C-[PTV]-[KERSA]-[LIVT]-[HPSC]-[PAE]-[KRASY]-[CP]-[TIVM]-[HKRE]-[VI]-[DESAKP]-[HTNRYG]-[NSTYHK](0,1)-[PA](0,1)-[TG](0,1)-[QGDES]-C-C-[PV]-[EQRDLV]-C; or
j) comprises an amino acid sequence selected from the group consisting of SEQ ID NO:2, SEQ ID NO:4, SEQ ID NO:39, SEQ ID NO:41, SEQ ID NO:43, and SEQ ID NO:45; or
k) comprises a fragment of the amino acid sequence of j), wherein said fragment is a member of the vWFC domain containing protein family, or has an antigenic determinant in common with the amino acid sequence of j); or
l) comprises a functional equivalent of j) or k); or
m) consists of an amino acid sequence selected from the group consisting of SEQ ID NO:2, SEQ ID NO:4, SEQ ID NO:39, SEQ ID NO:41, SEQ ID NO:43, and SEQ ID NO:45; or
n) comprises a fragment of the amino acid sequence of m), wherein said fragment is a member of the vWFC domain containing protein family, or has an antigenic determinant in common with the amino acid sequence of m); or
o) comprising a functional equivalent of m) or n); or
p) comprises an amino acid sequence selected from the group consisting of SEQ ID NO:6, SEQ ID NO:8, SEQ ID NO:10, SEQ ID NO:12, SEQ ID NO:14, SEQ ID NO:16, SEQ ID NO:47, SEQ ID NO:49, SEQ ID NO:51, and SEQ ID NO:53; or
q) comprises a fragment of the amino acid sequence of p), wherein said fragment is a member of the vWFC domain containing protein family, or has an antigenic determinant in common with the amino acid sequence of p); or
r) comprises a functional equivalent of p) or q); or
s) consists of an amino acid sequence selected from the group consisting of SEQ ID NO:6, SEQ ID NO:8, SEQ ID NO:10, SEQ ID NO:12, SEQ ID NO:14, SEQ ID NO:16, SEQ ID NO:47, SEQ ID NO:49, SEQ ID NO:51, and SEQ ID NO:53; or
t) comprises a fragment of the amino acid sequence of s), wherein said fragment is a member of the vWFC domain containing protein family, or has an antigenic determinant in common with the amino acid sequence of s); or
u) comprises a functional equivalent of s) or t); or
v) comprises the amino acid sequence selected from the group consisting of SEQ ID NO:18, SEQ ID NO:20, SEQ ID NO:22, SEQ ID NO:24, SEQ ID NO:26, SEQ ID NO:28, SEQ ID NO:30, SEQ ID NO:55, SEQ ID NO:57, SEQ ID NO:59, and SEQ ID NO:61; or
w) comprises a fragment of the amino acid sequence of w), wherein said fragment is a member of the vWFC domain containing protein family, or has an antigenic determinant in common with the amino acid sequence of w); or
x) comprises a functional equivalent of v) or w); or
y) consists of an amino acid sequence selected from the group consisting of SEQ ID NO:18, SEQ ID NO:20, SEQ ID NO:22, SEQ ID NO:24, SEQ ID NO:26, SEQ ID NO:28, SEQ ID NO:30, SEQ ID NO:55, SEQ ID NO:57, SEQ ID NO:59, and SEQ ID NO:61; or
z) comprises a fragment of the amino acid sequence of y), wherein said fragment is a member of the vWFC domain containing protein family, or has an antigenic determinant in common with the amino acid sequence of y); or
aa) comprises a functional equivalent of y) or z); or
bb) comprises the functional equivalent of any of c), e), h), l), o), r), u), x), or aa), characterised in that it is homologous to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, and SEQ ID NO:61, and is a member of the vWFC domain containing protein family; or
cc) comprises the fragment or functional equivalent of any of b), c), d), g), h), k), l), n), o), q), r), t), u), w), x), z), aa), or bb), wherein the fragment or functional equivalent has greater than 80% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, and SEQ ID NO:49, or with an active fragment of any of the foregoing; or
dd) comprises the functional equivalent of any of c), e), h), l), o), r), u), x), aa), bb, or cc), wherein the functional equivalent exhibits significant structural homology with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, and SEQ ID NO:61; or
ee) comprises the fragment of any of b), e), g), k), n), q), t), w), z), or cc), wherein the fragment has an antigenic determinant in common with the amino acid sequence of any one of a), d), e,), f), i), j), m), p), s), v), or y), which consists of 7 or more amino acid residues from the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59 or SEQ ID NO:61.

81: An isolated polypeptide comprising an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO: 22, SEQ ID NO: 24, SEQ ID NO: 26, SEQ ID NO: 28, SEQ ID NO:30, SEQ ID NO:39, SEQ ID NO: 41, SEQ ID NO: 43, SEQ ID NO: 45, SEQ ID NO: 47, SEQ ID NO:49, SEQ ID NO:51, SEQ ID NO: 53, SEQ ID NO: 55, SEQ ID NO: 57, SEQ ID NO: 59, and SEQ ID NO:61.

82. The isolated polypeptide of claim 81, wherein said polypeptide consists of an amino acid sequence selected from the group consisting of SEQ ID NO:2, SEQ ID NO:4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO:24, SEQ ID NO:26, SEQ ID NO:28, SEQ ID NO: 30, SEQ ID NO: 39, SEQ ID NO: 41, SEQ ID NO:43, SEQ ID NO:45, SEQ ID NO:47, SEQ ID NO: 49, SEQ ID NO: 51, SEQ ID NO: 53, SEQ ID NO:55, SEQ ID NO:57, SEQ ID NO:59, and SEQ ID NO: 61.
